(19) United States
(12) Patent Application Publication
(10) Pub. No.: US 2025/0197378 A1
(43) Pub. Date: Jun. 19, 2025

(54) SMALL MOLECULE MODULATORS OF IL-17

(71) Applicant: LEO PHARMA A/S, Ballerup (DK)

(72) Inventors: Mark David ANDREWS, Ballerup (DK); Jimmi Gerner SEITZBERG, Ballerup (DK); Mogens LARSEN, Ballerup (DK); Peter ANDERSEN, Ballerup (DK); Alan Stuart JESSIMAN, Ballerup (DK); Xifu LIANG, Ballerup (DK); Kevin Neil DACK, Ballerup (DK)

(21) Appl. No.: 18/843,474

(22) PCT Filed: Mar. 3, 2023
(86) PCT No.: PCT/EP2023/055425

§ 371 Date: Sep. 3, 2024

(30) Foreign Application Priority Data
Mar. 4, 2022 (EP) 22160159.4

(51) Int. Cl. C07D 403/14; A61K 31/4155; A61K 31/4178; A61K 31/4192; A61K 31/4196; A61K 31/422; A61K 31/4245; C07D 231/40; C07D 403/06; C07D 405/14; C07D 413/14

(52) U.S. Cl. CPC C07D 403/14; A61K 31/4155; A61K 31/4178; A61K 31/4192; A61K 31/4196; A61K 31/422; A61K 31/4245; C07D 231/40; C07D 403/06; C07D 405/14; C07D 413/14

(57) ABSTRACT

The present disclosure relates to a compound according to formula (I)

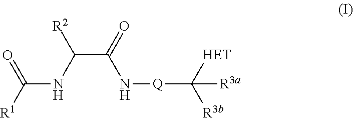

and pharmaceutically acceptable salts, hydrates, or solvates thereof. The disclosure further relates to said compounds for use in therapy, to pharmaceutical compositions comprising said compounds, to methods of treating diseases, e.g., dermal diseases, with said compounds, and to the use of said compounds in the manufacture of medicaments.

### FIELD OF THE INVENTION

This invention relates to novel amino-acid amides and derivatives thereof, to said compounds for use in therapy and to pharmaceutical compositions comprising said compounds.

### BACKGROUND OF THE INVENTION

IL-17 (also known as IL-17A or CTLA8) is a pro-inflammatory cytokine involved in anti-microbial defense at epithelial surfaces. IL-17 is comprised of two covalently joined IL-17A subunits (IL-17AA) with an approximate mass of 32 kDa, and signals through a receptor comprising IL17RA and IL17RC subunits. This receptor is predominantly expressed in epithelial and mesenchymal cells. The IL17RA/IL17RC receptor is also used by IL-17 variants IL-17AF and IL-17FF, which both are successively weaker, partial agonists on this receptor (Monin, L., Gaffen, S. L.; 2018, Cold Spring Harb. Perspect. Biol. 10. doi:10.1101/cshperspect.a028522). Crucial for signaling is the assembly of signaling complexes containing the multifunctional protein ACT1/CIKS, which in turn can recruit TRAF and other proteins.

Via these signaling complexes IL-17 induces cytokines, chemokines, antimicrobial peptides and growth factors via activation of transcription factor NFkB or via MAP kinase-dependent pathways (e.g. IL-6, IL-8, CXCL1, CXCL2, CXCL5, CCL20, G-CSF, BD4) and stabilizes the mRNAs of certain inflammatory cytokines, such as CXCL1. This leads to amplification of their effects. Further, IL-17 acts in concert with IL-1beta, IL-22 and IFNgamma (Amatya, N. et al., Trends in Immunology, 2017, 38, 310-322. doi:10.1016/j.it.2017.01.006; Onishi, R. M., Gaffen, S. L. Immunology, 2010, 129, 311-321. doi:10.1111/j.1365-2567.2009.03240.x).

IL-17 is secreted by a variety of immune cells, such as Th17 helper cells, Tc17 cytotoxic cells, ILC3 innate cells, NKT cells, TCRbeta+ natural T cells and gamma-deltaT-cells (Monin, L., Gaffen, S. L.; 2018, Cold Spring Harb. Perspect. Biol. 10. doi:10.1101/cshperspect.a028522). Increased, disease-provoking levels of IL-17 are observed in several autoimmune diseases, such as psoriasis, ankylosing spondylitis, spondyloarthritis and psoriatic arthritis. Other diseases where deregulation of IL-17 is observed are rheumatoid arthritis, systemic lupus erythematosus, asthma, inflammatory bowel disease, autoimmune uveitis, multiple sclerosis and certain cancers (Gaffen, S. L. et al., Nat Rev Immunol., 2014, 14, 585-600. doi:10.1038/nri3707; Monin, L., Gaffen, S. L.; 2018, Cold Spring Harb. Perspect. Biol. 10. doi:10.1101/cshperspect.a028522). Hence, IL-17 is a significant therapeutic target.

Therapeutic, neutralizing antibodies against IL-17A (Secukinumab, Ixekizumab) or receptor IL17RA (Brodalumab) have shown high efficacy in the treatment of psoriasis, ankylosing spondylitis and psoriatic arthritis. These antibodies have long half-lives in the body.

Although various antibodies against IL-17A or IL-17RA are approved, there are currently no approved, orally available modulators of IL-17.

The following patent applications describe small molecule modulators:
WO2013116682, WO2014066726, WO2018229079, WO2019223718, WO2019138017, WO2020011731, WO2020120140, WO2020120141, WO2020127685, WO2020146194, WO2020163554, WO2020182666, WO2020260426, WO2020260425, WO2020261141, WO2021055376, WO2021098844, WO2021204800A, WO2021204801A, WO2021170627, WO2021170631, WO2021220183, WO2021222404, WO2021239743, WO2021239745, WO2021250194, WO2021255174, WO2021255085, WO2021255086, WO2022091056, WO2022096412, WO2022128584, US20220235038, EP3943495, CN112824399A, CN112341429A, CN 112341435A, CN 112341439A, CN 112341440A, CN 112341441A, CN112341442A, CN 112341446A, CN 112341450A, CN 112341451A, CN 112341519A, CN113683598A, CN113880767, CN113880766, CN113999234, and CN113943278 all disclose Compounds for Modulating IL-17.

Scientific Reports (2016) 6, 30859 discloses Macrocyclic IL-17A Antagonists, Scientific Reports (2022) 12, 14561 discloses Identification and structure-based drug design of cell-active inhibitors of interleukin 17A at a novel C-terminal site and Leslie Dakin, 12^{th} Swiss Course on Medicinal Chemistry, Leysin, Oct. 9-14, 2016 discloses ‘Hit Identification, binding site elucidation and structure guided design of novel macrocyclic IL-17A antagonists’.

Orally available, highly efficacious small molecule IL-17 modulators which bind to IL-17 to decrease its functional ability to activate the IL-17 receptor complex may have a number of advantages compared to monoclonal antibodies. Oral administration and flexible treatment regimen may be two significant aspects in favour of patient convenience and the compounds may exhibit improved safety due to the possibility of faster withdrawal of the drug should adverse events occur.

Therefore, there is a continuous need to develop small molecule modulators of IL-17, particularly small molecules suitable for oral administration.

In addition, some patients may be treated by topical application of small molecule modulators of IL-17. This can be particularly suitable for patients with skin lesions that are readily accessible and limited in body surface area. Topical treatment may also be prescribed for certain patients who could benefit from avoiding systemic modulation of the IL-17 pathway, for example when undergoing treatment for infections or gastrointestinal problems.

### SUMMARY OF THE INVENTION

The inventors have surprisingly found that novel compounds of the present invention exhibit modulating effects on the IL-17 signalling pathway.

Compounds of the present invention may have advantageous properties such as high metabolic stability and/or membrane permeability properties that make them suitable for oral administration. Other compounds of the present invention may have advantageous properties for local topical therapy, such as high skin permeability and high metabolic instability.

Compounds of the present invention may be beneficial in preventing, treating or ameliorating a variety of diseases which involve up-regulation or de-regulation of IL-17, such as for example psoriasis, ankylosing spondylitis and psoriatic arthritis.

Accordingly, the present invention relates to a compound according to formula (I)

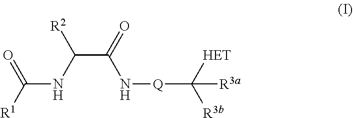

R¹ is selected from the group consisting of (C₁-C₆)alkyl, (C₃-C₇)cycloalkyl, (C₁-C₆)alkoxy, (C₃-C₇)cycloalkoxy, phenyl, phenyl-(C₁-C₄)alkyl, 5- or 6-membered heteroaryl, 9- or 10-membered bicyclic heteroaryl, 4-6-membered heterocycloalkyl and —NR^{c}R^{d}, wherein said (C₁-C₆)alkyl, (C₃-C₇)cycloalkyl, (C₁-C₆)alkoxy, (C₃-C₇)cycloalkoxy, phenyl, phenyl-(C₁-C₄)alkyl, 5- or 6-membered heteroaryl, 9- or 10-membered bicyclic heteroaryl, and 4-6-membered heterocycloalkyl is optionally substituted with one or more substituents independently selected from R^{a};
R¹ is deuterium, halogen, hydroxy, —NR^{c}R^{d}, (C₁-C₆)alkyl, (C₁-C₆)alkylcarbonyl, (C₃-C₇)cycloalkyl, (C₃-C₇)cycloalkyl-(C₁-C₆)alkyl, phenyl, 5- or 6-membered heteroaryl, 4-6-membered heterocycloalkyl, or 4-6-membered heterocycloalkyl-(C₁-C₆)alkyl, wherein said (C₁-C₆)alkyl, (C₁-C₆)alkylcarbonyl, (C₃-C₇)cycloalkyl, (C₃-C₇)cycloalkyl-(C₁-C₆)alkyl, phenyl, 5- or 6-membered heteroaryl, 4-6-membered heterocycloalkyl or 4-6-membered heterocycloalkyl-(C₁-C₆)alkyl, is optionally substituted with one or more substituents independently selected from deuterium, halogen, hydroxy, cyano, (C₁-C₄)alkyl, (C₃-C₇)cycloalkyl, (C₁-C₄)alkoxy, (C₁-C₄)alkyl-S—, (C₁-C₄)alkyl-SO—, (C₁-C₄)alkyl-SO₂— and —NR^{c}R^{d};
R^{c} and R^{d} each independently are selected from the group consisting of hydrogen and (C₁-C₆)alkyl, or R^{c} and R^{d} together form azetidinyl, pyrrolidinyl or piperidinyl, wherein said (C₁-C₆)alkyl, azetidinyl, pyrrolidinyl or piperidinyl is optionally substituted with one or more substituents independently selected from halogen, cyano and hydroxy; R² is selected from the group consisting of —CHR⁴R⁵, (C₃-C₁₀)cycloalkyl and G, wherein said (C₃-C₁₀)cycloalkyl and G are optionally substituted with one or more substituents independently selected from deuterium, halogen, cyano, (C₁-C₄)alkyl and halo(C₁-C₄)alkyl;
G is

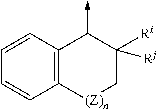

wherein Z is selected from CH, CH₂ and O; Rⁱ and R^{j} are hydrogen or Rⁱ and R^{j} together form a 3- or 4 membered carbocyclic ring; and n is 0 or 1;
R⁴ and R⁵ each independently represent hydrogen, phenyl, (C₁-C₆)alkyl, (C₃-C₇)cycloalkyl, and (C₃-C₇)cycloalkyl(C₁-C₆)alkyl wherein said phenyl, (C₁-C₆)alkyl, (C₃-C₇)cycloalkyl and (C₃-C₇)cycloalkyl(C₁-C₆)alkyl is optionally substituted with one or more substituents independently selected from halogen, cyano, and (C₁-C₄)alkyl; with the proviso that at least one of R⁴ and R⁵ is different from hydrogen; or one of R⁴ and R⁵ is (C₁-C₆)alkoxy, wherein said (C₁-C₆)alkoxy is optionally substituted with one or more fluorines;
R^{3a} is selected from hydrogen, (C₁-C₄)alkyl, (C₃-C₄)cycloalkyl and 4-6-membered heterocycloalkyl, wherein said (C₃-C₄)cycloalkyl may optionally be substituted with one or more substituents independently selected from deuterium, halogen, cyano and hydroxy, and wherein said (C₁-C₄)alkyl may optionally be substituted with one or more substituents independently selected from deuterium, halogen, cyano, hydroxy, (C₃-C₄)cycloalkyl, (C₁-C₃)alkoxy, (C₃-C₄)cycloalkoxy, fluoro(C₃-C₄)cycloalkyl, and fluoro(C₁-C₃)alkoxy and R^{3b} is hydrogen; or R^{3a} and R^{3b} together form a 3 membered carbocyclic ring;
Q is 5-membered heteroaryl, wherein said 5-membered heteroaryl is optionally substituted with one substituent independently selected from R^{e};
R^{e} is deuterium, halogen, (C₁-C₃)alkyl, wherein said (C₁-C₃)alkyl may optionally be substituted with one or more substituents independently selected from deuterium and halogen; and HET is a 5- or 6-membered heteroaryl, wherein said 5- or 6-membered heteroaryl is substituted with one or more substituents selected from (C₁-C₄)alkyl, (C₁-C₃)alkoxy, (C₃-C₄)cycloalkoxy and (C₃-C₄)cycloalkyl, wherein said (C₁-C₃)alkoxy, (C₃-C₄)cycloalkoxy and (C₃-C₄)cycloalkyl may optionally be substituted with one or more substituents independently selected from deuterium, halogen, cyano and hydroxy, and wherein said (C₁-C₄)alkyl may optionally be substituted with one or more substituents independently selected from deuterium, halogen, cyano, hydroxy, (C₃-C₄)cycloalkyl, (C₁-C₃)alkoxy, (C₃-C₄)cycloalkoxy, fluoro(C₃-C₄)cycloalkyl, and fluoro(C₁-C₃)alkoxy;
or pharmaceutically acceptable salts thereof.

In one embodiment the present invention relates to compounds of formula (II)

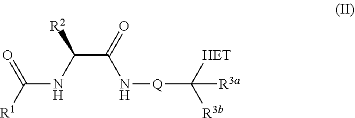

wherein R¹, R², R^{3a}, R^{3a}, Q and HET are as defined above; or pharmaceutically acceptable salts thereof.

### DETAILED DESCRIPTION OF THE INVENTION

### Definitions

The term “compound of formula (I)” means any of the compounds of formulas (Ia), (Ib), (Ic), (Id), (Ie) and (If) described herein.

The term “(Cₐ-C_{b})alkyl” is intended to indicate a hydrocarbon radical obtained when one hydrogen atom is removed from a branched or linear hydrocarbon. Said alkyl comprises (a-b) carbon atoms, such as 1-6, such as 1-4, such as 1-3, such as 2-3 or such as 1-2 carbon atoms. The term includes the subclasses normal alkyl (n-alkyl), secondary and tertiary alkyl, such as methyl, ethyl, n-propyl, isopropyl, n-butyl, isobutyl, sec.-butyl, tert.-butyl, n-pentyl, isopentyl, neopentyl, n-hexyl and isohexyl.

The term “(Cₐ-C_{b})alkoxy” is intended to indicate a radical of the formula —OR′, wherein R′ is (Cₐ-C_{b})alkyl as indicated herein, wherein the (Cₐ-C_{b})alkyl group is appended to the parent molecular moiety through an oxygen atom, e.g. methoxy (—OCH₃), ethoxy (—OCH₂CH₃), n-propoxy, isopropoxy, butoxy, tert-butoxy, and the like.

The term “cyano” is intended to indicate a —CN group attached to the parent molecular moiety through the carbon atom.

The term “(Cₐ-C_{b})cycloalkyl” is intended to indicate a saturated (Cₐ-C_{b})cycloalkane hydrocarbon radical, including polycyclic radicals such as bicyclic or tricyclic radicals, including spirocyclic radicals, comprising a-b carbon atoms, such as 3-10 carbon atoms, such as 3-8 carbon atoms, such as 3-7 carbon atoms, such as 3-6 carbon atoms, such as 3-5 carbon atoms or such as 3-4 carbon atoms, e.g. cyclopropyl, cyclobutyl, cyclopentyl, cyclohexyl, cycloheptyl, cyclooctanyl, adamantyl, spiro[2.5]octanyl, spiro[2.3]hexanyl, bicyclo[3,1,0]hexanyl, bicyclo[4,1,0]heptanyl and bicyclo[2,2,2]octanyl.

The term “(Cₐ-C_{b})cycloalkoxy” is intended to indicate a radical of the formula —OR′, wherein R′ is (Cₐ-C_{b})cycloalkyl as indicated herein, wherein the (Cₐ-C_{b})cycloalkyl group is appended to the parent molecular moiety through an oxygen atom, e.g. cyclopentyloxy or cyclobutyloxy.

The term “(Cₐ-C_{b})cycloalkyl(Cₐ-C_{b})alkyl” is intended to indicate an (Cₐ-C_{b})alkyl group as defined herein substituted with one or more (Cₐ-C_{b})cycloalkyl as defined herein, suitably the (Cₐ-C_{b})alkyl group is substituted with one (Cₐ-C_{b})cycloalkyl group.

The term “halo(Cₐ-C_{b})alkyl” is intended to indicate an (Cₐ-C_{b})alkyl group as defined herein substituted with one or more halogen atoms as defined herein, e.g. fluoro or chloro, such as difluoromethyl or trifluoromethyl.

The term “fluoro(C₁-C₄)alkyl” is for example intended to indicate a (C₁-C₄)alkyl group substituted with one or more fluoro atoms, e.g. trifluoromethyl, trifluoroethyl, difluoromethyl or difluoroethyl.

The term “halo(Cₐ-C_{b})alkoxy” is intended to indicate a (Cₐ-C_{b})alkoxy group as defined herein substituted with one or more halogen atoms as defined herein, e.g. fluoro or chloro, such as difluoromethoxy or trifluoromethyoxy.

The term “fluoro(C₁-C₃)alkoxy” is intended to indicate a (C₁-C₃)alkylalkoxy group substituted with one or more fluoro atoms, e.g. trifluoromethoxy.

The term “halogen” is intended to indicate a substituent from the 7^{th} main group of the periodic table, such as fluoro, chloro and bromo.

The term “5- or 6-membered heteroaryl” is intended to indicate radicals of monocyclic heteroaromatic rings comprising 5- or 6-membered ring which contains from 1-5 carbon atoms and from 1-4 heteroatoms selected from oxygen, sulphur and nitrogen; such as 2-5 carbon atoms and 1-3 heteroatoms, such as 3-5 carbon atoms and 1-2 heteroatoms, such as 4-5 carbon atoms and 1-2 heteroatoms selected from oxygen, sulphur and nitrogen, such as furanyl, imidazolyl, isothiazolyl, isoxazolyl, oxadiazolyl, oxazolyl, pyrazinyl, pyrazolyl, pyridazinyl, pyridyl, pyrimidinyl, pyrrolyl, tetrazolyl, thiadiazolyl, thiazolyl and triazolyl.

The term “5-membered heteroaryl” is intended to indicate radicals of 5-membered monocyclic heteroaromatic ring which contains from 1-4 carbon atoms and from 1-4 heteroatoms selected from oxygen, sulphur and nitrogen; such as 2-4 carbon atoms and 1-3 heteroatoms, such as 3-4 carbon atoms and 1-2 heteroatoms, such as 4 carbon atoms and 1 heteroatom selected from oxygen, sulphur and nitrogen; such as furanyl, imidazolyl, isothiazolyl, isoxazolyl, oxadiazolyl, oxazolyl, pyrazolyl, pyrrolyl, tetrazolyl, thiadiazolyl, thiazolyl and triazolyl. The term “5-membered heteroaryl” includes compounds wherein a ring member is a C(O) or carbonyl group.

The term “9- or 10-membered bicyclic heteroaryl” is intended to indicate fused bicyclic heteroaromatic radicals comprising 9- or 10-carbon or heteroatoms, which for example contain from 3-9 carbon atoms and 1-7 heteroatoms selected from oxygen, sulphur and nitrogen, such as 1-5 heteroatoms and 5-9 carbon atoms, such as 1-3 heteroatoms and 7-9 carbon atoms, such as 1-2 heteroatoms and 8-9 carbon atoms, such as 1 heteroatom and 8 carbon atoms, such as 1 heteroatom and 9 carbon atoms, such as 2 heteroatom and 7 carbon atoms, such as 2 heteroatom and 8 carbon atoms. Said bicyclic heteroaromatic radicals comprise a 5- or 6-membered heteroaromatic ring fused to phenyl and a 5- or 6-membered heteroaromatic ring fused to another 5- or 6-membered heteroaromatic ring, as defined herein. The heteroaryl radical may be connected to the parent molecular moiety through a carbon atom or a nitrogen atom contained anywhere within the heteroaryl group. Representative examples of 9- or 10-membered bicyclic heteroaryl include, but are not limited to azaindolyl, benzofuranyl, benzothiophenyl, benzimidazolyl, benzooxazolyl, benzothiazolyl, benzothienyl, cinnolyl, imidazopyridinyl, imidazopyrimidinyl, indazolyl, indolyl, isobenzofuranyl, isoquinolyl, quinolyl, pyrrolopyrimidinyl, thienopyridinyl, pyrrolo[2,3]pyridinyl, pyrrolo[2,3]pyridinyl, pyrazolo[1,5]pyridinyl, pyrazolo[1,5]pyridazinyl, imidazo[1,2]pyrimidinyl, pyrrolo[2,3-c]pyridinyl, pyrrolo[2,3-b]pyridinyl, pyrazolo[1,5-a]pyridinyl, pyrazolo[1,5-b]pyridazinyl, imidazo[1,2-a]pyrimidinyl, and imidazo[1,2]pyridazinyl.

The term (5- or 6-membered heteroaryl)-(Cₐ-C_{b})alkyl is intended to indicate a 5- or 6-membered heteroaryl appended to the parent molecular moiety through a (Cₐ-C_{b})alkyl group, as defined herein.

The term “(a-b) membered heterocycloalkyl” is intended to indicate a cycloalkane radical as described herein, including polycyclic radicals such as bicyclic or tricyclic radicals, including spirocyclic radicals, wherein one or more carbon atoms of said cycloalkane radical are replaced by heteroatoms, i.e. the a-b membered heterocycloalkyl comprise from a to b carbon- or hetero-atoms. Such a-b membered heterocycloalkyl could comprise for example 2-9 carbon atoms and 1-6 heteroatoms selected from O, N, or S, such as 3-8 carbon atoms and 1-4 heteroatoms, such as 3-7 carbon atoms and 1-3 heteroatoms, such as 3-6 carbon atoms and 1-2 heteroatom. The heterocycloalkyl radical may be connected to the parent molecular moiety through a carbon atom or a nitrogen atom contained anywhere within the heterocycloalkyl group. Representative examples of heterocycloalkyl groups include, but are not limited to azepanyl, azetidinyl, aziridinyl, dioxolanyl, dioxolyl, imidazolidinyl, morpholinyl, oxetanyl, piperazinyl, piperidinyl, pyrrolidinyl, tetrahydrofuranyl, tetrahydropyranyl, thietanyl, 2,6-diazaspiro[3.3]heptane, 2,6-diazaspiro[3.3]heptanyl, 2,5-diazabicyclo[2.2.1]heptanyl, 2-oxa-5-aza-[2.2.1]heptanyl, 2-oxa-8-azaspiro[3.5]nonanyl, 2-oxa-7-azaspiro[3.5]nonanyl, 2-oxa-8-azaspiro[3.5]nonanyl, 6-oxa-2-azaspiro[3.3]heptanyl, 2-oxa-7-azaspiro[3,4]octanyl, and 1, 3, 3a, 4, 6, 6a-hexahydrofuro[3,4-c]pyrrolyl. The term includes compounds wherein a ring member of said “(a-b) membered heterocycloalkyl” is a C(O) or carbonyl group and S(O) group.

The term “(a-b membered heterocycloalkyl)-(Cc-Cd)alkyl” is intended to indicate a a-b membered heterocycloalkyl radical appended to the parent molecular moiety through an (Cc-Cd)alkyl group, as defined herein.

The term “hydrocarbon radical” is intended to indicate a radical containing only hydrogen and carbon atoms, it may contain one or more double and/or triple carbon-carbon bonds, and it may comprise cyclic moieties in combination with branched or linear moieties. Said hydrocarbon comprises 1-6 carbon atoms, e.g. 1-5, e.g. 1-4, e.g. 1-3, e.g. 1-2 carbon atoms. The term includes alkyl and cycloalkyl as indicated herein.

The term “hydroxy(Cₐ-C_{b})alkyl” is intended to indicate an (Cₐ-C_{b})alkyl group as defined above substituted with one or more hydroxy, e.g. hydroxymethyl, hydroxyethyl, hydroxypropyl.

The term “oxo” is intended to indicate an oxygen atom which is connected to the parent molecular moiety via a double bond (═O).

The term “phenyl-(Cₐ-C_{b})alkyl” is intended to indicate a phenyl group appended to appended to the parent molecular moiety through an (Cₐ-C_{b})alkyl group, as defined herein.

When two or more of the above defined or similar terms are used in combination, such as cycloalkylalkyl or phenyl-(Cₐ-C_{b})alkyl and the like, it is to be understood that the first mentioned radical is a substituent on the latter mentioned radical, where the point of attachment to the parent molecular moiety is on the latter radical.

The group C(O) is intended to represent a carbonyl group (C═O).

If substituents are described as being independently selected from a group, each substituent is selected independent of the other. Each substituent may therefore be identical or different from the other substituent(s).

The term “optionally substituted” means “unsubstituted or substituted”, and therefore the general formulas described herein encompasses compounds containing the specified optional substituent(s) as well as compounds that do not contain the optional substituent(s).

As used herein whenever a molecular drawing of a substituent contains an arrow—the arrow indicates the bond attaching the substituent to the rest of the molecule.

The term “pharmaceutically acceptable salt” is intended to indicate salts prepared by reacting a compound of formula (I), which comprise a basic moiety, with a suitable inorganic or organic acid, such as hydrochloric, hydrobromic, hydroiodic, sulfuric, nitric, phosphoric, formic, acetic, 2,2-dichloroacetic, adipic, ascorbic, L-aspartic, L-glutamic, galactaric, lactic, maleic, L-malic, phthalic, citric, propionic, benzoic, glutaric, gluconic, D-glucuronic, methanesulfonic, salicylic, succinic, malonic, tartaric, benzenesulfonic, ethane-1,2-disulfonic, 2-hydroxyethanesulfonic acid, toluenesulfonic, sulfamic or fumaric acid.

Pharmaceutically acceptable salts of compounds of formula (I) comprising an acidic moiety may also be prepared by reaction with a suitable base such as sodium hydroxide, potassium hydroxide, magnesium hydroxide, calcium hydroxide, zinc hydroxide, barium hydroxide, ammonia or the like, or suitable non-toxic amines, such as lower alkylamines (such as diethylamine, tetraalkylammonium hydroxide), hydroxy-lower alkylamines (such as diethanolamine, 2-(diethylamino)-ethanol, ethanolamine, triethanolamine, tromethamine, deanol), cycloalkylamines, ethylene diamine, or benzylamines, (such as benethamine and benzathine), betaine, choline hydroxide, N-methyl-glucamine, hydrabamine, 1H-imidazole, 4-(2-hydroxyethyl)-morpholine, piperazine, 1-(2-hydroxyethyl)-pyrrolidine, L-arginine or L-lysine. Further examples of pharmaceutical acceptable salts are listed in Berge, S. M.; J. Pharm. Sci.; (1977), 66(1), 1-19, and Stahl, P. H. and in Wermuth, C. G, Handbook of Pharmaceutical Salts, Properties, Selection and Use, 2^{nd} Edition, Wiley-VCH, 2011 both of which are incorporated herein by reference.

The term “solvate” is intended to indicate a species formed by interaction between a compound, e.g. a compound of formula (I) or a pharmaceutically acceptable salt thereof, and a solvent, e.g. alcohol, glycerol or water, wherein said species are in a crystalline form. When water is the solvent, said species is referred to as a hydrate.

The term “treatment” as used herein means the management and care of a patient for the purpose of combating a disease, disorder or condition. The term is intended to include the delaying of the progression of the disease, disorder or condition, the amelioration, alleviation or relief of symptoms and complications, and/or the cure or elimination of the disease, disorder or condition. The term may also include prevention of the condition, wherein prevention is to be understood as the management and care of a patient for the purpose of combating the disease, condition or disorder and includes the administration of the active compounds to prevent the onset of the symptoms or complications. Nonetheless, prophylactic (preventive) and therapeutic (curative) treatments are two separate aspects.

In some embodiments the invention relates to a compound as above, wherein HET is selected from triazole, pyrazole, isoxazole, imidazole, oxazole, pyridyl, pyrimidinyl, pyridazinyl and pyrazinyl and wherein said triazole, pyrazole, isoxazole, imidazole, oxazole, pyridyl, pyrimidinyl, pyridazinyl and pyrazinyl is substituted with one or more substituents selected from (C₁-C₄)alkyl, halo(C₁-C₄)alkyl, (C₁-C₃)alkoxy, (C₃-C₄)cycloalkoxy and (C₃-C₄)cycloalkyl, wherein said (C₁-C₃)alkoxy, (C₃-C₄)cycloalkoxy and (C₃-C₄)cycloalkyl may optionally be substituted with one or more substituents independently selected from deuterium, halogen, cyano and hydroxy, and wherein said (C₁-C₄)alkyl may optionally be substituted with one or more substituents independently selected from deuterium, halogen, cyano, hydroxy, (C₃-C₄)cycloalkyl, (C₁-C₃)alkoxy, (C₃-C₄)cycloalkoxy, fluoro(C₃-C₄)cycloalkyl, and fluoro(C₁-C₃)alkoxy.

In another embodiment the invention relates to a compound as above, wherein R⁹ is 2,2,2-trifluoroethyl.

In another embodiment the invention relates to a compound as above, wherein HET is selected from pyridinyl, pyridazinyl and pyrimidyl wherein said pyridinyl, pyridazinyl and pyrimidyl is substituted by a substituent independently selected from hydrogen, (C₁-C₄)alkyl, (C₃-C₄)cycloalkyl, wherein said (C₃-C₄)cycloalkyl may optionally be substituted with one or more substituents independently selected from deuterium, halogen, cyano and hydroxy, and wherein said (C₁-C₄)alkyl may optionally be substituted with one or more substituents independently selected from deuterium, halogen, cyano, hydroxy, (C₃-C₄)cycloalkyl, (C₁-C₃)alkoxy, (C₃-C₄)cycloalkoxy, fluoro(C₃-C₄)cycloalkyl, and fluoro(C₁-C₃)alkoxy.

In another embodiment the invention relates to a compound as above, wherein R² is cyclohexyl wherein said cyclohexyl is optionally substituted with one or more substituents independently selected from deuterium, halogen, cyano, (C₁-C₄)alkyl and halo(C₁-C₄)alkyl.

In another embodiment the invention relates to a compound as above, wherein R² is trans 4-methylcyclohexyl.

In another embodiment the invention relates to a compound as above, wherein R² is 4,4-difluoro-cyclohexyl.

In another embodiment the invention relates to a compound as above, wherein R² is —CHR⁴R⁵, wherein R⁴ and R⁵ each independently represent hydrogen, phenyl, (C₁-C₆)alkyl, (C₃-C₇)cycloalkyl, and (C₃-C₇)cycloalkyl(C₁-C₆)alkyl wherein said phenyl, (C₁-C₆)alkyl, (C₃-C₇)cycloalkyl and (C₃-C₇)cycloalkyl(C₁-C₆)alkyl is optionally substituted with one or more substituents independently selected from halogen, cyano, and (C₁-C₄)alkyl; with the proviso that at least one of R⁴ and R⁵ is different from hydrogen.

In another embodiment the invention relates to a compound as above, wherein R² is —CHR⁴R⁵ and wherein R⁴ and R⁵ are each independently cyclopropyl or cyclobutyl.

In another embodiment the invention relates to a compound as above, wherein R² is —CHR⁴R⁵, wherein R⁴ and R⁵ are both cyclopropyl.

In another embodiment the invention relates to a compound as above, wherein R¹ is selected from pyrazolyl, isoxazolyl and oxadiazolyl, wherein said pyrazolyl, isoxazolyl and oxadiazolyl is optionally substituted with one or more substituents independently selected from deuterium, halogen, (C₁-C₄)alkyl, (C₃-C₄)cycloalkyl, wherein said (C₁-C₄)alkyl or (C₃-C₄)cycloalkyl may optionally be substituted with one or more substituents independently selected from deuterium and halogen.

In another embodiment the invention relates to a compound as above, wherein R¹ is selected from pyrazol-3-yl and 1,2,5-oxadiazol-4-yl wherein said pyrazol-3-yl and 1,2,5-oxadiazol-4-yl is optionally substituted with one or more substituents independently selected from deuterium, halogen, (C₁-C₃)alkyl, wherein said (C₁-C₃)alkyl may optionally be substituted with one or more substituents independently selected from deuterium and halogen.

In another embodiment the invention relates to a compound as above, wherein R³ is methyl, methoxymethyl or hydroxymethyl.

In another embodiment the invention relates to a compound as above, wherein R³ is methoxymethyl.

In another embodiment the invention relates to a compound as above for use in therapy.

In another embodiment the invention relates to a compound as above for use in the treatment of a disease, disorder or condition, which disease, disorder or condition is responsive of modulation of IL-17.

In another embodiment the invention relates to a compound as above for use in the treatment of autoimmune diseases.

In another embodiment the invention relates to a compound as above for use in the treatment of psoriasis, ankylosing spondylitis, spondyloarthritis or psoriatic arthritis.

In another embodiment the invention relates to a pharmaceutical composition comprising a compound as above together with a pharmaceutically acceptable vehicle or excipient or pharmaceutically acceptable carrier(s).

In another embodiment the invention relates to a pharmaceutical composition comprising a compound as above together with a pharmaceutically acceptable vehicle or excipient or pharmaceutically acceptable carrier(s) together with one or more other therapeutically active compound(s).

In one or more embodiments of the present invention, the compounds of general formula (I) have an (EC₅₀) value in a HEK BLue™ IL-17 assay of less than 1 micromolar, or of less than 100 nanomolar.

The compounds of formula (I) may be obtained in crystalline form either directly by concentration from an organic solvent or by crystallisation or recrystallisation from an organic solvent or mixture of said solvent and a co-solvent that may be organic or inorganic, such as water. The crystals may be isolated in essentially solvent-free form or as a solvate, such as a hydrate. The invention covers all crystalline forms, such as polymorphs and pseudo polymorphs, and mixtures thereof.

Compounds of formula (I) comprise asymmetrically substituted (chiral) carbon atoms which give rise to the existence of isomeric forms, e.g. enantiomers and possibly diastereomers. The present invention relates to all such isomers, either in optically pure form or as mixtures thereof (e.g. racemic mixtures or partially purified optical mixtures). Pure stereoisomeric forms of the compounds and the intermediates of this invention may be obtained by the application of procedures known in the art. The various isomeric forms may be separated by physical separation methods such as selective crystallization and chromatographic techniques, e.g. high pressure liquid chromatography using chiral stationary phases. Enantiomers may be separated from each other by selective crystallization of their diastereomeric salts which may be formed with optically active amines, or with optically active acids. Optically purified compounds may subsequently be liberated from said purified diastereomeric salts. Enantiomers may also be resolved by the formation of diastereomeric derivatives. Alternatively, enantiomers may be separated by chromatographic techniques using chiral stationary phases. Pure stereoisomeric forms may also be derived from the corresponding pure stereoisomeric forms of the appropriate starting materials, provided that the reaction occurs stereoselectively or stereospecifically. Preferably, if a specific stereoisomer is desired, said compound will be synthesized by stereoselective or stereospecific methods of preparation. These methods will advantageously employ chiral pure starting materials.

Furthermore, when a double bond or a fully or partially saturated ring system is present in the molecule geometric isomers may be formed. Any geometric isomer, as separated, pure or partially purified geometric isomers or mixtures thereof are included within the scope of the invention.

In the compounds of general formula (I), the atoms may exhibit their natural isotopic abundances, or one or more of the atoms may be artificially enriched in a particular isotope having the same atomic number, but an atomic mass or mass number different from the atomic mass or mass number found in nature. The present invention includes all suitable isotopic variations of the compounds of general formula (I). For example, different isotopic forms of hydrogen include ¹H, ²H and ³H, different isotopic forms of carbon include ¹²C, ¹³C and ¹⁴C and different isotopic forms of nitrogen include ¹⁴N and ¹⁵N. Enriching for deuterium (²H) may for example increase in-vivo half-life or reduce dosage regimens, or may provide a compound useful as a standard for characterization of biological samples. Isotopically enriched compounds within general formula (I) can be prepared by conventional techniques well known to a person skilled in the art or by processes analogous to those described in the general procedures and examples herein using appropriate isotopically enriched reagents and/or intermediates.

Solvates and hydrates form part of the invention claimed.

The compounds of the present invention may be useful for preventing, treating or ameliorating any of the following diseases: psoriasis, ankylosing spondylitis, spondyloarthritis or psoriatic arthritis, lichen planus, Sjögren's syndrome, acne, vitiligo, alopecia areata, ichthyosis, acute and chronic liver diseases, gout, osteoarthritis, systemic lupus erythematosus (SLE), lupus nephritis (LN), discoid lupus erythematosus (DLE)), multiple sclerosis, plaque psoriasis, pustular psoriasis, rheumatoid arthritis, pityriasis rubra pilaris, pyoderma gangrenosum, hidradenitis suppurativa, discoid lupus erythematosus, Papulopustolar rosacea, atopic dermatitis, Ichthyosis, bullous pemphigoid, scleroderma, tendinopathy, chronic wounds and cancer.

In an embodiment the invention relates to the use of a compound of general formula (I) as defined above, in the manufacture of a medicament for the prophylaxis, treatment or amelioration of any of the following diseases: psoriasis, ankylosing spondylitis, spondyloarthritis or psoriatic arthritis, lichen planus, lupus nephritis, Sjögren's syndrome, acne, vitiligo, alopecia areata, ichthyosis, acute and chronic liver diseases, gout, osteoarthritis, SLE, LN, DLE, multiple sclerosis, plaque psoriasis, pustular psoriasis, rheumatoid arthritis, pityriasis rubra pilaris, pyoderma gangrenosum, hidradenitis suppurativa, discoid lupus erythematosus, Papulopustolar rosacea, atopic dermatitis, Ichthyosis, bullous pemphigoid, scleroderma, tendinopathy, chronic wounds and cancer. In an embodiment the invention relates to the use of a compound of general formula (I) as defined above, in the manufacture of a medicament for the prophylaxis, treatment or amelioration of autoimmune diseases, such as psoriasis, ankylosing spondylitis, spondyloarthritis or psoriatic arthritis.

In an embodiment the invention relates to a method of preventing, treating or ameliorating autoimmune diseases, such as psoriatic arthritis, lichen planus, lupus nephritis, Sjögren's syndrome, acne, vitiligo, alopecia areata, ichthyosis, acute and chronic liver diseases, gout, osteoarthritis, SLE, LN, DLE, multiple sclerosis, plaque psoriasis, pustular psoriasis, rheumatoid arthritis, pityriasis rubra pilaris, pyoderma gangrenosum, hidradenitis suppurativa, discoid lupus erythematosus, Papulopustolar rosacea, atopic dermatitis, Ichthyosis, bullous pemphigoid, scleroderma, tendinopathy, chronic wounds and cancer, the method comprising administering to a person suffering from at least one of said diseases an effective amount of one or more compounds according to general formula (I), optionally together with a pharmaceutically acceptable carrier or one or more excipients, optionally in combination with other therapeutically active compounds.

In an embodiment the invention relates to a method of preventing, treating or ameliorating autoimmune diseases, such as psoriasis, ankylosing spondylitis, spondyloarthritis or psoriatic arthritis, the method comprising administering to a person suffering from at least one of said diseases an effective amount of one or more compounds according to general formula (I), optionally together with a pharmaceutically acceptable carrier or one or more excipients, optionally in combination with other therapeutically active compounds.

Besides being useful for human treatment, the compounds of the present invention may also be useful for veterinary treatment of animals including mammals such as horses, cattle, sheep, pigs, dogs, and cats.

### Pharmaceutical Compositions of the Invention

For use in therapy, compounds of the present invention are typically in the form of a pharmaceutical composition. The invention therefore relates to a pharmaceutical composition comprising a compound of Formula (I), optionally together with one or more other therapeutically active compound(s), together with a pharmaceutically acceptable excipient, vehicle or carrier(s). The excipient must be “acceptable” in the sense of being compatible with the other ingredients of the composition and not deleterious to the recipient thereof.

Conveniently, the active ingredient comprises from 0.0001-99.9% by weight of the formulation.

In the form of a dosage unit, the compound may be administered one or more times a day at appropriate intervals, always depending, however, on the condition of the patient, and in accordance with the prescription made by the medical practitioner. Conveniently, a dosage unit of a formulation contain between 0.001 mg and 1000 mg, preferably between 0.01 mg and 300 mg of a compound of Formula (I).

A suitable dosage of the compound of the invention will depend, inter alia, on the age and condition of the patient, the severity of the disease to be treated and other factors well known to the practising physician. The compound may be administered either orally, parenterally, topically, transdermally or intradermally and other routes according to different dosing schedules, e.g. daily, weekly or with monthly intervals. In general a single dose will be in the range from 0.001 to 400 mg/kg body weight.

If the treatment involves administration of another therapeutically active compound it is recommended to consult Goodman & Gilman's The Pharmacological Basis of Therapeutics, 9th Ed., J. G. Hardman and L. E. Limbird (Eds.), McGraw-Hill 1995, for useful dosages of said compounds.

The administration of a compound of the present invention with one or more other active compounds may be either concomitantly or sequentially.

The formulations include e.g. those in a form suitable for oral, rectal, parenteral transdermal, intradermal, ophthalmic, topical, nasal, sublingual or buccal administration.

The formulations may conveniently be presented in dosage unit form and may be prepared by but not restricted to any of the methods well known in the art of pharmacy, e.g. as disclosed in Remington, The Science and Practice of Pharmacy, 21ed ed., 2005. All methods include the step of bringing the active ingredient into association with the carrier, which constitutes one or more accessory ingredients. In general, the formulations are prepared by uniformly and intimately bringing the active ingredient into association with a liquid carrier, semisolid carrier or a finely divided solid carrier or combinations of these, and then, if necessary, shaping the product into the desired formulation.

Formulations of the present invention suitable for oral and buccal administration may be in the form of discrete units as capsules, sachets, tablets, chewing gum or lozenges, each containing a predetermined amount of the active ingredient.

A tablet may be made by compressing, moulding or freeze drying the active ingredient optionally with one or more accessory ingredients. Compressed tablets may be prepared by compressing, in a suitable machine, the active ingredient(s) in a free-flowing form; for example with a lubricant; a disintegrating agent or a dispersing agent. Moulded tablets may be made by moulding, in a suitable machine, a mixture of the powdered active ingredient and suitable carrier. Freeze dried tablets may be formed in a freeze-dryer from a solution of the drug substance.

Formulations suitable for parenteral administration conveniently comprise a sterile oily or aqueous preparation of the active ingredients, which is preferably isotonic with the blood of the recipient, e.g. isotonic saline, isotonic glucose solution or buffer solution. Liposomal formulations are also suitable for parenteral administration.

Transdermal formulations may be in the form of a plaster, patch, microneedles, liposomal or nanoparticulate delivery systems or other cutaneous formulations applied to the skin.

Formulations suitable for ophthalmic administration may be in the form of a sterile aqueous preparation of the active ingredients. Liposomal formulations or biodegradable polymer systems may also be used to present the active ingredient for ophthalmic administration.

Formulations suitable for topical, such as dermal, intradermal or ophthalmic administration include liquid or semi-solid preparations, solutions or suspensions.

Formulations suitable for nasal or buccal administration include powder, self-propelling and spray formulations, such as aerosols and atomisers.

All references, including publications, patent applications and patents, cited herein are hereby incorporated by reference in their entirety and to the same extent as if each reference were individually and specifically indicated to be incorporated by reference, regardless of any separately provided incorporation of particular documents made elsewhere herein.

### Methods of Preparation

The compounds of the present invention can be prepared in a number of ways well known to those skilled in the art of synthesis. The compounds of the invention could for example be prepared using the reactions and techniques outlined below together with methods known in the art of synthetic organic chemistry, or variations thereof as appreciated by those skilled in the art. Preferred methods include, but are not limited to, those described below. The reactions are carried out in solvents appropriate to the reagents and materials employed and suitable for the transformations being effected. Also, in the synthetic methods described below, it is to be understood that all proposed reaction conditions, including choice of solvent, reaction atmosphere, reaction temperature, duration of experiment and work-up procedures, are chosen to be conditions of standard for that reaction, which should be readily recognized by one skilled in the art. Not all compounds falling into a given class may be compatible with some of the reaction conditions required in some of the methods described. Such restrictions to the substituents which are compatible with the reaction conditions will be readily apparent to one skilled in the art and alternative methods can be used.

The compounds of the present invention or any intermediate could be purified, if required, using standard methods well known to a synthetic organist chemist, e.g. methods described in “Purification of Laboratory Chemicals”, 6^{th} ed. 2009, W. Amarego and C. Chai, Butterworth-Heinemann.

Starting materials are either known or commercially available compounds, or may be prepared by routine synthetic methods well known to a person skilled in the art.

Unless otherwise noted, reagents and solvents were used as received from commercial suppliers. The organic solvents used were usually anhydrous. The solvent ratios indicated refer to vol:vol unless otherwise noted. Thin layer chromatography was performed using Merck 6OF254 silica-gel TLC plates. Visualisation of TLC plates was performed using UV light (254 nm) or by an appropriate staining technique.

Proton nuclear magnetic resonance spectra were obtained at the stated frequencies in the solvents indicated. Tetramethylsilane was used as an internal standard for proton spectra. The value of a multiplet, either defined doublet (d), triplet (t), quartet (q) or (m) at the approximate midpoint is given unless a range is quoted. (br) indicates a broad peak, whilst (s) indicates a singlet.

Mass spectra were obtained using the following methods. LCMS Method 1 was used, unless otherwise stated.

### LCMS Method 1:

Column: Acquity UPLC HSS T3 1.8 μm; 2.1×50 mm
Flow: 0.7 mL/min
Column temp: 30° C.
Mobile phases: A: 10 mM Ammonium acetate+0.1% formic acid, B: 100% Acetonitrile+0.1% formic acid
UV: 240-400 nm
Injection volume: 1 μl

### Gradient:

| Time (min) | A % | B % |
|---|---|---|
| 0.0 | 99% | 1% |
| 0.5 | 94% | 6% |
| 1.0 | 94% | 6% |
| 2.6 | 5% | 95% |
| 3.8 | 5% | 95% |
| 3.81 | 99% | 1% |
| 4.8 | 99% | 1% |

UPLC (inlet method): XEV Metode 1 CM
MS—method: Pos_50_1000 or Neg_50_1000
Instruments: Waters Acquity UPLC, Waters XEVO G2-XS QTof, Waters PDA (Photodiode Array)

### LCMS Method 2:

Mass spectra were obtained on a Waters Quattro micro API/Waters SQD2/Waters Quattro Premier Spectrometer using electrospray ionization and atmospheric-pressure chemical ionization with the column and solvents indicated.

### LCMS Method 3:

Column: Waters Acquity UPLC HSS T3 1.8 μm, 2.1×50 mm.
Column temperature: 60° C.
UV: PDA 210-400 nm.
Injection volume: 2 μl.
Eluents: A: 10 mM Ammonium acetate with 0.1% formic acid, B: 100% Acetonitrile with 0.1% formic acid.

### Gradient:

| Time (min) | A % | B % | Flow (mL/min) |
|---|---|---|---|
| 0.0 | 95 | 5 | 1.2 |
| 0.9 | 5 | 95 | 1.2 |
| 0.91 | 5 | 95 | 1.3 |
| 1.2 | 5 | 95 | 1.3 |
| 1.21 | 5 | 95 | 1.2 |
| 1.4 | 95 | 5 | 1.2 |

MS: Electrospray switching between positive and negative ionisation.
Instruments: Waters ACQUITY, Waters SQD, Waters PDA (Photodiode array)

### LCMS Method 4:

Column: Waters ACQUITY BEH 1.7 μm, 2.1×50 mm.
Column temperature: 60° C.
UV: PDA 210-400 nm.
Injection volume: 2 μl.
Eluents: A: 10 mM Ammonium Bicarbonate, B: 100% Acetonitrile

### Gradient:

| Time (min) | % A | % B | Flow (mL/min) |
|---|---|---|---|
| 0.0 | 95 | 5 | 1.2 |
| 0.9 | 5 | 95 | 1.2 |
| 0.91 | 5 | 95 | 1.3 |
| 1.2 | 5 | 95 | 1.3 |
| 1.21 | 5 | 95 | 1.2 |
| 1.4 | 95 | 5 | 1.2 |

MS: Electrospray positive or negative ionisation.
Instruments: Waters ACQUITY, Waters QDa (MS detector), Waters PDA (Photodiode Array)

### Basic Preparative HPLC Conditions:

Column: XBridge Prep C18 5 μm OBD, 19×150 mm
Eluents: Ammonium formate (50 mM)/acetonitrile, 10-100% acetonitrile
Flow: 30 mL/min

### Acidic Preparative HPLC Conditions:

Column: XTerra® RP-18 5 μm OBD, 19×150 mm
Eluents: 0.1% formic acid in water/acetonitrile, 10-100% acetonitrile
Flow: 30 mL/min

The following abbreviations have been used throughout:
ABPR automated back pressure regulator
Boc tert-butoxycarbonyl
BOP (benzotriazol-1-yloxy)tris(dimethylamino)phosphonium hexafluorophosphate
CBz benzyloxycarbonyl
CDI carbonyldiimidazole
DAST diethylaminosulfur trifluoride
DBU 1,8-diazabicyclo[5.4.0]undec-7-ene
DCC dicyclohexylcarbodiimide
DCM dichloromethane
DEAD diethyl azodicarboxylate
DIAD diisopropyl azodicarboxylate
DIBAL diisobutylaluminium hydride
DIPEA diisopropylethylamine
DME dimethoxyethane
DMF N,N-dimethylformamide
DMAP 4-dimethylaminopyridine
DMSO dimethylsulfoxide
EDC N-(3-dimethylaminopropyl)-N′-ethylcarbodiimide
EtOAc ethyl acetate
EtOH ethanol
HATU 1-[bis(dimethylamino)methylene]-1H-1,2,3-triazolo[4,5-b]pyridinium 3-oxide hexafluorophosphate
HBTU N,N,N′,N′-tetramethyl-O-(1H-benzotriazol-1-yl)uronium hexafluorophosphate
HPLC high-performance liquid chromatography
IPA isopropanol
LCMS liquid chromatography-mass spectrometry
MeCN acetonitrile
MeOH methanol
MHz megahertz
Ms mesylate
NMR nuclear magnetic resonance
PCC pyridinium chlorochromate
ppm parts per million
PyBOP (benzotriazol-1-yloxy)tripyrrolidinophosphonium hexafluorophosphate
SFC supercritical fluid chromatography
TBAF tetra-n-butylammonium fluoride
TBME tert-butyl methyl ether
TEA triethylamine
TFA trifluoroacetic acid
THF tetrahydrofuran
TMS trimethylsilyl
TLC thin layer chromatography
Ts tosylate
T3P propanephosphonic acid anhydride

### General Methods

Compounds of the invention may be prepared according to the following non-limiting general methods and examples:

### Scheme 1

Synthesis of a compound of general formula (I), wherein Q, HET, R¹, R², R^{3a} and R^{3b} are as

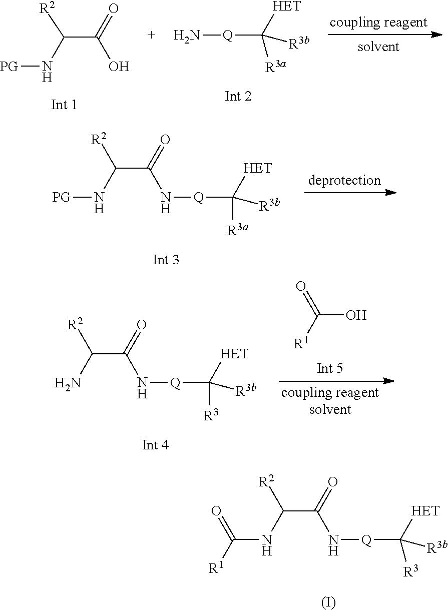

Compounds of general formula (I) can be prepared, as shown in Scheme 1. Compounds of general formula (Int 1), which are either commercially available or are synthesised in a racemic form or an enantiomerically pure form, are coupled with amines of general formula (Int 2), which are either commercially available or synthesised, in the presence of a coupling reagent such as T3P, CDI, DCC, HATU, HBTU and EDC and, in the majority of cases, in the presence of a base, such as DIPEA or TEA, in a suitable solvent, such as DMF or acetonitrile, to form compounds of formula (Int 3). Protecting groups (PG), such as Boc or Cbz, on compounds of general formula (Int 3) can be removed or selectively removed by methods known to those skilled in the art. Compounds of general formula (Int 4) are coupled with compounds of general formula (Int 5), which are either commercially available or synthesised, in the presence of a coupling reagent, such as HATU, HBTU, CDI, T3P, PyBOP, BOP, DCC or EDC, and, in most of the cases, in the presence of a base, such as DIPEA or triethylamine, in a suitable solvent, such as DMF or acetonitrile, to form compounds of general formula (I). Where the compounds of general formula (I) contain protecting groups, those protecting groups can be removed by methods known to those skilled in the art. Racemic compounds of general formula (Int 3), (Int 4) or (I) can be separated by chiral SFC, to give the S-enantiomers of compounds of general formula (Int 3), (Int 4) or (I).

### Scheme 2

Alternative synthesis of a compound of general formula (I), wherein Q, HET, R¹, R², R^{3a} and R^{3b} are as previously defined, LG represents a suitable leaving group and PG¹ and PG² represent suitable orthogonal protecting groups:

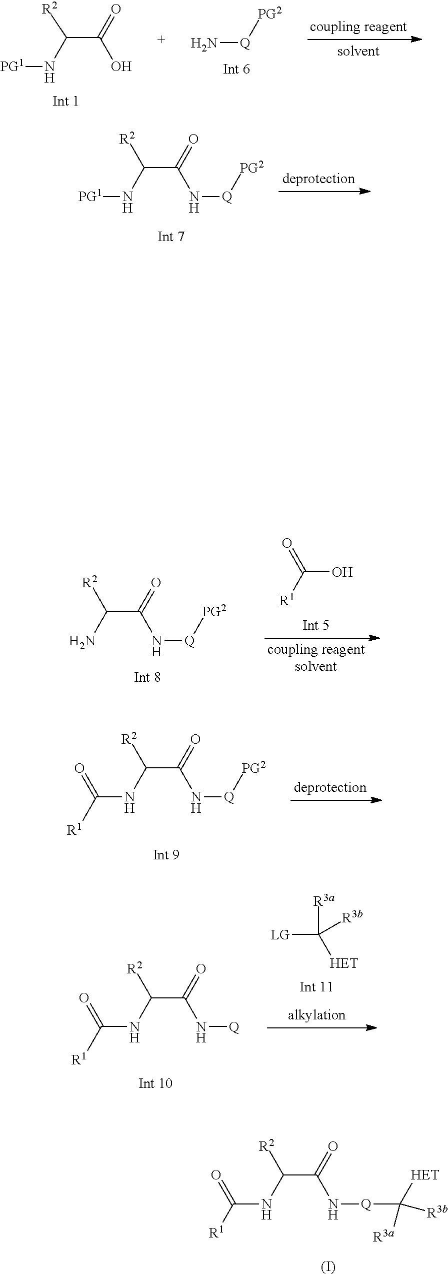

Compounds of general formula (I) can be prepared, as shown in Scheme 2. Compounds of general formula (Int 1), which are either commercially available or are synthesised in a racemic form or an enantiomerically pure form, are coupled with amines of general formula (Int 6), which are either commercially available or synthesised, in the presence of a coupling reagent, such as T3P, CDI, DCC, HATU, HBTU or EDC, and, in the majority of cases, in the presence of a base, such as DIPEA or TEA, in a suitable solvent, such as DMF or acetonitrile, to form compounds of formula (Int 7). Protecting group PG¹, such as Cbz, on compounds of general formula (Int 8) can be selectively removed by methods known to those skilled in the art. Compounds of general formula (Int 8) are coupled with compounds of general formula (Int 5), which are either commercially available or synthesised, in the presence of a coupling reagent, such as HATU, HBTU, CDI, T3P, PyBOP, BOP, DCC or EDC, and, in most of the cases, in the presence of a base, such as DIPEA or triethylamine, in a suitable solvent, such as DMF or acetonitrile, to form compounds of general formula (Int 9). Protecting group PG², such as Boc, on compounds of general formula (Int 9) can be removed by methods known to those skilled in the art. Alkylation of compounds of general formula (Int 10) with compounds of formula (Int 11), where LG represents a suitable leaving group such as Cl, Br, I, OMs or OTs, in a suitable solvent, such as DMF, DMSO or MeCN, in the presence of a suitable base, such as Cs₂CO₃, K₂CO₃ or TEA, gives compounds of formula (I). Racemic compounds of general formula (Int 7), (Int 8), (Int 9), (Int 10) or (I) can be separated by chiral SFC, to give the S-enantiomers of compounds of general formula (Int 7), (Int 8), (Int 9), (Int 10) or (I).

### Scheme 3

Preparation of a compound of formula (Int 1), wherein R² is as previously defined and PG represents a suitable protecting group:

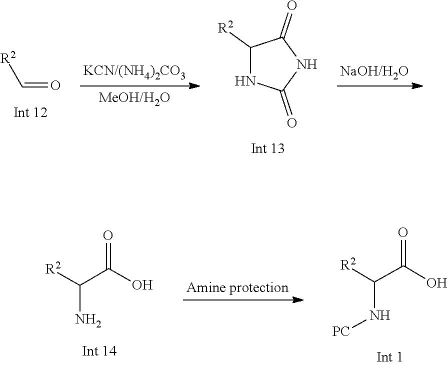

Compounds of formula (Int 1) can be prepared as shown in Scheme 3. The reaction of an aldehyde with potassium cyanide and ammonium carbonate in water and methanol forms compounds of formula (Int 13) (For Bucherer Bergs reaction, see: Chemical Reviews 2017 117 (23), 13757-13809). Compounds of formula (Int 14) can be prepared by treatment of compounds of formula (Int 13) with alkali hydroxides such as sodium hydroxide or potassium hydroxide in water. The amines of formula (Int 14) can be protected by methods known to those skilled in the art using, for example CbzCl or Boc anhydride. Alternatively compounds of the formula (Int 1) may be commercially available.

### Scheme 4

Preparation of a compound of formula (Int 1′), wherein R² is as previously defined and PG represents a suitable protecting group:

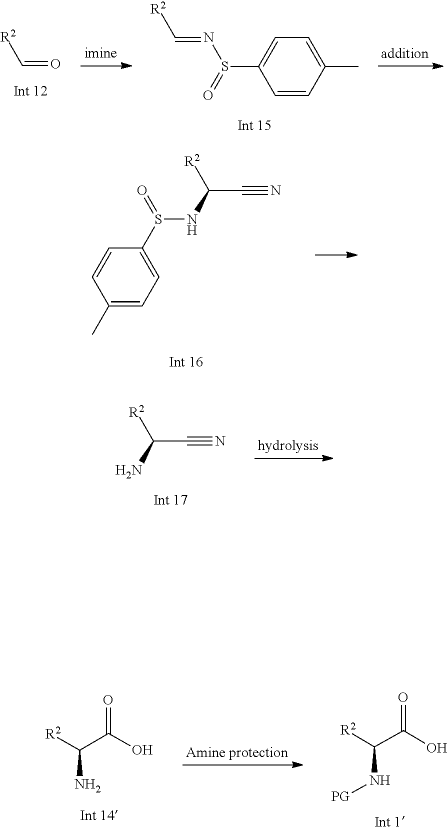

Compounds of formula (Int 1′) can be prepared as shown in Scheme 15. Compounds of formula (Int 36), that are commercially available or synthesised, can react with (S)-4-methylbenzenesulfinamide (other (S)-aromatic sulfinamides may be used) in the presence of a tetraalkoxytitanium species, such as Ti(OEt)₄, in a suitable solvent, such as DCM, to form compounds of formula (Int 15). Compounds of formula (Int 16) can be accessed from compounds of formula (Int 15) with TMSCN and CsF in a suitable solvent, such as hexane, at low temperature or preferably with Et₂AlCN in a solvent, such as THF, at low temperature. Cleavage of the sulfinamide to access compounds of formula (Int 17) can be achieved with HCl in a suitable solvent, such as THF or 1,4.dioxane, at reduced temperature. Further hydrolysis to compounds of formula (Int 14′) can be attained on treatment with concentrated acid, such as HCl in water, at elevated temperature. The amines of formula (Int 14′) can be protected by methods known to those skilled in the art, for example using CbzCl or Boc anhydride, to access compounds of formula (Int 1′).

### Scheme 5

Synthesis of a compound of formula (Int 1), wherein R² is as previously defined and PG represents a suitable protecting group:

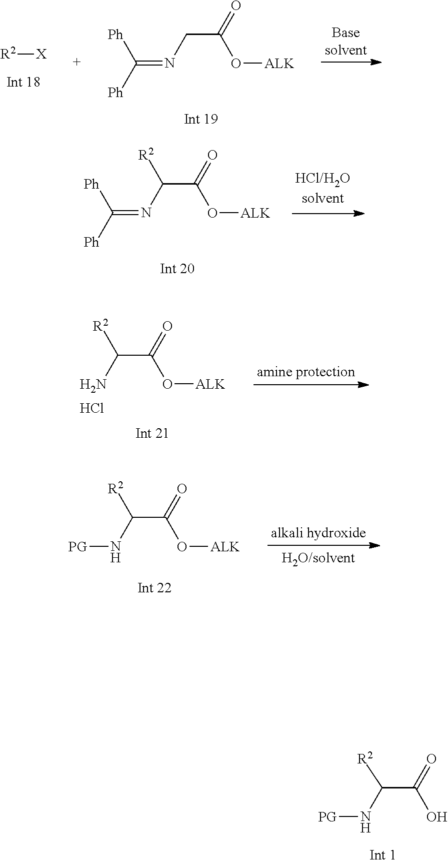

Alternatively, compounds of general formula (Int 1) can be prepared, as shown in Scheme 5. Compounds of formula (Int 18), where X is OTs, OMs, Cl, Br or I, are reacted with a commercially available compound (Int 19) in the presence of an alkali carbonate, such as sodium carbonate, potassium carbonate or caesium carbonate, in a suitable solvent, such as DMSO, DMF or acetonitrile, to form compounds of formula (Int 20). Hydrolysis of a compound of formula (Int 20) can be performed by using aqueous HCl in a suitable solvent, such as THF, to give compounds of general formula (Int 21). The amines of formula (Int 21) can be protected by methods known to those skilled in the art. The esters of formula (Int 22) are readily converted to compounds of formula (Int 1) in the presence of an alkali hydroxide such as sodium hydroxide, potassium hydroxide or lithium hydroxide. Racemic compounds of general formula (Int 22) can be separated by chiral SFC, to give the S-enantiomers of compounds of general formula (Int 22).

### Scheme 6

Synthesis of compounds of general formula (Int 28) and (Int 30), wherein R^{3a}, R^{3b}, R^{g} and Q are as previously defined and LG represents a suitable leaving group:

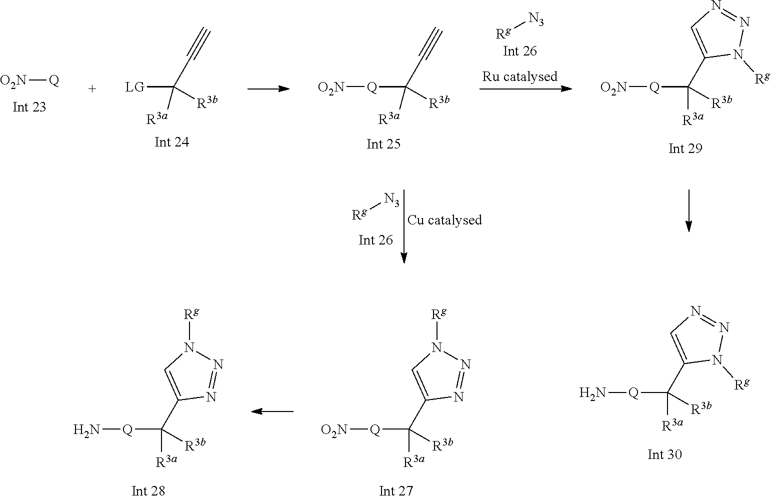

Compounds of formula (Int 28) and (Int 30) can be prepared, as shown in Scheme 6. Compounds of formula (Int 23), that are commercial or can be synthesised, are reacted with compounds of general formula (Int 24), that are commercial or synthesised, to form compounds of general formula (Int 25). For example, when LG is Cl, Br, I, OMs or OTs compounds of formula (Int 25) can be accessed in the presence of a base, such as Cs₂CO₃ or K₂CO₃, in a suitable solvent, such as DMSO, DMF or MeCN. When LG is OH, the compounds can be reacted in the presence of DEAD or DIAD and PPh₃ or P^{t}Bu₃ in a suitable solvent, such as toluene or THF, to form compounds of formula (Int 25). These compounds can be reacted with compounds of general formula (Int 26), that are commercial or can be synthesised under thermal conditions to access compounds of general formula (Int 27) and (Int 29). Alternatively, compounds of formula (Int 27) can be accessed with selective copper (I) catalysed triazole formation. Compounds of formula (Int 25) can be reacted with compounds of formula (Int 26) in the presence of copper sulphate and sodium ascorbate in a suitable aqueous solvent mix, such as H₂O/THF, to give compounds of general formula (Int 27). Alternatively compounds of formula (Int 29) can be accessed with selective ruthenium catalysed triazole formation. Compounds of formula (Int 25) can be reacted with compounds of formula (Int 26) in the presence of a ruthenium catalyst, such as chloro(1,5-cyclooctadiene)(pentamethylcyclopentadienyl)ruthenium (II), in a suitable solvent. such as TBME or THF, to give compounds of general formula (Int 29). Reduction to compounds of formula (Int 28) and (Int 30) can be achieved by the reaction of compounds of formula (Int 27) and (Int 29) under an atmosphere of hydrogen in the presence of a metal catalyst, such as Pd/C or Pt/C, in a solvent such as MeOH, EtOH or EtOAc. Alternatively, they can be reacted in the presence of triethylsilane and Pd/C with or without a base, such as TEA or DIPEA, in a suitable solvent, such as MeOH or EtOH.

### Scheme 7

Synthesis of a compound of general formula (Int 32) or (Int 33), wherein R¹, R², R^{3a}, R^{3b}, R^{g} and Q are as previously defined and LG represents a suitable leaving group:

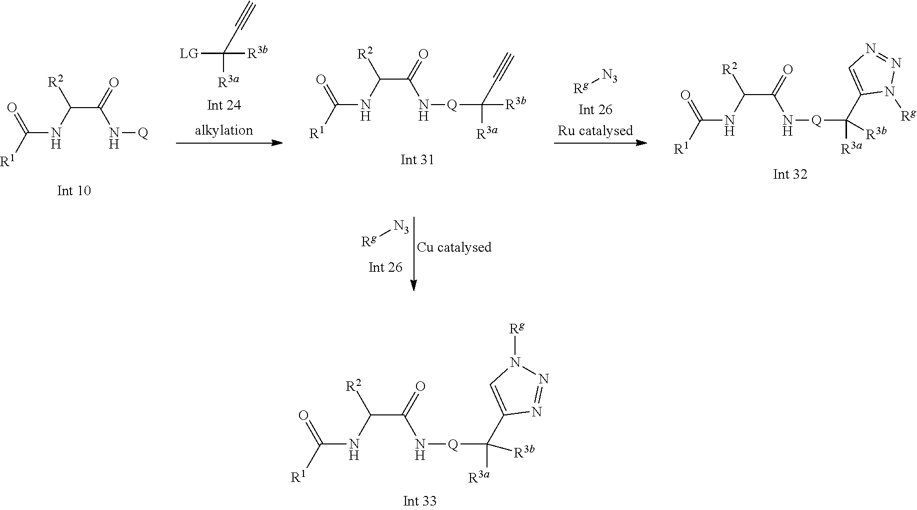

Alternatively, compounds of formula (Int 32) and (Int 33) can be prepared, as shown in Scheme 7. Compounds of formula (Int 10) that can be synthesised are reacted with compounds of general formula (Int 24) that are commercial or synthesised, to form compounds of general formula (Int 31). For example, when LG is Cl, Br, I, OMs or OTs compounds of formula (Int 31) can be accessed in the presence of a base, such as Cs₂CO₃ or K₂CO₃, in a suitable solvent, such as DMSO, DMF or MeCN. These compounds can be reacted with compounds of general formula (Int 26), that are commercial or can be synthesised, under thermal conditions to access compounds of general formula (Int 32) and (Int 33). Alternatively compounds of formula (Int 33) can be accessed with selective copper (I) catalysed triazole formation. Compounds of formula (Int 31) can be reacted with compounds of formula (Int 26) in the presence of copper sulphate and sodium ascorbate in a suitable aqueous solvent mix such as H₂O/THF to give compounds of general formula (Int 33). Alternatively compounds of formula (Int 32) can be accessed with selective ruthenium catalysed triazole formation. Compounds of formula (Int 31) can be reacted with compounds of formula (Int 26) in the presence of a ruthenium catalyst, such as chloro(1,5-cyclooctadiene)(pentamethylcyclopentadienyl)ruthenium (II), in a suitable solvent, such as TBME or THF, to give compounds of general formula (Int 32).

### Scheme 8

Preparation of compounds of formula (Int 29) wherein R⁹ is as previously defined.

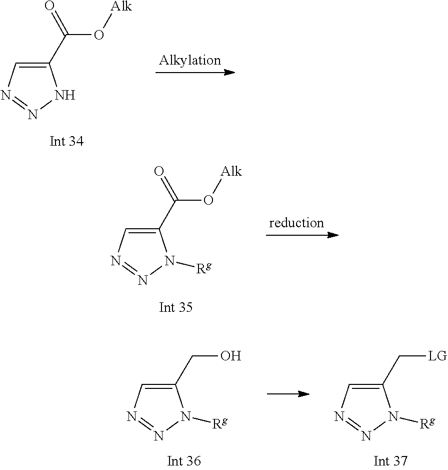

Compounds of formula (Int 37) can be prepared as shown in Scheme 8. Compounds of general formula (Int 34), which are either commercially available or can be synthesized, can be alkylated with alkyl halides, that are commercial or can be synthesized, in a suitable solvent, such as MeCN or DMF, in the presence of a suitable base, such as potassium carbonate. Alternatively, compounds of formula (Int 34) can be reacted with alcohols, that are commercial or synthesized, under Mitsunobu conditions, namely in the presence of a phosphine such as triphenylphosphine and a diazodicarboxylate such as DEAD or DIAD, in a suitable solvent, such as toluene or THF, to give compounds of formula (Int 35). Those skilled in the art will appreciate that some of the embodiments of R⁹ will undergo literature precedented transformations or deprotection before hydrolysis with an appropriate base, such as LiOH or NaOH, in a suitable solvent, such as MeOH or THF, to give compounds of general formula (Int 36). Compounds of general formula (Int 36) can be treated under methods known to those skilled in the art, to give compounds of general formula (Int 37).

### Scheme 9

Synthesis of a compound of general formula (Int 41), wherein R¹, R², R^{3a}, R^{3b}, Q and HET are as previously defined:

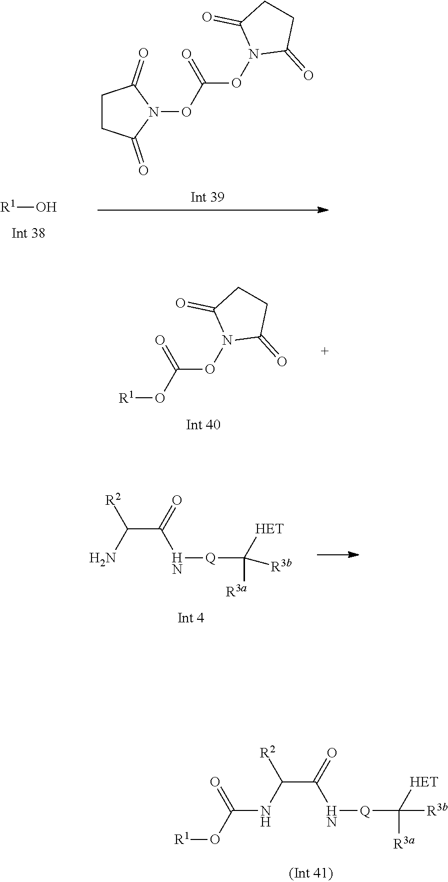

Compounds of formula (Int 41) can be prepared, as shown in Scheme 9. Compounds of general formula (Int 38), which are either commercially available or can be synthesized, can be reacted with a carbonate such as bis(2,5-dioxopyrrolidin-1-yl) carbonate (Int 39) in the presence of a base such as pyridine in a suitable solvent such as MeCN to give compounds of formula (Int 40). Compounds of general formula (Int 40) can be reacted with compounds of formula (Int 4) in the presence of a suitable base such as TEA in a suitable solvent such as MeCN to give compounds of formula (Int 41).

### Scheme 10

Synthesis of a compound of general formula (Int 49), wherein R^{3a}, R^{e} and R^{g} are as previously defined, X is a suitable leaving group such as a halogen, OMs or OTs and Alk is a suitable alkyl group, such as methyl or ethyl:

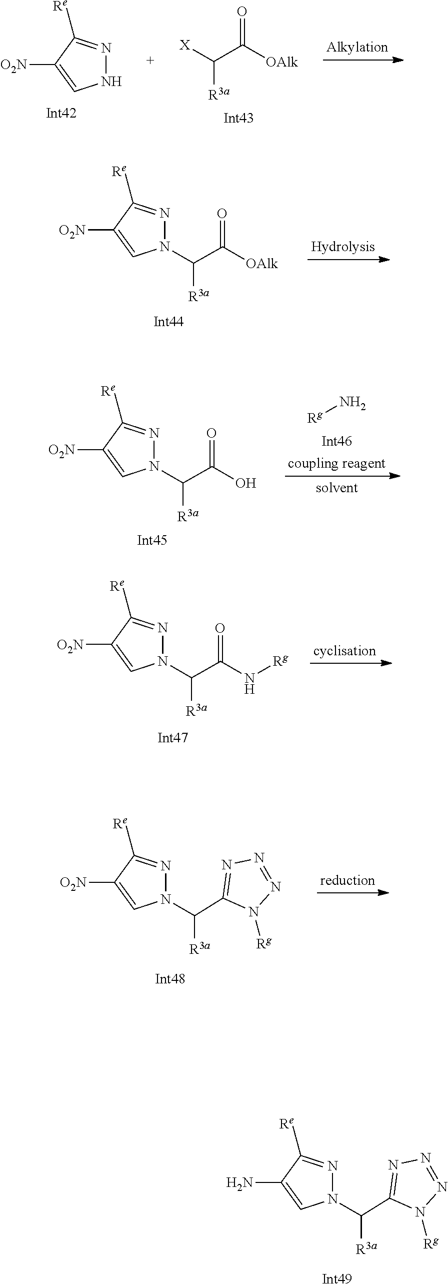

Compounds of formula (Int 48) can be prepared as shown in Scheme 10. Compounds of general formula (Int 42), which are either commercially available or can be synthesized, can be alkylated with alkylating agents of general formula (Int 43), that are commercial or can be synthesized, in a suitable solvent, such as THF, MeCN or DMF, in the presence of a suitable base, such as potassium carbonate or NaH, to give compounds of general formula (Int 44). Hydrolysis with an appropriate base, such as LiOH or NaOH, in a suitable solvent, such as MeOH or THF, gives compounds of general formula (Int 45). Compounds of general formula (Int 45) are coupled with amines of general formula (Int 46), which are either commercially available or synthesised, in the presence of a coupling reagent, such as HATU, HBTU, CDI, T3P, PyBOP, BOP, DCC or EDC, and, in most of the cases, in the presence of a base, such as DIPEA or triethylamine, in a suitable solvent, such as DMF or acetonitrile, to form compounds of general formula (Int 47). Alternatively, compounds of general formula (Int 44) can be converted directly to compounds of general formula (Int 47) by reaction with amines of general formula (Int 46) in the presence of a suitable reagent, such as tert-butylmagnesium chloride or trimethylaluminium, in a suitable solvent, such as THF or toluene.

Cyclisation of compounds of general formula (Int 47) can be accomplished by treatment with a suitable activating agent, such as CCl₄ in the presence of triphenylphosphine in a suitable solvent such as MeCN, followed by reaction with an azide source, such as trimethylsilyl azide, at an elevated temperature, to give compounds of general formula (Int 48). Reduction to compounds of formula (Int 49) can be achieved by the reaction of compounds of formula (Int 48) under an atmosphere of hydrogen in the presence of a metal catalyst, such as Pd/C or Pt/C, in a solvent such as MeOH, EtOH or EtOAc. Alternatively they can be reacted in the presence of triethylsilane and Pd/C with or without a base, such as TEA or DIPEA, in a suitable solvent, such as MeOH or EtOH.

### Scheme 11

Alternative synthesis of a compound of general formula (Int 44), wherein R^{3a} and R^{e} are as previously defined and Alk is a suitable alkyl group, such as methyl or ethyl:

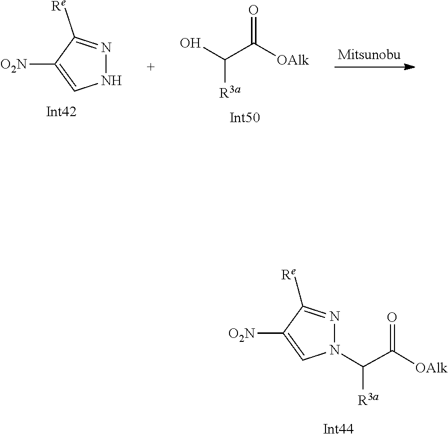

Compounds of formula (Int 44) can be prepared as shown in Scheme 11. Compounds of formula (Int 42) can be reacted with alcohols of general formula (Int 50), that are commercial or synthesized, under Mitsunobu conditions, namely in the presence of a phosphine such as triphenylphosphine and a diazodicarboxylate such as DEAD or DIAD, in a suitable solvent, such as toluene or THF, to give compounds of formula (Int 44).

### Scheme 12

Alternative synthesis of a compound of general formula (Int 45), wherein R^{3a} and R^{e} are as previously defined, X is a suitable leaving group such as a halogen, OMs or OTs and Alk is a suitable alkyl group, such as methyl or ethyl:

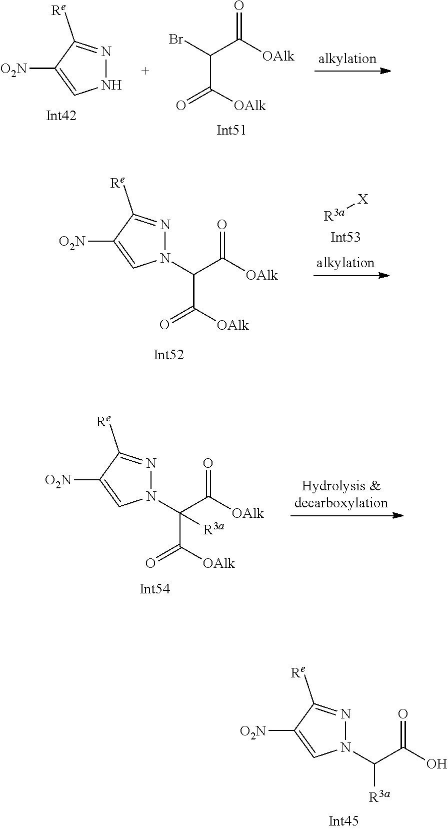

Compounds of formula (Int 45) can be prepared as shown in Scheme 12. Compounds of formula (Int 42) can be reacted with malonates of general formula (Int 51), that are commercial or synthesized, in a suitable solvent, such as THF, MeCN or DMF, in the presence of a suitable base, such as potassium carbonate or NaH, to give compounds of general formula (Int 52). Compounds of general formula (Int 52) can be alkylated with alkylating agents of general formula (Int 53), that are commercial or can be synthesized, in a suitable solvent, such as THF, MeCN or DMF, in the presence of a suitable base, such as potassium carbonate, to give compounds of general formula (Int 54). Hydrolysis with an appropriate base, such as LiOH or NaOH, in a suitable solvent, such as MeOH or THF, followed by treatment with acid, possibly at an elevated temperature, gives compounds of general formula (Int 45).

### Scheme 13

Synthesis of a compound of general formula (Int 60), wherein R^{3a}, R^{e} and R^{g} are as previously defined:

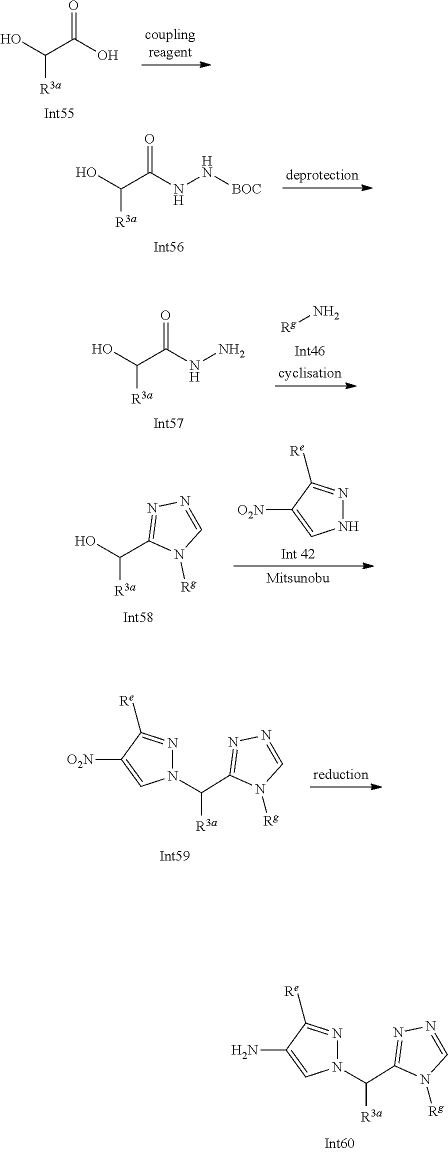

Compounds of formula (Int 60) can be prepared as shown in Scheme 13. Compounds of general formula (Int 55), which are either commercially available or can be synthesized, can be coupled with Boc hydrazine in the presence of a coupling reagent, such as HATU, HBTU, CDI, T3P, PyBOP, BOP, DCC or EDC, and, in most of the cases, in the presence of a base, such as DIPEA or triethylamine, in a suitable solvent, such as DMF or acetonitrile, to form compounds of general formula (Int 56). Treatment of compounds of general formula (Int 56) with a suitable acid, such as HCl in dioxane, in a suitable solvent, such as MeOH, gives compounds of general formula (Int 57). Cyclisation of compounds of general formula (Int 57) can be accomplished by treatment with DMF dimethyl acetal in a suitable solvent, such as MeCN, followed by reaction with an amine of general formula (Int 46) in the presence of acetic acid, at an elevated temperature, to give compounds of general formula (Int 58). Compounds of formula (Int 58) can be reacted with pyrazoles of general formula (Int 42), that are commercial or synthesized, under Mitsunobu conditions, namely in the presence of a phosphine such as triphenylphosphine and a diazodicarboxylate such as DEAD or DIAD, in a suitable solvent, such as toluene or THF, to give compounds of formula (Int 59). Reduction to compounds of formula (Int 60) can be achieved by the reaction of compounds of formula (Int 59) under an atmosphere of hydrogen in the presence of a metal catalyst, such as Pd/C or Pt/C, in a solvent such as MeOH, EtOH or EtOAc. Alternatively they can be reacted in the presence of triethylsilane and Pd/C with or without a base, such as TEA or DIPEA, in a suitable solvent, such as MeOH or EtOH.

### PREPARATIONS AND EXAMPLES

### Preparations

### Preparation 1: (1-Cyclopropyl-2-methoxy-vinyl)cyclopropane

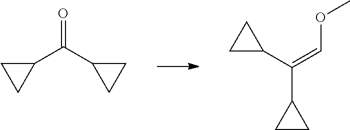

Potassium tert-butoxide (53.0 g, 472 mmol) was added slowly to a suspension of methoxymethyl-(triphenyl)phosphonium chloride (160 g, 467 mmol) in dry THF (700 mL) at 5-10° C. under argon (a weak exotherm was observed). The resulting deep red solution was stirred for 45 min at 5-10° C. Then dicyclopropylketone (39 mL, 37.6 g, 341 mmol) was added slowly (exothermic) The dark red colour quickly faded to give a yellow/orange solution. The reaction was stirred for 2 hours at room temperature under argon to give a red/orange mixture which was diluted with pentane (200 mL) and quenched with brine (50 mL) and water (100 mL) with vigorous stirring for 10 min. The organic layer was separated, dried (Na₂SO₄) and evaporated to give an orange syrup. Pentane (1000 mL) was added and stirred vigorously overnight and the mixture was filtered through a short pad of SiO₂ (height: 10 cm; diameter: 6 cm). The filtrate was concentrated in vacuo to give the title compound (44.0 g, 93% yield) as a clear liquid. ¹H NMR (300 MHz, CDCl₃) δ 5.86 (dd, J=1.6, 0.7 Hz, 1H), 3.57 (s, 3H), 1.87-1.74 (m, 1H), 0.89-0.78 (m, 1H), 0.76-0.67 (m, 2H), 0.64-0.57 (m, 2H), 0.51-0.41 (m, 2H), 0.27-0.19 (m, 2H).

### Preparation 2: 2,2-Dicyclopropylacetaldehyde

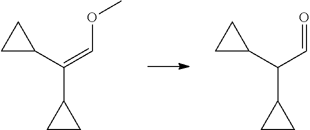

The compound of Preparation 1 (44.0 g, 318 mmol) was stirred vigorously in ether (100 mL) and 5M HCl (30 mL) overnight at room temperature (reaction monitored by GCMS). The ether layer was separated, the aqueous layer was extracted with ether (2×50 mL) and the combined ether layers were dried (Na₂SO₄) and concentrated in vacuo to give 2,2-dicyclopropylacetaldehyde (39.0 g, 98% yield) as a clear liquid which was used without any further purification. ¹H NMR (400 MHz, CDCl₃) δ 9.74 (d, J=2.9 Hz, 1H), 1.12-1.01 (m, 1H), 0.95-0.81 (m, 2H), 0.66-0.49 (m, 4H), 0.34-0.19 (m, 4H).

### Preparation 3: 2-(benzyloxycarbonylamino)-3,3-dicyclopropyl-propanoic acid

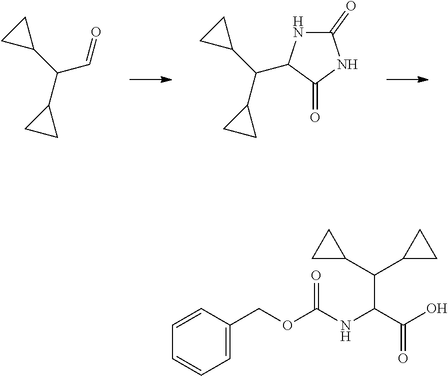 [SMILES: O=CC(C1CC1)C1CC1 ; O=C1NC(=O)C(C(C2CC2)C2CC2)N1 ; O=C(NC(C(=O)O)C(C1CC1)C1CC1)OCc1ccccc1]

Ammonium carbonate (100 g, 1.04 mol) was added to a solution of the compound of Preparation 2 (22.8 g, 184 mmol) and KCN (17.9 g, 275 mmol) in EtOH:H₂O (200 mL:200 mL) and the reaction mixture was stirred at 100 C for 6 hours. The cooled reaction mixture was concentrated in vacuo to low volume. The pH was adjusted to ˜5 with 5M HCl (aq.) and the resulting precipitate was filtered and dried to give crude hydantoin (22.16 g, 62% yield) as a colourless solid that was used without further purification.

The crude hydantoin (22.16 g, 114 mmol) was heated at reflux in 9M NaOH (200 mL) overnight, then cooled in an ice bath and 8M HCl (100 mL) was added slowly. Benzyl chloroformate (21.4 g, 126 mmol) was then added with vigorous stirring. The mixture was stirred at room temperature for 1 hour then 8M HCl was added carefully until the pH was between 3 and 4. The mixture was extracted with EtOAc (3×100 mL) and the combined organic extracts were dried (Na₂SO₄) and evaporated. Purification by column chromatography (silica gel, eluting with EtOAc:heptane) gave the title compound (21.0 g, 61% yield) as a colourless oil. ¹H NMR (400 MHz, DMSO-d6) δ 12.5 (br, s, 1H), 7.42-7.20 (m, 6H), 5.09-5.01 (q, J=12.4 Hz 2H), 4.19-4.16 (q, J=4.4 Hz 1H), 0.97-0.95 (m, 1H), 0.80-0.78 (m, 1H), 0.58-0.08 (m, 9H); LCMS (METHOD 3) (ES): m/z 304.2 [M+H]⁺, RT=0.72 min.

### Preparation 4: methyl (2S)-2-(benzyloxycarbonylamino)-3,3-dicyclopropyl-propanoate

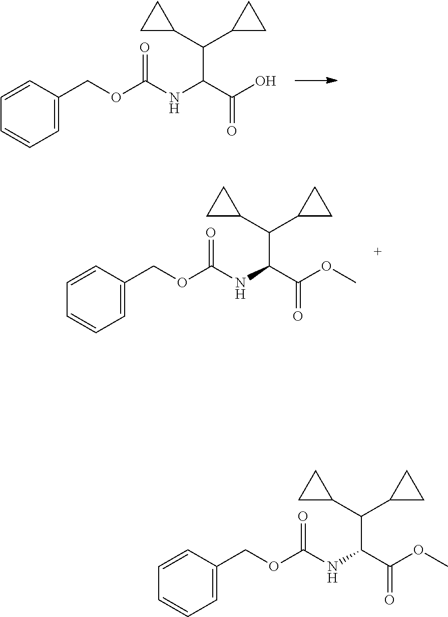 [SMILES: O=C(NC(C(=O)O)C(C1CC1)C1CC1)OCc1ccccc1 ; COC(=O)[C@@H](NC(=O)OCc1ccccc1)C(C1CC1)C1CC1 ; COC(=O)[C@H](NC(=O)OCc1ccccc1)C(C1CC1)C1CC1]

Thionyl chloride (75.9 g, 643 mmol) was added dropwise over 20 minutes to a solution of the compound of Preparation 3 (65 g, 214 mmol) in MeOH (650 mL) at 0° C. The reaction mixture was warmed to room temperature over 16 hours. The reaction mixture was concentrated under reduced pressure, diluted with saturated aq. NaHCO₃ (500 mL) and extracted with EtOAc (3×500 mL). The combined extracts were washed with H₂O (200 mL), brine solution (200 mL), dried over Na₂SO₄, filtered and concentrated in vacuo. The obtained crude compound was purified by silica gel (100-200 mesh) column chromatography (10% EtOAC/n-Hexane as eluent) to afford methyl 2-(((benzyloxy)carbonyl)amino)-3,3-dicyclopropylpropanoate as an off-white solid (50 g, 73%). The mixture of isomers were separated by SFC to afford methyl (S)-2-(((benzyloxy)carbonyl)amino)-3,3-dicyclopropylpropanoate, (24 g, 36%) and methyl (R)-2-(((benzyloxy)carbonyl)amino)-3,3-dicyclopropylpropanoate, (23 g, 33%) as colourless liquids.

Methyl (S)-2-(((benzyloxy)carbonyl)amino)-3,3-dicyclopropylpropanoate(4a): 1H NMR (400 MHz, CDCl₃) δ 7.37-7.31 (m, 5H), 5.5 (d, J=6 Hz, 1H), 5.12 (s, 2H), 4.61-4.58 (dd, J=3.2 Hz, J=6 Hz, 1H), 3.7 (s, 3H), 0.73-0.69 (m, 3H), 0.68-0.49 (m, 4H), 0.38-0.08 (m, 4H). LCMS (METHOD 2) (ESI): m/z: 318 [M+H]⁺, RT=2.22 min (ACQUITY BEH C18 (50 mm×2.1 mm) column, 0.1% Formic acid in water, 0.1% Formic acid in MeCN). Chiral purity: 99%; RT: 3.15 min, Column: CHIRALPAK IF (250×4.6 mm) 5 μm; Co-solvent: Methanol, Total flow: 3 mL/min, % of co solvent: 15%, ABPR: 100 bar, Temperature: 30° C. Methyl (R)-2-(((benzyloxy)carbonyl)amino)-3,3-dicyclopropylpropanoate(4b): 1H NMR (400 MHz, CDCl₃) δ 7.37-7.31 (m, 5H), 5.5 (d, J=6 Hz, 1H), 5.12 (s, 2H), 4.61-4.58 (dd, J=3.2 Hz, J=6 Hz, 1H), 3.7 (s, 3H), 0.73-0.70 (m, 3H), 0.68-0.49 (m, 4H), 0.38-0.17 (m, 4H). LCMS (METHOD 2) (ESI): m/z: 318 [M+H]⁺; 98%; RT=2.60 min (ACQUITY BEH C18 (50 mm×2.1 mm) column, 0.1% Formic acid in water, 0.1% Formic acid in MeCN). Chiral purity: 99%; RT: 4.50 min, Column: CHIRALPAK IF (250×4.6 mm) 5 μm; Co-solvent: Methanol, Total flow: 3 mL/min, % of co solvent: 15%, ABPR: 100 bar, Temperature: 30° C.

### Preparation 5: (2S)-2-(benzyloxycarbonylamino)-3,3-dicyclopropyl-propanoic acid

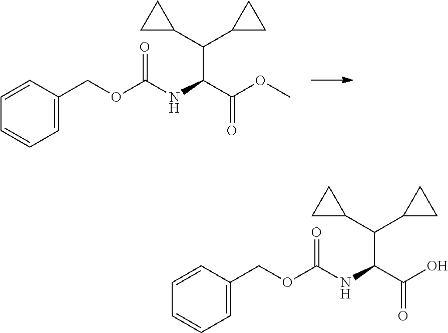 [SMILES: COC(=O)[C@@H](NC(=O)OCc1ccccc1)C(C1CC1)C1CC1 ; O=C(N[C@H](C(=O)O)C(C1CC1)C1CC1)OCc1ccccc1]

NaOH (4M aq. solution, 31.3 mmol) was added to a solution of the compound of Preparation 4a (4.96 g, 15.6 mmol) in MeOH (20 mL) and DCM (20 mL) and the reaction mixture was stirred at room temperature for 16 hours. H₂O (50 mL) was added and the mixture was extracted with TBME (2×100 mL). The aqueous phase was acidified to pH 2 with 4M aq. HCl, then extracted with EtOAc (3×100 mL). The combined EtOAc layers were dried over MgSO₄, filtered and concentrated in vacuo to leave the title compound as a colourless solid (4.56 g, 96% yield). ¹H NMR (600 MHz, CDCl₃) δ 7.41-7.28 (m, 5H), 5.53 (d, J=9.2 Hz, 1H), 5.13 (s, 2H), 4.64 (dd, J=9.2, 2.6 Hz, 1H), 0.84-0.67 (m, 3H), 0.62-0.33 (m, 4H), 0.33-0.05 (m, 4H).

### Preparation 6: 2-(tert-butoxycarbonylamino)-3,3-dicyclopropyl-propanoic acid

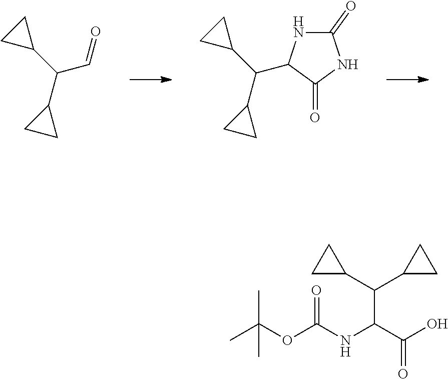 [SMILES: O=CC(C1CC1)C1CC1 ; O=C1NC(=O)C(C(C2CC2)C2CC2)N1 ; CC(C)(C)OC(=O)NC(C(=O)O)C(C1CC1)C1CC1]

A mixture of the compound of Preparation 2 (23.8 g, 192 mmol), KCN (18.7 g, 287 mmol) and ammonium carbonate (100 g, 1040 mmol) in water:EtOH (200 mL:200 mL) was heated at reflux for 6 hours. The resulting pale yellow solution was cooled to room temperature, most of the EtOH was removed in vacuo, the pH was adjusted to between 5 and 6 with 5M HCl and the precipitate was filtered off and dried in vacuo to give 5-(dicyclopropylmethyl)-imidazolidine-2,4-dione (26.4 g, 71% yield) as a white solid. ¹H NMR (400 MHz, DMSO-d6): δ 10.58 (s, 1H), 8.03 (s, 1H), 4.10-4.02 (m, 1H), 0.95-0.72 (m, 2H), 0.55-0.16 (m, 7H), 0.14-0.01 (m, 2H).

The crude hydantoin was heated at reflux in 6.7M NaOH (250 mL) for 48 hours, then cooled in an ice bath and 5M HCl (150 mL) was added slowly. To the cooled solution was then added over 5 min a solution of Boc anhydride (44.9 g, 206 mmol) in THF (60 mL). The mixture was stirred at room temperature for 2 hours, then 5M HCl was added carefully until the pH was between 3 and 4. The mixture was extracted with EtOAc (3×200 mL) and the combined organic extracts were dried (Na₂SO₄) and evaporated. Purification by column chromatography (silica gel, eluting with EtOAc:heptane) gave 2-(tert-butoxycarbonyl-amino)-3,3-dicyclopropyl-propanoic acid (31 g, 56% yield) as a clear oil that solidified on standing. ¹H NMR (300 MHz, CDCl₃) Mixture of rotamers δ 7.90 (br s, 1H), 5.78 (br, 0.15H), 5.26 (d, J=9.2 Hz, 0.85H), 4.55 (d, J=9.2 Hz, 0.85H), 4.37 (br, 0.15H), 1.46 (s, 9H), 1.33-1.21 (m, 1H), 0.85-0.64 (m, 2H), 0.61-0.36 (m, 4H), 0.32-0.13 (m, 4H); LCMS (METHOD 3) (ES): m/z 268.4 [M−H]⁻, RT=0.70 min.

### Preparation 7: (4-methoxyphenyl)methyl (2R)-2-(tert-butoxycarbonylamino)-3,3-dicyclopropyl-propanoate and (4-methoxyphenyl)methyl (2S)-2-(tert-butoxycarbonylamino)-3,3-dicyclopropyl-propanoate

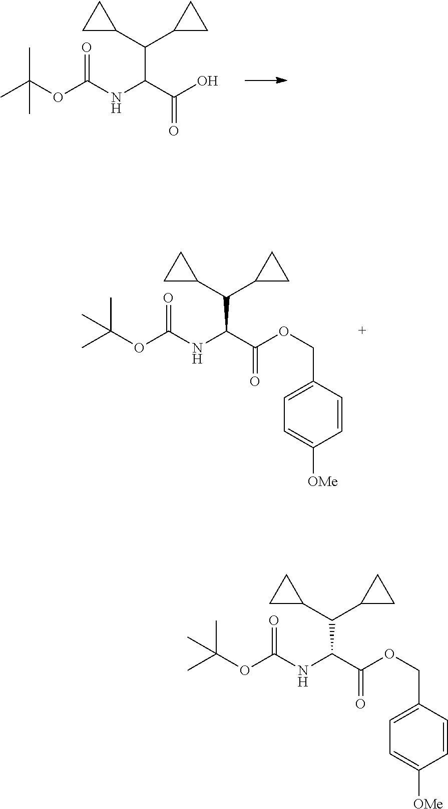 [SMILES: CC(C)(C)OC(=O)NC(C(=O)O)C(C1CC1)C1CC1 ; COc1ccc(COC(=O)[C@@H](NC(=O)OC(C)(C)C)C(C2CC2)C2CC2)cc1 ; COc1ccc(COC(=O)[C@H](NC(=O)OC(C)(C)C)C(C2CC2)C2CC2)cc1]

EDC (7.77 g, 40.5 mmol) was added to a mixture of the acid of Preparation 6 (7.28 g, 27.0 mmol), 4-methoxybenzylalcohol (4.48 g, 32.4 mmol) and DMAP (3.3 g, 27.0 mmol) in DCM (100 mL) and stirred overnight at room temperature. The reaction mixture was washed with 0.25M HCl (15 mL), dried (Na₂SO₄) and evaporated. Purification by column chromatography (silica, eluting with EtOAc:heptane) gave the racemic title compound (9.30 g, 88%) as a white solid. ¹H NMR (300 MHz, CDCl₃) δ 7.38-7.18 (m, 2H), 6.98-6.79 (m, 2H), 5.24 (d, J=9.3 Hz, 1H), 5.09 (s, 2H), 4.53 (d, J=9.3 Hz, 1H), 3.81 (s, 3H), 1.44 (s, 9H), 0.80-0.55 (m, 3H), 0.55-0.26 (m, 4H), 0.25-0.10 (m, 3H), 0.07-−0.05 (m, 1H); LCMS (METHOD 3) (ES): m/z 390.3 [M+H]⁺, RT=0.95 min. The two enantiomers were separated by preparative chiral SFC giving (4-methoxyphenyl)methyl (2R)-2-(tert-butoxycarbonylamino)-3,3-dicyclopropyl-propanoate (Preparation 7a) (Column: Lux A2 (4.6 mm×250 mm, 5 μm), Eluent: 20:80 IPA:CO₂ (0.2% v/v NH₃), Temp: 40° C., Flow rate: 4 mL/min, BPR: 125 Bar, retention time: 1.4 min) and (4-methoxyphenyl)methyl (2S)-2-(tert-butoxycarbonylamino)-3,3-dicyclopropyl-propanoate (Preparation 7b) (Column: Lux A2 (4.6 mm×250 mm, 5 μm), Eluent: 20:80 IPA:CO₂ (0.2% v/v NH₃), Temp: 40° C., Flow rate: 4 mL/min, BPR: 125 Bar, retention time: 1.9 min).

### Preparation 8: (2S)-2-(Tert-butoxycarbonylamino)-3,3-dicyclopropyl-propanoic acid

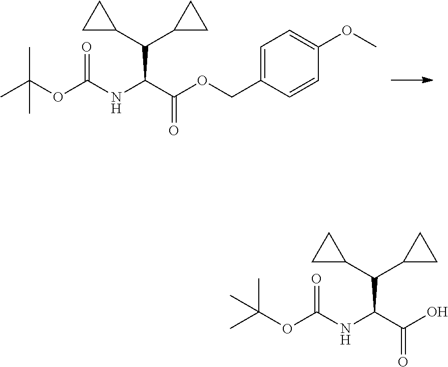

A solution of (4-methoxyphenyl)methyl (2S)-2-(tert-butoxycarbonylamino)-3,3-dicyclopropyl-propanoate (Preparation 7b) (5.30 g, 13.6 mmol) in MeOH (25 mL) was hydrogenated over 10% Pd/C (250 mg) using a hydrogen balloon. After 2½ hours the reaction mixture was filtered and evaporated. Purification by column chromatography (silica, eluting with EtOAc:heptane) gave the title compound (3.50 g, 96%) as a clear syrup. ¹H NMR (400 MHz, DMSO-d6) Mixture of rotamers δ 12.41 (s, 1H), 6.81 (d, J=9.0 Hz, 0.82H), 6.48 (d, J=8.2 Hz, 0.18H), 4.12 (dd, J=9.0, 4.4 Hz, 0.82H), 4.05 (s, 0.18H), 1.39 (s, 7.4H), 1.25 (s, 1.6H), 1.02-0.88 (m, 1H), 0.83-0.72 (m, 1H), 0.56-0.42 (m, 2H), 0.41-0.20 (m, 4H), 0.19-0.01 (m, 3H); LCMS (METHOD 3) (ES): m/z 268.4 [M−H]⁻, RT=0.71 min.

### Preparation 9: benzyl N-[(1S)-3-bromo-1-(4,4-difluorocyclohexyl)-2-oxo-propyl]carbamate

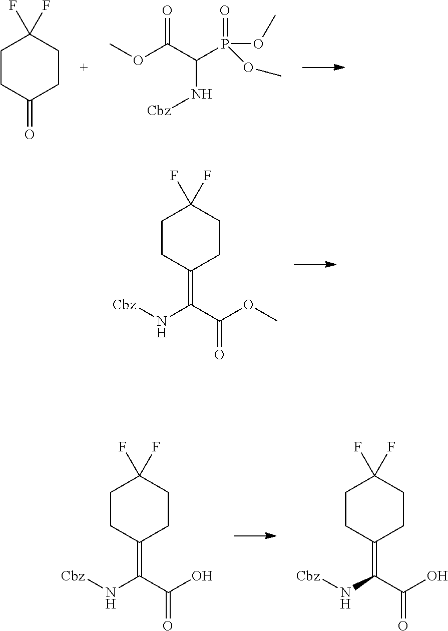

Compound prepared as described in WO2020146194.

### Preparation 10: [1-(2,2,2-trifluoroethyl)triazol-4-yl]methanol

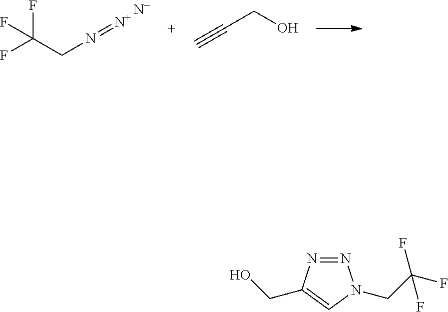

CuSO₄ (10.0 mg, 0.06 mmol) in H₂O (0.5 mL) was added to a solution of 2-azido-1,1,1-trifluoro-ethane (0.6M soln. in TBME, 2.5 mL), prop-2-yn-1-ol (0.07 mL, 1.25 mmol) in THF (2.0 mL) and sodium ascorbate (61.8 mg, 0.31 mmol) in H₂O (1.0 mL) and the reaction mixture was stirred at room temperature for 18 hours. The reaction mixture was diluted with H₂O (5 mL) and extracted with DCM (3×10 mL). The combined organic phase was dried over MgSO₄, filtered and concentrated in vacuo. The obtained crude compound was purified by silica column chromatography (230-400 mesh), eluting with EtOAc in heptane, to afford the title compound as a crystalline solid. (121 mg, 53% yield). ¹H NMR (400 MHz, CDCl₃) δ 7.70 (s, 1H), 4.98 (q, J=8.2 Hz, 2H), 4.85 (d, J=5.9 Hz, 2H), 2.23 (t, J=6.0 Hz, 1H).

### Preparation 10: 4-[(4-nitropyrazol-1-yl)methyl]-1-(2,2,2-trifluoroethyl)triazole

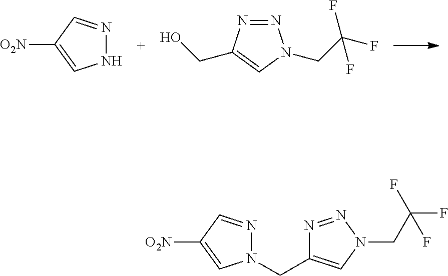 [SMILES: O=[N+]([O-])c1cn[nH]c1 ; OCc1cn(CC(F)(F)F)nn1 ; O=[N+]([O-])c1cnn(Cc2cn(CC(F)(F)F)nn2)c1]

DIAD (0.098 mL, 0.50 mmol) was added to a solution of the compound from Preparation 9 (60.0 mg, 0.33 mmol), 4-nitro-1H-pyrazole (45.0 mg, 0.40 mmol) and triphenylphosphine (130 mg, 0.50 mmol) in THF (2 mL) at room temperature and stirred for 30 minutes. The reaction mixture was concentrated in vacuo and re-dissolved in DMSO (1 mL) and purified by prep. basic HPLC, to afford the title compound as a colourless solid (105 mg, assume 100% yield). ¹H NMR (400 MHz, DMSO-d6) δ 9.00 (s, 1H), 8.32 (s, 1H), 8.28 (s, 1H), 5.60-5.46 (m, 4H).

### Preparation 11: 1-[[1-(2,2,2-trifluoroethyl)triazol-4-yl]methyl]pyrazol-4-amine

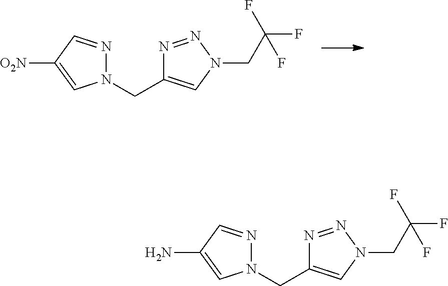 [SMILES: O=[N+]([O-])c1cnn(Cc2cn(CC(F)(F)F)nn2)c1 ; Nc1cnn(Cc2cn(CC(F)(F)F)nn2)c1]

Triethylsilane (0.5 mL) was added dropwise to a degassed solution of the compound of Preparation 10 (105 mg, 0.40 mmol) and Pd/C (10%, 25 mg, 0.024 mmol) in MeOH (5 mL) at room temperature. The mixture was stirred for 1 hour. The reaction mixture was filtered through Celite, and the cake was washed well with MeOH. The combined filtrate was concentrated in vacuo to afford the title compound (100 mg, 86% yield). ¹H NMR (400 MHz, CDCl₃) δ 7.62 (s, 1H), 7.18 (d, J=0.9 Hz, 1H), 7.13 (d, J=0.9 Hz, 1H), 5.33 (s, 2H), 5.03-4.87 (m, 2H).

### Preparation 12: tert-butyl N-[(1S)-1-(dicyclopropylmethyl)-2-oxo-2-[[1-[[1-(2,2,2-trifluoroethyl)triazol-4-yl]methyl]pyrazol-4-yl]amino]ethyl]carbamate

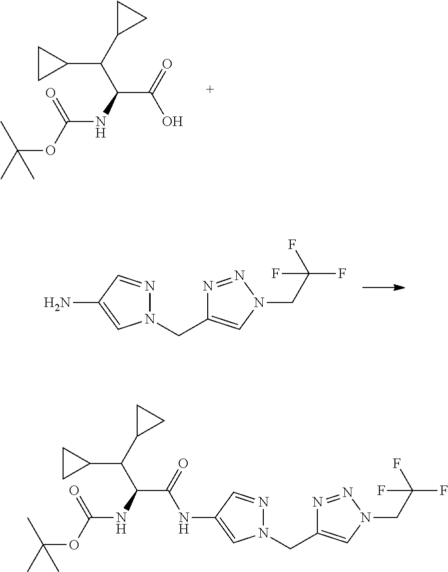 [SMILES: CC(C)(C)OC(=O)N[C@H](C(=O)O)C(C1CC1)C1CC1 ; Nc1cnn(Cc2cn(CC(F)(F)F)nn2)c1 ; CC(C)(C)OC(=O)N[C@H](C(=O)Nc1cnn(Cc2cn(CC(F)(F)F)nn2)c1)C(C1CC1)C1CC1]

HATU (138 mg, 0.37 mmol) was added to a solution of the compound of Preparation 8 (98.5 mg, 0.37 mmol), the compound of Preparation 11 (90.0 mg, 0.37 mmol) and DIPEA (0.07 ml, 0.40 mmol) in DMF (2 mL) and stirred at room temperature for 30 minutes. The obtained crude compound was purified directly by prep. acidic HPLC to afford the title compound as a colourless solid (131 mg, 72% yield). ¹H NMR (400 MHz, CDCl₃) δ 8.12 (s, 1H), 7.92 (s, 1H), 7.62 (s, 1H), 7.49 (s, 1H), 5.47-5.29 (m, 3H), 4.95 (q, J=8.3 Hz, 2H), 4.37 (dd, J=8.5, 4.7 Hz, 1H), 1.45 (s, 9H), 0.98-0.64 (m, 3H), 0.59-0.15 (m, 8H); LCMS (METHOD 3) (ES): m/z 498.4 [M+H]⁺, RT=0.72 min.

### Preparation 13: (2S)-2-amino-3,3-dicyclopropyl-N-[1-[[1-(2,2,2-trifluoroethyl)triazol-4-yl]methyl]pyrazol-4-yl]propanamide

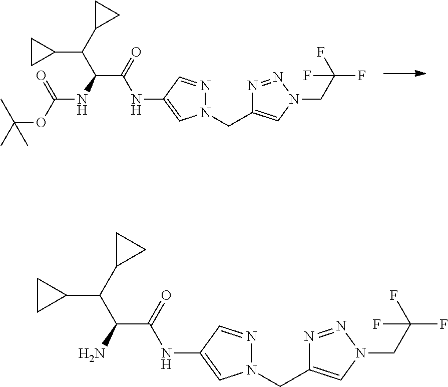 [SMILES: CC(C)(C)OC(=O)N[C@H](C(=O)Nc1cnn(Cc2cn(CC(F)(F)F)nn2)c1)C(C1CC1)C1CC1 ; N[C@H](C(=O)Nc1cnn(Cc2cn(CC(F)(F)F)nn2)c1)C(C1CC1)C1CC1]

HCl (4M solution in 1,4-dioxane, 2.0 mL) was added to a solution of the compound of Preparation 12 (120 mg, 0.24 mmol) in MeOH (2 mL) and stirred at room temperature for 1 hour. The reaction mixture was concentrated in vacuo to afford the crude title compound that was used without further purification (95.9 mg assume 100% yield). LCMS (METHOD 3) (ES): m/z 398.3 [M+H]⁺, RT=0.47 min.

### Preparation 14: [3-(2,2,2-trifluoroethyl)triazol-4-yl]methanol

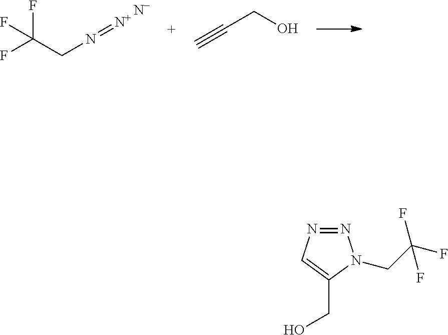

2-Azido-1,1,1-trifluoro-ethane (0.6M soln. in DME, 1.6 mL) was added to a solution of prop-2-yn-1-ol (0.056 mL, 0.96 mmol) in toluene (2.0 mL) and the reaction mixture was stirred at 120° C. in a sealed vial for 4 hours. The cooled reaction mixture was concentrated in vacuo, dissolved in DCM and purified by silica column chromatography (230-400 mesh), eluting with MeOH in DCM, to afford the title compound as a colourless oil. (40 mg, 23% yield). ¹H NMR (400 MHz, CDCl₃) δ 7.52 (s, 1H), 5.15 (q, J=8.4 Hz, 2H), 4.84 (d, J=5.0 Hz, 2H), 4.09 (t, J=5.6 Hz, 1H).

### Preparation 15: 5-[(4-nitropyrazol-1-yl)methyl]-1-(2,2,2-trifluoroethyl)triazole

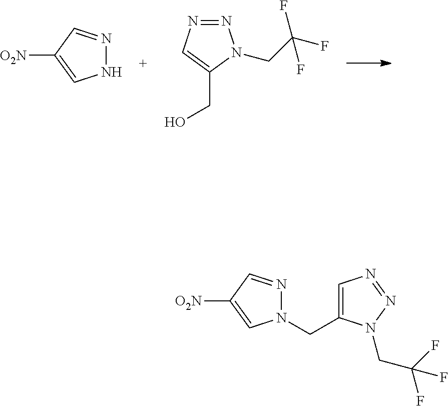 [SMILES: O=[N+]([O-])c1cn[nH]c1 ; OCc1cnnn1CC(F)(F)F ; O=[N+]([O-])c1cnn(Cc2cnnn2CC(F)(F)F)c1]

According to the method of Preparation 10 the compound of Preparation 14 (40.0 mg, 0.22 mmol) was reacted with 4-nitro-1H-pyrazole (30.0 mg, 0.27 mmol) to afford the title compound as a colourless oil after prep. acidic HPLC (74 mg, 66% yield). ¹H NMR (400 MHz, DMSO-d6) δ 9.05 (s, 1H), 8.32 (s, 1H), 7.82 (s, 1H), 7.67-7.50 (m, 2H), 5.73 (s, 2H), 5.63 (q, J=9.0 Hz, 2H); LCMS (METHOD 3) (ES): m/z 277.1 [M+H]⁺, RT=0.54 min.

### Preparation 16: 1-[[3-(2,2,2-trifluoroethyl)triazol-4-yl]methyl]pyrazol-4-amine

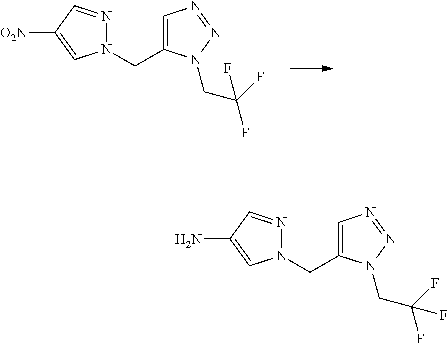 [SMILES: O=[N+]([O-])c1cnn(Cc2cnnn2CC(F)(F)F)c1 ; Nc1cnn(Cc2cnnn2CC(F)(F)F)c1]

According to the method of Preparation 11 the compound of Preparation 15 (68.0 mg, 0.135 mmol) was reacted to afford the title compound as a reddish solid (63 mg, 74% yield). ¹H NMR (400 MHz, CDCl₃) δ 7.70 (s, 1H), 7.17 (d, J=0.8 Hz, 1H), 6.97 (d, J=0.9 Hz, 1H), 6.34 (s, 2H), 5.31 (s, 2H), 5.13 (q, J=8.3 Hz, 2H); LCMS (METHOD 3) (ES): m/z 247.1 [M+H]⁺, RT=0.30 min.

### Preparation 17: tert-butyl N-[(1S)-1-(dicyclopropylmethyl)-2-oxo-2-[[1-[[3-(2,2,2-trifluoroethyl)triazol-4-yl]methyl]pyrazol-4-yl]amino]ethyl]carbamate

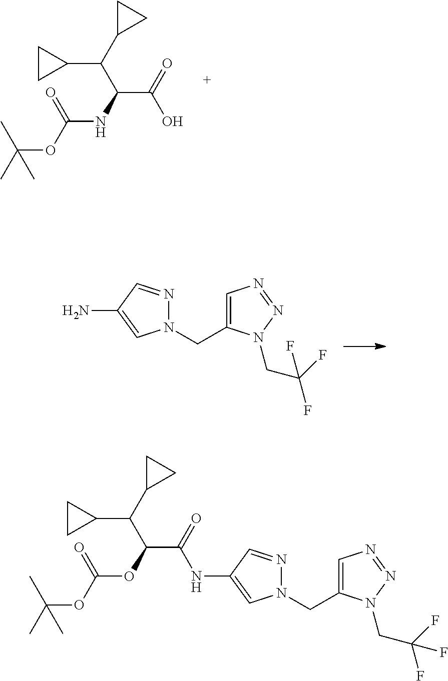 [SMILES: CC(C)(C)OC(=O)N[C@H](C(=O)O)C(C1CC1)C1CC1 ; Nc1cnn(Cc2cnnn2CC(F)(F)F)c1 ; CC(C)(C)OC(=O)O[C@H](C(=O)Nc1cnn(Cc2cnnn2CC(F)(F)F)c1)C(C1CC1)C1CC1]

According to the method of Preparation 12 the compound of Preparation 8 (36.0 mg, 0.14 mmol) was reacted with the compound of Preparation 16 (58.0 mg, 0.12 mmol) to afford the title compound as a colourless oil after prep. acidic HPLC (58 mg, 99% yield). ¹H NMR (400 MHz, CDCl₃) δ 8.12 (s, 1H), 7.96 (s, 1H), 7.72 (s, 1H), 7.42 (s, 1H), 5.41-5.27 (m, 3H), 5.21 (q, J=8.3 Hz, 2H), 4.36 (dd, J=8.3, 4.6 Hz, 1H), 1.45 (s, 9H), 0.98-0.62 (m, 3H), 0.59-0.13 (m, 8H); LCMS (METHOD 3) (ES): m/z 498.4 [M+H]⁺, RT=0.74 min.

### Preparation 18: (2S)-2-amino-3,3-dicyclopropyl-N-[1-[[3-(2,2,2-trifluoroethyl)triazol-4-yl]methyl]pyrazol-4-yl]propanamide

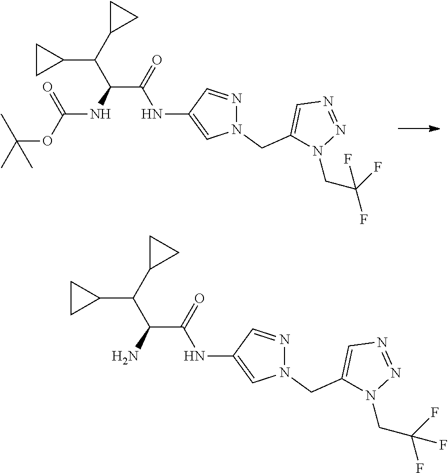 [SMILES: CC(C)(C)OC(=O)N[C@H](C(=O)Nc1cnn(Cc2cnnn2CC(F)(F)F)c1)C(C1CC1)C1CC1 ; N[C@H](C(=O)Nc1cnn(Cc2cnnn2CC(F)(F)F)c1)C(C1CC1)C1CC1]

According to the method of Preparation 13 the compound of Preparation 17 (54.0 mg, 0.11 mmol) was reacted to afford the title compound as a reddish solid (43 mg, assume 100% yield). LCMS (METHOD 3) (ES): m/z 398.4 [M+H]⁺, RT=0.47 min.

### Preparation 19: (3,3-difluorocyclobutyl) (2,5-dioxopyrrolidin-1-yl) carbonate

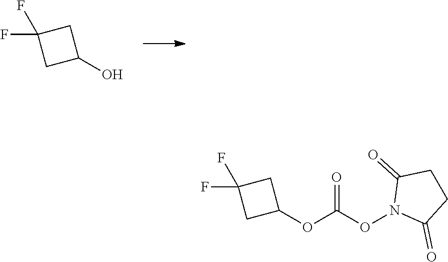 [SMILES: OC1CC(F)(F)C1 ; O=C(OC1CC(F)(F)C1)ON1C(=O)CCC1=O]

Bis(2,5-dioxopyrrolidin-1-yl) carbonate (59.2 mg, 0.23 mmol) was added to a mixture of 3,3-difluorocyclobutanol (25.0 mg, 0.23 mmol) and pyridine (0.022 mL, 0.278 mmol) in MeCN (0.4 mL) and stirred at room temperature for 16 hours. The reaction mixture was quenched with saturated aqueous NaHCO₃ (0.75 mL) and extracted with EtOAc (2×1 mL). The combined organic phase was dried over Na₂SO₄, filtered and concentrated in vacuo, to give the title compound as a colourless solid (40.6 mg, 67% yield). ¹H NMR (400 MHz, DMSO-d6) δ 5.23-5.10 (m, 1H), 3.25-3.10 (m, 2H), 2.94-2.82 (m, 2H), 2.82 (s, 4H).

### Preparation 20: methyl 3-(difluoromethyl)triazole-4-carboxylate

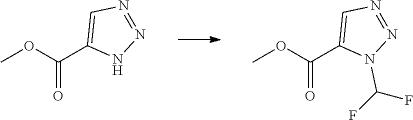

Cs₂CO₃ (6.36 g, 19.5 mmol) was added to a solution of methyl 1H-triazole-4-carboxylate (1.24 g, 9.76 mmol) and sodium chlorodifluoroacetate (2.97 g, 19.5 mmol) in DMF (30 mL) and stirred at 70° C. for 16 hours. The cooled reaction mixture was diluted with H₂O (50 mL) and extracted with TBME (2×50 mL). The combined organic phase was dried over MgSO₄, filtered and concentrated in vacuo. The obtained crude mixture of regioisomers was purified by silica column chromatography (230-400 mesh), eluting with EtOAc in heptane, to afford the title compound as a crystalline solid. (367 mg, 21% yield). ¹H NMR (600 MHz, CDCl₃) δ 8.51 (s, 1H), 7.62 (t, J=58.9 Hz, 1H), 4.00 (s, 3H).

### Preparation 21: [3-(difluoromethyl)triazol-4-yl]methanol

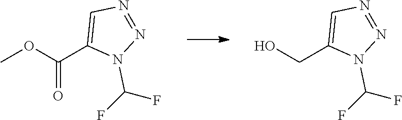

DIBAL (1.5M soln. in toluene, 3.0 mL) was added dropwise to a solution of the compound of Preparation 20 (360 mg, 2.03 mmol) in DCM (10 mL) at 5° C. On complete addition the reaction mixture was stirred at 5-10° C. for 2 hours. The reaction mixture was quenched with saturated NH₄Cl solution (aq., 5 mL) and slurried for 10 minutes. The mixture was extracted with TBME (3×10 mL). The combined organic phase was dried over MgSO₄, filtered and concentrated in vacuo. The obtained crude compound was purified by silica column chromatography (230-400 mesh), eluting with EtOAc in heptane, to afford the title compound as a colourless solid (131 mg, 43% yield). ¹H NMR (600 MHz, CDCl₃) δ 7.97 (s, 1H), 7.55 (t, J=59.1 Hz, 1H), 4.86 (s, 2H), 3.20 (d, J=8.5 Hz, 1H).

### Preparation 22: 5-(chloromethyl)-1-(difluoromethyl)triazole

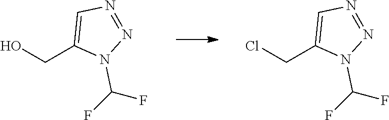 [SMILES: OCc1cnnn1C(F)F ; FC(F)n1nncc1CCl]

SOCl₂ (0.5 mL) was added dropwise to a solution of the compound of Preparation 21 (12.0 mg, 0.08 mmol) in MeCN (1.0 mL) and stirred at room temperature for 1 hour. The reaction mixture was concentrated in vacuo to afford the crude title compound (13.4 mg, assume 100% yield). The material was used directly in the next step without purification.

### Preparation 23: tert-butyl 4-[[(2S)-2-(benzyloxycarbonylamino)-3,3-dicyclopropyl-propanoyl]amino]pyrazole-1-carboxylate

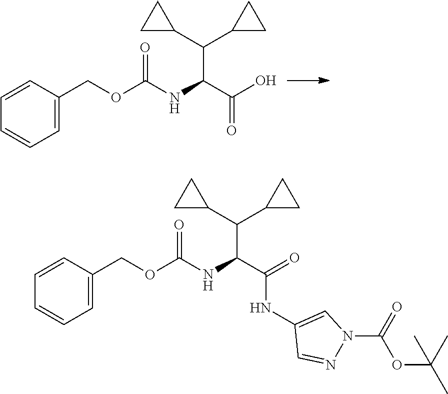 [SMILES: O=C(N[C@H](C(=O)O)C(C1CC1)C1CC1)OCc1ccccc1 ; CC(C)(C)OC(=O)n1cc(NC(=O)[C@@H](NC(=O)OCc2ccccc2)C(C2CC2)C2CC2)cn1]

HATU (2.10 g, 5.4 mmol) was added to a solution of the compound of Preparation 5 (1.10 g, 3.60 mmol), tert-butyl 4-aminopyrazole-1-carboxylate (0.65 g, 3.50 mmol) and TEA (1.0 mL) in DMF (15 mL) at room temperature. The reaction mixture was stirred for 4 hours, then quenched with H₂O (50 mL). The precipitate was collected and washed with H₂O (3×20 mL), then dissolved in DCM, dried over MgSO₄, filtered and concentrated in vacuo to afford the title compound as a tan oil (1.54 g, 91% yield). LCMS (METHOD 3) (ES): m/z 469.3 [M+H]⁺, RT=0.83 min.

### Preparation 24: tert-butyl 4-[[(2S)-2-amino-3,3-dicyclopropyl-propanoyl]amino]pyrazole-1-carboxylate

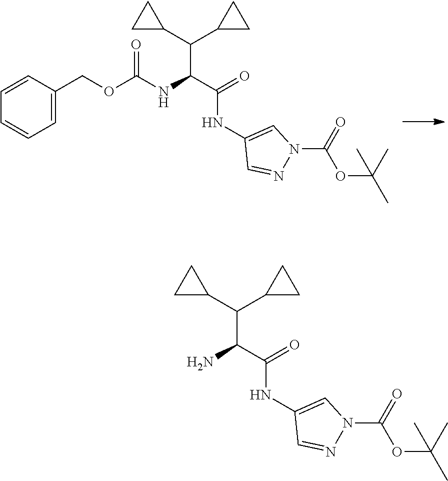 [SMILES: CC(C)(C)OC(=O)n1cc(NC(=O)[C@@H](NC(=O)OCc2ccccc2)C(C2CC2)C2CC2)cn1 ; CC(C)(C)OC(=O)n1cc(NC(=O)[C@@H](N)C(C2CC2)C2CC2)cn1]

Triethylsilane (1.0 mL) was added dropwise to a thoroughly degassed solution of the compound of Preparation 23 (1.50 g, 3.2 mmol) and Pd/C (200 mg, 0.19 mmol) in MeOH (20 mL) under balloon pressure of nitrogen. On complete addition the reaction was stirred for 10 minutes, then filtered through Celite. The cake was washed with MeOH (3×20 mL). The combined organic phase was concentrated in vacuo to afford the title compound as a thick oil (0.87 g, 81% yield). LCMS (METHOD 3) (ES): m/z 333.2 [M−H]⁻, RT=0.55 min.

### Preparation 25: tert-butyl 4-[[(2S)-3,3-dicyclopropyl-2-[(2-isopropylpyrazole-3-carbonyl)amino]propanoyl]amino]pyrazole-1-carboxylate

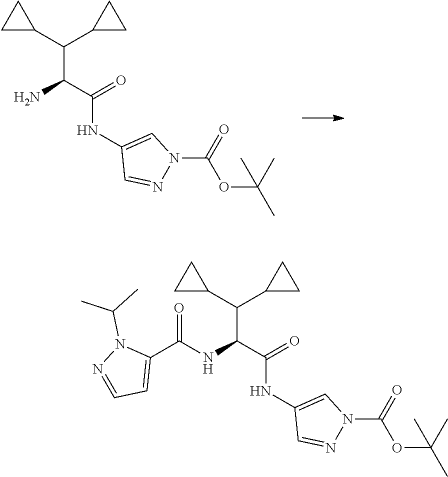

HATU (1.30 g, 3.42 mmol) was added to a solution of the compound of Preparation 24 (870 mg, 2.60 mmol), 2-isopropylpyrazole-3-carboxylic acid (450 mg, 2.92 mmol) and TEA (1 mL) in DMF (10 mL) and stirred at room temperature for 10 minutes. H₂O (30 mL) was added and the reaction mixture was stirred for 20 minutes. The solid was collected by filtration and dried in vacuo, to afford the title compound as a light yellow solid (0.82 g, 1.7 mmol). LCMS (METHOD 3) (ES): m/z 471.4 [M+H]⁺, RT=0.79 min.

### Preparation 26: N-[(1S)-1-(dicyclopropylmethyl)-2-oxo-2-(1H-pyrazol-4-ylamino)ethyl]-2-isopropyl-pyrazole-3-carboxamide

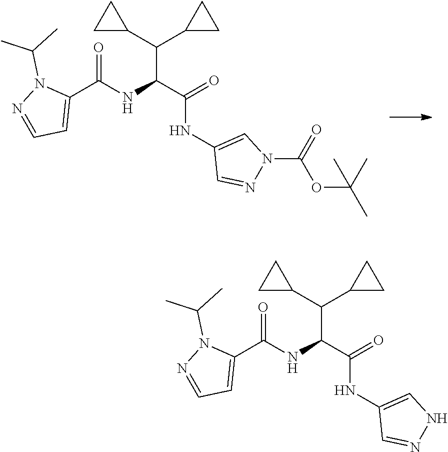

TFA (2 mL) was added to a solution of the compound of Preparation 25 (0.82 g, 1.7 mmol) in DCM (5 mL) and stirred at room temperature for 1 hour. The reaction mixture was concentrated in vacuo. The residue was dissolved in H₂O (20 mL) and basified with solid K₂CO₃, and the reaction mixture was stirred for 30 minutes. The solid was collected by filtration. The obtained crude compound was purified by silica column chromatography (230-400 mesh), eluting with EtOAc, to afford the title compound as a colourless solid (401 mg, 62% yield). LCMS (METHOD 3) (ES): m/z 371.3 [M+H]⁺, RT=0.60 min.

### Preparation 27: N,2-dimethoxy-N-methyl-acetamide

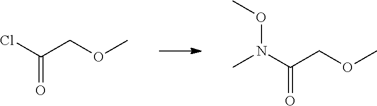

TEA (20 mL, 143 mmol) was added dropwise to a solution of 2-methoxyacetyl chloride (5.0 g, 46.1 mmol) and N-methoxymethanamine hydrochloride (4.95 g, 50.7 mmol) in DCM (50 mL) at 5° C. On complete addition the reaction mixture was stirred at room temperature for 16 hours. The reaction mixture was diluted with H₂O (50 mL) and extracted with DCM (3×40 mL). The combined organic phase was washed with aqueous HCl solution (1M, 30 mL), then with saturated NaHCO₃ aqueous solution (20 mL), dried over MgSO₄, filtered and concentrated in vacuo to afford the title compound as a yellow oil (4.35 g, 64% yield). ¹H NMR (400 MHz, CDCl₃) δ 4.22 (s, 2H), 3.69 (d, J=1.7 Hz, 3H), 3.57-3.36 (m, 3H), 3.19 (d, J=2.1 Hz, 3H).

### Preparation 28: 1-methoxybut-3-yn-2-one

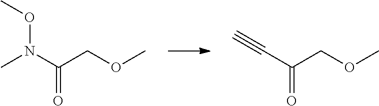

Bromo(ethynyl)magnesium (0.5M soln. in THF, 90 mL) was added slowly to a solution of the compound of Preparation 27 (5.2 g, 39 mmol) in THF (20 mL) at 5° C. On complete addition the reaction mixture was stirred for 2 hours. The reaction mixture was quenched with aqueous NH₄Cl solution (40 mL) and extracted with TBME (2×30 mL). The combined organic phase was dried over MgSO₄ and filtered to afford the crude title compound that was used directly in the next step (3.8 g, assume 100% yield).

### Preparation 29: 1-methoxybut-3-yn-2-ol

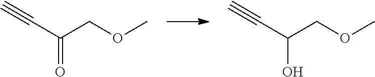

LiBH₄ (1.0 g, 45.9 mmol) was added portion-wise to the TBME solution of the compound of Preparation 28 (3.8 g, 39 mmol) at room temperature. On complete addition the reaction mixture was stirred for 16 hours at room temperature. The reaction mixture was quenched with aqueous NH₄Cl solution (40 mL) and the phases separated. The aqueous phase was extracted with TBME (20 mL). The combined organic phase was dried over MgSO₄, filtered and concentrated in vacuo to afford the title compound (1.5 g, 38% yield). ¹H NMR (400 MHz, CDCl₃) δ 4.54 (q, J=5.4 Hz, 1H), 3.62-3.49 (m, 2H), 3.45 (s, 3H), 2.50 (d, J=5.1 Hz, 1H), 2.47 (d, J=2.2 Hz, 1H).

### Preparation 30: 3-fluoro-1-[1-(methoxymethyl)prop-2-ynyl]-4-nitro-pyrazole

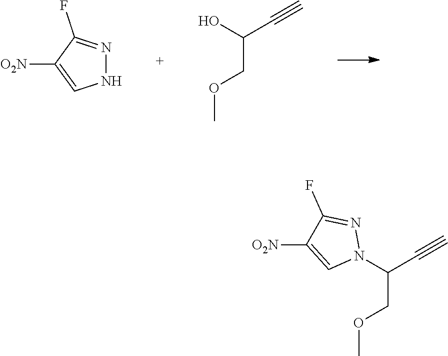

According to the method of Preparation 10 the compound of Preparation 29 (850 mg, 6.8 mmol) was reacted with 3-fluoro-4-nitro-1H-pyrazole (890 mg, 6.8 mmol) to afford the title compound as a colourless oil after prep. acidic HPLC (550 mg, 38% yield). ¹H NMR (600 MHz, DMSO-d6) δ 8.92 (d, J=1.2 Hz, 1H), 5.54 (dddd, J=8.4, 4.2, 2.4, 1.3 Hz, 1H), 3.90-3.69 (m, 3H), 3.26 (s, 3H).

### Preparation 31: 5-[1-(3-fluoro-4-nitro-pyrazol-1-yl)-2-methoxy-ethyl]-1-(2,2,2-trifluoroethyl)triazole

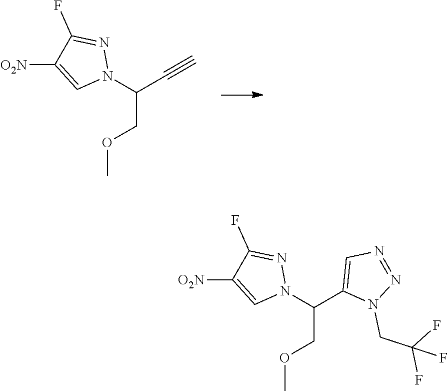 [SMILES: C#CC(COC)n1cc([N+](=O)[O-])c(F)n1 ; COCC(c1cnnn1CC(F)(F)F)n1cc([N+](=O)[O-])c(F)n1]

Chloro(1,5-cyclooctadiene)(pentamethylcyclopentadienyl)ruthenium (II) (10 mg, 0.026 mmol) was added to a solution of the compound of Preparation 30 (56 mg, 0.26 mmol) and 2-azido-1,1,1-trifluoro-ethane (0.6M soln. in DME, 1.6 mL) in TBME (5 mL) and the reaction mixture was stirred at 60° C. under microwave conditions for 40 minutes. The reaction mixture was purified directly by prep. acidic HPLC to afford the title compound as a light brown oil (55 mg, 61% yield). ¹H NMR (600 MHz, CDCl₃) δ 8.17 (d, J=1.1 Hz, 1H), 8.01 (s, 1H), 5.53 (dd, J=5.5, 4.1 Hz, 1H), 5.23-5.12 (m, 1H), 5.04 (dq, J=15.9, 8.0 Hz, 1H), 4.12-3.95 (m, 2H), 3.48 (s, 3H).

### Preparation 32: 3-fluoro-1-[2-methoxy-1-[3-(2,2,2-trifluoroethyl)triazol-4-yl]ethyl]pyrazol-4-amine

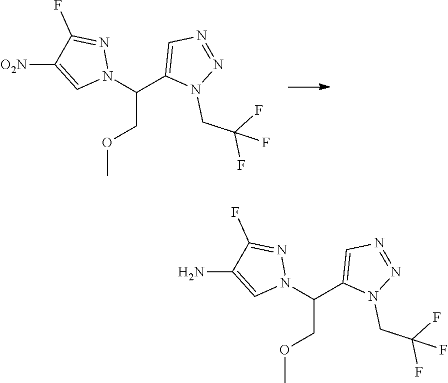 [SMILES: COCC(c1cnnn1CC(F)(F)F)n1cc([N+](=O)[O-])c(F)n1 ; COCC(c1cnnn1CC(F)(F)F)n1cc(N)c(F)n1]

According to the method of Preparation 11 the compound of Preparation 31 (55.0 mg, 0.16 mmol) was reacted to afford the title compound as a reddish solid (50 mg, assume 100% yield). LCMS (METHOD 3) (ES): m/z 309.2 [M+H]⁺, RT=0.45 min.

### Preparation 33: benzyl N-[(1S)-1-(dicyclopropylmethyl)-2-[[3-fluoro-1-[2-methoxy-1-[3-(2,2,2-trifluoroethyl)triazol-4-yl]ethyl]pyrazol-4-yl]amino]-2-oxo-ethyl]carbamate

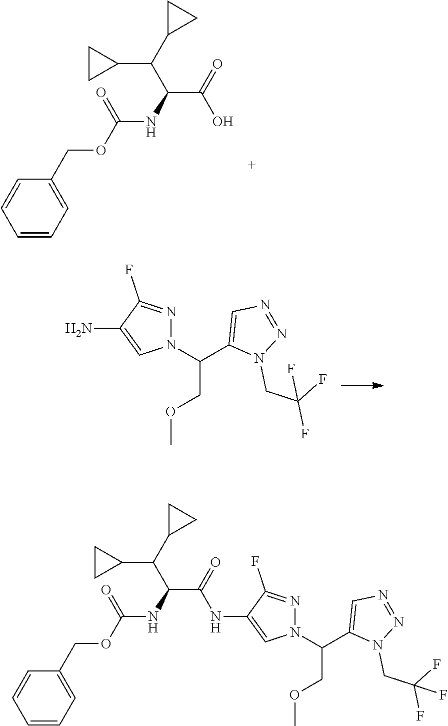 [SMILES: O=C(N[C@H](C(=O)O)C(C1CC1)C1CC1)OCc1ccccc1 ; COCC(c1cnnn1CC(F)(F)F)n1cc(N)c(F)n1 ; COCC(c1cnnn1CC(F)(F)F)n1cc(NC(=O)[C@@H](NC(=O)OCc2ccccc2)C(C2CC2)C2CC2)c(F)n1]

According to the method of Preparation 12 the compound of Preparation 5 (49.3 mg, 0.16 mmol) was reacted with the compound of Preparation 32 (50 mg, 0.16 mmol) to afford the title compound as a colourless oil after prep. acidic HPLC (51 mg, 52% yield). ¹H NMR (400 MHz, CDCl₃) δ 7.94 (q, J=2.1 Hz, 1H), 7.90 (s, 1H), 7.85 (s, 1H), 7.32 (s, 5H), 5.63 (d, J=8.1 Hz, 1H), 5.45 (t, J=5.5 Hz, 1H), 5.12 (d, J=14.8 Hz, 3H), 4.86 (ddt, J=14.4, 8.5, 7.2 Hz, 1H), 4.48 (dd, J=8.1, 4.7 Hz, 1H), 4.05-3.92 (m, 2H), 3.40 (s, 3H), 0.94-0.78 (m, 1H), 0.70 (qd, J=8.2, 4.8 Hz, 2H), 0.63-0.34 (m, 4H), 0.34-0.08 (m, 4H). LCMS (METHOD 3) (ES): m/z 594.5 [M+H]⁺, RT=0.80 min.

### Preparation 34: (2S)-2-amino-3,3-dicyclopropyl-N-[3-fluoro-1-[2-methoxy-1-[3-(2,2,2-trifluoroethyl)triazol-4-yl]ethyl]pyrazol-4-yl]propanamide

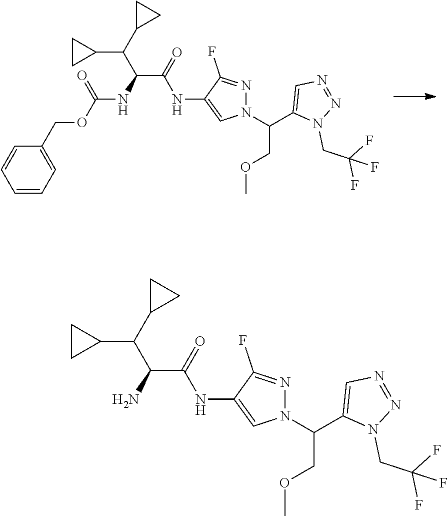 [SMILES: COCC(c1cnnn1CC(F)(F)F)n1cc(NC(=O)[C@@H](NC(=O)OCc2ccccc2)C(C2CC2)C2CC2)c(F)n1 ; COCC(c1cnnn1CC(F)(F)F)n1cc(NC(=O)[C@@H](N)C(C2CC2)C2CC2)c(F)n1]

According to the method of Preparation 24 the compound of Preparation 33 (51 mg, 0.086 mmol) was reacted to afford the title compound (39.5 mg, assume 100% yield). LCMS (METHOD 3) (ES): m/z 460.4 [M+H]⁺, RT=0.54 min.

### Preparation 35: benzyl N-[(1S)-1-(4,4-difluorocyclohexyl)-2-[[3-fluoro-1-[2-methoxy-1-[3-(2,2,2-trifluoroethyl)triazol-4-yl]ethyl]pyrazol-4-yl]amino]-2-oxo-ethyl]carbamate

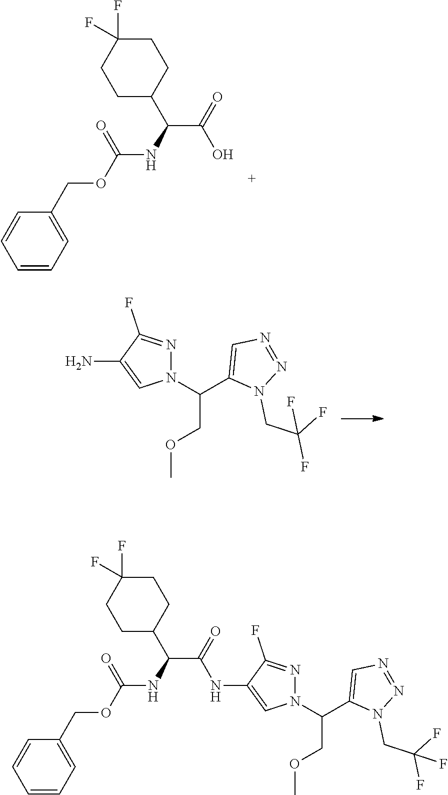 [SMILES: O=C(N[C@H](C(=O)O)C1CCC(F)(F)CC1)OCc1ccccc1 ; COCC(c1cnnn1CC(F)(F)F)n1cc(N)c(F)n1 ; COCC(c1cnnn1CC(F)(F)F)n1cc(NC(=O)[C@@H](NC(=O)OCc2ccccc2)C2CCC(F)(F)CC2)c(F)n1]

According to the method of Preparation 12 the compound of Preparation 9 (30 mg, 0.09 mmol) was reacted with the compound of Preparation 32 (23.7 mg, 0.08 mmol) to afford the title compound after prep. acidic HPLC (39 mg, 82% yield). LCMS (METHOD 3) (ES): m/z 618.4 [M+H]⁺, RT=0.71 min.

### Preparation 36: (2S)-2-amino-2-(4,4-difluorocyclohexyl)-N-[3-fluoro-1-[2-methoxy-1-[3-(2,2,2-trifluoroethyl)triazol-4-yl]ethyl]pyrazol-4-yl]acetamide

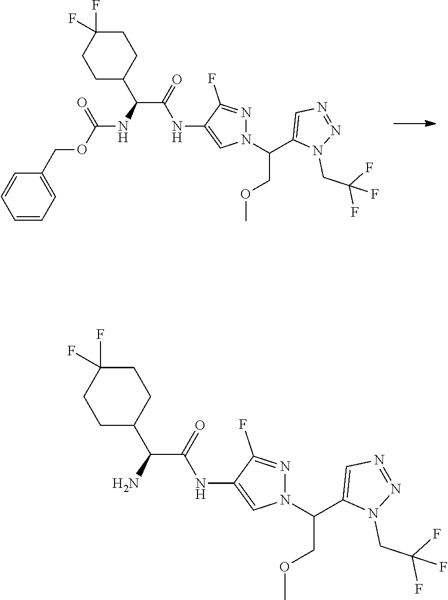 [SMILES: COCC(c1cnnn1CC(F)(F)F)n1cc(NC(=O)[C@@H](NC(=O)OCc2ccccc2)C2CCC(F)(F)CC2)c(F)n1 ; COCC(c1cnnn1CC(F)(F)F)n1cc(NC(=O)[C@@H](N)C2CCC(F)(F)CC2)c(F)n1]

According to the method of Preparation 24 the compound of Preparation 35 (51 mg, 0.083 mmol) was reacted to afford the title compound (40 mg, assume 100% yield). LCMS (METHOD 3) (ES): m/z 484.3 [M+H]⁺, RT=0.57 min.

### Preparation 37: methyl 2-(3-fluoro-4-nitro-pyrazol-1-yl)butanoate

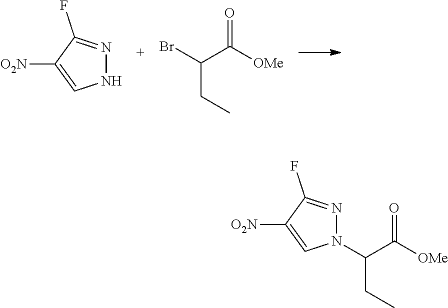

K₂CO₃ (1.19 g, 8.64 mmol) was added to a solution of 3-fluoro-4-nitro-1H-pyrazole (1.03 g, 7.86 mmol) in dry DMF (10 mL) at room temperature under N₂. After stirring for 20 min methyl 2-bromobutanoate (1.56 g, 0.995 mL, 8.64 mmol) was added and the mixture was stirred at 90° C. for 2 hours. Most of the DMF was removed under reduced pressure and the residue was diluted with water (60 mL) and extracted with ether (3×30 mL). The combined organic extracts were dried (Na₂SO₄) and evaporated. Purification by column chromatography (EtOAc:heptane) gave the title compound (1.35 g, 74% Yield) as a yellow oil. ¹H NMR (400 MHz, CDCl₃) δ 8.21 (d, J=1.2 Hz, 1H), 4.70 (dd, J=9.5, 5.6 Hz, 1H), 3.80 (s, 3H), 2.36-2.07 (m, 2H), 0.98 (t, J=7.4 Hz, 3H).

### Preparation 38: 2-(3-fluoro-4-nitro-pyrazol-1-yl)butanoic acid

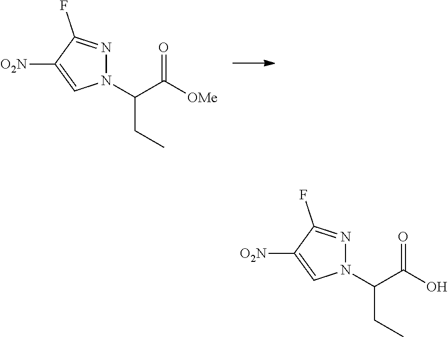 [SMILES: CCC(C(=O)OC)n1cc([N+](=O)[O-])c(F)n1 ; CCC(C(=O)O)n1cc([N+](=O)[O-])c(F)n1]

5M NaOH (1.4 mL, 7.00 mmol) was added to a solution of the ester of Preparation 37 (1.35 g, 5.848 mmol) in MeOH (20 mL) and the mixture was stirred at room temperature for 1 hour. The mixture was concentrated in vacuo, the pH of the residue was adjusted to 3 with 4M HCl and then extracted with ether (2×50 mL). The combined extracts were dried (Na₂SO₄) and evaporated to give the title compound (1.19 g, 94% Yield) as a yellow oil. LCMS (METHOD 3) (ES): m/z 216.3 [M−H]⁻, RT=0.49 min.

### Preparation 39: 2-(3-fluoro-4-nitro-pyrazol-1-yl)-N-(2,2,2-trifluoroethyl)butanamide

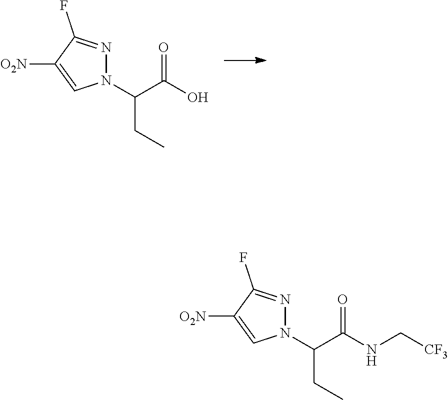 [SMILES: CCC(C(=O)O)n1cc([N+](=O)[O-])c(F)n1 ; CCC(C(=O)NCC(F)(F)F)n1cc([N+](=O)[O-])c(F)n1]

HATU (2.29 g, 6.03 mmol) was added to a solution of the acid of Preparation 38 (1.19 g, 5.48 mmol) and 2,2,2-trifluoroethylamine (1.63 g, 1.31 mL, 16.4 mmol) in MeCN (20 mL) at room temperature under N₂. The mixture was stirred for 1 hour, evaporated, water (20 mL) was added and the resulting solid was filtered off and air dried to give the title compound (1.46 g, 89% Yield) as a white solid. ¹H NMR (400 MHz, DMSO-d6) δ 9.00 (d, J=1.2 Hz, 1H), 8.98 (s, 1H), 4.88 (t, J=7.7 Hz, 1H), 4.11-3.84 (m, 2H), 2.10 (p. J=7.4 Hz, 2H), 0.86 (t, J=7.3 Hz, 3H); LCMS (METHOD 4) (ES): m/z 297.0 [M−H]⁻, RT=0.49 min.

### Preparation 40: 5-[1-(3-fluoro-4-nitro-pyrazol-1-yl)propyl]-1-(2,2,2-trifluoroethyl)tetrazole

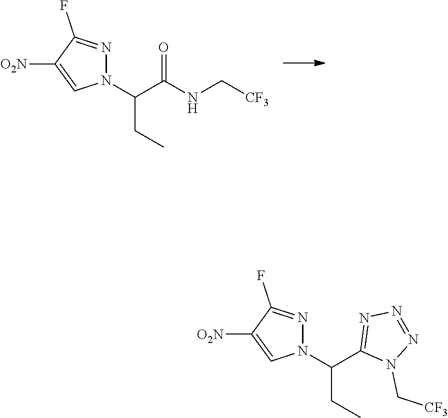 [SMILES: CCC(C(=O)NCC(F)(F)F)n1cc([N+](=O)[O-])c(F)n1 ; CCC(c1nnnn1CC(F)(F)F)n1cc([N+](=O)[O-])c(F)n1]

Triphenylphosphine (2.30 g, 8.75 mmol) was added to a solution of the amide of Preparation 39 (1.74 g, 5.84 mmol) in dry MeCN (12 mL) in a 20 mL microwave vial and the mixture was stirred for 5 min. CCl₄ (1350 mg, 0.847 mL, 8.75 mmol) was added and the mixture was stirred at 85° C., with conventional heating, for 2 hours. The vial was cooled to room temperature, trimethylsilyl azide (1010 mg, 1.16 mL, 8.75 mmol) was added and the vial was heated at 85° C. for 18 hours. After cooling to room temperature the solvent was removed in vacuo and the residue was purified by column chromatography (EtOAc:heptane) to give a mixture of the desired product and triphenylphosphine oxide. Prep. basic HPLC purification gave the title compound (612 mg, 32% Yield) as a yellow oil. LCMS (METHOD 4) (ES): m/z 322.0 [M−H]⁻, RT=0.70 min.

### Preparation 41: methyl 2-(4-nitropyrazol-1-yl)butanoate

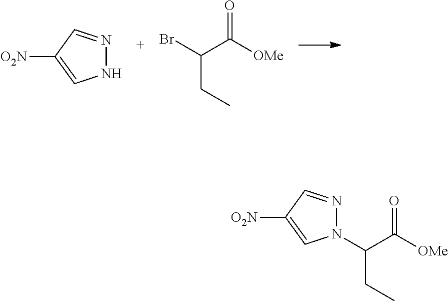

According to the method of Preparation 37, 4-nitropyrazole was alkylated to give the title compound (5.44 g, 71% Yield) as a clear oil. ¹H NMR (400 MHz, CDCl₃) δ 8.35 (d, J=0.6 Hz, 1H), 8.09 (d, J=0.6 Hz, 1H), 4.91 (dd, J=9.5, 5.7 Hz, 1H), 3.80 (s, 3H), 2.42-2.08 (m, 2H), 0.96 (t, J=7.4 Hz, 3H).

### Preparation 42: 2-(4-nitropyrazol-1-yl)-N-(2,2,2-trifluoroethyl)butanamide

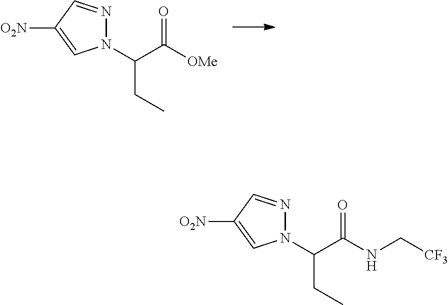 [SMILES: CCC(C(=O)OC)n1cc([N+](=O)[O-])cn1 ; CCC(C(=O)NCC(F)(F)F)n1cc([N+](=O)[O-])cn1]

2,2,2-Trifluoroethylamine (4.01 mL, 5.06 g, 51.0 mmol9 was added slowly to a solution of tert-butylmagnesium chloride in THF (2M, 25.5 mL 51.0 mmol) at 0° C. under N₂ (exothermic reaction). The mixture was stirred for 30 min, then the ester of Preparation 41 (5.44 g, 25.5 mmol) in dry THF (15 mL) was added slowly over 5 min (very exothermic) while keeping the temperature below 15° C. The reaction was stirred for 1 hour, quenched with sat. aq. NH₄Cl (25 mL) and extracted with ether (2×70 mL) The combined organic extracts were dried (Na₂SO₄) and evaporated to give the title compound (2.75 g, 39% Yield) as an off-white solid. ¹H NMR (400 MHz, CDCl₃) δ 8.31 (s, 1H), 8.18 (s, 1H), 7.05 (s, 1H), 4.72 (dd, J=9.3, 6.0 Hz, 1H), 4.01-3.85 (m, 2H), 2.35-2.12 (m, 2H), 0.93 (t, J=7.4 Hz, 3H); LCMS (METHOD 3) (ES): m/z 281.4 [M+H]⁺, RT=0.66 min.

### Preparation 43: 5-[1-(4-nitropyrazol-1-yl)propyl]-1-(2,2,2-trifluoroethyl)tetrazole

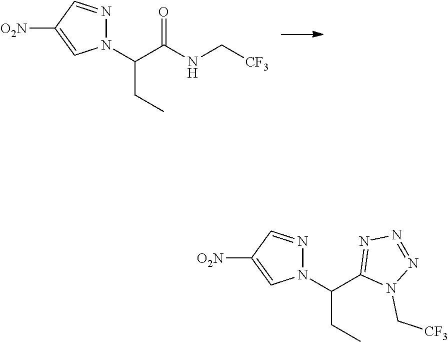 [SMILES: CCC(C(=O)NCC(F)(F)F)n1cc([N+](=O)[O-])cn1 ; CCC(c1nnnn1CC(F)(F)F)n1cc([N+](=O)[O-])cn1]

According to the method of Preparation 40 the compound of Preparation 42 was reacted to give the title compound (75 mg, 61%). GCMS (EI): m/z 305.1 [M]+, RT=12.10 min.

### Preparation 44: 4,4-difluoro-2-hydroxy-N-(2,2,2-trifluoroethyl)butanamide

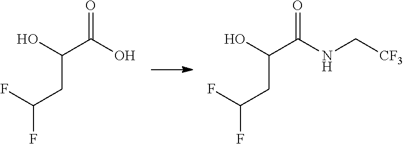 [SMILES: O=C(O)C(O)CC(F)F ; O=C(NCC(F)(F)F)C(O)CC(F)F]

According to the method of Preparation 39 4,4-difluoro-2-hydroxy-butanoic acid was reacted to give the title compound (780 mg, 36% Yield) as a yellow oil. ¹H NMR (600 MHz, DMSO-d6) δ 8.52 (t, J=6.6 Hz, 1H), 6.33-5.94 (m, 2H), 4.17-4.04 (m, 1H), 3.99-3.63 (m, 2H), 2.32-2.14 (m, 1H), 2.14-1.90 (m, 1H).

### Preparation 45: 4,4-difluoro-2-(4-nitropyrazol-1-yl)-N-(2,2,2-trifluoroethyl)butanamide

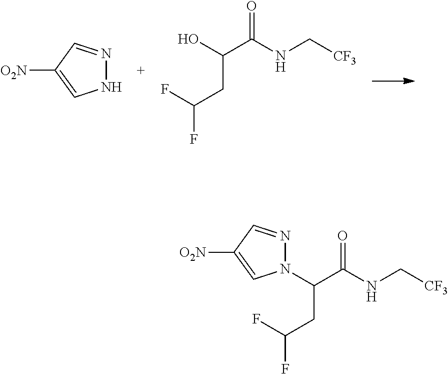 [SMILES: O=[N+]([O-])c1cn[nH]c1 ; O=C(NCC(F)(F)F)C(O)CC(F)F ; O=C(NCC(F)(F)F)C(CC(F)F)n1cc([N+](=O)[O-])cn1]

To a solution of 4-nitro-1H-pyrazole (159 mg, 1.40 mmol), the compound of Preparation 44 (310 mg, 1.40 mmol), and triphenylphosphine (441 mg, 1.68 mmol) in THF (3 mL) was added DIAD (340 mg, 0.331 mL, 1.68 mmol) at room temperature. The solution was stirred for 40 min then concentrated in vacuo, dissolved in DCM (3 mL) and purified by column chromatography (heptane:EtOAc) to give the title compound (569 mg, 68% Yield). ¹H NMR (400 MHz, CDCl₃) δ 8.35 (s, 1H), 8.21 (s, 1H), 7.03 (s, 1H), 5.88 (tt, J=55.3, 4.0 Hz, 1H), 5.18 (dd, J=8.5, 5.9 Hz, 1H), 3.99-3.84 (m, 2H), 2.89-2.61 (m, 2H); LCMS (METHOD 4) (ES): m/z 315.0 [M−H]⁻, RT=0.55 min.

### Preparation 46: 5-[3,3-difluoro-1-(4-nitropyrazol-1-yl)propyl]-1-(2,2,2-trifluoroethyl)tetrazole

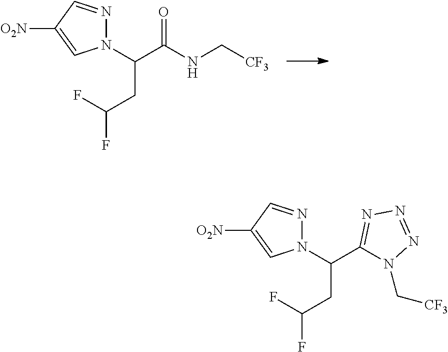 [SMILES: O=C(NCC(F)(F)F)C(CC(F)F)n1cc([N+](=O)[O-])cn1 ; O=[N+]([O-])c1cnn(C(CC(F)F)c2nnnn2CC(F)(F)F)c1]

According to the method of Preparation 40 the compound of Preparation 45 was reacted to give the title compound. ¹H NMR (400 MHz, CDCl₃) δ 8.48 (s, 1H), 8.08 (d, J=0.6 Hz, 1H), 6.22-5.86 (m, 2H), 5.25 (dq, J=16.0, 8.0 Hz, 1H), 5.10 (dq, J=15.6, 7.8 Hz, 1H), 3.37-3.06 (m, 2H); LCMS (METHOD 3) (ES): no ionisation, RT=0.72 min.

### Preparation 47: diethyl 2-(4-nitropyrazol-1-yl)propanedioate

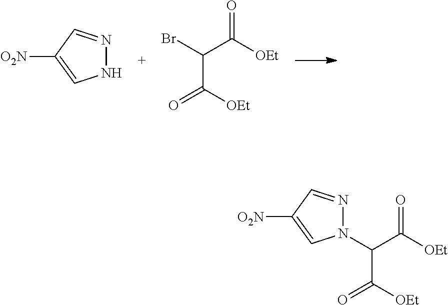 [SMILES: O=[N+]([O-])c1cn[nH]c1 ; CCOC(=O)C(Br)C(=O)OCC ; CCOC(=O)C(C(=O)OCC)n1cc([N+](=O)[O-])cn1]

NaH (C, 53 mmol, 2.1 g) was added to a solution of 4-nitropyrazole (6.0 g, 53 mmol) in dry DMF (40 mL) at room temperature under N₂. The mixture was stirred for 20 min, then diethyl 2-bromopropanedioate (8.6 mL, 12.0 g, 50 mmol) was added and the yellow mixture was stirred at 100° C. for 2 hours. The mixture was poured into water (250 mL), the pH was adjusted to neutral with citric acid and the mixture was extracted with ether (3×80 mL). The combined organic extracts were dried (Na₂SO₄) and evaporated and the residue was purified by column chromatography (EtOAc:heptane) to give the title compound (12.4 g, 86% Yield) as a pale yellow oil. LCMS (METHOD 3) (ES): m/z 270.3 [M−H]⁻, RT=0.74 min.

### Preparation 48: diethyl 2-(2,2-difluoroethyl)-2-(4-nitropyrazol-1-yl)propanedioate

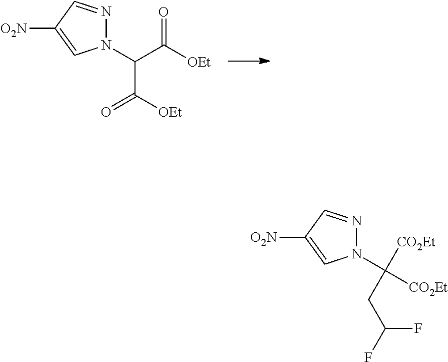 [SMILES: CCOC(=O)C(C(=O)OCC)n1cc([N+](=O)[O-])cn1 ; CCOC(=O)C(CC(F)F)(C(=O)OCC)n1cc([N+](=O)[O-])cn1]

Potassium tert-butoxide (5.64 g, 50.3 mmol) was added to a solution of the compound of Preparation 47 (12.4 g, 45.7 mmol) in dry THF (200 mL) at room temperature under N₂ and the mixture was stirred for 20 min. 2,2-Difluoroethyl trifluoromethanesulfonate (7.3 mL, 11.7 g, 54.9 mmol) was added slowly over 5 min and the suspension was heated at reflux for 2 hours. The resulting orange solution was evaporated, water (100 mL) was added and the mixture was extracted with EtOAc (2×100 mL). The organic extracts were washed with 1M NaOH (40 mL), brine (50 mL), dried (Na₂SO₄) and evaporated to give the title compound (7.4 g, 48% Yield). ¹H NMR (400 MHz, CDCl₃) δ 8.56 (d, J=2.0 Hz, 1H), 8.20 (dt, J=2.0, 0.9 Hz, 1H), 6.16 (tt, J=55.9, 4.2 Hz, 1H), 4.49-4.24 (m, 4H), 3.87 (td, J=13.3, 4.2 Hz, 2H), 1.36 (t, J=7.2 Hz, 6H); LCMS (METHOD 3) (ES): m/z 336.5 [M+H]⁺, RT=0.72 min.

### Preparation 49: 4,4-difluoro-2-(4-nitropyrazol-1-yl)butanoic acid

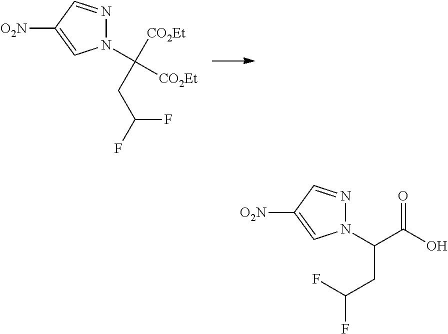 [SMILES: CCOC(=O)C(CC(F)F)(C(=O)OCC)n1cc([N+](=O)[O-])cn1 ; O=C(O)C(CC(F)F)n1cc([N+](=O)[O-])cn1]

10M NaOH (6.6 mL, 66 mmol) was added to a solution of the compound of Preparation 48 (7.4 g, 22 mmol) in MeOH (40 mL) cooled in an ice bath (exothermic). The mixture was stirred for 2 hours, then most of the MeOH was evaporated in vacuo and the pH was adjusted to 2-3 with 5M HCl (CO₂ evolution). The mixture was stirred for 2 hours at room temperature then extracted with EtOAc (3×50 mL). The organic extracts were washed with brine (30 mL), dried (Na₂SO₄) and concentrated in vacuo to give crude title compound (5.04 g, 97% Yield) as a brown solid, that was used without any further purification. LCMS (METHOD 4) (ES): m/z 234.0 [M+H]⁺, RT=0.27 min.

### Preparation 50: N-(3,3-difluorocyclobutyl)-4,4-difluoro-2-(4-nitropyrazol-1-yl)butanamide

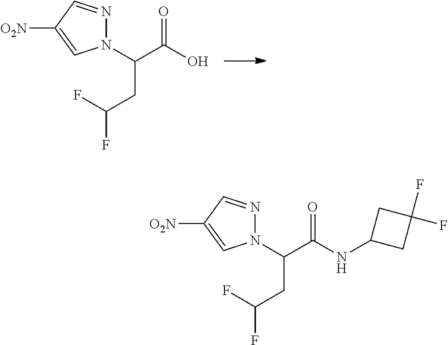 [SMILES: O=C(O)C(CC(F)F)n1cc([N+](=O)[O-])cn1 ; O=C(NC1CC(F)(F)C1)C(CC(F)F)n1cc([N+](=O)[O-])cn1]

According to the method of Preparation 12 the acid of Preparation 49 was reacted with 3,3-difluorocyclobutanamine to give the title compound (361 mg, 68% Yield) as a colourless solid. ¹H NMR (600 MHz, CDCl₃) δ 8.32 (s, 1H), 8.21 (s, 1H), 6.70 (d, J=6.3 Hz, 1H), 5.88 (tt, J=55.5, 3.9 Hz, 1H), 5.06 (dd, J=8.9, 5.5 Hz, 1H), 4.27-4.18 (m, 1H), 3.06-2.94 (m, 2H), 2.87-2.67 (m, 2H), 2.53-2.40 (m, 2H); LCMS (METHOD 3) (ES): m/z 323.2 [M−H]⁻, RT=0.69 min.

### Preparation 51: 1-(3,3-difluorocyclobutyl)-5-[3,3-difluoro-1-(4-nitropyrazol-1-yl)propyl]tetrazole

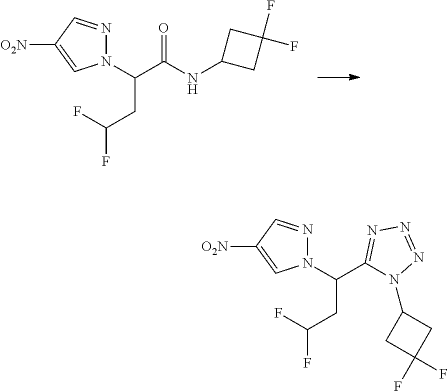 [SMILES: O=C(NC1CC(F)(F)C1)C(CC(F)F)n1cc([N+](=O)[O-])cn1 ; O=[N+]([O-])c1cnn(C(CC(F)F)c2nnnn2C2CC(F)(F)C2)c1]

According to the method of Preparation 40 the compound of Preparation 50 was reacted to give the title compound (195 mg, 81% Yield) as a pale yellow solid. ¹H NMR (600 MHz, CDCl₃) δ 8.36 (s, 1H), 8.11 (s, 1H), 6.13-5.89 (m, 2H), 5.00-4.89 (m, 1H), 3.55-3.40 (m, 1H), 3.37-3.16 (m, 3H), 3.15-2.99 (m, 2H); LCMS (METHOD 3) (ES): m/z 348.2 [M−H]⁻, RT=0.75 min.

### Preparation 52: methyl 4,4-difluoro-2-(3-fluoro-4-nitro-pyrazol-1-yl)butanoate

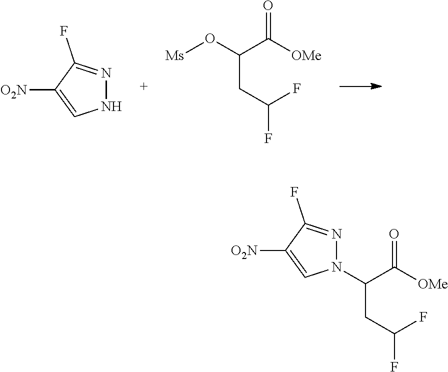 [SMILES: O=[N+]([O-])c1c[nH]nc1F ; COC(=O)C(CC(F)F)OS(C)(=O)=O ; COC(=O)C(CC(F)F)n1cc([N+](=O)[O-])c(F)n1]

To a stirred solution of 3-fluoro-4-nitro-1H-pyrazole (400 mg, 3.05 mmol) in MeCN (15 mL) was added K₂CO₃ (842 mg, 6.10 mmol) at 0° C., then after 5 mins methyl 4,4-difluoro-2-methylsulfonyloxy-butanoate (567 mg, 2.44 mmol). The reaction mixture was stirred at 60° C. for 16 hours, then cooled and filtered. The filtrate was concentrated under reduced pressure to get crude compound which was purified by column chromatography (EtOAc\hexane) to afford the title compound (163 mg, 20% Yield) as a light yellow liquid. LCMS (METHOD 2) (ESI): m/z: 266.3 [M−H]⁻, RT=1.72 min (YMC-Triart C₁₈ (50×2.1 mm, D.S-1.9 μm) column, 0.05% Formic acid in water, 0.05% Formic acid in MeCN).

### Preparation 53: methyl 4,4-difluoro-2-(3-fluoro-4-nitro-pyrazol-1-yl)butanoate

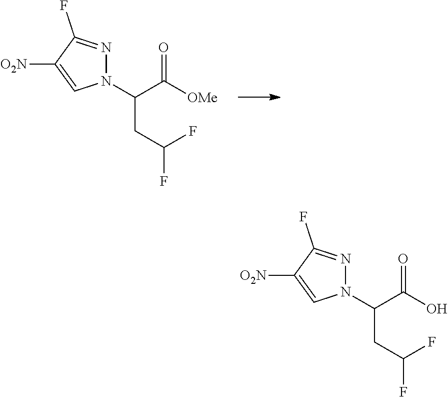 [SMILES: COC(=O)C(CC(F)F)n1cc([N+](=O)[O-])c(F)n1 ; O=C(O)C(CC(F)F)n1cc([N+](=O)[O-])c(F)n1]

To a stirred solution of the ester of Preparation 52 (350 mg, 1.31 mmol) in 1,4-dioxane (2 mL) at 0° C. was added 6N HCl (2 mL). The reaction was heated at 60° C. for 6 hours then cooled and concentrated in vacuo to give the title compound (assume 100% Yield) as an off-white solid. LCMS (METHOD 2) (ESI): m/z: 252.3 [M−H]⁻, RT=1.84 min (YMC-Triart C₁₈ (50×2.1 mm, D.S-1.9 μm) column, 0.05% Formic acid in water, 0.05% Formic acid in MeCN).

### Preparation 54: methyl 4,4-difluoro-2-(3-fluoro-4-nitro-pyrazol-1-yl)butanoate

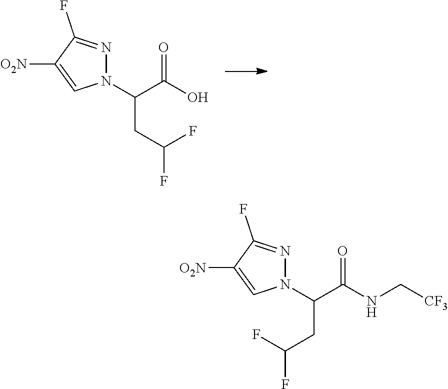 [SMILES: O=C(O)C(CC(F)F)n1cc([N+](=O)[O-])c(F)n1 ; O=C(NCC(F)(F)F)C(CC(F)F)n1cc([N+](=O)[O-])c(F)n1]

According to the method of Preparation 39 the acid of Preparation 53 was reacted to give the title compound (1.15 g, 66% Yield) as a pinkish solid. ¹H NMR (400 MHz, DMSO-d6) δ 9.11 (d, J=1.2 Hz, 1H), 9.00 (t, J=6.3 Hz, 1H), 6.14 (tt, J=55.5, 4.3 Hz, 1H), 5.30 (dd, J=9.8, 4.8 Hz, 1H), 4.06-3.85 (m, 2H), 2.91-2.60 (m, 2H); LCMS (METHOD 4) (ES): m/z 330.0 [M−H]⁻, RT=0.60 min.

### Preparation 55: 5-[3,3-difluoro-1-(3-fluoro-4-nitro-pyrazol-1-yl)propyl]-1-(2,2,2-trifluoroethyl)tetrazole

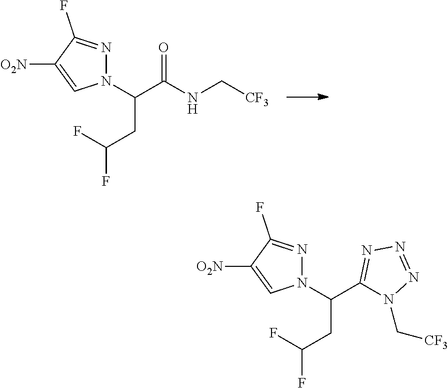 [SMILES: O=C(NCC(F)(F)F)C(CC(F)F)n1cc([N+](=O)[O-])c(F)n1 ; O=[N+]([O-])c1cn(C(CC(F)F)c2nnnn2CC(F)(F)F)nc1F]

According to the method of Preparation 40 the compound of Preparation 54 was reacted to give the title compound (751 mg, 80% Yield) as a brown oil. ¹H NMR (400 MHz, CDCl₃) δ 8.33 (d, J=1.2 Hz, 1H), 6.23-5.85 (m, 2H), 5.23 (dq, J=16.0, 8.0 Hz, 1H), 5.09 (dq, J=15.5, 7.7 Hz, 1H), 3.34-2.98 (m, 2H); LCMS (METHOD 4) (ES): m/z 360.2 [M+H]⁺, RT=0.64 min.

### Preparation 56: methyl 4,4,4-trifluoro-2-(4-nitropyrazol-1-yl)butanoate

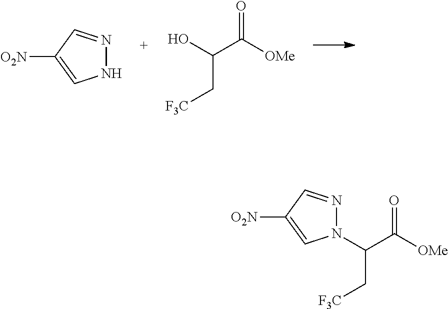 [SMILES: O=[N+]([O-])c1cn[nH]c1 ; COC(=O)C(O)CC(F)(F)F ; COC(=O)C(CC(F)(F)F)n1cc([N+](=O)[O-])cn1]

To a stirred solution of 4-nitropyrazole (600 mg, 5.31 mmol) and methyl 4,4,4-trifluoro-2-hydroxy-butanoate (1.10 g, 6.37 mmol) in THF (100 mL) at 0° C. was added triphenylphosphine (1.67 g, 6.37 mmol) then DIAD (1.29 g, 6.37 mmol). The mixture was stirred at 50° C. for 3 hours then quenched with water. The mixture was partitioned between EtOAc (100 mL) and water (100 mL) and the organic layer was washed with water (2×100 mL) then brine (50 mL), dried over Na₂SO₄ and concentrated in vacuo. The crude mixture was purified by column chromatography (EtOAc/Hexane) to give the title compound (350 mg, 25% Yield). LCMS (METHOD 2) (ESI): m/z: 268.2 [M+H]⁺, RT=1.69 min (YMC-Triart C₁₈ (50×2.1 mm, D.S-1.9 μm) column, 0.05% Formic acid in water, 0.05% Formic acid in MeCN).

### Preparation 57: 4,4,4-trifluoro-2-(4-nitropyrazol-1-yl)-N-(2,2,2-trifluoroethyl)butanamide

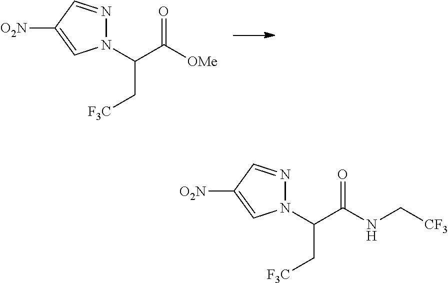 [SMILES: COC(=O)C(CC(F)(F)F)n1cc([N+](=O)[O-])cn1 ; O=C(NCC(F)(F)F)C(CC(F)(F)F)n1cc([N+](=O)[O-])cn1]

To a stirred solution of the compound of Preparation 56 (300 mg, 1.12 mmol) in toluene (3 mL) was added 2,2,2-trifluoroethylamine (122 mg, 1.24 mmol) and AIMe₃ (2M soln. in toluene, 0.84 mL, 1.68 mmol) at 0° C. and the mixture was then stirred for 16 hours at 80° C. with microwave heating. The mixture was partitioned between EtOAc (100 mL) and water (100 mL) and the organic layer was washed with water (2×200 mL) then brine (50 mL), dried over Na₂SO₄ and concentrated in vacuo. The crude mixture was purified by column chromatography (EtOAc/Hexane) to give the title compound (280 mg, 75% Yield). LCMS (METHOD 2) (ESI): m/z: 335.2 [M+H]⁺, RT=1.69 min (YMC-Triart C₁₈ (50×2.1 mm, D.S-1.9 μm) column, 0.05% Formic acid in water, 0.05% Formic acid in MeCN).

### Preparation 58: 1-(2,2,2-trifluoroethyl)-5-[3,3,3-trifluoro-1-(4-nitropyrazol-1-yl)propyl]tetrazole

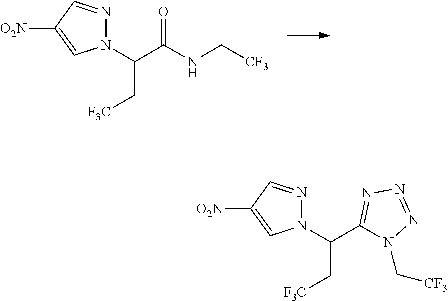 [SMILES: O=C(NCC(F)(F)F)C(CC(F)(F)F)n1cc([N+](=O)[O-])cn1 ; O=[N+]([O-])c1cnn(C(CC(F)(F)F)c2nnnn2CC(F)(F)F)c1]

According to the method of Preparation 40 the compound of Preparation 57 was reacted to give the title compound (250 mg, 78% Yield). ¹H NMR (400 MHz, CDCl₃) δ 8.37 (s, 1H), 8.14 (s, 1H), 6.11 (dd, 1H), 5.22-5.16 (m, 1H), 4.86-4.80 (m, 1H), 3.56-3.44 (m, 2H).

### Preparation 59: 1-(1-methylprop-2-ynyl)-4-nitro-pyrazole

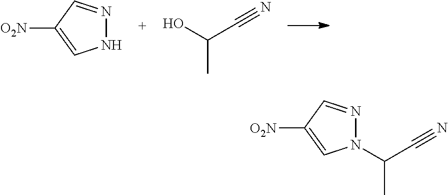

40% DEAD in toluene (4.8 mL, 4.60 g, 11 mmol) was added dropwise to a solution of 4-nitropyrazole (1.00 g, 8.8 mmol), 2-hydroxypropanenitrile (0.76 mL, 750 mg, 11 mmol) and triphenylphosphine (2.80 g, 11 mmol) in dry THF (30 mL) at 10° C. under N₂. The yellow solution was stirred for 2 hours, then all the volatiles were evaporated in vacuo and the residue was purified by column chromatography (EtOAc:heptane) to give the title compound (1.40 g, 95% Yield) as a yellow oil. ¹H NMR (400 MHz, CDCl₃) δ 8.39 (s, 1H), 8.16 (s, 1H), 5.37 (q, J=7.2 Hz, 1H), 1.99 (d, J=7.2 Hz, 3H); LCMS (METHOD 4) (ES): no ionisation, RT=0.41 min.

### Preparation 60: 5-[1-(4-nitropyrazol-1-yl)ethyl]-1H-tetrazole

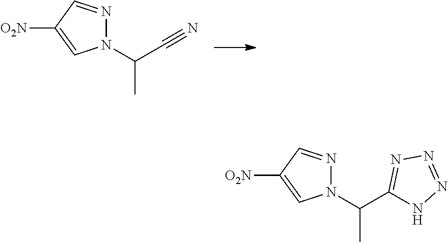

A mixture of the compound of Preparation 59 (1.40 g, 8.43 mmol), sodium azide (603 mg, 9.27 mmol) and ammonium chloride (496 mg, 9.27 mmol) in dry degassed DMF (10 mL) was stirred at 90° C. for 4 hours under N₂. Most of the DMF was removed in vacuo, water (60 mL) was added and the mixture was extracted with EtOAc (3×50 mL). The combined organic extracts were dried (Na₂SO₄) and evaporated to give crude title compound (1.70 g, 97% Yield) as an orange syrup containing ca. 0.8 equivalents DMF. ¹H NMR (400 MHz, DMSO-d6) δ 9.14 (d, J=0.7 Hz, 1H), 8.33 (d, J=0.6 Hz, 1H), 6.22 (q, J=7.0 Hz, 1H), 1.96 (d, J=7.1 Hz, 3H); LCMS (METHOD 4) (ES): m/z 208.1 [M−H]⁻, RT=0.23 min.

### Preparation 61: 5-[1-(4-nitropyrazol-1-yl)ethyl]-1-(2,2,2-trifluoroethyl)tetrazole

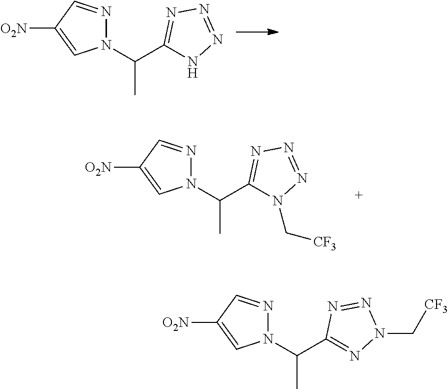

NaH (110 mg, 2.7 mmol) was added to a solution of the compound of Preparation 60 (520 mg, 2.5 mmol) in dry THF (10 mL) at room temperature in a microwave vial. The mixture was stirred for 30 min, then 2,2,2-trifluoroethyl trifluoromethanesulfonate (0.54 mL, 870 mg, 3.7 mmol) was added, the vial was capped and the mixture was stirred at 80° C. in an alu-block for 2 hours. The mixture was cooled and all the volatiles were removed in vacuo. Purification by column chromatography (EtOAc:heptane) gave the title compound (121 mg, 17% Yield) and the regioisomeric product 5-[1-(4-nitropyrazol-1-yl)ethyl]-2-(2,2,2-trifluoroethyl)tetrazole (363 mg, 50% Yield).

Preparation 61: 1H NMR (600 MHz, CDCl₃) δ 8.40 (s, 1H), 8.06 (s, 1H), 5.93 (q, J=7.2 Hz, 1H), 5.36 (dq, J=15.6, 7.8 Hz, 1H), 5.26-5.19 (m, 1H), 2.23 (d, J=7.1 Hz, 3H); LCMS (METHOD 3) (ES): no ionisation, RT=0.69 min. 5-[1-(4-Nitropyrazol-1-yl)ethyl]-2-(2,2,2-trifluoroethyl)tetrazole: 1H NMR (400 MHz, CDCl₃) δ 8.37 (s, 1H), 8.10 (s, 1H), 5.95 (q, J=7.2 Hz, 1H), 5.23 (q, J=7.7 Hz, 2H), 2.09 (d, J=7.2 Hz, 3H); LCMS (METHOD 3) (ES): no ionisation, RT=0.72 min.

### Preparations 62-78

The nitro compounds of Preparations 62-78 were prepared using the appropriate starting materials by the following methods, as indicated in the table below.
Method A—method as described for Preparation 51
Method B—method as described for Preparation 40
Method C—method as described for Preparation 43
Method C—method as described for Preparation 55

| Prep. | | | |
|---|---|---|---|
| No. | Method | Structure | Name |
| 62 | A | 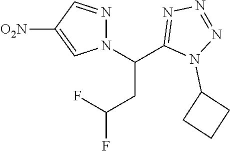 [SMILES: O=[N+]([O-])c1cnn(C(CC(F)F)c2nnnn2C2CCC2)c1] | 1-cyclobutyl-5-[3,3-difluoro-1-(4- nitropyrazol-1-yl)propyl]tetrazole |
| 63 | A | 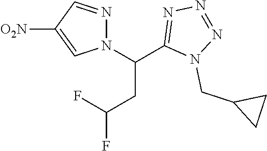 [SMILES: O=[N+]([O-])c1cnn(C(CC(F)F)c2nnnn2CC2CC2)c1] | 1-(cyclopropylmethyl)-5-[3,3-difluoro-1- (4-nitropyrazol-1-yl)propyl]tetrazole |
| 64 | A | 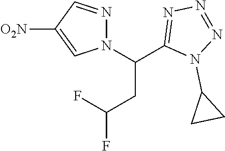 [SMILES: O=[N+]([O-])c1cnn(C(CC(F)F)c2nnnn2C2CC2)c1] | 1-cyclopropyl-5-[3,3-difluoro-1-(4- nitropyrazol-1-yl)propyl]tetrazole |
| 65 | A | 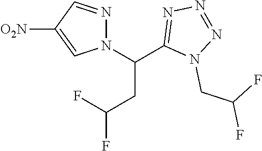 [SMILES: O=[N+]([O-])c1cnn(C(CC(F)F)c2nnnn2CC(F)F)c1] | 1-(2,2-difluoroethyl)-5-[3,3-difluoro-1- (4-nitropyrazol-1-yl)propyl]tetrazole |
| 66 | A | 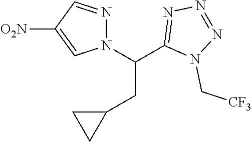 [SMILES: O=[N+]([O-])c1cnn(C(CC2CC2)c2nnnn2CC(F)(F)F)c1] | 5-[2-cyclopropyl-1-(4-nitropyrazol-1- yl)ethyl]-1-(2,2,2-trifluoroethyl)tetrazole |
| 67 | B | 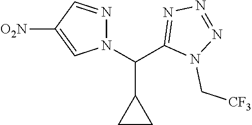 [SMILES: O=[N+]([O-])c1cnn(C(c2nnnn2CC(F)(F)F)C2CC2)c1] | 5-[cyclopropyl-(4-nitropyrazol-1- yl)methyl]-1-(2,2,2- trifluoroethyl)tetrazole |
| 68 | B | 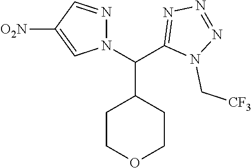 [SMILES: O=[N+]([O-])c1cnn(C(c2nnnn2CC(F)(F)F)C2CCOCC2)c1] | 5-[(4-nitropyrazol-1-yl)- tetrahydropyran-4-yl-methyl]-1-(2,2,2- trifluoroethyl)tetrazole |
| 69 | B | 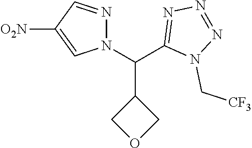 [SMILES: O=[N+]([O-])c1cnn(C(c2nnnn2CC(F)(F)F)C2COC2)c1] | 5-[(4-nitropyrazol-1-yl)-(oxetan-3- yl)methyl]-1-(2,2,2- trifluoroethyl)tetrazole |
| 70 | C | 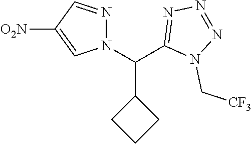 [SMILES: O=[N+]([O-])c1cnn(C(c2nnnn2CC(F)(F)F)C2CCC2)c1] | 5-[cyclobutyl-(4-nitropyrazol-1- yl)methyl]-1-(2,2,2- trifluoroethyl)tetrazole |
| 71 | D | 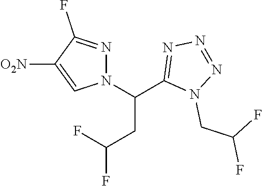 [SMILES: O=[N+]([O-])c1cn(C(CC(F)F)c2nnnn2CC(F)F)nc1F] | 1-(2,2-difluoroethyl)-5-[3,3-difluoro-1- (3-fluoro-4-nitro-pyrazol-1- yl)propyl]tetrazole |
| 72 | D | 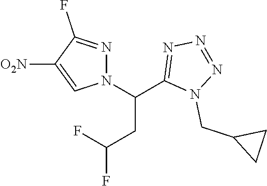 [SMILES: O=[N+]([O-])c1cn(C(CC(F)F)c2nnnn2CC2CC2)nc1F] | 1-(cyclopropylmethyl)-5-[3,3-difluoro-1- (3-fluoro-4-nitro-pyrazol-1- yl)propyl]tetrazole |
| 73 | B | 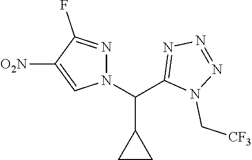 [SMILES: O=[N+]([O-])c1cn(C(c2nnnn2CC(F)(F)F)C2CC2)nc1F] | 5-[cyclopropyl-(3-fluoro-4-nitro-pyrazol- 1-yl)methyl]-1-(2,2,2- trifluoroethyl)tetrazole |
| 74 | B | 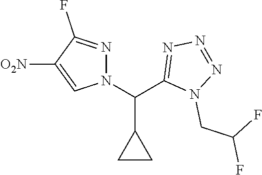 [SMILES: O=[N+]([O-])c1cn(C(c2nnnn2CC(F)F)C2CC2)nc1F] | 5-[cyclopropyl-(3-fluoro-4-nitro-pyrazol- 1-yl)methyl]-1-(2,2- difluoroethyl)tetrazole |
| 75 | B | 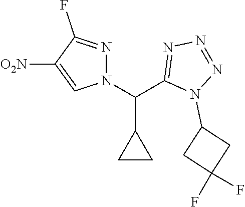 [SMILES: O=[N+]([O-])c1cn(C(c2nnnn2C2CC(F)(F)C2)C2CC2)nc1F] | 5-[cyclopropyl-(3-fluoro-4-nitro-pyrazol- 1-yl)methyl]-1-(3,3- difluorocyclobutyl)tetrazole |
| 76 | B | 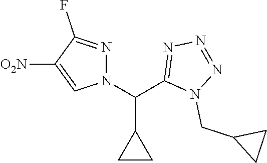 [SMILES: O=[N+]([O-])c1cn(C(c2nnnn2CC2CC2)C2CC2)nc1F] | 5-[cyclopropyl-(3-fluoro-4-nitro-pyrazol- 1-yl)methyl]-1- (cyclopropylmethyl)tetrazole |
| 77 | B | 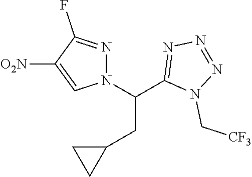 [SMILES: O=[N+]([O-])c1cn(C(CC2CC2)c2nnnn2CC(F)(F)F)nc1F] | 5-[2-cyclopropyl-1-(3-fluoro-4-nitro- pyrazol-1-yl)ethyl]-1-(2,2,2- trifluoroethyl)tetrazole |
| 78 | B | 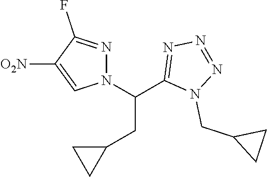 [SMILES: O=[N+]([O-])c1cn(C(CC2CC2)c2nnnn2CC2CC2)nc1F] | 5-[2-cyclopropyl-1-(3-fluoro-4-nitro- pyrazol-1-yl)ethyl]-1- (cyclopropylmethyl)tetrazole |

### Preparation 79: 9H-fluoren-9-ylmethyl N-[(1S)-1-(tert-butoxymethyl)-2-[[l-[³,3-difluoro-1-[1-(2,2,2-trifluoroethyl)tetrazol-5-yl]propyl]-3-fluoro-pyrazol-4-yl]amino]-2-oxo-ethyl]carbamate

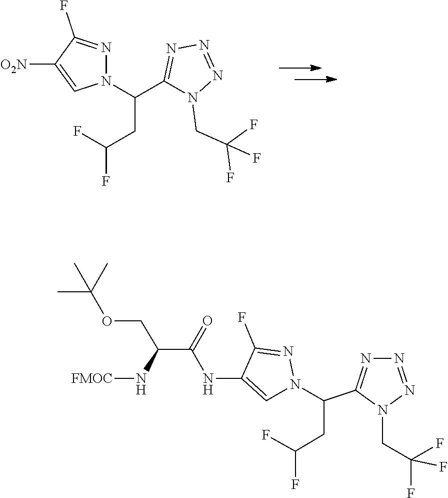

According to the methods of Preparations 11 and 12 the nitro compound of Preparation 55 was reacted with (2S)-3-tert-butoxy-2-(9H-fluoren-9-ylmethoxycarbonylamino)propanoic acid to give the title compound (52 mg, 77% Yield). LCMS (METHOD 4) (ES): m/z 695.3 [M+H]⁺, RT=0.85 min.

### Preparation 80: (2S)-2-amino-3-tert-butoxy-N-[1-[3,3-difluoro-1-[1-(2,2,2-trifluoroethyl)tetrazol-5-yl]propyl]-3-fluoro-pyrazol-4-yl]propanamide

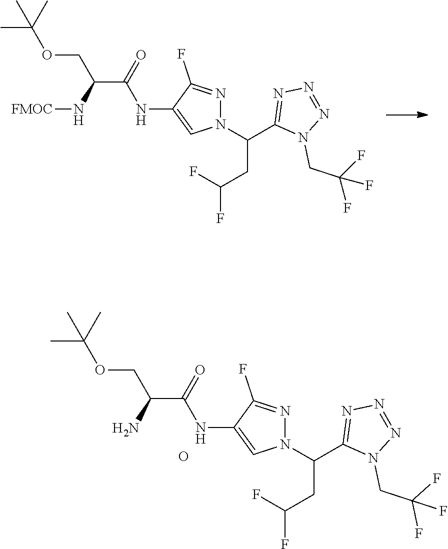

Piperidine (1 mL) was added to a solution of the compound of Preparation 79 (52 mg, 0.0749 mmol) in DCM (2 mL) and the mixture was stirred at room temperature for 30 minutes. The solvent was removed in vacuo, the residue was dissolved in DMF (1 mL) and MeOH (2 mL) and purified by prep. basic to give the title compound (15 mg, 42% Yield). LCMS (METHOD 4) (ES): m/z 473.2 [M+H]⁺, RT=0.63 min.

### Preparation 81: methyl (2S)-2-(benzyloxycarbonylamino)-3-(2,2,2-trifluoro-1,1-dimethyl-ethoxy)propanoate

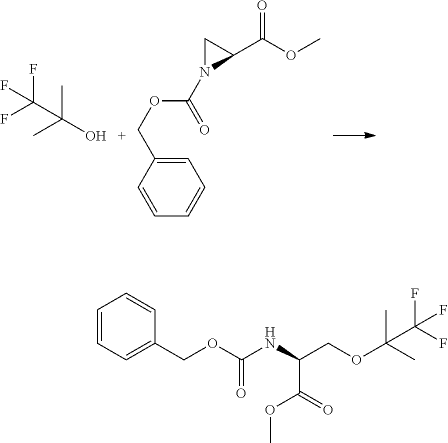

To a solution of 01-benzyl 02-methyl (2S)-aziridine-1,2-dicarboxylate (2 g, 8.50 mmol) in 1,1,1-trifluoro-2-methyl-propan-2-ol (10 g, 78.1 mmol) and DCM (10 mL) was added boron trifluoride diethyl etherate (0.1 mL) at room temperature under N₂. The solution was stirred at room temperature in a sealed vial for 1 hour. then quenched with sat aq. sodium bicarbonate (10 mL). The mixture was extracted with DCM (2×20 mL). The combined organic phases were dried over MgSO₄ and concentrated in vacuo. The residue was purified by flash chromatography (heptane/ethyl acetate 2:1) to give the title compound (380 mg, 12% Yield) as a colourless oil. ¹H NMR (600 MHz, CDCl₃) δ 7.41-7.30 (m, 6H), 5.59 (d, J=8.9 Hz, 1H), 5.19-5.10 (m, 2H), 4.52 (dt, J=8.9, 2.9 Hz, 1H), 4.01 (dd, J=9.0, 2.8 Hz, 1H), 3.76 (s, 3H), 1.30 (s, 6H).

### Preparation 82: (2S)-2-(benzyloxycarbonylamino)-3-(2,2,2-trifluoro-1,1-dimethyl-ethoxy)propanoic acid

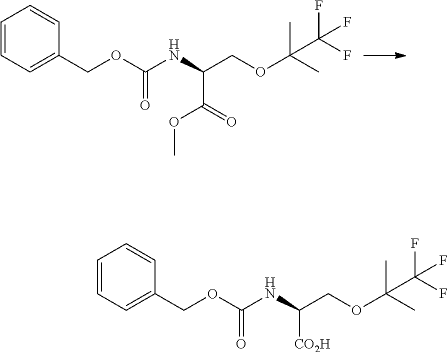 [SMILES: COC(=O)[C@H](COC(C)(C)C(F)(F)F)NC(=O)OCc1ccccc1 ; CC(C)(OC[C@H](NC(=O)OCc1ccccc1)C(=O)O)C(F)(F)F]

To a solution of the ester of Preparation 81 (77 mg, 0.212 mmol) in THF (2 mL) and water (2 mL) was added LiOH (10 mg, 0.418 mmol) at room temperature. The solution was stirred at room temperature for 2 hours then concentrated in vacuo. The residue was redissolved in water (10 mL), acidified to pH 2 and extracted with DCM (3×15 mL). The combined organic phases were dried over Na₂SO₄ and concentrated in vacuo, giving the title compound (70 mg, 95% Yield) as a colourless oil.

### Preparation 83: diethyl 2-(2,2-difluoroethyl)propanedioate

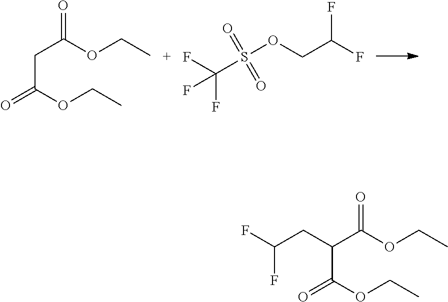

Sodium hydride (4.61 g, 115 mmol) was added in small portions to a solution of diethyl malonate (18.5 g, 115 mmol) in dry THF (300 mL) at 10° C. under N₂. The mixture was stirred for 30 min at 10° C., then 2,2-difluoroethyl trifluoromethanesulfonate (25.0 g, 117 mmol) was added (slightly exothermic). The mixture was stirred overnight at 60° C. under N₂. The clear yellow solution was cooled to room temperature, sat. aq. NH₄Cl (50 mL) and water (20 mL) were added and the layers were separated. The organic layer was dried (Na₂SO₄) and evaporated. Purification by column chromatography (EtOAc:heptane) gave the title compound (13.1 g, 51% Yield) as a colourless oil containing approximately 20% of the dialkylated product. ¹H NMR (400 MHz, CDCl₃) δ 5.97 (tt, J=56.4, 4.5 Hz, 1H), 4.33-4.15 (m, 4H), 3.58 (t, J=7.2 Hz, 1H), 2.47 (tdd, J=16.9, 7.2, 4.3 Hz, 2H), 1.28 (t, J=7.1 Hz, 6H); GCMS (EI): m/z 224.1 [M]+, RT=7.40 min.

### Preparation 84: 2-ethoxycarbonyl-4,4-difluoro-butanoic acid

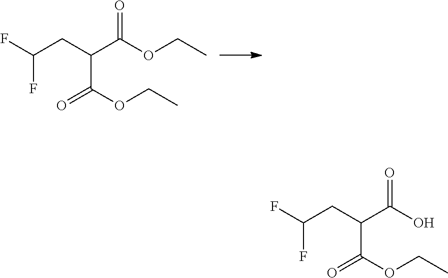

A mixture of the ester of Preparation 83 (9.91 g, 44.2 mmol) and NaOH (1.90 g, 47.5 mmol) in EtOH/water (60 mL:20 mL) was stirred overnight at room temperature. The reaction mixture was concentrated in vacuo, diluted with water (60 ml) and extracted with pentane (to remove most of the dialkylated biproduct from the previous step). The aq. layer was adjusted to pH=3 with 5M HCl, extracted with ether (3×60 mL) and the combined organic extracts were dried (Na₂SO₄) and evaporated to give the title compound (7.25 g, 84% Yield) as a clear oil. ¹H NMR (400 MHz, CDCl₃) δ 9.11 (br s, 1H), 5.99 (tt, J=56.2, 4.2 Hz, 1H), 4.26 (q, J=7.1 Hz, 2H), 3.67 (t, J=7.1 Hz, 1H), 2.58-2.42 (m, 2H), 1.30 (t, J=7.1 Hz, 3H).

### Preparation 85: ethyl 4,4-difluoro-2-(2,2,2-trifluoroethylcarbamoyl)butanoate

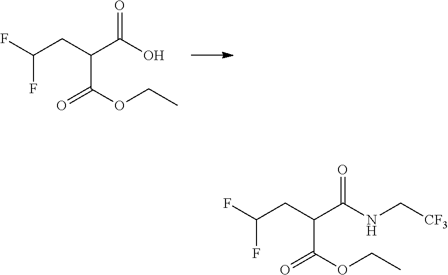 [SMILES: CCOC(=O)C(CC(F)F)C(=O)O ; CCOC(=O)C(CC(F)F)C(=O)NCC(F)(F)F]

Oxalyl chloride (4.69 mL, 7.04 g, 55.4 mmol) was added slowly over 5 min to a solution of the acid of Preparation 84 (7.25 g, 37.0 mmol) and DMF (1 drop) in DCM (50 mL) at room temperature and the mixture was stirred for 2 hours. The reaction mixture was concentrated in vacuo, redissolved in DCM (10 mL) and added dropwise to a solution of 2,2,2-trifluoroethylamine (4.41 mL, 5.49 g, 55.4 mmol) and DIPEA (19.3 mL, 14.3 g, 111 mmol) in DCM (150 mL) at 5-10° C. under N₂. The yellow solution was stirred at room temperature for 1 hour. The reaction mixture was washed with 1M HCl (80 mL), dried (Na₂SO₄) and concentrated in vacuo. Purification by column chromatography (EtOAc:heptane) gave the title compound (7.93 g, 77% Yield) as an orange solid. ¹H NMR (400 MHz, CDCl₃) δ 6.90 (s, 1H), 5.95 (tt, J=56.5, 4.3 Hz, 1H), 4.31-4.18 (m, 2H), 4.08-3.80 (m, 2H), 3.52 (t, J=7.0 Hz, 1H), 2.61-2.42 (m, 2H), 1.29 (t, J=7.1 Hz, 3H).

### Preparation 86: ethyl 4,4-difluoro-2-[1-(2,2,2-trifluoroethyl)tetrazol-5-yl]butanoate

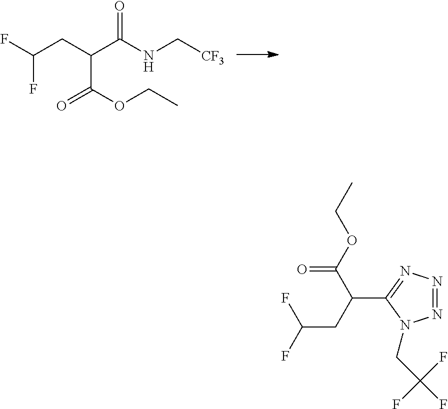 [SMILES: CCOC(=O)C(CC(F)F)C(=O)NCC(F)(F)F ; CCOC(=O)C(CC(F)F)c1nnnn1CC(F)(F)F]

According to the method of Preparation 40 the amide of Preparation 85 was reacted to give the title compound (1.64 g, 80% Yield) as a yellow oil. ¹H NMR (400 MHz, CDCl₃) δ 6.02 (tt, J=56.1, 4.1 Hz, 1H), 5.22-5.04 (m, 2H), 4.35-4.12 (m, 3H), 3.08-2.75 (m, 2H), 1.25 (t, J=7.1 Hz, 3H).

### Preparation 87: 4,4-difluoro-2-[1-(2,2,2-trifluoroethyl)tetrazol-5-yl]butanoic acid

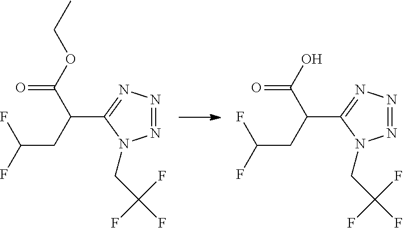 [SMILES: CCOC(=O)C(CC(F)F)c1nnnn1CC(F)(F)F ; O=C(O)C(CC(F)F)c1nnnn1CC(F)(F)F]

A solution of NaOH (295 mg, 7.38 mmol) in water (4 mL) was added to a solution of the ester of Preparation 86 (1.64 g, 5.44 mmol) in MeOH (20 mL) and the mixture was stirred at room temperature for 18 hours. The reaction mixture was neutralised with 5M HCl, concentrated in vacuo and then acidified to pH=3 with further 5M HCl. The mixture was extracted with ether (2×40 mL), dried (Na₂SO₄) and evaporated to give the title compound (1.45 g, 97% Yield) as an orange oil. ¹H NMR (400 MHz, CDCl₃) δ 6.02 (tt, J=56.0, 4.0 Hz, 1H), 5.91 (br s, 1H), 5.22-5.07 (m, 2H), 4.26 (dd, J=8.4, 5.8 Hz, 1H), 3.03-2.73 (m, 2H).

### Preparation 88: 6,6-difluoro-3-oxo-4-[1-(2,2,2-trifluoroethyl)tetrazol-5-yl]hexanenitrile

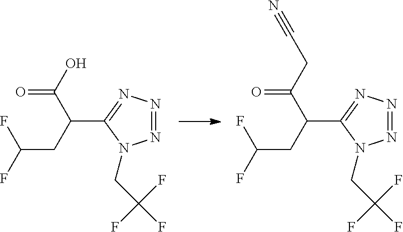 [SMILES: O=C(O)C(CC(F)F)c1nnnn1CC(F)(F)F ; N#CCC(=O)C(CC(F)F)c1nnnn1CC(F)(F)F]

Oxalyl chloride (1340 mg, 0.895 mL, 10.6 mmol) was added to a solution of the acid of Preparation 87 (1450 mg, 5.29 mmol) and DMF (1 drop) in DCM (20 mL) at room temperature. The mixture was stirred for 1 hour at room temperature, then all the volatiles were removed in vacuo to give the crude acid chloride.

Isopropylmagnesium chloride (2M solution in THF, 15.9 mL, 31.8 mmol) was added slowly to a solution of 2-cyanoacetic acid (1350 mg, 15.9 mmol) in dry THF (50 mL) at −50° C. under N₂. The solution was stirred for 30 min, then the acid chloride from above in dry THF (8 mL) was added slowly. The cooling bath was removed and the yellow mixture was stirred for 1 hour at room temperature. The reaction was quenched with 1M HCl (25 mL, to pH=3-4), the layers were separated and the THF layer was dried (Na₂SO₄) and evaporated to give the title compound (1500 mg, 95% Yield) as an orange oil. LCMS (METHOD 4) (ES): m/z 296.0 [M−H]⁻, RT=0.37 min.

### Preparation 89: 3-[3,3-difluoro-1-[1-(2,2,2-trifluoroethyl)tetrazol-5-yl]propyl]isoxazol-5-amine

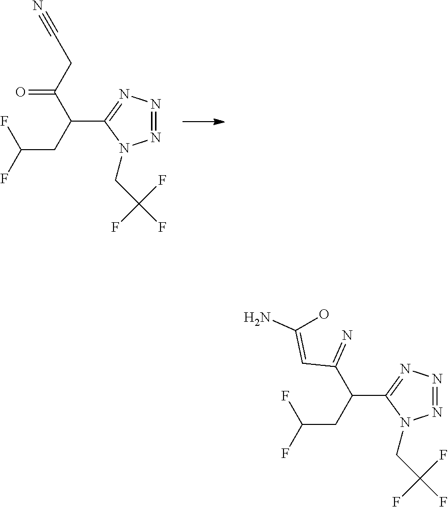 [SMILES: N#CCC(=O)C(CC(F)F)c1nnnn1CC(F)(F)F ; N=C(/C=C(/N)O)C(CC(F)F)c1nnnn1CC(F)(F)F]

A mixture of the compound of Preparation 88 (1500 mg, 5.05 mmol), hydroxylamine-HCl (1050 mg, 15.1 mmol) and KOAc (1490 mg, 15.1 mmol) in MeOH (25 mL) was stirred overnight at room temperature. The resulting yellow suspension was evaporated and the residue was neutralised with sat. aq. NaHCO₃ and extracted with ether (2×40 mL). The combined organic extracts were dried (Na₂SO₄) and evaporated and the residue was purified by column chromatography (EtOAc:heptane) to give the title compound (896 mg, 57% Yield) as a pale yellow oil. ¹H NMR (400 MHz, CDCl₃) δ 5.96 (tt, J=56.0, 4.5 Hz, 1H), 5.12-5.00 (m, 3H), 4.70 (br s, 2H), 4.62-4.52 (m, 1H), 3.10-2.92 (m, 1H), 2.78-2.61 (m, 1H); LCMS (METHOD 4) (ES): m/z 311.0 [M−H]⁻, RT=0.49 min.

### Preparation 90: (4-nitrophenyl) (2S)-2-(tert-butoxycarbonylamino)-3,3-dicyclopropyl-propanoate

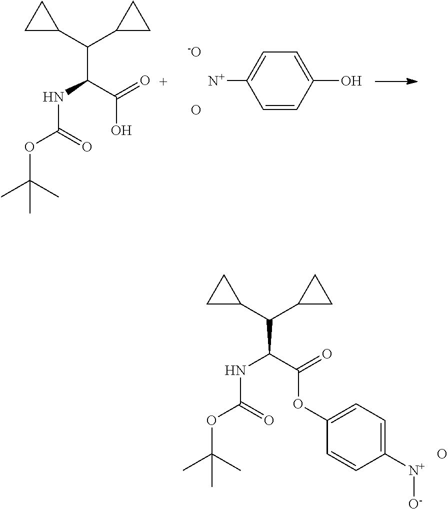

DIC (0.520 g, 0.64 mL, 4.117 mmol) was added to a solution of the acid of Preparation 8 (1.01 g, 3.74 mmol) and 4-nitrophenol (0.547 g, 3.93 mmol) in DCM (9 mL) at room temperature and stirred for 2 hours to give a yellow suspension. The solvent was removed in vacuo and the residue was purified by column chromatography to give the title compound (0.91 g, 62% Yield) as a clear oil.

### Preparation 91: tert-butyl N-[(1S)-1-(dicyclopropylmethyl)-2-[[3-[3,3-difluoro-1-[1-(2,2,2-trifluoroethyl)tetrazol-5-yl]propyl]isoxazol-5-yl]amino]-2-oxo-ethyl]carbamate

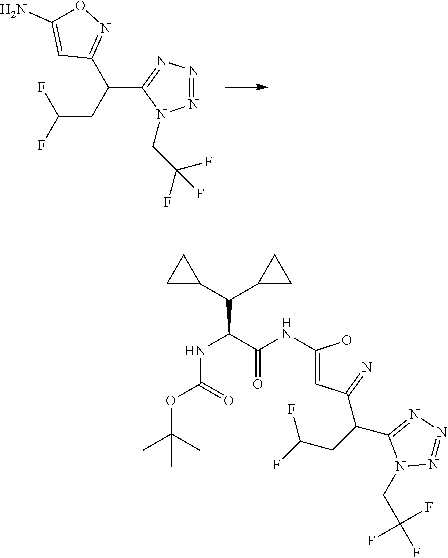 [SMILES: Nc1cc(C(CC(F)F)c2nnnn2CC(F)(F)F)no1 ; CC(C)(C)OC(=O)N[C@H](C(=O)N/C(O)=C/C(=N)C(CC(F)F)c1nnnn1CC(F)(F)F)C(C1CC1)C1CC1]

tert-Butylmagnesium chloride in THF (1M, 0.487 mL, 0.487 mmol) was added dropwise to a solution of the compound of Preparation 90 (47.5 mg, 0.122 mmol) and the compound of Preparation 89 (38 mg, 0.122 mmol) in dry THF (4 mL) under N₂ at 5° C. The yellow solution was stirred at room temperature for 2 hours, then quenched by adding AcOH (0.1 mL). The reaction mixture was concentrated in vacuo and the residue was purified by basic prep. HPLC to give the title compound (28 mg, 41% Yield) as a beige solid. LCMS (METHOD 4) (ES): m/z 562.2 [M−H]⁻, RT=0.77 min.

### Preparation 92: 2-[(4R)-2,2-dimethyl-5-oxo-1,3-dioxolan-4-yl]acetaldehyde

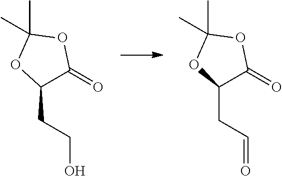

(5R)-5-(2-hydroxyethyl)-2,2-dimethyl-1,3-dioxolan-4-one (2.0 g, 12.5 mmol) was dissolved in DCM (80 mL) and cooled to 0° C. PCC (13.5 g, 62.4 mmol) was added in one portion and the cooling bath was removed. The reaction mixture was stirred at room temperature overnight then filtered through a short pad of silica gel (20 g) and washed with heptane/ethyl acetate (1:1). The dark filtrate was again filtered through a short pad of silica gel to remove the colour and the pad was washed with heptane/ethyl acetate (1:1) until no product was detected. The filtrate was concentrated in vacuo, to give the title compound (1.35 g, 68% Yield) as a reddish oil, which was used directly in the next step without characterisation.

### Preparation 93: (5R)-5-(2,2-difluoroethyl)-2,2-dimethyl-1,3-dioxolan-4-one

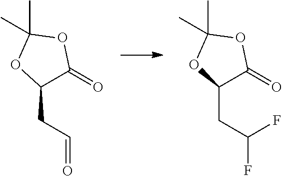

DAST (1.2 mL, 1.50 g, 9.1 mmol) was slowly added to a solution of the aldehyde of Preparation 92 (1.35 g, 8.54 mmol) in DCM (15 mL) at 5° C. The solution was stirred at room temperature overnight then quenched with sat. aq. NaHCO₃ solution (10 mL). The mixture was extracted with DCM (3×20 mL). The combined organic phases were dried over MgSO₄ and concentrated in vacuo. The residue was purified by flash chromatography (heptane/ethyl acetate 3:1) to give the title compound (0.67 g, 44% Yield) as a brown oil. ¹H NMR (400 MHz, CDCl₃) δ 5.96 (tdd, J=56.1, 5.5, 4.1 Hz, 1H), 4.48 (ddd, J=8.5, 4.1, 1.1 Hz, 1H), 2.48-2.31 (m, 1H), 2.28-2.10 (m, 1H), 1.57 (s, 3H), 1.50 (s, 3H).

### Preparation 94: (2R)-4,4-difluoro-2-hydroxy-butanehydrazide

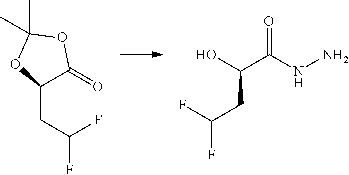

To the compound of Preparation 93 (0.71 g, 3.9 mmol) was added hydrazine hydrate (3 mL) at room temperature. The mixture was stirred at room temperature for 30 min, then concentrated in vacuo and dried in vacuo at 60° C. for 1 hour. The residue was purified by column chromatography (DCM/MeOH 10:1), to give the title compound (0.423 g, 70% Yield) as an oil, which was used directly in the next step.

### Preparation 95: (1R)-3,3-difluoro-1-[4-(2,2,2-trifluoroethyl)-1,2,4-triazol-3-yl]propan-1-ol

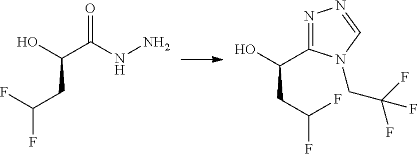 [SMILES: NNC(=O)[C@H](O)CC(F)F ; O[C@H](CC(F)F)c1nncn1CC(F)(F)F]

The acyl hydrazide of Preparation 94 (423 mg, 2.74 mmol) and DMF dimethyl acetal (0.4 mL, 400 mg, 3 mmol) were dissolved in MeCN (20 mL). The solution was stirred at 50° C. for 30 min then 2,2,2-trifluoroethanamine (544 mg, 5.49 mmol) and acetic acid (2 mL) were added. The solution was then stirred for 2 hours at 120° C. in a sealed vial. After cooling to room temperature the reaction was concentrated in vacuo. The residue was diluted with water (30 mL) and basified to pH8 using sat aq. Na₂CO₃. The mixture was extracted with ethyl acetate (3×30 mL) and the combined organic phases were dried over MgSO₄ and concentrated in vacuo. The residue was purified by column chromatography (ethyl acetate). Further purification by column chromatography (DCM/MeOH 10:1) gave the title compound (0.350 g, 52% Yield) as a white solid. ¹H NMR (600 MHz, CDCl₃) δ 8.16 (s, 1H), 6.15 (tt, J=56.3, 4.5 Hz, 1H), 5.10 (dd, J=9.1, 4.3 Hz, 1H), 4.97 (dq, J=16.6, 8.4 Hz, 1H), 4.73 (dq, J=16.6, 8.4 Hz, 1H), 2.75-2.44 (m, 2H).

### Preparation 96: 3-[(1S)-3,3-difluoro-1-(3-fluoro-4-nitro-pyrazol-1-yl)propyl]-4-(2,2,2-trifluoroethyl)-1,2,4-triazole

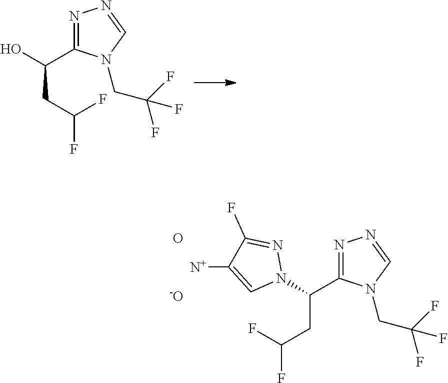

To a solution of 3-fluoro-4-nitro-1H-pyrazole (176 mg, 1.35 mmol), the alcohol of Preparation 95 (330 mg, 1.35 mmol) and triphenylphosphine (530 mg, 2.02 mmol) at room temperature was added DIAD (408 mg, 2.02 mmol). The solution was stirred at room temperature for 1 hour and then concentrated in vacuo. The residue was purified by acidic prep. HPLC to give the title compound (273 mg, 57% Yield) as yellow solid. ¹H NMR (400 MHz, DMSO) δ 9.23 (d, J=1.2 Hz, 1H), 8.73 (s, 1H), 6.50-5.80 (m, 2H), 5.19 (m, 2H), 3.15 (dtdd, J=18.9, 14.9, 8.9, 4.1 Hz, 1H), 2.96 (qt, J=15.2, 5.0 Hz, 1H).

### Preparations 97-101

Preparations 97-101 were synthesised according to the methods of Preparations 95 and 96 from the appropriate acyl hydrazide and primary amine.

| Prep. | | | |
|---|---|---|---|
| No. | LCMS details | Structure | Name |
| 97 | LCMS (METHOD 3) (ES): m/z 291.3 [M + H]⁺, RT = 0.50 min. | 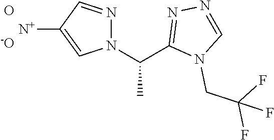 | 3-[(1S)-1-(4- nitropyrazol-1-yl)ethyl]- 4-(2,2,2-trifluoroethyl)- 1,2,4-triazole |
| 98 | LCMS (METHOD 3) (ES): m/z 291.4 [M + H]⁺, RT = 0.50 min. | 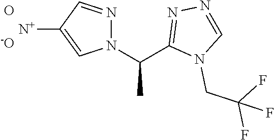 | 3-[(1R)-1-(4- nitropyrazol-1-yl)ethyl]- 4-(2,2,2-trifluoroethyl)- 1,2,4-triazole |
| 99 | LCMS (METHOD 2) (ESI): m/z: 323.2 [M + H]⁺; RT = 1.87 min (ACQUITY BEH C18 (50 mm × 2.1 mm) column, 0.05% Formic acid in water, 0.05% Formic acid in MeCN). | 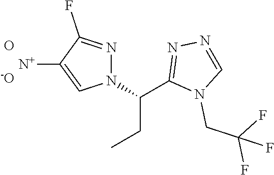 | 3-[(1S)-1-(3-fluoro-4- nitro-pyrazol-1- yl)propyl]-4-(2,2,2- trifluoroethyl)-1,2,4- triazole |
| 100 | LCMS (METHOD 2) (ESI): m/z: 323.3 [M + H]⁺; RT = 1.87 min (ACQUITY BEH C18 (50 mm × 2.1 mm) column, 0.05% Formic acid in water, 0.05% Formic acid in MeCN). | 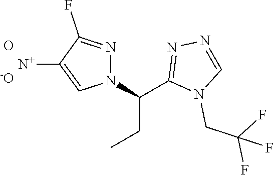 | 3-[(1R)-1-(3-fluoro-4- nitro-pyrazol-1- yl)propyl]-4-(2,2,2- trifluoroethyl)-1,2,4- triazole |
| 101 | LCMS (METHOD 4) (ES): m/z 329.1 [M − H]⁻, RT = 0.55 min. | 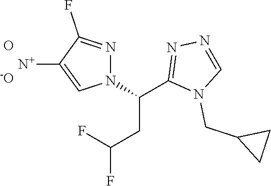 | 4-(cyclopropylmethyl)- 3-[(1S)-3,3-difluoro-1- (3-fluoro-4-nitro- pyrazol-1-yl)propyl]- 1,2,4-triazole |

### Preparation 102: methyl 1-(3-fluoro-4-nitro-pyrazol-1-yl)cyclopropanecarboxylate

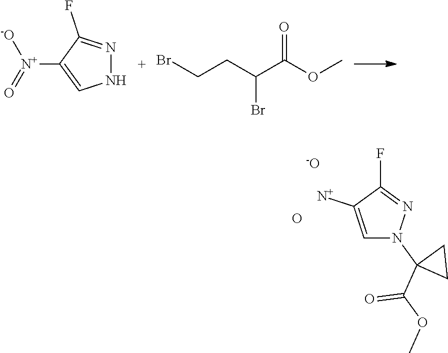

To a stirred solution of 3-fluoro-4-nitro-1H-pyrazole (1.7 g, 13 mmol) in MeCN (15 mL) under N₂ at 0° C. was added K₂CO₃ (5.4 g, 39 mmol). After being stirred for 10 min methyl 2,4-dibromobutanoate (4.0 g, 16 mmol) was added dropwise at 0° C., then the reaction mixture was stirred at room temperature for 16 hours. The reaction mixture was diluted with ice water (100 mL) and the aqueous layer was extracted with EtOAc (100 mL). The organic layer was washed with sat. aq. NaCl (50 mL), dried over anhydrous Na₂SO₄, filtered and concentrated in vacuo. The crude compound was purified by column chromatography (EtOAc\hexane) to give the title compound (1.8 g, 61%/Yield). LCMS (METHOD 2) (ESI): m/z: 230.2 [M+H]⁺; RT=1.88 min (ACQUITY BEH C18 (50 mm×2.1 mm) column, 0.05% Formic acid in water, 0.05% Formic acid in MeCN).

### Preparation 103: 1-(3-fluoro-4-nitro-pyrazol-1-yl)cyclopropanecarboxylic acid

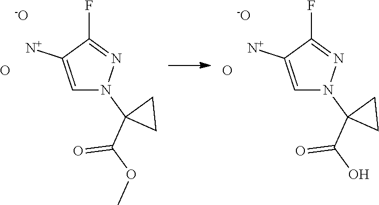

According to the method of Preparation 82 the ester of Preparation 102 was reacted to give the title compound (1.0 g, 59% Yield). LCMS (METHOD 2) (ESI): m/z: 214.3 [M−H]⁻; RT=1.41 min (ACQUITY BEH C18 (50 mm×2.1 mm) column, 0.05% Formic acid in water, 0.05% Formic acid in MeCN).

### Preparation 104: tert-butyl N-[[1-(3-fluoro-4-nitro-pyrazol-1-yl)cyclopropanecarbonyl]-amino]carbamate

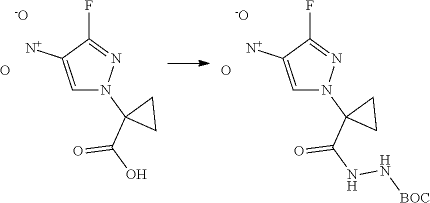

According to the method of Preparation 12 the acid of Preparation 103 was reacted with tert-butyl N-aminocarbamate to give the title compound (900 mg, 59% Yield) after purification by column chromatography (EtOAc/hexane). LCMS (METHOD 2) (ESI): m/z: 328.4 [M−H]⁻; RT=1.66 min (ACQUITY BEH C18 (50 mm×2.1 mm) column, 0.05% Formic acid in water, 0.05% Formic acid in MeCN).

### Preparation 105: 1-(3-fluoro-4-nitro-pyrazol-1-yl)cyclopropanecarbohydrazide

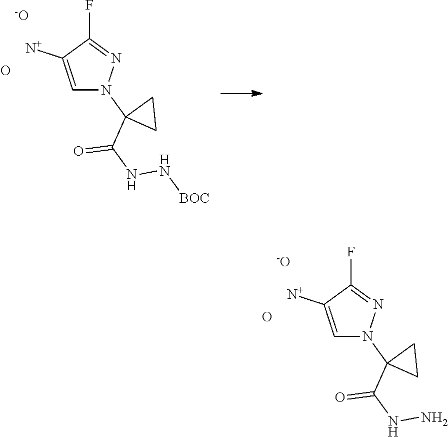

According to the method of Preparation 13 the compound of Preparation 104 was reacted to give the title compound. LCMS (METHOD 2) (ESI): m/z: 230.1 [M+H]⁺; RT=1.45 min (ACQUITY BEH C18 (50 mm×2.1 mm) column, 0.05% Formic acid in water, 0.05% Formic acid in MeCN).

### Preparation 106: 3-[1-(3-fluoro-4-nitro-pyrazol-1-yl)cyclopropyl]-4-(2,2,2-trifluoroethyl)-1,2,4-triazole

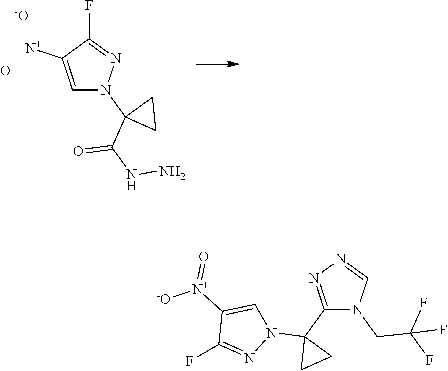

According to the method of Preparation 95 the compound of Preparation 105 was reacted to give the title compound. LCMS (METHOD 2) (ESI): m/z: 321.3 [M+H]⁺; RT=1.81 min (ACQUITY BEH C18 (50 mm×2.1 mm) column, 0.05% Formic acid in water, 0.05% Formic acid in MeCN).

### Preparation 107: 3-fluoro-1-[1-[4-(2,2,2-trifluoroethyl)-1,2,4-triazol-3-yl]cyclopropyl]-pyrazol-4-amine

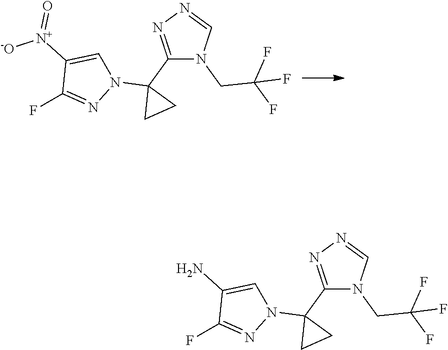

According to the method of Preparation 11 the compound of Preparation 106 was reacted to give the title compound. LCMS (METHOD 2) (ESI): m/z: 291.3 [M+H]⁺; RT=1.47 min (ACQUITY BEH C18 (50 mm×2.1 mm) column, 0.05% Formic acid in water, 0.05% Formic acid in MeCN).

### Preparation 108: (2,3,4,5,6-pentafluorophenyl) (2S)-2-(benzyloxycarbonylamino)-3,3-dicyclopropyl-propanoate

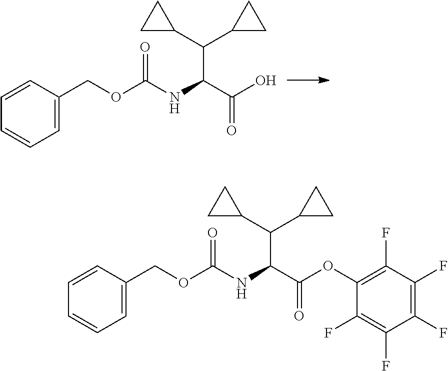 [SMILES: O=C(N[C@H](C(=O)O)C(C1CC1)C1CC1)OCc1ccccc1 ; O=C(N[C@H](C(=O)Oc1c(F)c(F)c(F)c(F)c1F)C(C1CC1)C1CC1)OCc1ccccc1]

To a solution of the compound of Preparation 5 (1.6 g, 5.3 mmol) and (2,3,4,5,6-pentafluorophenyl) 2,2,2-trifluoroacetate (1.05 g, 3.75 mmol) in DCM (25 mL) was added TEA (1 mL) at room temperature. The solution was stirred at room temperature for 4 hours then concentrated in vacuo. The residue was purified by column chromatography (heptane/ethyl acetate 6:1) to give the title compound (1.28 g, 73% Yield) as colourless oil which was used without characterisation.

### Preparation 109: benzyl N-[(1S)-1-(dicyclopropylmethyl)-2-[[3-fluoro-1-[1-[4-(2,2,2-trifluoroethyl)-1,2,4-triazol-3-yl]cyclopropyl]pyrazol-4-yl]amino]-2-oxo-ethyl]carbamate

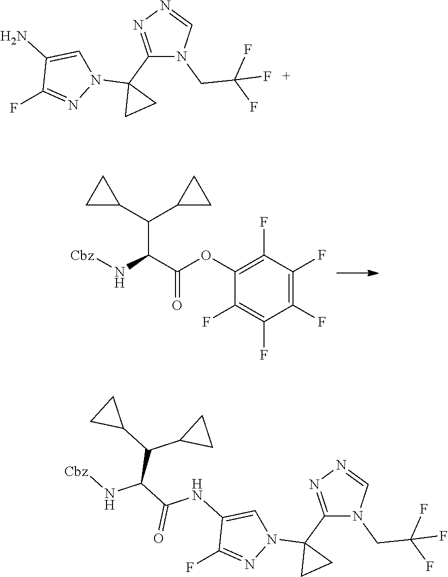

To a stirred solution of the compound of Preparation 107 (79 mg, 0.272 mmol) and the compound of Preparation 108 (153 mg, 0.327 mmol) in THF (5 mL) at 0° C. under N₂, was added tert-butyl magnesium chloride (1M soln. in THF, 0.82 mL, 0.82 mmol). After stirring for 10 min at 0° C. the reaction was stirred at room temperature for 1 hour. The reaction was concentrated in vacuo and ice water (10 mL) was added. The mixture was extracted with DCM (2×100 mL). The combined organic layer was washed with NaCl solution (10 mL) dried over anhydrous Na₂SO₄, filtered and concentrated to give crude title compound (150 mg) as a brown gum that was used without further purification. LCMS (METHOD 2) (ESI): m/z: 574.6 [M−H]⁻; RT=1.96 min (ACQUITY BEH C18 (50 mm×2.1 mm) column, 0.05% Formic acid in water, 0.05% Formic acid in MeCN).

### Preparation 110: ethyl 1-(4-nitropyrazol-1-yl)cyclopropanecarboxylate

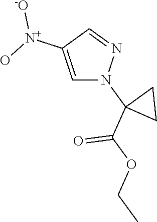

According to the method of Preparation 102 4-nitropyrazole was reacted with ethyl 2,4-dibromobutanoate to give the title compound. LCMS (METHOD 2) (ESI): m/z: 226.2 [M+H]⁺; RT=1.62 min (ACQUITY BEH C18 (50 mm×2.1 mm) column, 0.05% Formic acid in water, 0.05% Formic acid in MeCN).

### Preparation 111: 3-[1-(4-nitropyrazol-1-yl)cyclopropyl]-4-(2,2,2-trifluoroethyl)-1,2,4-triazole

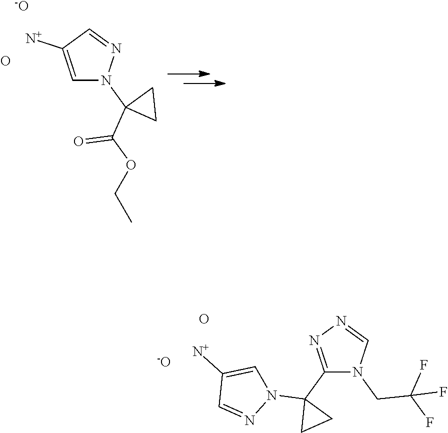

According to the methods of Preparation 103, 104, 105 and 106 the compound of Preparation 110 was converted to the title compound. LCMS (METHOD 2) (ESI): m/z: 303.3 [M+H]⁺; RT=1.45 min (ACQUITY BEH C18 (50 mm×2.1 mm) column, 0.05% Formic acid in water, 0.05% Formic acid in MeCN).

### Preparation 112: 5-[1-(4-nitropyrazol-1-yl)cyclopropyl]-1-(2,2,2-trifluoroethyl)tetrazole

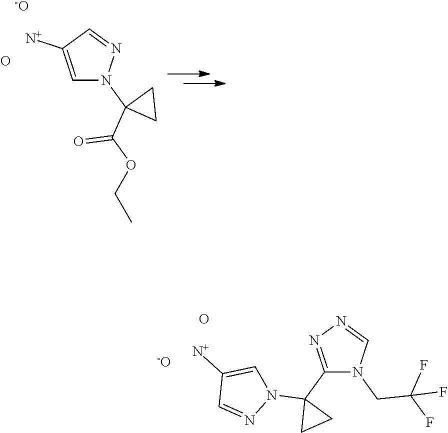

According to the methods of Preparation the compound of Preparation 110 was converted to the title compound. LCMS (METHOD 2) (ESI): m/z: 304.3 [M+H]⁺; RT=1.94 min (ACQUITY BEH C18 (50 mm×2.1 mm) column, 0.05% Formic acid in water, 0.05% Formic acid in MeCN).

### Preparation 113: methyl (2E)-2-(dimethylaminomethylene)-5,5,5-trifluoro-3-oxo-pentanoate

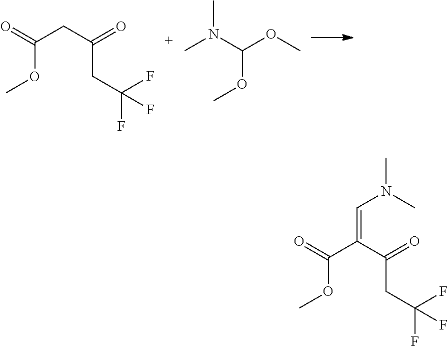

A solution of methyl 5,5,5-trifluoro-3-oxo-pentanoate (1.84 g, 9.99 mmol) and DMF dimethyl acetal (1.2 g, 10 mmol) was heated at 70° C. for 2 hours. The solution was concentrated in vacuo, giving the title compound (assumed quantitative yield) as a pink oil, which was used in the next step without further purification. ¹H NMR (600 MHz, CDCl₃) δ 7.83 (s, 1H), 3.75 (s, 3H), 3.70 (q, J=10.8 Hz, 2H), 3.31 (s, 3H), 2.87 (s, 3H).

### Preparation 114: methyl 3-(2,2,2-trifluoroethyl)-1H-pyrazole-4-carboxylate

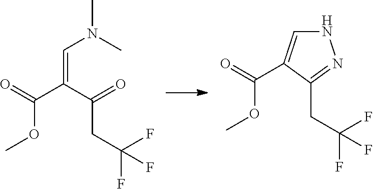 [SMILES: COC(=O)/C(=C/N(C)C)C(=O)CC(F)(F)F ; COC(=O)c1c[nH]nc1CC(F)(F)F]

To a solution of the compound of Preparation 113 (9.99 mmol) in EtOH (20 mL) at room temperature was added hydrazine hydrate (500 mg, 9.99 mmol). The solution was stirred at room temperature overnight then concentrated in vacuo. The residue was purified by column chromatography (ethyl acetate), giving the title compound (1.65 g, 79% Yield) as a yellow solid. ¹H NMR (600 MHz, CDCl₃) δ 8.07 (s, 1H), 3.93 (q, J=10.5 Hz, 2H), 3.87 (s, 3H); LCMS (METHOD 3) (ES): m/z 207.3 [M−H]⁻, RT=0.58 min.

### Preparation 115: methyl 3-(2,2,2-trifluoroethyl)-1-(2-trimethylsilylethoxymethyl)pyrazole-4-carboxylate and methyl 5-(2,2,2-trifluoroethyl)-1-(2-trimethylsilylethoxymethyl)pyrazole-4-carboxylate

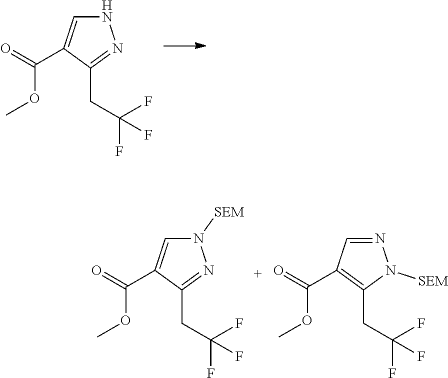

SEM chloride (1.60 g, 9.4 mmol) was added to a mixture of the compound of Preparation 114 (1.5 g, 7.2 mmol) and caesium carbonate (3.50 g, 11 mmol) in DMF (15 mL) at room temperature. The mixture was stirred at room temperature for 3 hours then partitioned between TBME (50 mL) and water (50 mL). The aqueous phase was extracted with TBME (50 mL) and the combined organic phases were dried over MgSO₄ and concentrated in vacuo. The residue was purified by column chromatography (heptane/ethyl acetate 7:1) to give a mixture of the title compounds (1.65 g, 68% Yield). LCMS (METHOD 3) (ES): m/z 339.5 [M+H]⁺, RT=0.97 and 0.99 min.

### Preparation 116: 1-[3-(2,2,2-trifluoroethyl)-1-(2-trimethylsilylethoxymethyl)pyrazol-4-yl]propan-1-one and 1-[5-(2,2,2-trifluoroethyl)-1-(2-trimethylsilylethoxymethyl)pyrazol-4-yl]propan-1-one

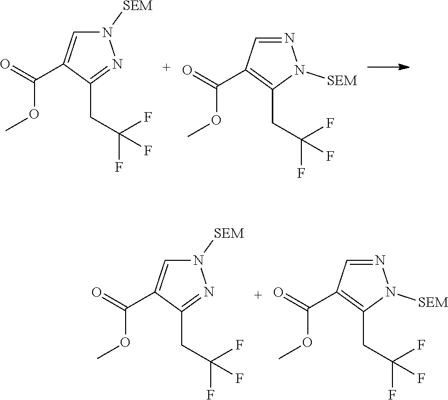

To a stirred solution of ethyl magnesium bromide (3M soln. in diethyl ether, 2.22 mL., 6.66 mmol) in toluene (15 mL, 100 mass %) at −30° C. was added TEA (3.5 mL, 25 mmol). After 15 min, a solution of the compounds of Preparation 115 (750 mg, 2.22 mmol) in toluene (5 mL) was added and the reaction was stirred for 1 hour at −10° C. and then at 5° C. for 4 hours. The reaction was quenched with sat. aq. ammonium chloride solution (20 mL) and extracted with TBME (3×30 mL). The organic extracts were combined, dried (MgSO₄), filtered and concentrated, giving a mixture of the title compounds (assumed quantitative yield) which was used directly in the next step. LCMS (METHOD 3) (ES): m/z 337.5 [M+H]⁺, RT=0.97 and 1.00 min.

### Preparation 117: 1-[3-(2,2,2-trifluoroethyl)-1-(2-trimethylsilylethoxymethyl)pyrazol-4-yl]propan-1-ol and 1-[5-(2,2,2-trifluoroethyl)-1-(2-trimethylsilylethoxymethyl)pyrazol-4-yl]propan-1-ol

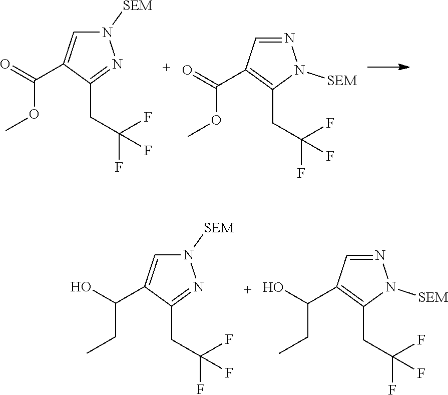

To a solution of the compounds of Preparation 116 (2.22 mmol) in MeOH (10 mL) at room temperature was added sodium borohydride (84 mg, 2.22 mmol) in several portions. The reaction mixture was stirred at room temperature for 2 hours and then purified directly by acidic prep. HPLC to give a mixture of the title compounds (495 mg, 66% Yield) as a colourless oil. LCMS (METHOD 3) (ES): m/z 339.6 [M+H]⁺, RT=0.90 and 0.92 min.

### Preparation 118: 2-[[4-[1-(3-fluoro-4-nitro-pyrazol-1-yl)propyl]-3-(2,2,2-trifluoroethyl)pyrazol-1-yl]methoxy]ethyl-trimethyl-silane and 2-[[4-[1-(3-fluoro-4-nitro-pyrazol-1-yl)propyl]-5-(2,2,2-trifluoroethyl)pyrazol-1-yl]methoxy]ethyl-trimethyl-silane

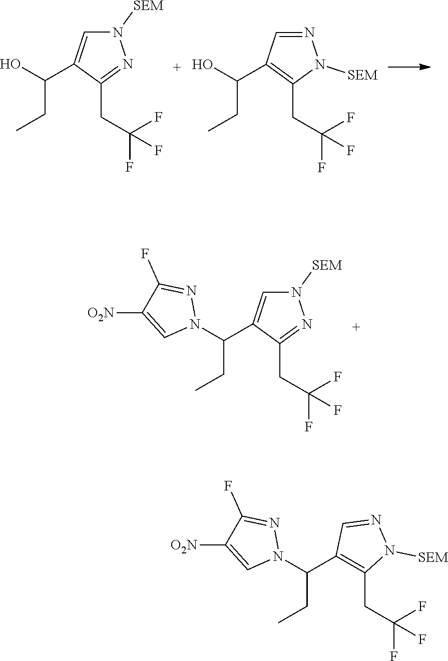

According to the method of Preparation 96 the alcohols of Preparation 117 were reacted with 3-fluoro-4-nitro-1H-pyrazole to give a mixture of the title compounds.

### Preparation 119: cyclopropyl (2,5-dioxopyrrolidin-1-yl) carbonate

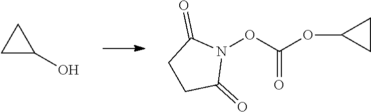

According to the method of Preparation 19 cyclopropanol was reacted to give the title compound.

### Preparation 120: ethyl 5-methyl-2-(2-trimethylsilylethoxymethyl)pyrazole-3-carboxylate

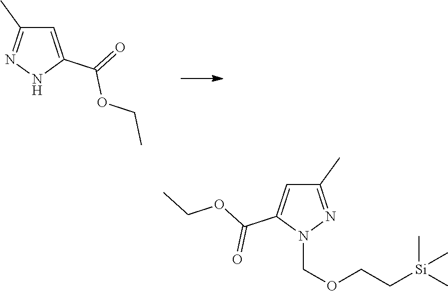 [SMILES: CCOC(=O)c1cc(C)n[nH]1 ; CCOC(=O)c1cc(C)nn1COCC[Si](C)(C)C]

SEM-chloride (2.89 g, 17.3 mmol) was added to a mixture of ethyl 3-methyl-1H-pyrazole-5-carboxylate (2.67 g, 17.3 mmol) and caesium carbonate (5.64 g, 17.3 mmol) in dry DMF (20 mL) at room temperature and the mixture was stirred for 3 hours. The mixture was diluted with water (200 mL), extracted with ether (2×50 mL) and the combined extracts were dried (Na₂SO₄) and evaporated. Purification by column chromatography gave the title compound (1.58 g, 32% Yield) as a clear oil. ¹H NMR (400 MHz, CDCl₃) δ 6.73 (s, 1H), 5.81 (s, 2H), 4.38 (q, J=7.1 Hz, 2H), 3.68-3.58 (m, 2H), 2.33 (s, 3H), 1.41 (t, J=7.1 Hz, 3H), 1.01-0.85 (m, 2H), 0.00 (s, 9H); LCMS (METHOD 4) (ES): m/z 283 [M−H]⁻, RT=0.88 min.

### Preparation 121: [5-methyl-2-(2-trimethylsilylethoxymethyl)pyrazol-3-yl]methanol

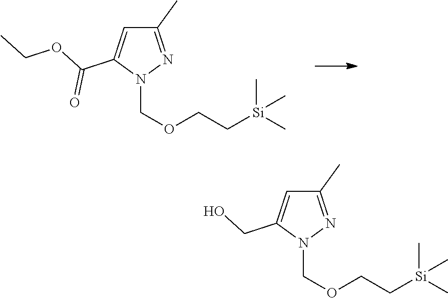 [SMILES: CCOC(=O)c1cc(C)nn1COCC[Si](C)(C)C ; Cc1cc(CO)n(COCC[Si](C)(C)C)n1]

LiAIH₄ (1M soln. in THF, 5.56 mL, 5.56 mmol) was added dropwise to a solution of the compound of Preparation 120 (1.58 g, 5.56 mmol) in dry ether (60 mL) at 10° C. under N₂.

The mixture was stirred for 30 min at room temperature, then carefully quenched with sat. aq. NH₄Cl (20 mL). The layers were separated and the ether layer was dried (Na₂SO₄) and evaporated to give the title compound (1.27 g, 94% Yield) as a clear oil. LCMS (METHOD 3) (ES): m/z 243.2 [M+H]⁺, RT=0.65 min.

### Preparation 122: trimethyl-[2-[[3-methyl-5-[(4-nitropyrazol-1-yl)methyl]pyrazol-1-yl]methoxy]ethyl]silane

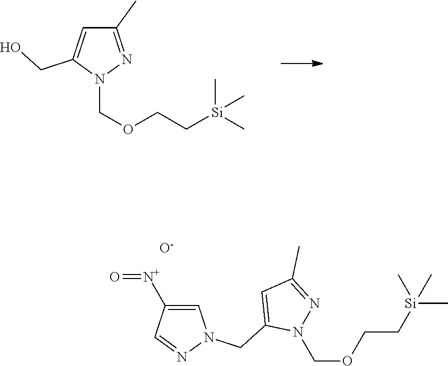

According to the method of Preparation 59 the alcohol of Preparation 121 was reacted with 4-nitropyrazole to give the title compound (1.23 g, 70% Yield) as a colourless oil. ¹H NMR (400 MHz, CDCl₃) δ 8.17 (s, 1H), 8.10 (s, 1H), 6.23 (s, 1H), 5.44 (s, 2H), 5.43 (s, 2H), 3.61-3.47 (m, 2H), 2.29 (s, 3H), 0.93-0.80 (m, 2H), 0.00 (s, 9H); LCMS (METHOD 3) (ES): m/z 338.4 [M+H]⁺, RT=0.78 min.

### Preparation 123: N-[(1S)-1-(dicyclopropylmethyl)-2-[[1-[[5-methyl-2-(2-trimethylsilylethoxymethyl)pyrazol-3-yl]methyl]pyrazol-4-yl]amino]-2-oxo-ethyl]-2-isopropyl-pyrazole-3-carboxamide

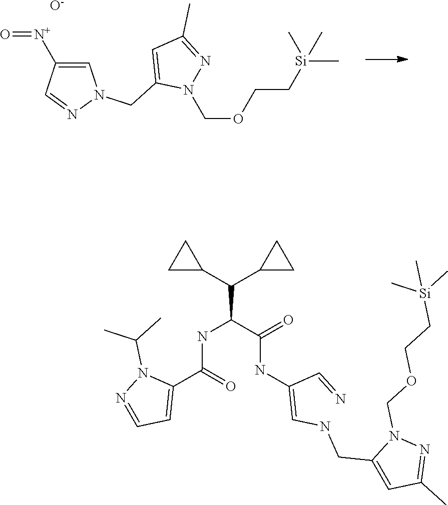

According to the methods of Preparations 11, 23, 24 and 25 the compound of Preparation 122 was reacted to give the title compound (80 mg) as an orange oil. LCMS (METHOD 3) (ES): m/z 595.5 [M+H]⁺, RT=0.84 min.

### Preparation 124: ethyl 3,5-dimethyl-1-(2-trimethylsilylethoxymethyl)pyrazole-4-carboxylate

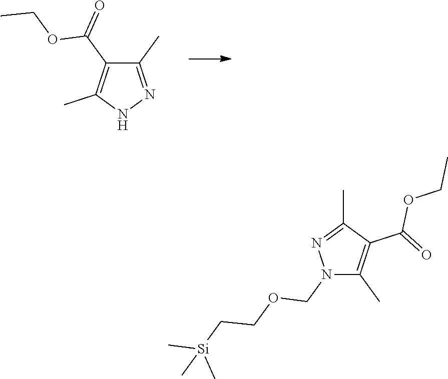 [SMILES: CCOC(=O)c1c(C)n[nH]c1C ; CCOC(=O)c1c(C)nn(COCC[Si](C)(C)C)c1C]

According to the method of Preparation 120 ethyl 3,5-dimethyl-1H-pyrazole-4-carboxylate was reacted with SEM chloride to give the title compound (0.30 g, 30% Yield) as a colourless oil. LCMS (METHOD 3) (ES): m/z 299.3 [M+H]⁺, RT=0.92 min.

### Preparation 125: [3,5-dimethyl-1-(2-trimethylsilylethoxymethyl)pyrazol-4-yl]methanol

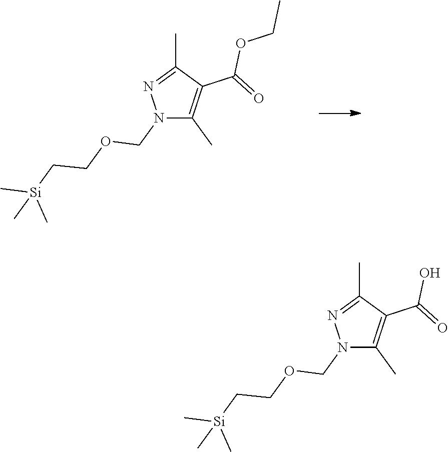

DIBAL (25 wt % in toluene, 2.0 g, 3.52 mmol) was added dropwise to a solution of the compound of Preparation 124 (0.30 g, 1.0 mmol) in THF (10 mL) at 0° C. The reaction was warmed to room temperature and stirred for 1 hour. The reaction was quenched with 1M HCl (10 mL) and extracted with TBME (3×10 mL). The combined organic phases were dried over MgSO₄ and concentrated in vacuo. The residue was purified by column chromatography (heptane/ethyl acetate 1:1), to give the title compound (135 mg, 52% Yield) as a colourless oil. LCMS (METHOD 3) (ES): m/z 257.2 [M+H]⁺, RT=0.67 min.

### Preparation 126: lithium 4-(2,2,2-trifluoroethyl)isoxazole-3-carboxylate

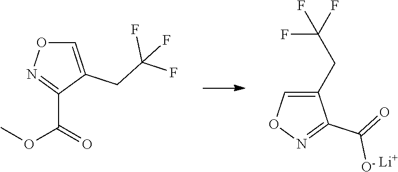

To methyl 4-(2,2,2-trifluoroethyl)isoxazole-3-carboxylate (1080 mg, 5.16 mmol) in MeOH (4 mL) was added LiOH (148 mg, 6.20 mmol) in water (1 mL). After stirring at room temperature for 10 minutes the reaction mixture was concentrated in vacuo and dried further in vacuo to give the title compound (1040 mg, 100% Yield). LCMS (METHOD 4) (ES): m/z 150.0 [M−H−CO₂]⁻, RT=0.21 min.

### Preparation 127: N-methoxy-N-methyl-4-(2,2,2-trifluoroethyl)isoxazole-3-carboxamide

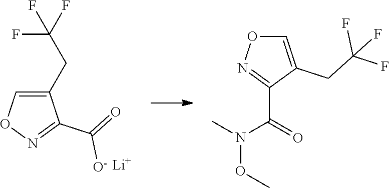 [SMILES: O=C([O-])c1nocc1CC(F)(F)F ; [Li+] ; CON(C)C(=O)c1nocc1CC(F)(F)F]

To a solution of the compound of Preparation 126 (1040 mg, 5.16 mmol), N-methoxymethanamine (605 mg, 6.20 mmol) and DIPEA (801 mg, 1.08 mL, 6.20 mmol) in DMF (10 mL) was added HATU (2160 mg, 5.68 mmol). The mixture was stirred at room temperature for 2 hours then diluted with 20 ml of EtOAc and 10 ml of water. The layers were separated and the aqueous phase was extracted with EtOAc (20 mL). The combined organic phases were washed with sat. aq. NaHCO₃ (10 mL), water (10 mL), 10% aq. NaHSO₄ (10 mL) and brine (10 mL), dried (MgSO₄), filtered and concentrated in vacuo. The residue was purified by column chromatography (heptane:EtOAc) to give the title compound (772 mg, 63% Yield) as a colourless oil. ¹H NMR (400 MHz, DMSO-d6) δ 9.15 (s, 1H), 3.84-3.50 (m, 5H), 3.30 (s, 3H); GCMS (EI): m/z 238.1 [M]+, RT=8.01 min.

### Preparation 128: 1-[4-(2,2,2-trifluoroethyl)isoxazol-3-yl]propan-1-one

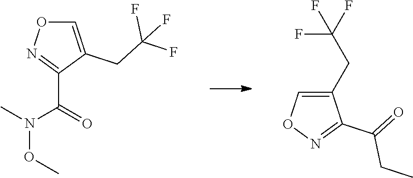 [SMILES: CON(C)C(=O)c1nocc1CC(F)(F)F ; CCC(=O)c1nocc1CC(F)(F)F]

To a solution of the compound of Preparation 127 (380 mg, 1.60 mmol) in THF (4 mL) under N₂ at 0° C. was added ethyl magnesium bromide (3M soln. in diethyl ether, 0.80 mL, 2.40 mmol) dropwise. The mixture was stirred 30 min at 0° C. then quenched with of sat. aq. NH₄Cl (2 mL). The layers were separated and the aqueous layer was extracted with THF (2×5 mL). The combined organic phases were dried (MgSO₄), filtered and concentrated in vacuo to give the title compound (149 mg, 45% Yield) as a yellowish oil. ¹H NMR (600 MHz, DMSO-d6) δ 9.20 (s, 1H), 3.87-3.69 (m, 2H), 3.07 (q, J=7.3 Hz, 2H), 1.09 (t, J=7.2 Hz, 3H); GCMS (EI): m/z 207.1 [M]+, RT=6.20 min.

### Preparation 129: 1-[4-(2,2,2-trifluoroethyl)isoxazol-3-yl]propan-1-ol

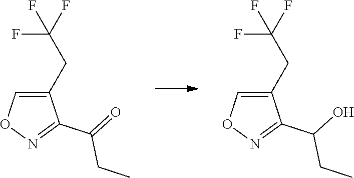

To the compound of Preparation 128 (140 mg, 0.676 mmol) dissolved in EtOH (4 mL) was added sodium borohydride (15 mg, 0.41 mmol) and the mixture was stirred at room temperature for 45 minutes. The reaction was quenched with 5 mL of sat. aq. NH₄Cl, most of the ethanol was removed in vacuo, and the mixture was extracted with of EtOAc (2×10 mL). The combined organic phases were washed with brine (2 mL), dried (MgSO₄), filtered and concentrated in vacuo to give the title compound (105 mg, 74% Yield) as a yellowish oil. GCMS (EI): m/z 209.1 [M]+, RT=7.31 min.

### Preparation 130: 3-[1-(3-fluoro-4-nitro-pyrazol-1-yl)propyl]-4-(2,2,2-trifluoroethyl)isoxazole

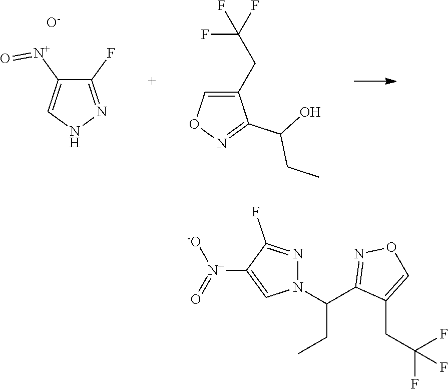

According to the Method of Preparation 96 the compound of Preparation 129 was reacted with 3-fluoro-4-nitro-1H-pyrazole to give the title compound (58 mg, 38% Yield). ¹H NMR (400 MHz, CDCl₃) δ 8.49 (s, 1H), 8.19 (d, J=1.2 Hz, 1H), 5.27 (t, J=7.8 Hz, 1H), 3.46-3.26 (m, 2H), 2.48-2.34 (m, 2H), 1.01 (t, J=7.4 Hz, 3H).

Preparations 131-134 were synthesised according to the methods of Preparations 127-130 from the indicated carboxylic acids, using methyl magnesium bromide instead of ethyl magnesium bromide.

| Prep. | | | | |
|---|---|---|---|---|
| No. | LCMS details | Starting acid | Structure | Name |
| 131 | LCMS (METHOD 3) (ES): m/z no mass ion, RT = 0.63 min. | 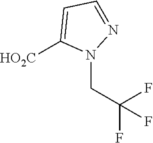 [SMILES: O=C(O)c1ccnn1CC(F)(F)F] | 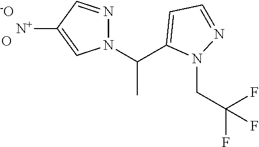 | 5-[1-(4-nitro- pyrazol-1-yl)ethyl]- 1-(2,2,2-trifluoro- ethyl)pyrazole |
| 132 | LCMS (METHOD 3) (ES): m/z no mass ion, RT = 0.57 min | 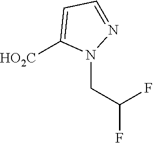 [SMILES: O=C(O)c1ccnn1CC(F)F] | 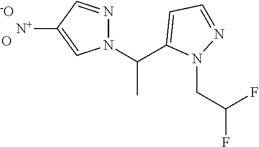 | 1-(2,2-difluoro- ethyl)-5-[1-(4- nitropyrazol-1- yl)ethyl]pyrazole |
| 133 | LCMS (METHOD 3) (ES): m/z no mass ion, RT = 0.79 min | 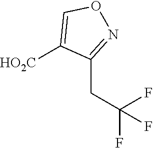 [SMILES: O=C(O)c1conc1CC(F)(F)F] | 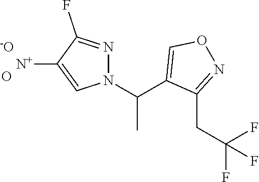 | 4-[1-(3-fluoro-4- nitro-pyrazol-1- yl)ethyl]-3-(2,2,2- trifluoroethyl)isoxazole |
| 134 | LCMS (METHOD 3) (ES): m/z 289.3 [M − H]⁻, RT = 0.69 min. | 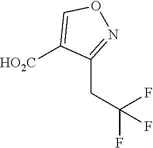 [SMILES: O=C(O)c1conc1CC(F)(F)F] | 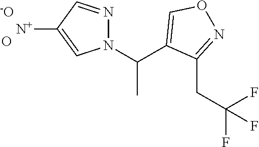 | 4-[1-(4-nitro- pyrazol-1-yl)ethyl]- 3-(2,2,2-trifluoro- ethyl)ixoazole |

### Preparation 135: N-[(15)-1-(dicyclopropylmethyl)-2-oxo-2-[(1-prop-2-ynylpyrazol-4-yl)amino]ethyl]-2-isopropyl-pyrazole-3-carboxamide

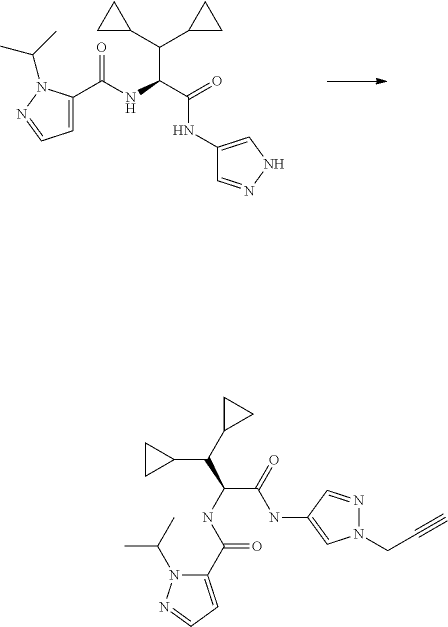 [SMILES: CC(C)n1nccc1C(=O)N[C@H](C(=O)Nc1cn[nH]c1)C(C1CC1)C1CC1 ; C#CCn1cc(NC(=O)[C@@H](NC(=O)c2ccnn2C(C)C)C(C2CC2)C2CC2)cn1]

To a stirred solution of the compound of Preparation 26 (500 mg, 1.35 mmol) in DMF (10 ml) at 0° C. was added TEA (0.19 mL, 136 mg, 1.35 mmol) and K₂CO₃ (373 mg, 2.70 mmol), followed by propargyl bromide (193 mg, 1.62 mmol). The reaction mixture was stirred at 80° C. for 16 hours, then diluted with water (50 mL) and extracted with DCM (2×50 mL). The combined organic layer was dried over anhydrous Na₂SO₄, filtered and concentrated under reduced pressure. The obtained crude compound was purified by column chromatography (40-60%/EtOAc in hexane) to give the title compound (200 mg, 36%/Yield) as a colourless solid. LCMS (METHOD 2) (ESI): m/z: 409.5 [M+H]⁺; RT=1.81 min (ACQUITY BEH C18 (50 mm×2.1 mm) column, 0.050/Formic acid in water, 0.050/Formic acid in MeCN).

### Preparation 136: N-[(1S)-1-(dicyclopropylmethyl)-2-[[1-(1-methylprop-2-ynyl)pyrazol-4-yl]amino]-2-oxo-ethyl]-2-isopropyl-pyrazole-3-carboxamide

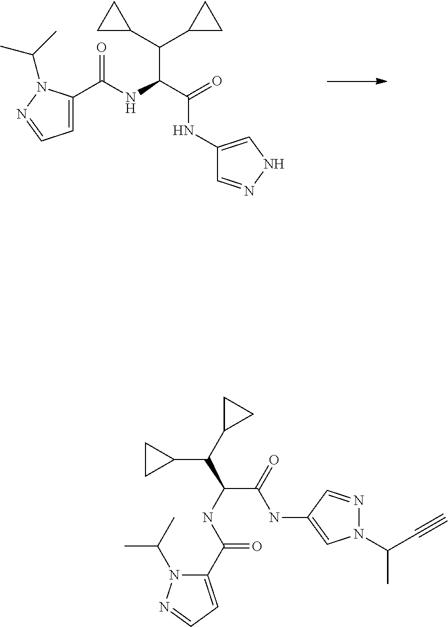 [SMILES: CC(C)n1nccc1C(=O)N[C@H](C(=O)Nc1cn[nH]c1)C(C1CC1)C1CC1 ; C#CC(C)n1cc(NC(=O)[C@@H](NC(=O)c2ccnn2C(C)C)C(C2CC2)C2CC2)cn1]

According to the method of Preparation 135 the compound of Preparation 26 was reacted with 1-methylprop-2-ynyl methanesulfonate to give the title compound (190 mg, 29% Yield) as a colourless solid. LCMS (METHOD 2) (ESI): m/z: 423.4 [M+H]⁺; RT=1.84 min (ACQUITY BEH C18 (50 mm×2.1 mm) column, 0.05% Formic acid in water, 0.05% Formic acid in MeCN).

### Preparation 137: 2-azido-1,1,1-trifluoro-propane

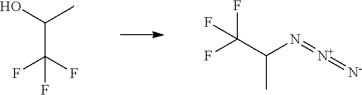

To a stirred solution of 1,1,1-trifluoropropan-2-ol (2.5 g, 22 mmol) in diethyl ether (200 mL) at 0° C. was added TEA (9.2 mL, 6.6 g, 66 mmol and then tosyl chloride (8.4 g, 44 mmol) portion-wise. The reaction was stirred for 48 hours at room temperature, then filtered, washing with diethyl ether. The filtrate was concentrated under reduced pressure and the residue was purified by column chromatography (2-3% diethyl ether in pet. ether) to give (2,2,2-trifluoro-1-methyl-ethyl) 4-methylbenzenesulfonate (2.0 g, 34% Yield) as a colourless viscous liquid.

The tosylate (2 g, 7.46 mmol) was dissolved in DMSO (10 mL) and NaN₃ (2.42 g, 37.3 mmol) was added at room temperature. The mixture was stirred for 16 hours at 120° C. then ice water was added and the mixture was extracted with diethyl ether. The organic layer was dried over Na₂SO₄ and this diethyl ether solution was used directly without further purification.

### Preparation 138: 1-cyclopropyl-4-trimethylsilyl-but-3-yn-2-one

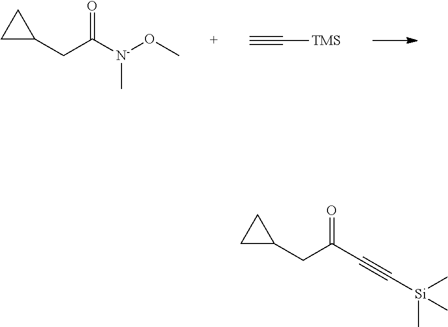

ⁿBuLi (2.7M soln in hexanes, 11 mL, 30.7 mmol) was added dropwise over 10 min to a solution of ethynyl(trimethyl)silane (2740 mg, 3.87 mL, 27.9 mmol) in dry THF (40 mL) at −40° C. to −45° C. The mixture was stirred for 15 min at −40° C., after which a solution of 2-cyclopropyl-N-methoxy-N-methyl-acetamide (4000 mg, 27.9 mmol) in dry THF (30 mL) was added dropwise over 15 min at −40° C. to −50° C. The mixture was stirred and allowed to warm to room temperature over 2 h. The reaction was quenched by the addition of sat. aq.

NH₄Cl (50 mL) and the layers were separated. The aqueous phase was extracted with EtOAc 82×30 mL) and the combined organic phases were dried (MgSO₄), filtered and concentrated in vacuo. Purification by column chromatography (heptane/EtOAc) gave the title compound (1910 mg, 32% Yield) as a yellowish oil containing ˜20% of the desilylated material, 1-cyclopropylbut-3-yn-2-one.

### Preparation 139: 2-cyclopropyl-1-[3-(2,2,2-trifluoroethyl)triazol-4-yl]ethanone

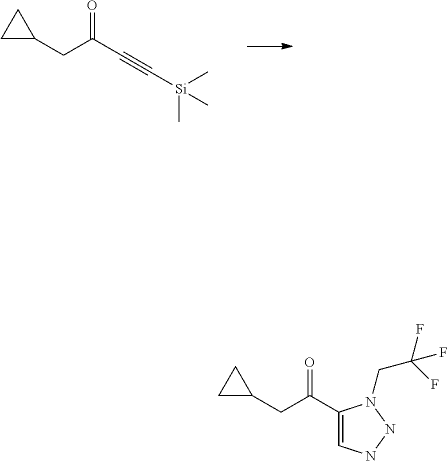 [SMILES: C[Si](C)(C)C#CC(=O)CC1CC1 ; O=C(CC1CC1)C1=CNNN1CC(F)(F)F]

A mixture of 2-azido-1,1,1-trifluoro-ethane (0.5M soln. in TBME, 11 mL, 5.5 mmol), the compound of Preparation 138 (830 mg, 4.6 mmol) and chloro(1,5-cyclooctadiene)-(pentamethylcyclopentadienyl)ruthenium (II) (35 mg, 0.092 mmol) in toluene (5 mL) was heated under N₂ in a microwave vial at 60° C. for 1 hour. After cooling, the reaction mixture was concentrated in vacuo and purified by column chromatography to give the title compound (82 mg, 8%). ¹H NMR (400 MHz, CDCl₃) δ 7.94 (s, 1H), 5.26 (q, J=8.0 Hz, 2H), 2.57 (d, J=6.9 Hz, 2H), 1.01-0.71 (m, 1H), 0.45-0.36 (m, 2H), −0.01 (dt, J=6.2, 4.9 Hz, 2H); LCMS (METHOD 3) (ES): m/z 234.3 [M+H]⁺, RT=0.68 min.

### Preparation 140: 5-[2-cyclopropyl-1-(3-fluoro-4-nitro-pyrazol-1-yl)ethyl]-1-(2,2,2-trifluoroethyl)triazole

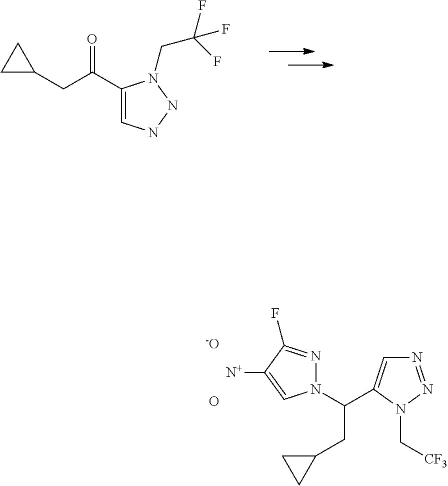

According to the methods of Preparations 129 and 96 the compound of Preparation 139 was converted to the title compound (41 mg). ¹H NMR (400 MHz, CDCl₃) δ 8.07 (s, 1H), 7.91 (s, 1H), 5.50-5.35 (m, 1H), 5.23-5.09 (m, 1H), 5.08-4.90 (m, 1H), 2.21 (m, 2H), 0.66-0.44 (m, 3H), 0.16 (m, 2H); LCMS (METHOD 3) (ES): m/z 349.4 [M+H]⁺, RT=0.76 min.

### Preparation 141: 3,3-difluoro-1-[3-(2,2,2-trifluoroethyl)triazol-4-yl]propan-1-ol

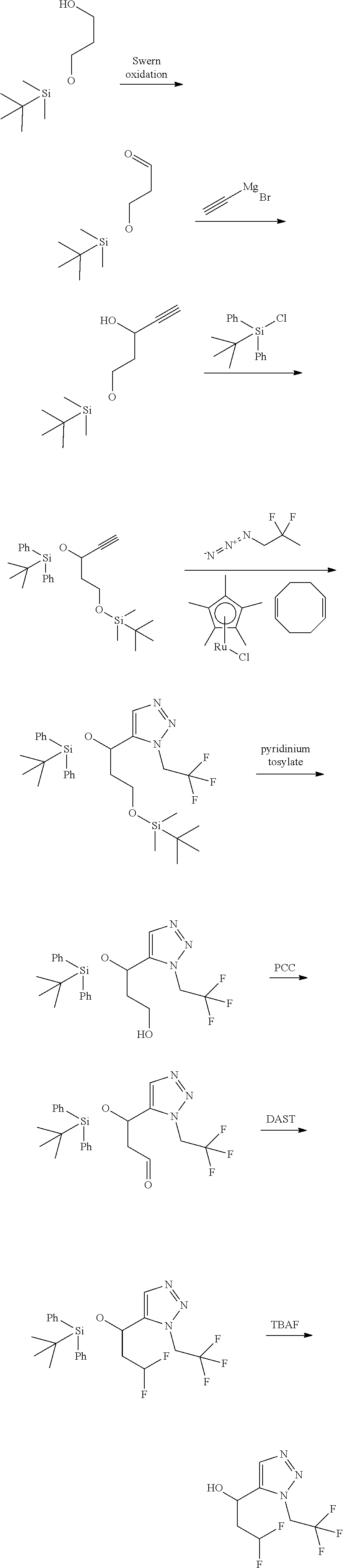

The title compound (87 mg) was synthesised as a colourless oil as outlined in the scheme above. ¹H NMR (600 MHz, CDCl₃) δ 7.55 (s, 1H), 6.13 (tdd, J=56.0, 5.7, 3.1 Hz, 1H), 5.27-5.19 (m, 2H), 5.13 (dq, J=15.2, 8.4 Hz, 1H), 3.36 (br s, 1H), 2.60-2.34 (m, 2H).

### Preparation 142: 5-[3,3-difluoro-1-(3-fluoro-4-nitro-pyrazol-1-yl)propyl]-1-(2,2,2-trifluoroethyl)triazole

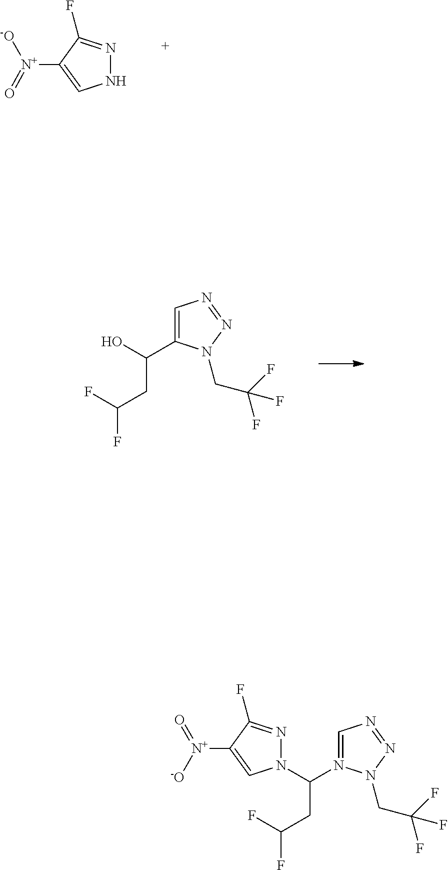

According to the Method of Preparation 96 the compound of Preparation 141 was reacted with 3-fluoro-4-nitro-1H-pyrazole to give the title compound (58 mg, 38% Yield). ¹H NMR (600 MHz, CDCl₃) δ 8.23 (d, J=1.0 Hz, 1H), 8.00 (s, 1H), 5.89 (tt, J=55.0, 3.7 Hz, 1H), 5.77 (dd, J=8.8, 5.7 Hz, 1H), 5.19-5.03 (m, 2H), 3.14-2.98 (m, 1H), 2.81-2.65 (m, 1H).

### Preparation 143: 4-methyl-5-[(4-nitropyrazol-1-yl)methyl]-1-(2,2,2-trifluoroethyl)triazole

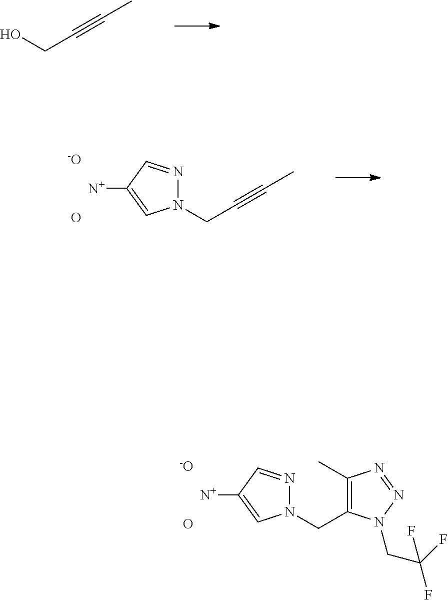

The title compound was synthesised from propargyl alcohol and 4-nitropyrazole according to the methods of Preparations 96 and 31. ¹H NMR (400 MHz, CDCl₃) δ 8.22 (s, 1H), 8.07 (s, 1H), 5.44 (s, 2H), 5.28 (q, J=8.3 Hz, 2H), 2.42 (s, 3H).

### Preparation 144: 5-[1-(3-fluoro-4-nitro-pyrazol-1-yl)ethyl]-1-(2,2,2-trifluoroethyl)triazole

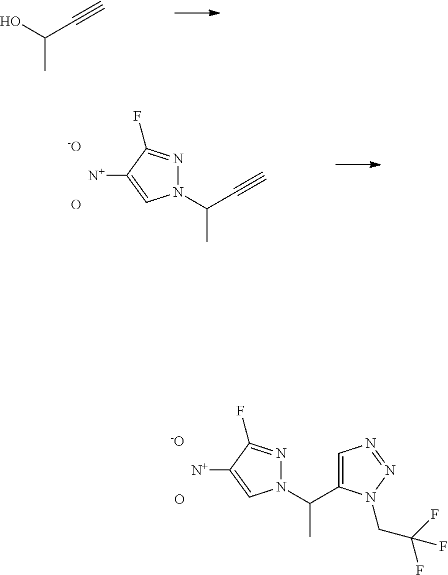

The title compound was synthesised from but-3-yn-2-ol and 3-fluoro-4-nitro-1H-pyrazole according to the methods of Preparations 96 and 31. ¹H NMR (600 MHz, CDCl₃) δ 8.09 (d, J=1.1 Hz, 1H), 7.88 (s, 1H), 5.61 (q, J=7.1 Hz, 1H), 5.30-5.07 (m, 2H), 2.04 (d, J=7.0 Hz, 3H).

### Preparation 145: 1-(2,2-diethoxyethyl)-5-[1-(4-nitropyrazol-1-yl)ethyl]triazole

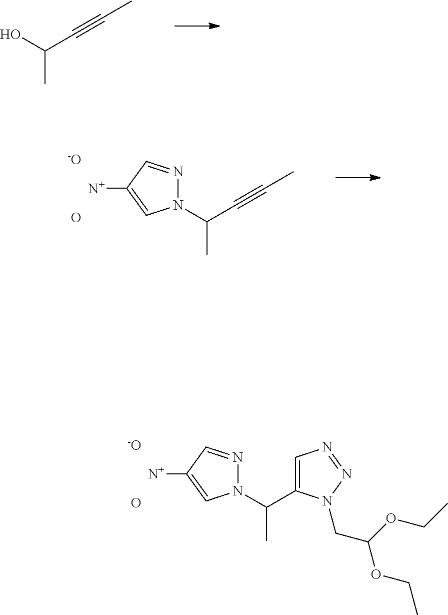

The title compound (3.12 g) was synthesised as an orange oil from but-3-yn-2-ol and 4-nitro-1H-pyrazole according to the methods of Preparations 96 and 31, using 2-azido-1,1-diethoxy-ethane in the cycloaddition step. LCMS (METHOD 4) (ES): m/z 325.3 [M+H]⁺, RT=0.58 min.

### Preparation 146: 1-(2,2-difluoroethyl)-5-[1-(4-nitropyrazol-1-yl)ethyl]triazole

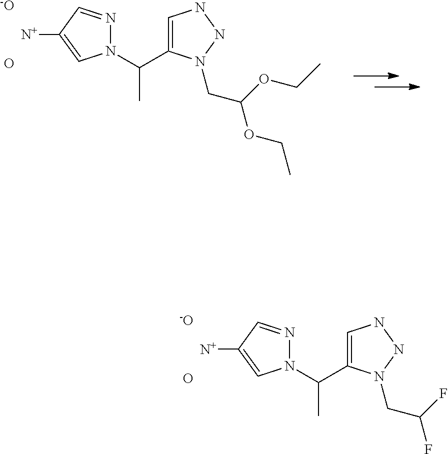

A mixture of the compound of Preparation 145 (9.62 mmol, 3120 mg), TFA (20 mL) and water (1 mL) was stirred at 50° C. for 2 hours. All the volatiles were evaporated, water (20 mL) was added and the pH was adjusted to neutral with solid NaHCO₃. The mixture was extracted with EtOAc (2×50 mL), dried (Na₂SO₄) and evaporated to give crude 2-[5-[1-(4-nitropyrazol-1-yl)ethyl]triazol-1-yl]acetaldehyde (1450 mg, 60% Yield) as a brown syrup. The crude aldehyde (1.92 mmol, 480 mg) was dissolved in DCM (30 mL) and DAST (4.80 mmol, 773 mg) was added and the mixture was stirred at room temperature for 18 hours. The reaction was diluted with DCM (40 mL, carefully washed with sat. NaHCO₃ (until neutral pH), dried (Na₂SO₄) and evaporated. Purification of the residue by column chromatography (EtOAc:heptane) gave the title compound (308 mg, 59% Yield) as a pale yellow syrup. ¹H NMR (400 MHz, CDCl₃) δ 8.15 (s, 1H), 8.08 (s, 1H), 7.86 (s, 1H), 6.16 (tdd, J=55.0, 5.1, 3.1 Hz, 1H), 5.80 (q, J=7.1 Hz, 1H), 4.94-4.66 (m, 2H), 2.03 (d, J=7.1 Hz, 3H); LCMS (METHOD 3) (ES): m/z 273.3 [M+H]⁺, RT=0.54 min.

### Preparation 147: 4-(3-fluoro-4-nitro-pyrazol-1-yl)-4-[3-(2,2,2-trifluoroethyl)triazol-4-yl]butanenitrile

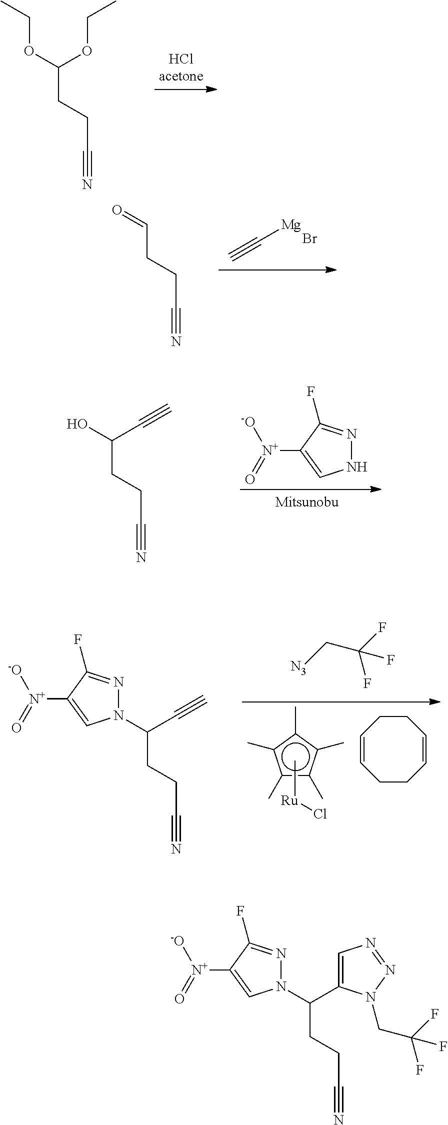

The title compound (110 mg) was synthesised as a colourless oil as outlined in the scheme above. ¹H NMR (400 MHz, MeOD) δ 8.76 (s, 1H), 8.13 (s, 1H), 5.81 (dd, J=10.4, 3.7 Hz, 1H), 5.57-5.29 (m, 2H), 2.89-2.76 (m, 2H), 2.59-2.46 (m, 2H); LCMS (METHOD 3) (ES): no ionisation, RT=0.65 min.

### Preparation 148: tert-butyl-[2-(3-fluoro-4-nitro-pyrazol-1-yllbut-3-ynoxy]-dimethyl-silane

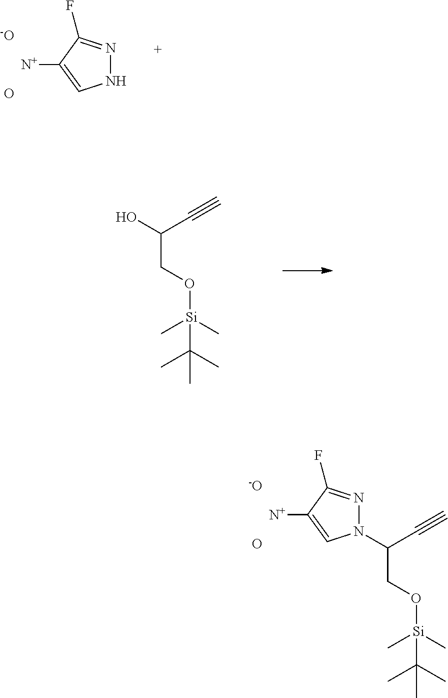

The title compound (1.70 g, 72%) was synthesised as an orange oil according to the method of Preparation 96. ¹H NMR (400 MHz, DMSO-d6) δ 8.90 (s, 1H), 5.42-5.35 (m, 1H), 4.00 (dd, J=10.6, 4.6 Hz, 1H), 3.91 (dd, J=10.6, 8.2 Hz, 1H), 3.82 (d, J=2.3 Hz, 1H), 0.77 (s, 9H), 0.00 (s, 3H), −0.06 (s, 3H).

### Preparation 149: tert-butyl-[2-(3-fluoro-4-nitro-pyrazol-1-yl)-2-[3-(2,2,2-trifluoroethyl)triazol-4-yl]ethoxy]-dimethyl-silane

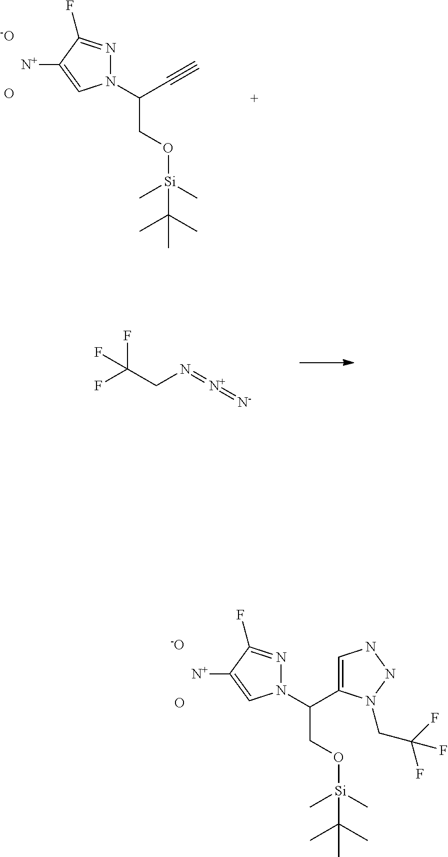

The title compound (138 mg, 62% Yield) was prepared as a brown oil from the alkyne of Preparation 148 according to the method of Preparation 31. LCMS (METHOD 3) (ES): m/z 439.5 [M+H]⁺, RT=0.93 min.

### Preparation 150: N-[(1S)-1-[[1-[2-[tert-butyl(dimethyl)silyl]oxy-1-[3-(2,2,2-trifluoroethyl)triazol-4-yl]ethyl]-3-fluoro-pyrazol-4-yl]carbamoyl]-2,2-dicyclopropyl-ethyl]-2-isopropyl-pyrazole-3-carboxamide

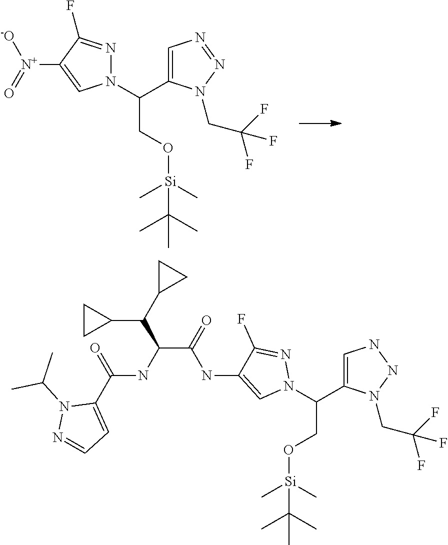

Preparation 150 was prepared from the compounds of Preparation 149 and 5 and 2-isopropylpyrazole-3-carboxylic acid according to the methods of Preparations 11, 33 and 24 and Example 1. LCMS (METHOD 3) (ES): m/z 697.1 [M+H]⁺, RT=0.98 min.

### Preparation 151: 2-(3-fluoro-4-nitro-pyrazol-1-yl)-2-[3-(2,2,2-trifluoroethyl)triazol-4-yl]ethanol

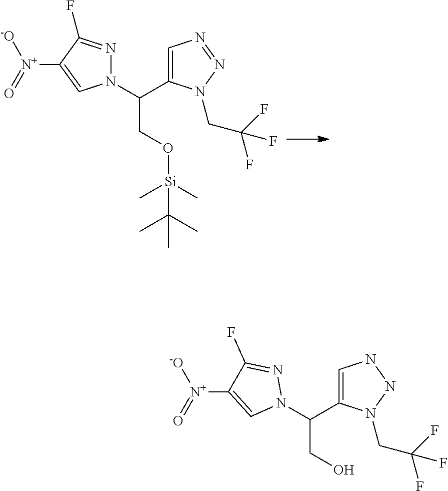

To a solution of the compound of Preparation 149 (0.700 g, 1.60 mmol) in DCM (10 mL) was added 1M TBAF in THF (3 mL, 3 mmol). The solution was stirred at room temperature for 1 hour and then the reaction solution was diluted with DCM (10 mL) and washed with water (20 mL). The aq. phase was extracted with DCM (2×20 mL). The combined organic phases were dried over MgSO₄ and concentrated in vacuo. The residue was purified by column chromatography (heptane/ethyl acetate 1:1 to 0:1), to give the title compound (0.36 g, 70% Yield) as a brown oil. LCMS (METHOD 3) (ES): m/z 325.3 [M+H]⁺, RT=0.57 min.

### Preparation 152: 5-[2-(difluoromethoxy)-1-(3-fluoro-4-nitro-pyrazol-1-yl)ethyl]-1-(2,2,2-trifluoroethyl)triazole

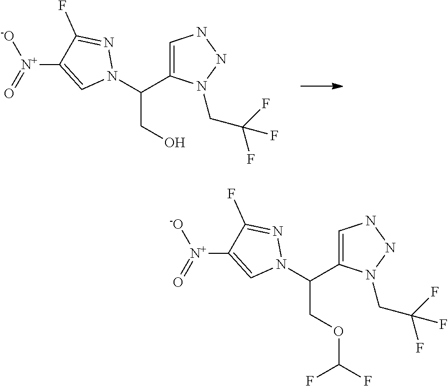

The compound of Preparation 151 (320 mg, 0.987 mmol) was dissolved in MeCN (10 mL) and CuI (40 mg, 0.21 mmol) was added. The mixture was heated to 50° C. and a solution of 2,2-difluoro-2-fluorosulfonyl-acetic acid (264 mg, 1.48 mmol) in MeCN (1 mL) was added dropwise over a period of 30 min. The reaction mixture was heated for an additional 30 min at 50° C. and stirred at room temperature for 3 days. The reaction mixture was purified directly by acidic prep. HPLC to give the title compound (31 mg, 8% Yield). ¹H NMR (400 MHz, CDCl₃) δ 8.08 (s, 1H), 8.05 (s, 1H), 6.31 (t, J=71.9 Hz, 1H), 5.62 (dd, J=7.0, 4.5 Hz, 1H), 5.15 (dq, J=16.3, 8.2 Hz, 1H), 4.95 (dq, J=15.7, 7.9 Hz, 1H), 4.54 (dd, J=11.2, 7.0 Hz, 1H), 4.46 (dd, J=11.2, 4.5 Hz, 1H); LCMS (METHOD 3) (ES): m/z 375.3 [M+H]⁺, RT=0.70 min.

### Preparation 153: 4-nitro-1-[[3-(2,2,2-trifluoroethyl)imidazol-4-yl]methyl]pyrazole

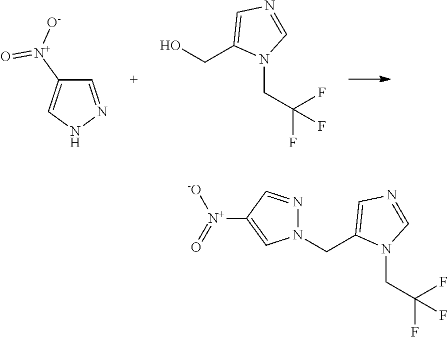

According to the method of Preparation 96 the 4-nitropyrazole was reacted with [3-(2,2,2-trifluoroethyl)imidazol-4-yl]methanol to give the title compound (75 mg, 49%). LCMS (METHOD 3) (ES): m/z 276.1 [M+H]⁺, RT=0.49 min.

### EXAMPLES

### Example 1: N-[(1S)-1-(dicyclopropylmethyl)-2-oxo-2-[[1-[[1-(2,2,2-trifluoroethyl)triazol-4-yl]methyl]pyrazol-4-yl]amino]ethyl]-2-isopropyl-pyrazole-3-carboxamide

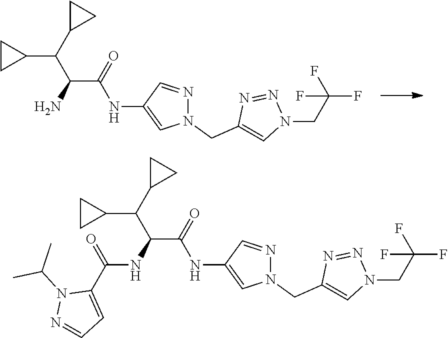

HATU (30.0 mg, 0.08 mmol) was added to a solution of the compound of Preparation 13 (32.0 mg, 0.08 mmol), 2-isopropylpyrazole-3-carboxylic acid (12.0 mg, 0.08 mmol) and DIPEA (0.056 mL, 0.32 mmol) in DMF (1 mL) at room temperature. The reaction mixture was stirred for 30 minutes, then purified directly by prep. basic HPLC to afford the title compound as a colourless solid (35.3 mg, 83% yield). ¹H NMR (400 MHz, DMSO-d6) δ 10.20 (s, 1H), 8.35 (d, J=8.8 Hz, 1H), 8.17 (s, 1H), 8.00 (s, 1H), 7.49 (d, J=1.9 Hz, 1H), 7.48 (s, 1H), 6.93 (d, J=2.0 Hz, 1H), 5.51 (q, J=9.1 Hz, 2H), 5.46-5.34 (m, 3H), 4.77-4.65 (m, 1H), 1.37 (d, J=6.6 Hz, 3H), 1.35 (d, J=6.7 Hz, 3H), 0.91-0.66 (m, 3H), 0.52-0.03 (m, 8H). LCMS (ES): m/z 534.255 [M+H]⁺, RT=2.24 min.

### Example 2: N-[(1S)-1-(dicyclopropylmethyl)-2-oxo-2-[[1-[[3-(2,2,2-trifluoroethyl)triazol-4-yl]methyl]pyrazol-4-yl]amino]ethyl]-2-isopropyl-pyrazole-3-carboxamide

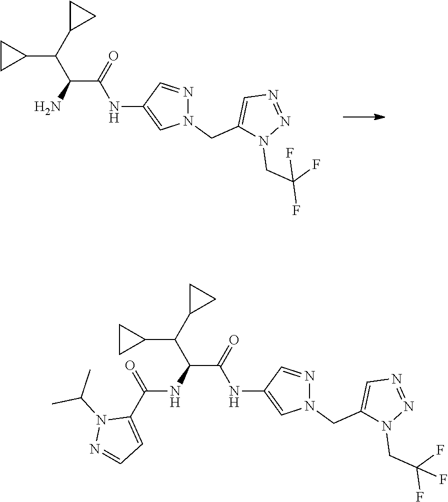 [SMILES: N[C@H](C(=O)Nc1cnn(Cc2cnnn2CC(F)(F)F)c1)C(C1CC1)C1CC1 ; CC(C)n1nccc1C(=O)N[C@H](C(=O)Nc1cnn(Cc2cnnn2CC(F)(F)F)c1)C(C1CC1)C1CC1]

According to the method of Example 1 the compound of Preparation 18 (14.0 mg, 0.036 mmol) was reacted with 2-isopropylpyrazole-3-carboxylic acid (5.6 mg, 0.036 mmol) to afford the title compound as a colourless solid after prep. acidic HPLC (14.2 mg, 74% yield). ¹H NMR (400 MHz, DMSO-d6) δ 10.25 (s, 1H), 8.37 (d, J=8.8 Hz, 1H), 8.10 (s, 1H), 7.68 (s, 1H), 7.52 (s, 1H), 7.49 (d, J=2.0 Hz, 1H), 6.92 (d, J=2.0 Hz, 1H), 5.67-5.50 (m, 4H), 5.40 (p, J=6.6 Hz, 1H), 4.78-4.63 (m, 1H), 1.37 (d, J=6.6 Hz, 3H), 1.34 (d, J=6.6 Hz, 3H), 0.93-0.65 (m, 3H), 0.49-0.02 (m, 8H). LCMS (ES): m/z 534.256 [M+H]⁺, RT=2.26 min.

### Example 3: N-[(1S)-1-(dicyclopropylmethyl)-2-oxo-2-[[1-[[3-(2,2,2-trifluoroethyl)triazol-4-yl]methyl]pyrazol-4-yl]amino]ethyl]-4-methyl-1,2,5-oxadiazole-3-carboxamide

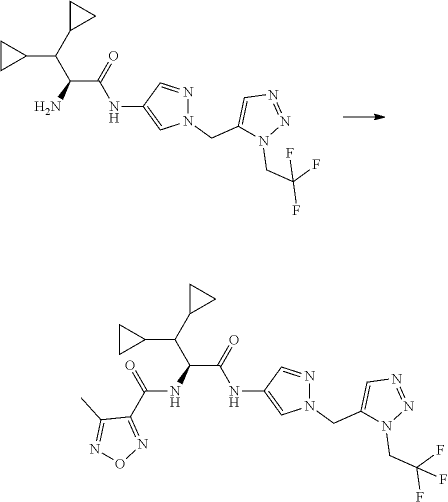 [SMILES: N[C@H](C(=O)Nc1cnn(Cc2cnnn2CC(F)(F)F)c1)C(C1CC1)C1CC1 ; Cc1nonc1C(=O)N[C@H](C(=O)Nc1cnn(Cc2cnnn2CC(F)(F)F)c1)C(C1CC1)C1CC1]

According to the method of Example 1 the compound of Preparation 18 (14.0 mg, 0.036 mmol) was reacted with 4-methyl-1,2,5-oxadiazole-3-carboxylic acid (4.6 mg, 0.036 mmol) to afford the title compound as a colourless solid after prep. acidic HPLC (11.7 mg, 64% yield). ¹H NMR (400 MHz, DMSO-d6) δ 10.27 (s, 1H), 8.98 (d, J=8.9 Hz, 1H), 8.11 (s, 1H), 7.69 (s, 1H), 7.51 (s, 1H), 5.68-5.48 (m, 4H), 4.78 (dd, J=8.9, 6.2 Hz, 1H), 2.48 (s, 3H), 0.94-0.67 (m, 3H), 0.52-0.05 (m, 8H). LCMS (ES): m/z 508.203 [M+H]⁺, RT=2.31 min.

### Example 4: (3,3-difluorocyclobutyl)N-[(1S)-1-(dicyclopropylmethyl)-2-oxo-2-[[1-[[3-(2,2,2-trifluoroethyl)triazol-4-yl]methyl]pyrazol-4-yl]amino]ethyl]carbamate

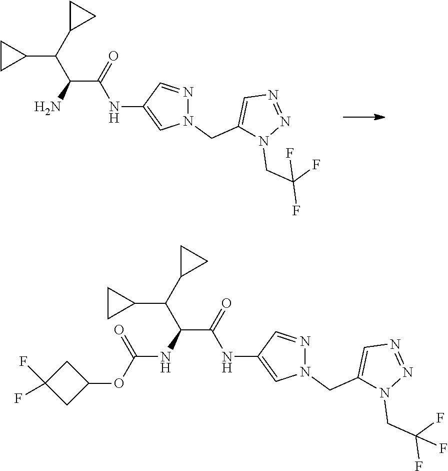 [SMILES: N[C@H](C(=O)Nc1cnn(Cc2cnnn2CC(F)(F)F)c1)C(C1CC1)C1CC1 ; O=C(N[C@H](C(=O)Nc1cnn(Cc2cnnn2CC(F)(F)F)c1)C(C1CC1)C1CC1)OC1CC(F)(F)C1]

DIPEA (0.013 mL, 0.072 mmol) was added to a solution of the compound of Preparation 18 (14.0 mg, 0.036 mmol) and the compound of Preparation 19 (9.0 mg, 0.036 mmol) in DMF (1.0 mL) and the reaction mixture was stirred at room temperature for 1 hour then purified directly by prep. acidic HPLC, to afford the title compound as a colourless solid (13.8 mg, 72% yield). ¹H NMR (400 MHz, DMSO-d6) δ 10.02 (s, 1H), 8.07 (s, 1H), 7.68 (s, 1H), 7.49 (s, 1H), 7.46 (d, J=8.9 Hz, 1H), 5.65-5.50 (m, 4H), 4.93-4.74 (m, 1H), 4.22 (dd, J=8.9, 6.4 Hz, 1H), 3.14-2.92 (m, 2H), 2.70-2.54 (m, 2H), 0.94-0.80 (m, 1H), 0.77-0.63 (m, 1H), 0.59-0.02 (m, 9H). LCMS (ES): m/z 532.210 [M+H]⁺, RT=2.29 min.

### Example 5: N-[(1S)-1-(dicyclopropylmethyl)-2-[[1-[[3-(difluoromethyl)triazol-4-yl]methyl]pyrazol-4-yl]amino]-2-oxo-ethyl]-2-isopropyl-pyrazole-3-carboxamide

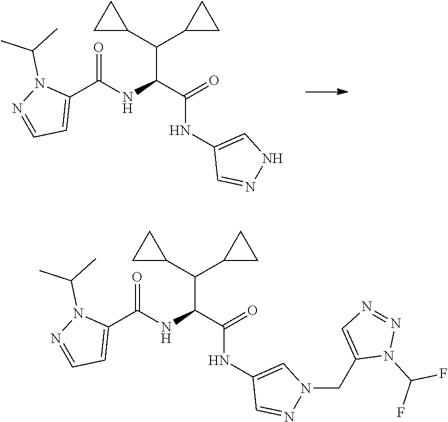

Cs₂CO₃ (30.0 mg, 0.092 mmol) was added to a solution of the compound of Preparation 26 (20.0 mg, 0.054 mmol) and the compound of Preparation 22 (7.0 mg, 0.041 mmol) in DMSO (1 mL) and the reaction mixture was stirred at room temperature for 4 hours. The reaction mixture was purified directly by prep. acidic HPLC to afford the title compound as a colourless solid (8.4 mg, 31% yield). ¹H NMR (600 MHz, DMSO-d6) δ 10.19 (s, 1H), 8.65 (s, 1H), 8.34 (d, J=8.8 Hz, 1H), 8.21 (t, J=58.2 Hz, 1H), 8.03 (s, 1H), 7.49 (d, J=1.9 Hz, 1H), 7.47 (s, 1H), 6.92 (d, J=2.0 Hz, 1H), 5.45 (d, J=2.5 Hz, 2H), 5.39 (p, J=6.6 Hz, 1H), 4.71 (dd, J=8.8, 7.2 Hz, 1H), 1.36 (dd, J=16.2, 6.6 Hz, 6H), 0.83 (dtd, J=13.3, 8.2, 5.0 Hz, 1H), 0.73 (td, J=6.5, 3.5 Hz, 2H), 0.55-0.05 (m, 8H). LCMS (METHOD 4) (ES): m/z 502.4 [M+H]⁺, RT=0.63 min.

### Example 6: N-[(1S)-1-(dicyclopropylmethyl)-2-[[3-fluoro-1-[2-methoxy-1-[3-(2,2,2-trifluoroethyl)triazol-4-yl]ethyl]pyrazol-4-yl]amino]-2-oxo-ethyl]-2-isopropyl-pyrazole-3-carboxamide

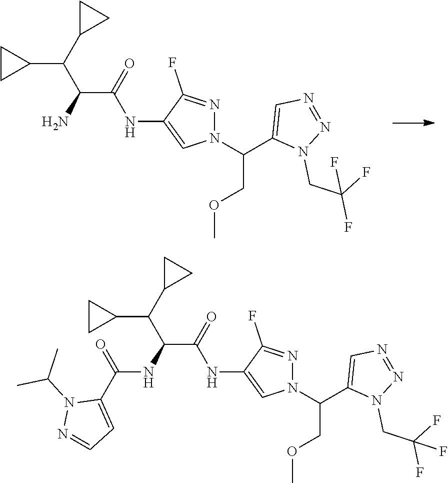

According to the method of Example 1 the compound of Preparation 34 (20.0 mg, 0.043 mmol) was reacted with 2-isopropylpyrazole-3-carboxylic acid (6.6 mg, 0.043 mmol) to afford the title compound as a colourless solid after prep. acidic HPLC (15.9 mg, 62% yield). ¹H NMR (400 MHz, DMSO-d6) δ 9.85 (d, J=22.4 Hz, 1H), 8.17 (t, J=9.2 Hz, 1H), 8.02 (dd, J=18.5, 2.0 Hz, 1H), 7.84 (d, J=5.3 Hz, 1H), 7.31 (d, J=1.9 Hz, 1H), 6.72 (t, J=1.8 Hz, 1H), 5.83 (ddd, J=9.4, 5.7, 3.0 Hz, 1H), 5.53-5.30 (m, 2H), 5.20 (pd, J=6.7, 4.4 Hz, 1H), 4.61 (t, J=8.3 Hz, 1H), 3.87 (dd, J=10.5, 8.8 Hz, 1H), 3.58 (dd, J=10.5, 4.9 Hz, 1H), 3.07 (d, J=3.6 Hz, 3H), 1.18 (ddd, J=11.5, 6.6, 2.4 Hz, 7H), 0.72-0.45 (m, 3H), 0.34-−0.15 (m, 8H). LCMS (ES): m/z 596.272 [M+H]⁺, RT=2.40 min.

### Example 7: N-[(1S)-1-(dicyclopropylmethyl)-2-[[3-fluoro-1-[2-methoxy-1-[3-(2,2,2-trifluoroethyl)triazol-4-yl]ethyl]pyrazol-4-yl]amino]-2-oxo-ethyl]-4-methyl-1,2,5-oxadiazole-3-carboxamide

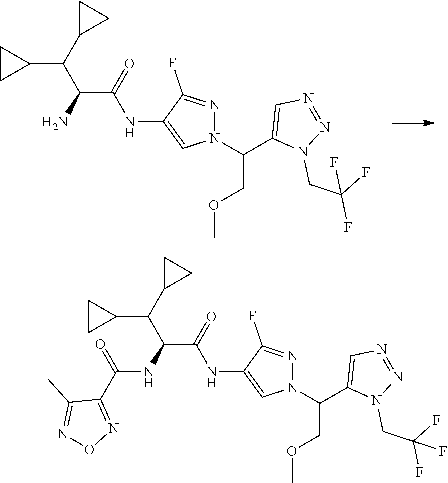

According to the method of Example 1 the compound of Preparation 34 (20.0 mg, 0.043 mmol) was reacted with 4-methyl-1,2,5-oxadiazole-3-carboxylic acid (7.0 mg, 0.054 mmol) to afford the title compound as a colourless solid after prep. acidic HPLC (18 mg, 72% yield). ¹H NMR (400 MHz, DMSO-d6) δ 9.88 (d, J=21.1 Hz, 1H), 8.78 (t, J=8.5 Hz, 1H), 8.01 (dd, J=17.3, 1.9 Hz, 1H), 7.82 (d, J=4.3 Hz, 1H), 5.91-5.70 (m, 1H), 5.54-5.25 (m, 2H), 4.66 (ddd, J=9.4, 6.5, 3.2 Hz, 1H), 3.84 (dd, J=10.5, 8.8 Hz, 1H), 3.55 (dd, J=10.5, 5.0 Hz, 1H), 3.05 (d, J=3.3 Hz, 3H), 2.27 (d, J=1.6 Hz, 3H), 0.75-0.43 (m, 3H), 0.38-−0.12 (m, 8H). LCMS (ES): m/z 570.220 [M+H]⁺, RT=2.45 min.

### Example 8: N-[(1S)-1-(4,4-difluorocyclohexyl)-2-[[3-fluoro-1-[2-methoxy-1-[3-(2,2,2-trifluoroethyl)triazol-4-yl]ethyl]pyrazol-4-yl]amino]-2-oxo-ethyl]-2-isopropyl-pyrazole-3-carboxamide

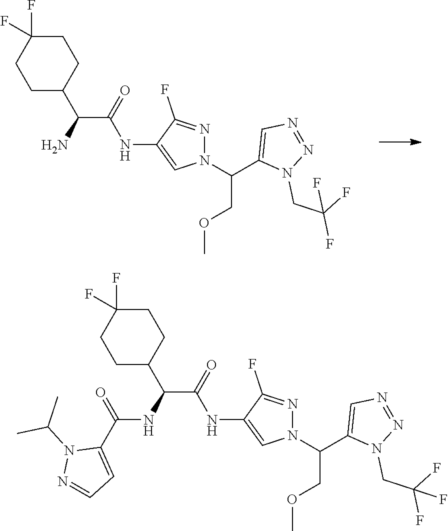 [SMILES: COCC(c1cnnn1CC(F)(F)F)n1cc(NC(=O)[C@@H](N)C2CCC(F)(F)CC2)c(F)n1 ; COCC(c1cnnn1CC(F)(F)F)n1cc(NC(=O)[C@@H](NC(=O)c2ccnn2C(C)C)C2CCC(F)(F)CC2)c(F)n1]

According to the method of Example 1 the compound of Preparation 36 (21.0 mg, 0.043 mmol) was reacted with 2-isopropylpyrazole-3-carboxylic acid (6.6 mg, 0.043 mmol) to afford the title compound as a colourless solid after prep. acidic HPLC (5.4 mg, 20% yield). ¹H NMR (600 MHz, DMSO-d6) δ 10.15 (d, J=7.4 Hz, 1H), 8.55 (dd, J=8.2, 3.0 Hz, 1H), 8.23 (dd, J=4.4, 1.9 Hz, 1H), 8.03 (d, J=1.8 Hz, 1H), 7.49 (t, J=1.7 Hz, 1H), 6.92 (t, J=1.9 Hz, 1H), 6.05-5.91 (m, 1H), 5.72-5.51 (m, 2H), 5.36 (dp, J=13.7, 6.8 Hz, 1H), 4.50 (td, J=8.5, 2.6 Hz, 1H), 4.06 (ddd, J=10.5, 8.9, 4.0 Hz, 1H), 3.74 (dt, J=10.4, 4.5 Hz, 1H), 3.26 (d, J=2.4 Hz, 3H), 2.11-1.91 (m, 3H), 1.87 (d, J=13.3 Hz, 1H), 1.74 (ddd, J=24.9, 13.9, 5.6 Hz, 2H), 1.62 (t, J=14.5 Hz, 1H), 1.50-1.26 (m, 8H). LCMS (METHOD 4) (ES): m/z 620.4 [M+H]⁺, RT=0.68 min.

### Example 9: N-[(1S)-1-(4,4-difluorocyclohexyl)-2-[[3-fluoro-1-[2-methoxy-1-[3-(2,2,2-trifluoroethyl)triazol-4-yl]ethyl]pyrazol-4-yl]amino]-2-oxo-ethyl]-4-methyl-1,2,5-oxadiazole-3-carboxamide

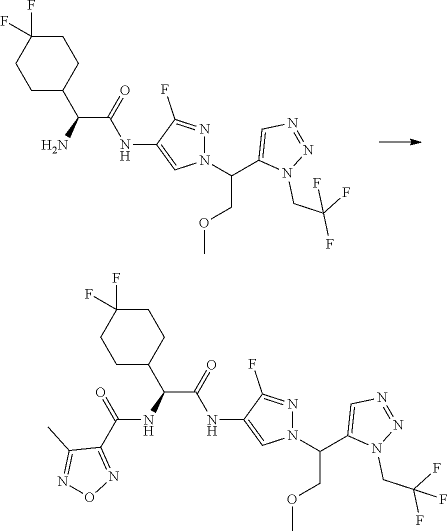 [SMILES: COCC(c1cnnn1CC(F)(F)F)n1cc(NC(=O)[C@@H](N)C2CCC(F)(F)CC2)c(F)n1 ; COCC(c1cnnn1CC(F)(F)F)n1cc(NC(=O)[C@@H](NC(=O)c2nonc2C)C2CCC(F)(F)CC2)c(F)n1]

According to the method of Example 1 the compound of Preparation 36 (20.0 mg, 0.042 mmol) was reacted with 4-methyl-1,2,5-oxadiazole-3-carboxylic acid (7.0 mg, 0.054 mmol) to afford the title compound as a colourless solid after prep. acidic HPLC (11 mg, 44% yield). ¹H NMR (600 MHz, DMSO-d6) δ 8.24 (dd, J=4.9, 1.9 Hz, 1H), 8.03 (d, J=1.6 Hz, 1H), 6.04-5.92 (m, 1H), 5.68-5.46 (m, 2H), 4.57 (dd, J=8.4, 2.2 Hz, 1H), 4.06 (ddd, J=10.4, 8.8, 3.6 Hz, 1H), 3.74 (ddd, J=10.5, 4.9, 3.5 Hz, 1H), 3.26 (d, J=2.6 Hz, 3H), 2.47 (d, J=3.7 Hz, 3H), 2.07-1.95 (m, 3H), 1.89-1.59 (m, 5H), 1.47-1.27 (m, 2H). LCMS (METHOD 4) (ES): m/z 594.3 [M+H]⁺, RT=0.70 min.

### Example 10: N-[(1S)-1-(4,4-difluorocyclohexyl)-2-[[3-fluoro-1-[1-[1-(2,2,2-trifluoroethyl)tetrazol-5-yl]propyl]pyrazol-4-yl]amino]-2-oxo-ethyl]-2-isopropyl-pyrazole-3-carboxamide

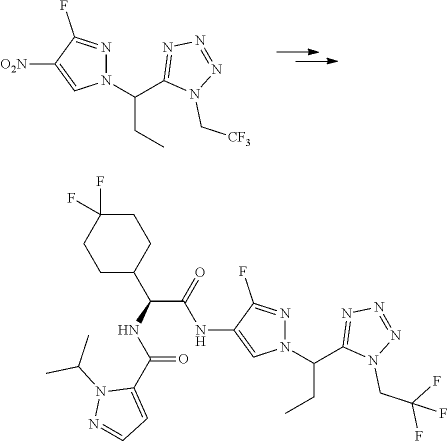 [SMILES: CCC(c1nnnn1CC(F)(F)F)n1cc([N+](=O)[O-])c(F)n1 ; CCC(c1nnnn1CC(F)(F)F)n1cc(NC(=O)[C@@H](NC(=O)c2ccnn2C(C)C)C2CCC(F)(F)CC2)c(F)n1]

Following the methods of Preparations 11, 35 and 24 and Example 1 the nitro compound of Preparation 40 was reacted to give the title compound (19 mg) as a colourless solid. ¹H NMR (400 MHz, DMSO-d6) δ 10.20 (d, J=2.6 Hz, 1H), 8.56 (d, J=8.1 Hz, 1H), 8.31 (t, J=2.1 Hz, 1H), 7.50 (d, J=2.0 Hz, 1H), 6.93 (dd, J=2.0, 1.2 Hz, 1H), 5.91 (dd, J=9.3, 5.8 Hz, 1H), 5.82-5.55 (m, 2H), 5.37 (hept, J=6.6 Hz, 1H), 4.52 (t, J=8.5 Hz, 1H), 2.48-2.32 (m, 1H), 2.29-2.14 (m, 1H), 2.14-1.57 (m, 7H), 1.48-1.22 (m, 8H), 0.82 (t, J=7.3 Hz, 3H); LCMS (ES): m/z 605.254 [M+H]⁺, RT=2.43 min.

In a similar fashion to Example 10, Examples 11-50 were synthesised starting from the indicated nitropyrazoles.

| | Precursor | | | | |
|---|---|---|---|---|---|
| Ex. | Prep. | | | Mass | LCMS |
| No. | number | Structure | Name | ion | RT |
| 11 | 40 | 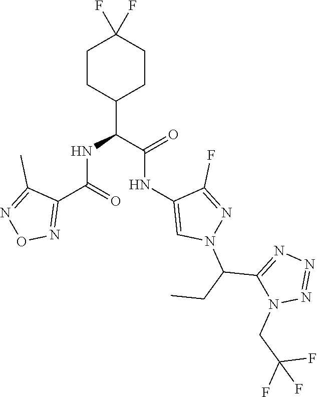 [SMILES: CCC(c1nnnn1CC(F)(F)F)n1cc(NC(=O)[C@@H](NC(=O)c2nonc2C)C2CCC(F)(F)CC2)c(F)n1] | N-[(1S)-1-(4,4-difluorocyclo- hexyl)-2-[3-fluoro-1-[1-[1- (2,2,2-trifluoroethyl)-tetrazol- 5-yl]propyl]pyrazol-4-yl]- amino]-2-oxo-ethyl]-4-methyl- 1,2,5-oxadiazole-3- carboxamide | 579.202 | 2.46 |
| 12 | 62 | 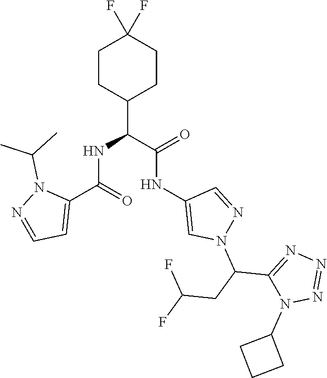 [SMILES: CC(C)n1nccc1C(=O)N[C@H](C(=O)Nc1cnn(C(CC(F)F)c2nnnn2C2CCC2)c1)C1CCC(F)(F)CC1] | N-[(1S)-2-[[1-[1-(1-cyclo- butyltetrazol-5-yl)-3,3-di- fluoropropyl]pyrazol-4-yl]- amino]-1-(4,4-difluorocyclo- hexyl)-2-oxo-ethyl]-2-iso- propyl-pyrazole-3-carboxamide | 595.288 | 2.39 |
| 13 | 62 | 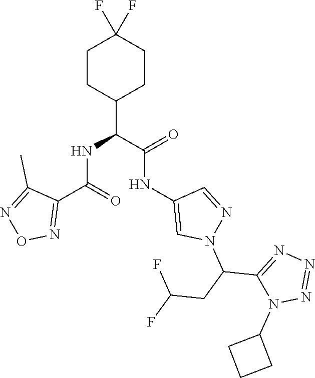 [SMILES: Cc1nonc1C(=O)N[C@H](C(=O)Nc1cnn(C(CC(F)F)c2nnnn2C2CCC2)c1)C1CCC(F)(F)CC1] | N-[(1S)-2-[[1-[1-(1-cyclo- butyltetrazol-5-yl)-3,3-di- fluoro-propyl]pyrazol-4-yl]- amino]-1-(4,4-difluoro- cyclohexyl)-2-oxo-ethyl]-4- methyl-1,2,5-oxadiazole-3- carboxamide | 569.236 | 2.42 |
| 14 | 63 | 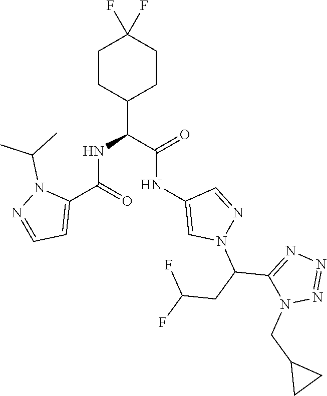 [SMILES: CC(C)n1nccc1C(=O)N[C@H](C(=O)Nc1cnn(C(CC(F)F)c2nnnn2CC2CC2)c1)C1CCC(F)(F)CC1] | N-[(1S)-2-[[1-[1-[1- (cyclopropylmethyl)tetrazol-5- yl]-3,3-difluoro-propyl]pyrazol- 4-yl]amino]-1-(4,4- difluorocyclohexyl)-2-oxo- ethyl]-2-isopropyl-pyrazole-3- carboxamide | 595.290 | 2.37 |
| 15 | 63 | 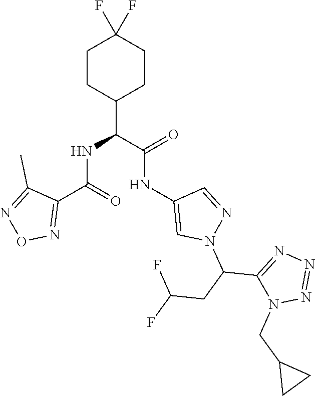 [SMILES: Cc1nonc1C(=O)N[C@H](C(=O)Nc1cnn(C(CC(F)F)c2nnnn2CC2CC2)c1)C1CCC(F)(F)CC1] | N-[(1S)-2-[[1-[1-[1- (cyclopropylmethyl)tetrazol-5- yl]-3,3-difluoro-propyl]pyrazol- 4-yl]amino]-1-(4,4- difluorocyclohexyl)-2-oxo- ethyl]-4-methyl-1,2,5- oxadiazole-3-carboxamide | 569.237 | 2.36 |
| 16 | 64 | 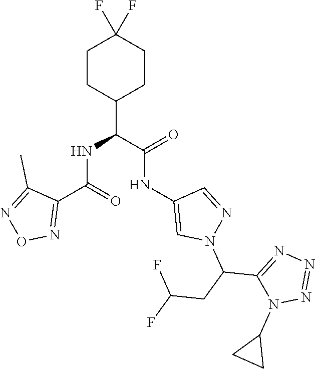 [SMILES: Cc1nonc1C(=O)N[C@H](C(=O)Nc1cnn(C(CC(F)F)c2nnnn2C2CC2)c1)C1CCC(F)(F)CC1] | N-[(1S)-2-[1-[1-(1- cyclopropyltetrazol-5-yl)-3,3- difluoro-propyl]pyrazol-4- yl]amino]-1-(4,4- difluorocyclohexyl)-2-oxo- ethyl]-4-methyl-1,2,5- oxadiazole-3-carboxamide | 553.205 | 2.36 |
| 17 | 64 | 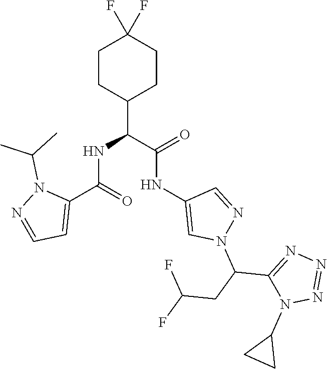 [SMILES: CC(C)n1nccc1C(=O)N[C@H](C(=O)Nc1cnn(C(CC(F)F)c2nnnn2C2CC2)c1)C1CCC(F)(F)CC1] | N-[(1S)-2-[[1-[1-(1-cyclo- propyltetrazol-5-yl)-3,3- difluoro-propyl]pyrazol-4- yl]amino]-1-(4,4-difluorocyclo- hexyl)-2-oxo-ethyl]-2-iso- propyl-pyrazole-3-carboxamide | 581.273 | 2.32 |
| 18 | 51 | 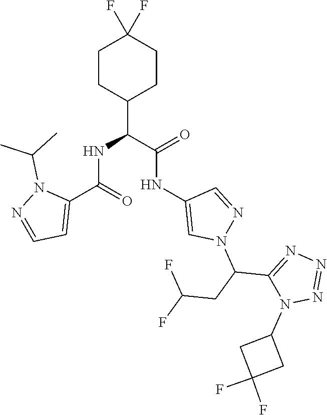 [SMILES: CC(C)n1nccc1C(=O)N[C@H](C(=O)Nc1cnn(C(CC(F)F)c2nnnn2C2CC(F)(F)C2)c1)C1CCC(F)(F)CC1] | N-[(1S)-2-[[1-[1-[1-(3,3- difluorocyclobutyl)tetrazol-5- yl]-3,3-difluoro-propyl]pyrazol- 4-yl]amino]-1-(4,4- difluorocyclohexyl)-2-oxo- ethyl]-2-isopropyl-pyrazole-3- carboxamide | 631.269 | 2.40 |
| 19 | 51 | 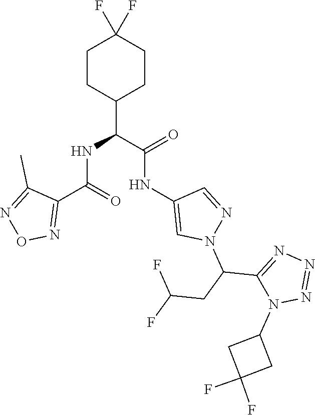 [SMILES: Cc1nonc1C(=O)N[C@H](C(=O)Nc1cnn(C(CC(F)F)c2nnnn2C2CC(F)(F)C2)c1)C1CCC(F)(F)CC1] | N-[(1S)-2-[[1-[1-[1-(3,3- difluorocyclobutyl)tetrazol-5- yl]-3,3-difluoro-propyl]pyrazol- 4-yl]amino]-1-(4,4- difluorocyclohexyl)-2-oxo- ethyl]-4-methyl-1,2,5- oxadiazole-3-carboxamide | 605.217 | 2.43 |
| 20 | 65 | 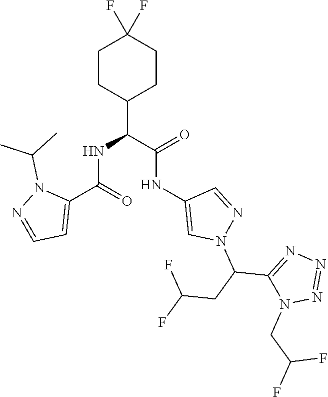 [SMILES: CC(C)n1nccc1C(=O)N[C@H](C(=O)Nc1cnn(C(CC(F)F)c2nnnn2CC(F)F)c1)C1CCC(F)(F)CC1] | N-[(1S)-1-(4,4-difluorocyclo-hexyl)- 2-[[1-[1-[1-(2,2-difluoro- ethyl)tetrazol-5-yl]-3,3-difluoro- propyl]pyrazol-4-yl]amino]-2-oxo- ethyl]-2-isopropyl-pyrazole-3- carboxamide | 605.254 | 2.33 |
| 21 | 65 | 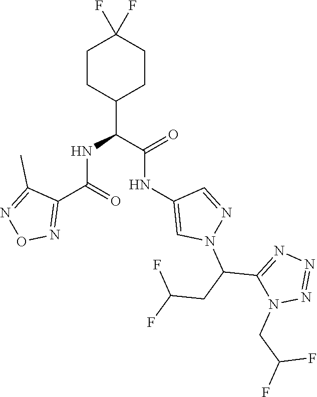 [SMILES: Cc1nonc1C(=O)N[C@H](C(=O)Nc1cnn(C(CC(F)F)c2nnnn2CC(F)F)c1)C1CCC(F)(F)CC1] | N-[(1S)-1-(4,4-difluorocyclo-hexyl)- 2-[[1-[1-[1-(2,2-difluoro- ethyl)tetrazol-5-yl]-3,3-difluoro- propyl]pyrazol-4-yl]amino]-2-oxo- ethyl]-4-methyl-1,2,5-oxadiazole-3- carboxamide | 579.201 | 2.37 |
| 22 | 55 | 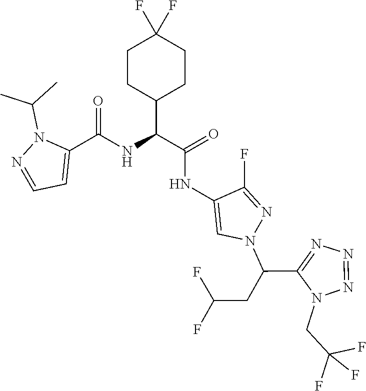 [SMILES: CC(C)n1nccc1C(=O)N[C@H](C(=O)Nc1cn(C(CC(F)F)c2nnnn2CC(F)(F)F)nc1F)C1CCC(F)(F)CC1] | N-[(1S)-1-(4,4- difluorocyclohexyl)-2-[[1-[3,3- difluoro-1-[1-(2,2,2- trifluoroethyl)tetrazol-5- yl]propyl]-3-fluoro-pyrazol-4- yl]amino]-2-oxo-ethyl]-2- isopropyl-pyrazole-3- carboxamide | 639.219 [M − H]⁻ | 2.44 |
| 23* | 55 | 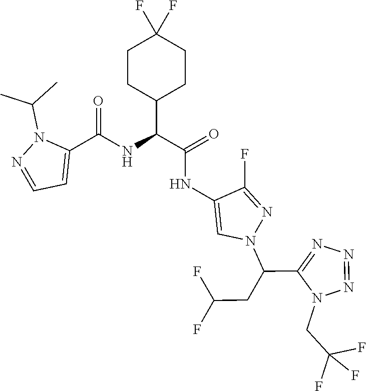 [SMILES: CC(C)n1nccc1C(=O)N[C@H](C(=O)Nc1cn(C(CC(F)F)c2nnnn2CC(F)(F)F)nc1F)C1CCC(F)(F)CC1] | Diastereomer 1 of N-[(1S)-1- (4,4-difluorocyclohexyl)-2-[[1- [3,3-difluoro-1-[1-(2,2,2- trifluoroethyl)tetrazol-5- yl]propyl]-3-fluoro-pyrazol-4- yl]amino]-2-oxo-ethyl]-2- isopropyl-pyrazole-3- carboxamide | 641.235 | 2.44 |
| 24* | 55 | 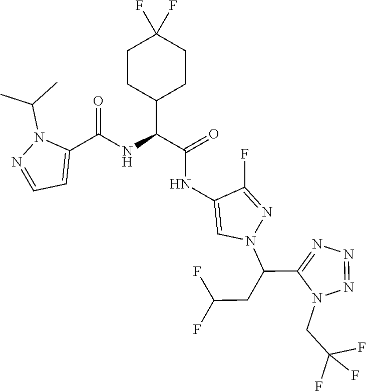 [SMILES: CC(C)n1nccc1C(=O)N[C@H](C(=O)Nc1cn(C(CC(F)F)c2nnnn2CC(F)(F)F)nc1F)C1CCC(F)(F)CC1] | Diastereomer 2 of N-[(1S)-1- (4,4-difluorocyclohexyl)-2-[[1- [3,3-difluoro-1-[1-(2,2,2- trifluoroethyl)tetrazol-5- yl]propyl]-3-fluoro-pyrazol-4- yl]amino]-2-oxo-ethyl]-2- isopropyl-pyrazole-3- carboxamide | 641.235 | 2.44 |
| 25 | 55 | 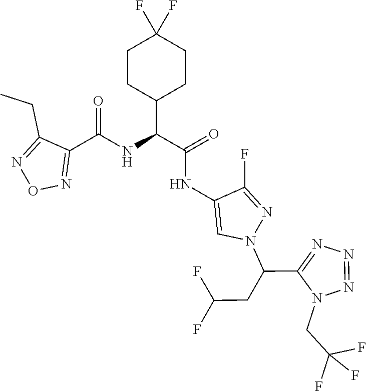 [SMILES: CCc1nonc1C(=O)N[C@H](C(=O)Nc1cn(C(CC(F)F)c2nnnn2CC(F)(F)F)nc1F)C1CCC(F)(F)CC1] | N-[(1S)-1-(4,4- difluorocyclohexyl)-2-[[1-[3,3- difluoro-1-[1-(2,2,2- trifluoroethyl)tetrazol-5- yl]propyl]-3-fluoro-pyrazol-4- yl]amino]-2-oxo-ethyl]-4- ethyl-1,2,5-oxadiazole-3- carboxamide | 627.182 [M − H]⁻ | 2.52 |
| 26 | 55 | 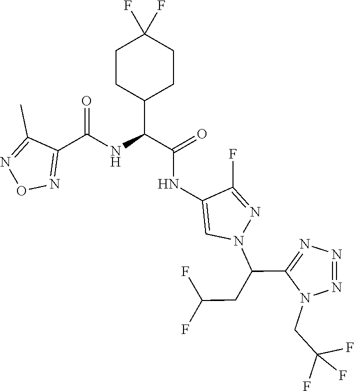 [SMILES: Cc1nonc1C(=O)N[C@H](C(=O)Nc1cn(C(CC(F)F)c2nnnn2CC(F)(F)F)nc1F)C1CCC(F)(F)CC1] | N-[(1S)-1-(4,4- difluorocyclohexyl)-2-[[1-[3,3- difluoro-1-[1-(2,2,2- trifluoroethyl)tetrazol-5- yl]propyl]-3-fluoro-pyrazol-4- yl]amino]-2-oxo-ethyl]-4- methyl-1,2,5-oxadiazole-3- carboxamide | 613.166 [M − H]⁻ | 2.46 |
| 27^{#} | 67 | 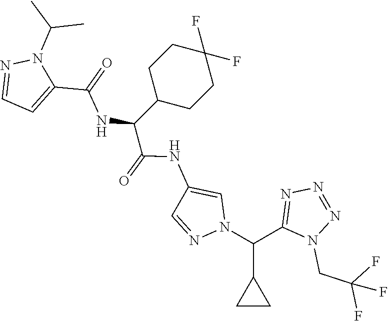 [SMILES: CC(C)n1nccc1C(=O)N[C@H](C(=O)Nc1cnn(C(c2nnnn2CC(F)(F)F)C2CC2)c1)C1CCC(F)(F)CC1] | Diastereomer 1 of N-[(1S)-2- [[1-[cyclopropyl-[1-(2,2,2- trifluoroethyl)tetrazol-5- yl]methyl]pyrazol-4-yl]amino]- 1-(4,4-difluorocyclohexyl)-2- oxo-ethyl]-2-isopropyl- pyrazole-3-carboxamide | 599.263 | 2.39 |
| 28^{#} | 67 | 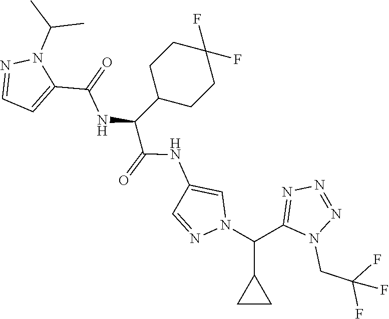 [SMILES: CC(C)n1nccc1C(=O)N[C@H](C(=O)Nc1cnn(C(c2nnnn2CC(F)(F)F)C2CC2)c1)C1CCC(F)(F)CC1] | Diastereomer 2 of N-[(1S)-2- [[1-[cyclopropyl-[1-(2,2,2- trifluoroethyl)tetrazol-5- yl]methyl]pyrazol-4-yl]amino]- 1-(4,4-difluorocyclohexyl)-2- oxo-ethyl]-2-isopropyl- pyrazole-3-carboxamide | 599.263 | 2.39 |
| 29^{#} | 70 | 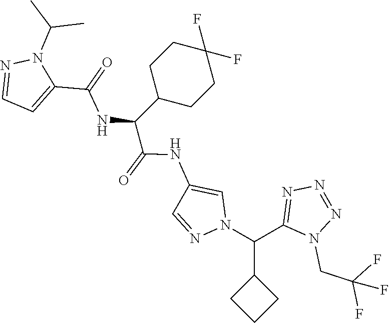 [SMILES: CC(C)n1nccc1C(=O)N[C@H](C(=O)Nc1cnn(C(c2nnnn2CC(F)(F)F)C2CCC2)c1)C1CCC(F)(F)CC1] | Diastereomer 1 of N-[(1S)-2- [[1-[cyclobutyl-[1-(2,2,2- trifluoroethyl)tetrazol-5- yl]methyl]pyrazol-4-yl]amino]- 1-(4,4-difluorocyclohexyl)-2- oxo-ethyl]-2-isopropyl- pyrazole-3-carboxamide | 613.279 | 2.46 |
| 30^{#} | 70 | 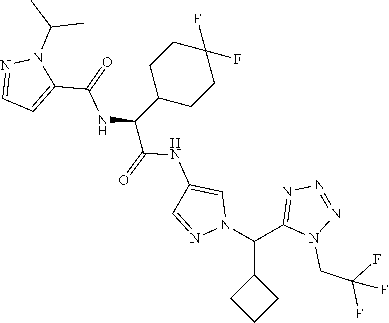 [SMILES: CC(C)n1nccc1C(=O)N[C@H](C(=O)Nc1cnn(C(c2nnnn2CC(F)(F)F)C2CCC2)c1)C1CCC(F)(F)CC1] | Diastereomer 2 of N-[(1S)-2- [[1-[cyclobutyl-[1-(2,2,2- trifluoroethyl)tetrazol-5- yl]methyl]pyrazol-4-yl]amino]- 1-(4,4-difluorocyclohexyl)-2- oxo-ethyl]-2-isopropyl- pyrazole-3-carboxamide | 613.280 | 2.46 |
| 31^{#} | 66 | 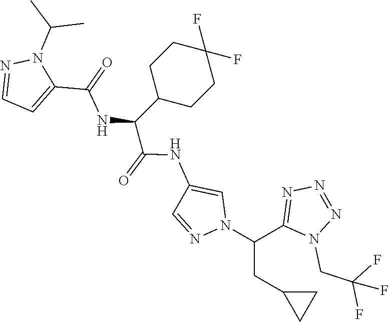 [SMILES: CC(C)n1nccc1C(=O)N[C@H](C(=O)Nc1cnn(C(CC2CC2)c2nnnn2CC(F)(F)F)c1)C1CCC(F)(F)CC1] | Diastereomer 1 of N-[(1S)-2- [[1-[2-cyclopropyl-1-[1-(2,2,2- trifluoroethyl)tetrazol-5- yl]ethyl]pyrazol-4-yl]amino]-1- (4,4-difluorocyclohexyl)-2-oxo- ethyl]-2-isopropyl-pyrazole-3- carboxamide | 613.279 | 2.44 |
| 32^{#} | 66 | 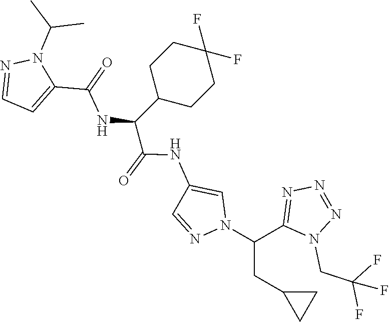 [SMILES: CC(C)n1nccc1C(=O)N[C@H](C(=O)Nc1cnn(C(CC2CC2)c2nnnn2CC(F)(F)F)c1)C1CCC(F)(F)CC1] | Diastereomer 2 of N-[(1S)-2- [[1-[2-cyclopropyl-1-[1-(2,2,2- trifluoroethyl)tetrazol-5- yl]ethyl]pyrazol-4-yl]amino]-1- (4,4-difluorocyclohexyl)-2-oxo- ethyl]-2-isopropyl-pyrazole-3- carboxamide | 613.278 | 2.44 |
| 33^{#} | 58 | 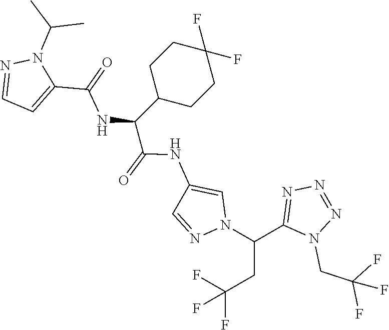 [SMILES: CC(C)n1nccc1C(=O)N[C@H](C(=O)Nc1cnn(C(CC(F)(F)F)c2nnnn2CC(F)(F)F)c1)C1CCC(F)(F)CC1] | Diastereomer 1 of N-[(1S)-1- (4,4-difluorocyclohexyl)-2-oxo- 2-[[1-[3,3,3-trifluoro-1-[1- (2,2,2-trifluoroethyl)-tetrazol- 5-yl]propyl]pyrazol-4- yl]amino]ethyl]-2-isopropyl- pyrazole-3-carboxamide | 641.235 | 2.42 |
| 34^{#} | 58 | 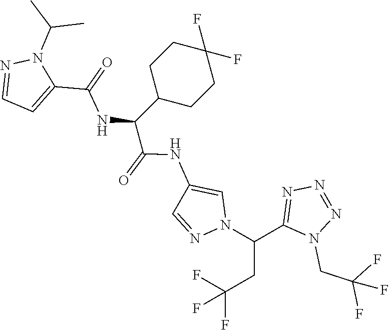 [SMILES: CC(C)n1nccc1C(=O)N[C@H](C(=O)Nc1cnn(C(CC(F)(F)F)c2nnnn2CC(F)(F)F)c1)C1CCC(F)(F)CC1] | Diastereomer 2 of N-[(1S)-1- (4,4-difluorocyclohexyl)-2-oxo- 2-[[1-[3,3,3-trifluoro-1-[1- (2,2,2-trifluoroethyl)-tetrazol- 5-yl]propyl]pyrazol-4- yl]amino]ethyl]-2-isopropyl- pyrazole-3-carboxamide | 641.235 | 2.43 |
| 35^{#} | 68 | 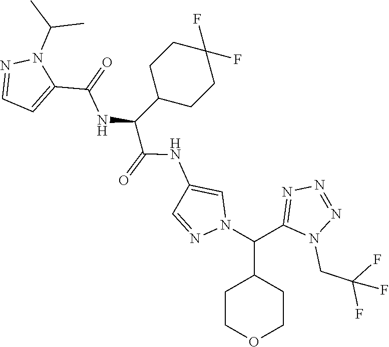 [SMILES: CC(C)n1nccc1C(=O)N[C@H](C(=O)Nc1cnn(C(c2nnnn2CC(F)(F)F)C2CCOCC2)c1)C1CCC(F)(F)CC1] | Diastereomer 1 of N-[(1S)-1- (4,4-difluorocyclohexyl)-2-oxo- 2-[[1-[tetrahydropyran-4-yl- [1-(2,2,2- trifluoroethyl)tetrazol-5- yl]methyl]pyrazol-4- yl]amino]ethyl]-2-isopropyl- pyrazole-3-carboxamide | 643.289 | 2.33 |
| 36^{#} | 69 | 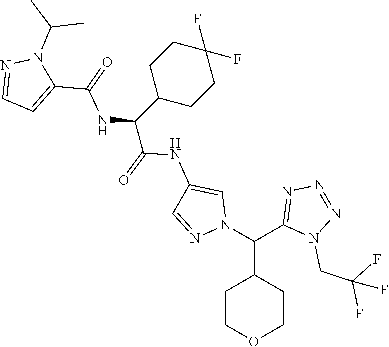 [SMILES: CC(C)n1nccc1C(=O)N[C@H](C(=O)Nc1cnn(C(c2nnnn2CC(F)(F)F)C2CCOCC2)c1)C1CCC(F)(F)CC1] | Diastereomer 2 of N-[(1S)-1- (4,4-difluorocyclohexyl)-2-oxo- 2-[[1-[tetrahydropyran-4-yl- [1-(2,2,2- trifluoroethyl)tetrazol-5- yl]methyl]pyrazol-4- yl]amino]ethyl]-2-isopropyl- pyrazole-3-carboxamide | 643.290 | 2.33 |
| 37^{#} | 73 | 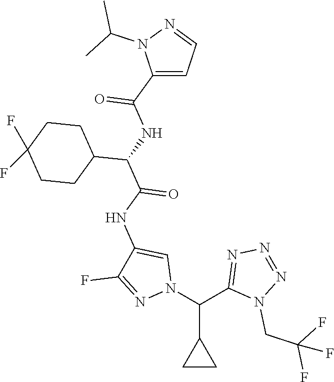 [SMILES: CC(C)n1nccc1C(=O)N[C@H](C(=O)Nc1cn(C(c2nnnn2CC(F)(F)F)C2CC2)nc1F)C1CCC(F)(F)CC1] | Diastereomer 1 of N-[(1S)-2- [[1-[cyclopropyl-[1-(2,2,2- trifluoroethyl)tetrazol-5- yl]methyl]-3-fluoro-pyrazol-4- ylamino]-1-(4,4- difluorocyclohexyl)-2-oxo- ethyl]-2-isopropyl-pyrazole-3- carboxamide | 617.254 | 2.44 |
| 38^{#} | 73 | 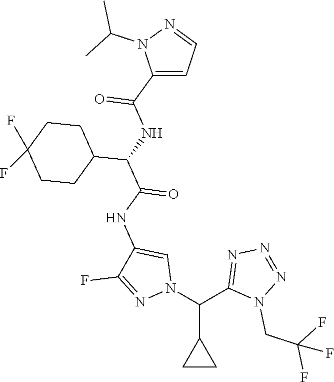 [SMILES: CC(C)n1nccc1C(=O)N[C@H](C(=O)Nc1cn(C(c2nnnn2CC(F)(F)F)C2CC2)nc1F)C1CCC(F)(F)CC1] | Diastereomer 2 of N-[(1S)-2- [[1-[cyclopropyl-[1-(2,2,2- trifluoroethyl)tetrazol-5- yl]methyl]-3-fluoro-pyrazol-4- yl]amino]-1-(4,4- difluorocyclohexyl)-2-oxo- ethyl]-2-isopropyl-pyrazole-3- carboxamide | 617.254 | 2.44 |
| 39^{#} | 76 | 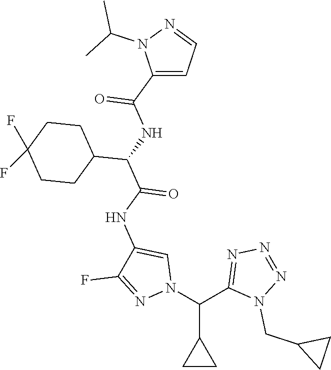 [SMILES: CC(C)n1nccc1C(=O)N[C@H](C(=O)Nc1cn(C(c2nnnn2CC2CC2)C2CC2)nc1F)C1CCC(F)(F)CC1] | Diastereomer 1 of N-[(1S)-2- [[1-[cyclopropyl-[1- (cyclopropylmethyl)tetrazol-5- yl]methyl]-3-fluoro-pyrazol-4- yl]amino]-1-(4,4- difluorocyclohexyl)-2-oxo- ethyl]-2-isopropyl-pyrazole-3- carboxamide | 589.298 | 2.42 |
| 40^{#} | 76 | 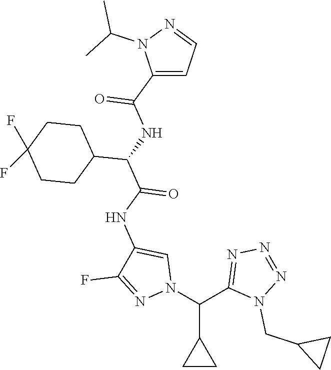 [SMILES: CC(C)n1nccc1C(=O)N[C@H](C(=O)Nc1cn(C(c2nnnn2CC2CC2)C2CC2)nc1F)C1CCC(F)(F)CC1] | Diastereomer 2 of N-[(1S)-2- [[1-[cyclopropyl-[1- (cyclopropylmethyl)tetrazol-5- yl]methyl]-3-fluoro-pyrazol-4- yl]amino]-1-(4,4- difluorocyclohexyl)-2-oxo- ethyl]-2-isopropyl-pyrazole-3- carboxamide | 589.298 | 2.42 |
| 41^{#} | 72 | 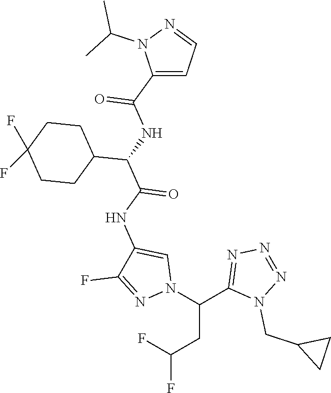 [SMILES: CC(C)n1nccc1C(=O)N[C@H](C(=O)Nc1cn(C(CC(F)F)c2nnnn2CC2CC2)nc1F)C1CCC(F)(F)CC1] | Diastereomer 1 of N-[(1S)-2- [[1-[1-[1-(cyclopropyl- methyl)tetrazol-5-yl]-3,3- difluoro-propyl]-3-fluoro- pyrazol-4-yl]amino]-1-(4,4- difluorocyclohexyl)-2-oxo- ethyl]-2-isopropyl-pyrazole-3- carboxamide | 613.279 | 2.42 |
| 42^{#} | 72 | 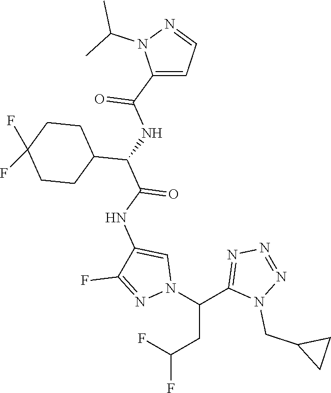 [SMILES: CC(C)n1nccc1C(=O)N[C@H](C(=O)Nc1cn(C(CC(F)F)c2nnnn2CC2CC2)nc1F)C1CCC(F)(F)CC1] | Diastereomer 2 of N-[(1S)-2- [[1-[1-[1-(cyclopropyl- methyl)tetrazol-5-yl]-3,3- difluoro-propyl]-3-fluoro- pyrazol-4-yl]amino]-1-(4,4- difluorocyclohexyl)-2-oxo- ethyl]-2-isopropyl-pyrazole-3- carboxamide | 613.279 | 2.43 |
| 43^{#} | 75 | 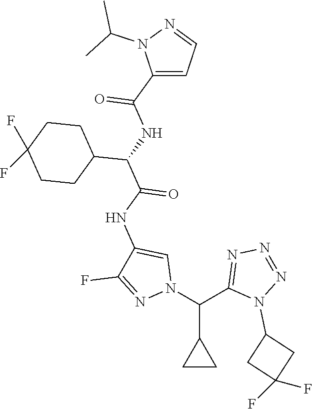 [SMILES: CC(C)n1nccc1C(=O)N[C@H](C(=O)Nc1cn(C(c2nnnn2C2CC(F)(F)C2)C2CC2)nc1F)C1CCC(F)(F)CC1] | Diastereomer 1 of N-[(1S)-2- [[1-[cyclopropyl-[1-(3,3- difluorocyclobutyl)tetrazol-5- yl]methyl]-3-fluoro-pyrazol-4- yl]amino]-1-(4,4- difluorocyclohexyl)-2-oxo- ethyl]-2-isopropyl-pyrazole-3- carboxamide | 625.279 | 2.44 |
| 44^{#} | 75 | 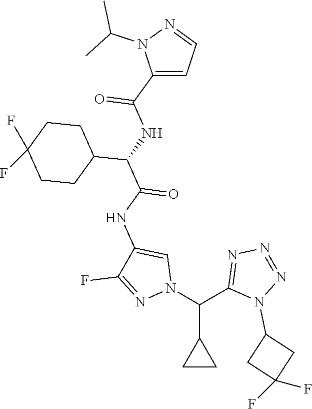 [SMILES: CC(C)n1nccc1C(=O)N[C@H](C(=O)Nc1cn(C(c2nnnn2C2CC(F)(F)C2)C2CC2)nc1F)C1CCC(F)(F)CC1] | Diastereomer 2 of N-[(1S)-2- [[1-[cyclopropyl-[1-(3,3- difluorocyclobutyl)tetrazol-5- yl]methyl]-3-fluoro-pyrazol-4- yl]amino]-1-(4,4-difluorocyclo- hexyl)-2-oxo-ethyl]-2-iso- propyl-pyrazole-3-carboxamide | 625.280 | 2.44 |
| 45^{#} | 74 | 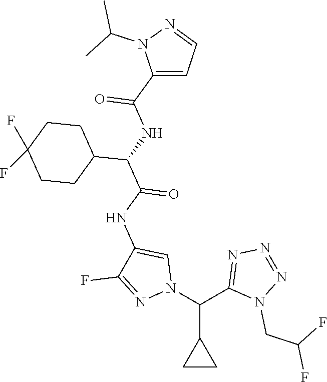 [SMILES: CC(C)n1nccc1C(=O)N[C@H](C(=O)Nc1cn(C(c2nnnn2CC(F)F)C2CC2)nc1F)C1CCC(F)(F)CC1] | Diastereomer 1 of N-[(1S)-2- [[1-[cyclopropyl-[1-(2,2- difluoroethyl)tetrazol-5- yl]methyl]-3-fluoro-pyrazol-4- yl]amino]-1-(4,4- difluorocyclohexyl)-2-oxo- ethyl]-2-isopropyl-pyrazole-3- carboxamide | 599.263 | 2.38 |
| 46^{#} | 74 | 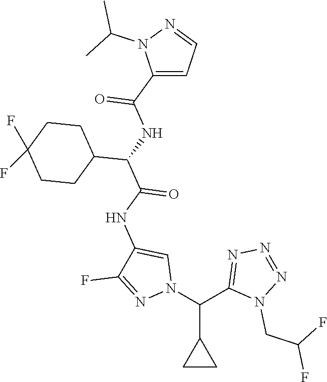 [SMILES: CC(C)n1nccc1C(=O)N[C@H](C(=O)Nc1cn(C(c2nnnn2CC(F)F)C2CC2)nc1F)C1CCC(F)(F)CC1] | Diastereomer 2 of N-[(1S)-2- [[1-[cyclopropyl-[1-(2,2- difluoroethyl)tetrazol-5- yl]methyl]-3-fluoro-pyrazol-4- yl]amino]-1-(4,4- difluorocyclohexyl)-2-oxo- ethyl]-2-isopropyl-pyrazole-3- carboxamide | 599.263 | 2.38 |
| 47^{#} | 69 | 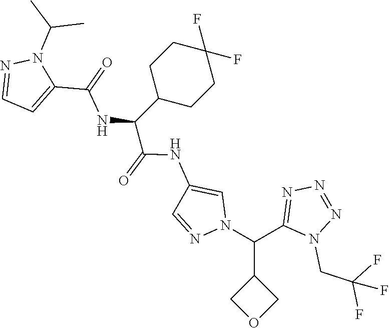 [SMILES: CC(C)n1nccc1C(=O)N[C@H](C(=O)Nc1cnn(C(c2nnnn2CC(F)(F)F)C2COC2)c1)C1CCC(F)(F)CC1] | Diastereomer 1 of N-[(1S)-1- (4,4-difluorocyclohexyl)-2-[[1- [oxetan-3-yl-[1-(2,2,2- trifluoroethyl)tetrazol-5-yl]- methyl]pyrazol-4-yl]amino]-2- oxo-ethyl]-2-isopropyl- pyrazole-3-carboxamide | 615.258 | 2.29 |
| 48^{#} | 69 | 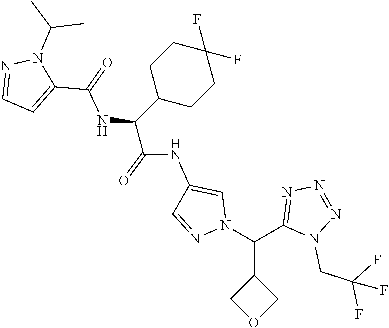 [SMILES: CC(C)n1nccc1C(=O)N[C@H](C(=O)Nc1cnn(C(c2nnnn2CC(F)(F)F)C2COC2)c1)C1CCC(F)(F)CC1] | Diastereomer 2 of N-[(1S)-1- (4,4-difluorocyclohexyl)-2-[[1- [oxetan-3-yl-[1-(2,2,2- trifluoroethyl)tetrazol-5-yl]- methyl]pyrazol-4-yl]amino]-2- oxo-ethyl]-2-isopropyl- pyrazole-3-carboxamide | 615.258 | 2.29 |
| 49^{#} | 71 | 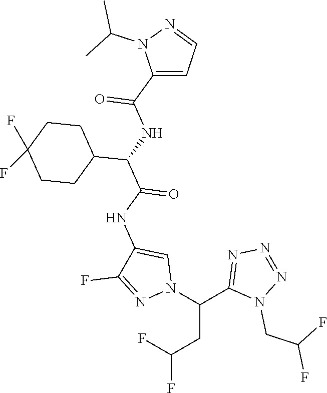 [SMILES: CC(C)n1nccc1C(=O)N[C@H](C(=O)Nc1cn(C(CC(F)F)c2nnnn2CC(F)F)nc1F)C1CCC(F)(F)CC1] | Diastereomer 1 of N-[(1S)-1- (4,4-difluorocyclohexyl)-2-[[1- [1-[1-(2,2- difluoroethyl)tetrazol-5-yl]- 3,3-difluoro-propyl]-3-fluoro- pyrazol-4-yl]amino]-2-oxo- ethyl]-2-isopropyl-pyrazole-3- carboxamide | 623.245 | 2.40 |
| 50^{#} | 71 | 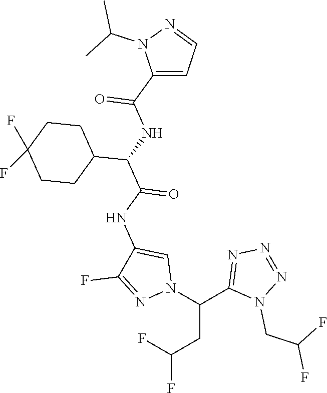 [SMILES: CC(C)n1nccc1C(=O)N[C@H](C(=O)Nc1cn(C(CC(F)F)c2nnnn2CC(F)F)nc1F)C1CCC(F)(F)CC1] | Diastereomer 2 of N-[(1S)-1- (4,4-difluorocyclohexyl)-2-[[1- [1-[1-(2,2- difluoroethyl)tetrazol-5-yl]- 3,3-difluoro-propyl]-3-fluoro- pyrazol-4-yl]amino]-2-oxo- ethyl]-2-isopropyl-pyrazole-3- carboxamide | 623.245 | 2.40 |

*Diastereomers of the CBz protected precursor were separated by chiral SFC
^{#}Diastereomers were separated by chiral SFC

### Example 51: N-[(1S)-2,2-dicyclopropyl-1-[[1-[1-[1-(cyclopropylmethyl)tetrazol-5-yl]-3,3-difluoro-propyl]pyrazol-4-yl]carbamoyl]ethyl]-2-isopropyl-pyrazole-3-carboxamide

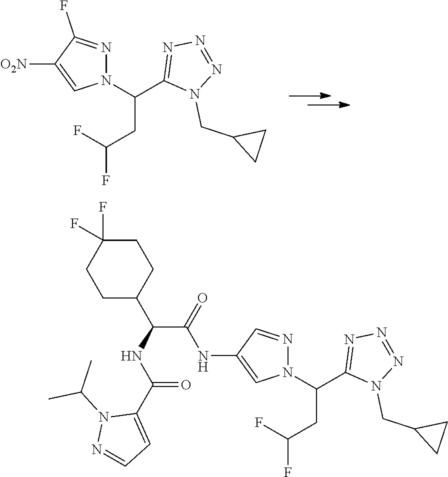

Following the methods of Preparations 11, 12 and 13 and Example 1 the nitro compound of Preparation 63 was reacted to give the title compound (7.3 mg) as a colourless solid. H NMR (600 MHz, DMSO-d6) δ 10.12 and 10.07 (2×s, 1H), 8.21 and 8.18 (2 d, J=8.8 Hz, 1H), 8.13 and 8.04 (2×s, 1H), 7.40 and 7.36 (2×s, 1H), 7.32 (d, J=2.0 Hz, 1H), 6.78-6.74 (m, 1H), 6.25-6.18 (m, 1H), 5.84 (ttd, J=55.7, 4.5, 1.7 Hz, 1H), 5.31-5.19 (m, 1H), 4.58-4.50 (m, 1H), 4.13-4.04 (m, 1H), 3.89-3.77 (m, 1H), 2.98-2.78 (m, 2H), 1.20 (d, J=6.6 Hz, 3H), 1.18 (d, J=6.6 Hz, 3H), 0.72-0.50 (m, 4H), 0.29-0.04 (m, 7H), 0.04-−0.05 (in, 4H), −0.10-−0.18 (in, 1H); LCMS (ES): m/z 571.307 [M+H]⁺, RT=2.42 mmx.

In a similar fashion to Example 51, Examples 52-76 were synthesised starting from the indicated nitropyrazoles.

| | Precursor | | | | |
|---|---|---|---|---|---|
| Ex. | Prep. | | | Mass | LCMS |
| No. | number | Structure | Name | ion | RT |
| 52 | 61 | 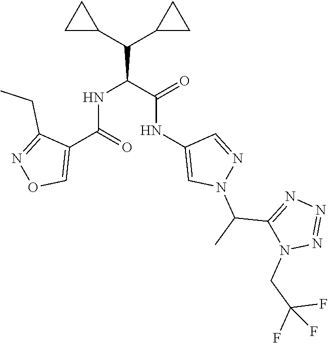 [SMILES: CCc1nocc1C(=O)N[C@H](C(=O)Nc1cnn(C(C)c2nnnn2CC(F)(F)F)c1)C(C1CC1)C1CC1] | N-[(1S)-1-(dicyclopropyl- methyl)-2-oxo-2-[[1-[1-[1- (2,2,2-trifluoroethyl)tetrazol-5- yl]ethyl]pyrazol-4- yl]amino]ethyl]-3-ethyl- isoxazole-4-carboxamide | 536.234 | 2.35 |
| 53 | 61 | 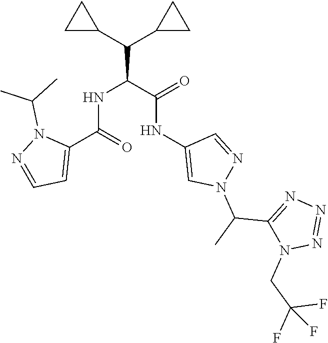 [SMILES: CC(c1nnnn1CC(F)(F)F)n1cc(NC(=O)[C@@H](NC(=O)c2ccnn2C(C)C)C(C2CC2)C2CC2)cn1] | N-[(1S)-1-(dicyclopropyl- methyl)-2-oxo-2-[[1-[1-[1- (2,2,2-trifluoroethyl)tetrazol-5- yl]ethyl]pyrazol-4- yl]amino]ethyl]-2-isopropyl- pyrazole-3-carboxamide | 549.266 | 2.37 |
| 54 | 46 | 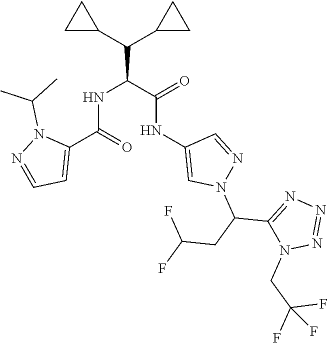 [SMILES: CC(C)n1nccc1C(=O)N[C@H](C(=O)Nc1cnn(C(CC(F)F)c2nnnn2CC(F)(F)F)c1)C(C1CC1)C1CC1] | N-[(1S)-1-(dicyclopropyl- methyl)-2-[[1-[3,3-difluoro-1- [1-(2,2,2-trifluoroethyl)- tetrazol-5-yl]propyl]pyrazol-4- yl]amino]-2-oxo-ethyl]-2-iso- propyl-pyrazole-3-carboxamide | 599.263 | 2.44 |
| 55 | 46 | 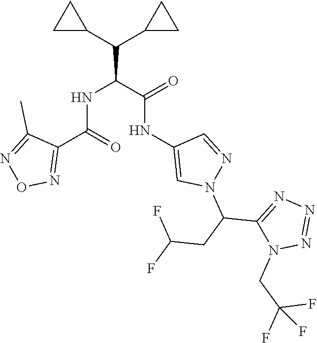 [SMILES: Cc1nonc1C(=O)N[C@H](C(=O)Nc1cnn(C(CC(F)F)c2nnnn2CC(F)(F)F)c1)C(C1CC1)C1CC1] | N-[(1S)-1-(dicyclopropyl- methyl)-2-[[1-[3,3-difluoro-1- [1-(2,2,2-trifluoroethyl)- tetrazol-5-yl]propyl]pyrazol-4- yl]amino]-2-oxo-ethyl]-4- methyl-1,2,5-oxadiazole-3- carboxamide | 573.211 | 2.49 |
| 56 | 43 | 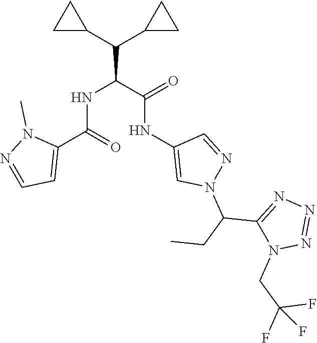 [SMILES: CCC(c1nnnn1CC(F)(F)F)n1cc(NC(=O)[C@@H](NC(=O)c2ccnn2C)C(C2CC2)C2CC2)cn1] | N-[(1S)-1-(dicyclopropyl- methyl)-2-oxo-2-[[1-[1-[1- (2,2,2-trifluoroethyl)tetrazol-5- yl]propyl]pyrazol-4- yl]amino]ethyl]-2-isopropyl- pyrazole-3-carboxamide | 563.282 | 2.43 |
| 57 | 43 | 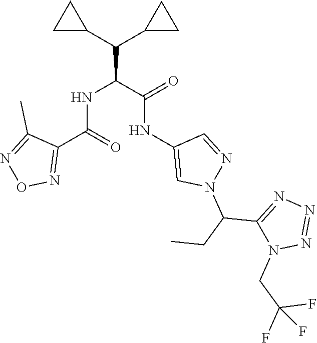 [SMILES: CCC(c1nnnn1CC(F)(F)F)n1cc(NC(=O)[C@@H](NC(=O)c2nonc2C)C(C2CC2)C2CC2)cn1] | N-[(1S)-1-(dicyclopropyl- methyl)-2-oxo-2-[[1-[1-[1- (2,2,2-trifluoroethyl)tetrazol-5- yl]propyl]pyrazol-4-yl]amino]- ethyl]-4-methyl-1,2,5- oxadiazole-3-carboxamide | 537.230 | 2.48 |
| 58 | 40 | 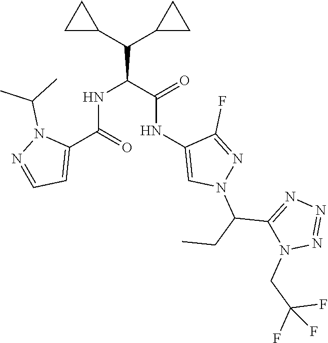 [SMILES: CCC(c1nnnn1CC(F)(F)F)n1cc(NC(=O)[C@@H](NC(=O)c2ccnn2C(C)C)C(C2CC2)C2CC2)c(F)n1] | N-[(1S)-1-(dicyclopropyl- methyl)-2-[[3-fluoro-1-[1-[1- (2,2,2-trifluoroethyl)tetrazol-5- yl]propyl]pyrazol-4-yl]amino]- 2-oxo-ethyl]-2-isopropyl- pyrazole-3-carboxamide | 581.273 | 2.49 |
| 59 | 62 | 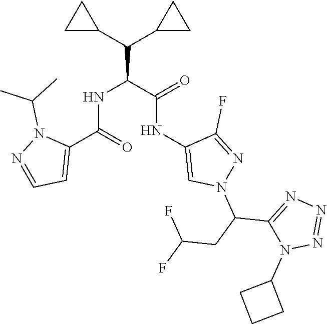 [SMILES: CC(C)n1nccc1C(=O)N[C@H](C(=O)Nc1cn(C(CC(F)F)c2nnnn2C2CCC2)nc1F)C(C1CC1)C1CC1] | N-[(1S)-1-[[1-[1-(1- cyclobutyltetrazol-5-yl)-3,3- difluoro-propyl]pyrazol-4- yl]carbamoyl]-2,2-dicyclo- propyl-ethyl]-2-isopropyl- pyrazole-3-carboxamide | 571.307 | 2.44 |
| 60 | 62 | 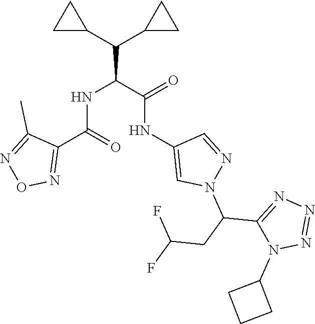 [SMILES: Cc1nonc1C(=O)N[C@H](C(=O)Nc1cnn(C(CC(F)F)c2nnnn2C2CCC2)c1)C(C1CC1)C1CC1] | N-[(1S)-1-[[1-[1-(1-cyclo- butyltetrazol-5-yl)-3,3- difluoro-propyl]pyrazol-4- yl]carbamoyl]-2,2-dicyclo- propyl-ethyl]-4-methyl-1,2,5- oxadiazole-3-carboxamide | 545.255 | 2.49 |
| 61 | 63 | 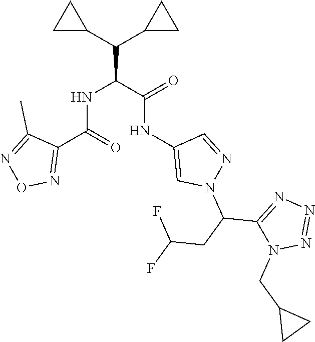 [SMILES: Cc1nonc1C(=O)N[C@H](C(=O)Nc1cnn(C(CC(F)F)c2nnnn2CC2CC2)c1)C(C1CC1)C1CC1] | N-[(1S)-2,2-dicyclopropyl-1- [[1-[1-[1-(cyclopropylmethyl)- tetrazol-5-yl]-3,3- difluoropropyl]pyrazol-4- yl]carbamoyl]ethyl]-4-methyl- 1,2,5-oxadiazole-3- carboxamide | 545.255 | 2.47 |
| 62 | 64 | 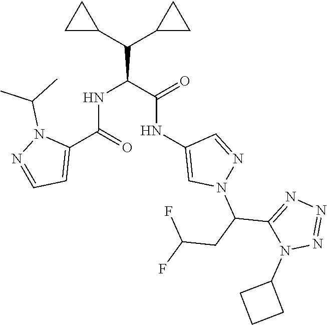 [SMILES: CC(C)n1nccc1C(=O)N[C@H](C(=O)Nc1cnn(C(CC(F)F)c2nnnn2C2CCC2)c1)C(C1CC1)C1CC1] | N-[(1S)-2,2-dicyclopropyl-1- [[1-[1-(1-cyclopropyltetrazol- 5-yl)-3,3-difluoropropyl]- pyrazol-4-yl]carbamoyl]ethyl]- 2-isopropyl-pyrazole-3- carboxamide | 557.292 | 2.37 |
| 63 | 64 | 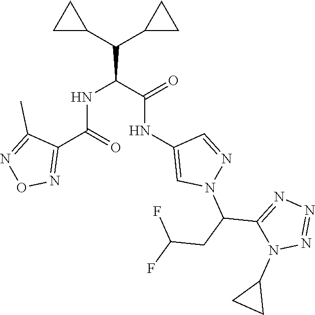 [SMILES: Cc1nonc1C(=O)N[C@H](C(=O)Nc1cnn(C(CC(F)F)c2nnnn2C2CC2)c1)C(C1CC1)C1CC1] | N-[(1S)-2,2-dicyclopropyl-1- [[1-[1-(1-cyclopropyltetrazol- 5-yl)-3,3-difluoropropyl]- pyrazol-4-yl]carbamoyl]ethyl]- 4-methyl-1,2,5-oxadiazole-3- carboxamide | 531.239 | 2.43 |
| 64 | 51 | 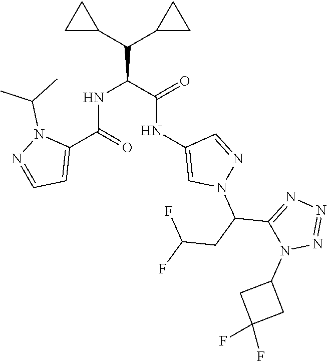 [SMILES: CC(C)n1nccc1C(=O)N[C@H](C(=O)Nc1cnn(C(CC(F)F)c2nnnn2C2CC(F)(F)C2)c1)C(C1CC1)C1CC1] | N-[(1S)-1-(dicyclopropyl- methyl)-2-[[1-[1-[1-(3,3- difluorocyclobutyl)tetrazol-5- yl]-3,3-difluoro-propyl]pyrazol- 4-yl]amino]-2-oxo-ethyl]-2- isopropyl-pyrazole-3- carboxamide | 607.288 | 2.45 |
| 65 | 51 | 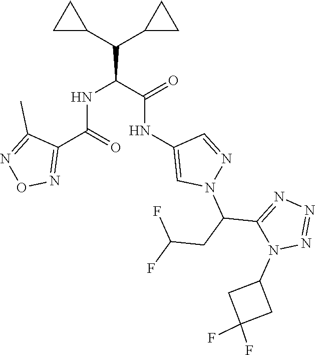 [SMILES: Cc1nonc1C(=O)N[C@H](C(=O)Nc1cnn(C(CC(F)F)c2nnnn2C2CC(F)(F)C2)c1)C(C1CC1)C1CC1] | N-[(1S)-1-(dicyclopropyl- methyl)-2-[[1-[1-[1-(3,3- difluorocyclobutyl)tetrazol-5- yl]-3,3-difluoro-propyl]pyrazol- 4-yl]amino]-2-oxo-ethyl]-4- methyl-1,2,5-oxadiazole-3- carboxamide | 581.236 | 2.50 |
| 66 | 65 | 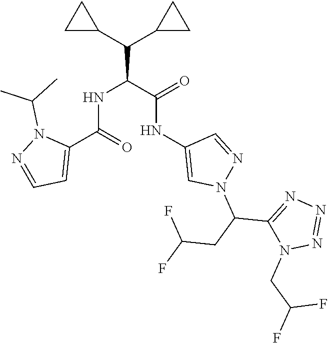 [SMILES: CC(C)n1nccc1C(=O)N[C@H](C(=O)Nc1cnn(C(CC(F)F)c2nnnn2CC(F)F)c1)C(C1CC1)C1CC1] | N-[(1S)-1-(dicyclopropyl- methyl)-2-[[1-[1-[1-(2,2- difluoroethyl)tetrazol-5-yl]- 3,3-difluoro-propyl]pyrazol-4- yl]amino]-2-oxo-ethyl]-2- isopropyl-pyrazole-3- carboxamide | 581.273 | 2.38 |
| 67 | 65 | 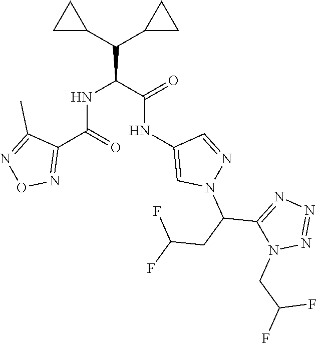 [SMILES: Cc1nonc1C(=O)N[C@H](C(=O)Nc1cnn(C(CC(F)F)c2nnnn2CC(F)F)c1)C(C1CC1)C1CC1] | N-[(1S)-1-(dicyclopropyl- methyl)-2-[[1-[1-[1-(2,2- difluoroethyl)tetrazol-5-yl]- 3,3-difluoro-propyl]pyrazol-4- yl]amino]-2-oxo-ethyl]-4- methyl-1,2,5-oxadiazole-3- carboxamide | 555.223 | 2.43 |
| 68^{#} | 73 | 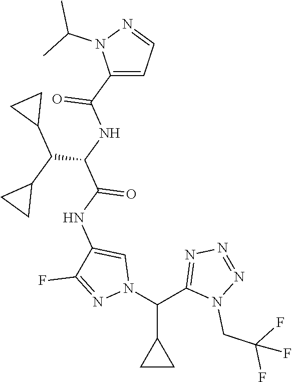 [SMILES: CC(C)n1nccc1C(=O)N[C@H](C(=O)Nc1cn(C(c2nnnn2CC(F)(F)F)C2CC2)nc1F)C(C1CC1)C1CC1] | Diastereomer 1 of N-[(1S)-2,2- dicyclopropyl-1-[[1-[cyclo- propyl-[1-(2,2,2-trifluoro- ethyl)tetrazol-5-yl]methyl]-3- fluoro-pyrazol-4-yl]- carbamoyl]ethyl]-2-isopropyl- pyrazole-3-carboxamide | 593.272 | 2.49 |
| 69^{#} | 73 | 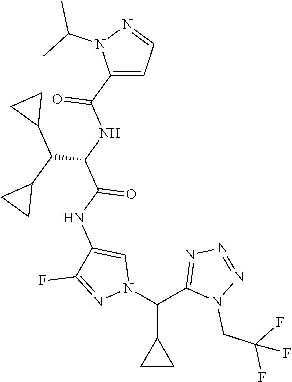 [SMILES: CC(C)n1nccc1C(=O)N[C@H](C(=O)Nc1cn(C(c2nnnn2CC(F)(F)F)C2CC2)nc1F)C(C1CC1)C1CC1] | Diastereomer 2 of N-[(1S)-2,2- dicyclopropyl-1-[[1-[cyclo- propyl-[1-(2,2,2-trifluoro- ethyl)tetrazol-5-yl]methyl]-3- fluoro-pyrazol-4-yl]- carbamoyl]ethyl]-2-isopropyl- pyrazole-3-carboxamide | 593.273 | 2.49 |
| 70^{#} | 76 | 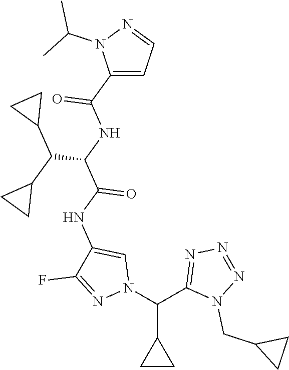 [SMILES: CC(C)n1nccc1C(=O)N[C@H](C(=O)Nc1cn(C(c2nnnn2CC2CC2)C2CC2)nc1F)C(C1CC1)C1CC1] | Diastereomer 1 of N-[(1S)-2,2- dicyclopropyl-1-[[1-[cyclo- propyl-[1-(cyclopropylmethyl)- tetrazol-5-yl]methyl]-3-fluoro- pyrazol-4-yl]carbamoyl]ethyl]- 2-isopropyl-pyrazole-3- carboxamide | 565.317 | 2.47 |
| 71^{#} | 76 | 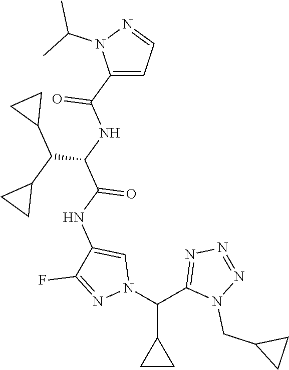 [SMILES: CC(C)n1nccc1C(=O)N[C@H](C(=O)Nc1cn(C(c2nnnn2CC2CC2)C2CC2)nc1F)C(C1CC1)C1CC1] | Diastereomer 2 of N-[(1S)-2,2- dicyclopropyl-1-[1-[cyclo- propyl-[1-(cyclopropylmethyl)- tetrazol-5-yl]methyl]-3-fluoro- pyrazol-4-yl]carbamoyl]ethyl]- 2-isopropyl-pyrazole-3- carboxamide | 565.317 | 2.47 |
| 72 | 67 | 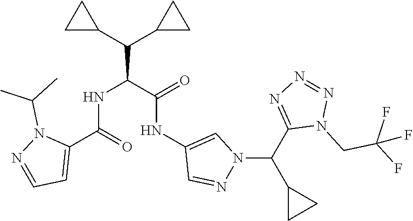 [SMILES: CC(C)n1nccc1C(=O)N[C@H](C(=O)Nc1cnn(C(c2nnnn2CC(F)(F)F)C2CC2)c1)C(C1CC1)C1CC1] | N-[(1S)-2,2-dicyclopropyl-1- [[1-[cyclopropyl-[1-(2,2,2- trifluoroethyl)tetrazol-5- yl]methyl]pyrazol-4- yl]carbamoyl]ethyl]-2- isopropyl-pyrazole-3- carboxamide | 575.281 | 2.45 |
| 73^{#} | 77 | 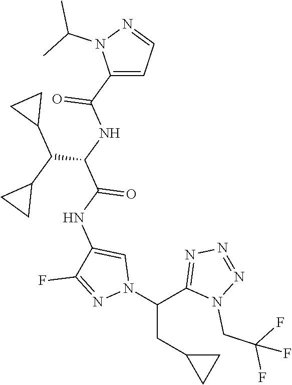 [SMILES: CC(C)n1nccc1C(=O)N[C@H](C(=O)Nc1cn(C(CC2CC2)c2nnnn2CC(F)(F)F)nc1F)C(C1CC1)C1CC1] | Diastereomer 1 of N-[(1S)-2,2- dicyclopropyl-1-[[1-[2-cyclo- propyl-1-[1-(2,2,2- trifluoroethyl)tetrazol-5- yl]ethyl]-3-fluoro-pyrazol-4- yl]carbamoyl]ethyl]-2- isopropyl-pyrazole-3- carboxamide | 607.5* | 2.17 |
| 74^{#} | 77 | 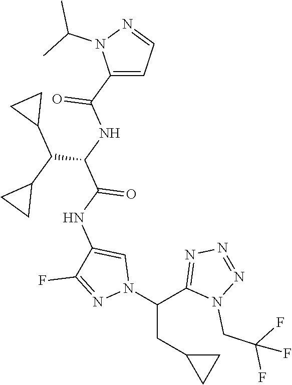 [SMILES: CC(C)n1nccc1C(=O)N[C@H](C(=O)Nc1cn(C(CC2CC2)c2nnnn2CC(F)(F)F)nc1F)C(C1CC1)C1CC1] | Diastereomer 2 of N-[(1S)-2,2- dicyclopropyl-1-[[1-[2-cyclo- propyl-1-[1-(2,2,2- trifluoroethyl)tetrazol-5- yl]ethyl]-3-fluoro-pyrazol-4- yl]carbamoyl]ethyl]-2- isopropyl-pyrazole-3- carboxamide | 607.5* | 2.17 |
| 75^{#} | 78 | 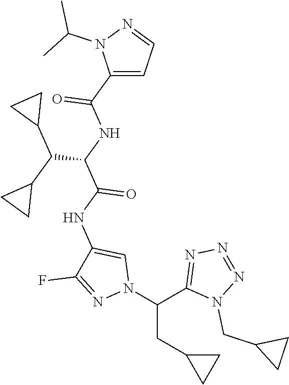 [SMILES: CC(C)n1nccc1C(=O)N[C@H](C(=O)Nc1cn(C(CC2CC2)c2nnnn2CC2CC2)nc1F)C(C1CC1)C1CC1] | Diastereomer 1 of N-[(1S)-2,2- dicyclopropyl-1-[[1-[2- cyclopropyl-1-[1- (cyclopropylmethyl)tetrazol-5- yl]ethyl]-3-fluoro-pyrazol-4- yl]carbamoyl]ethyl]-2- isopropyl-pyrazole-3- carboxamide | 577.6* | 2.14 |
| 76^{#} | 78 | 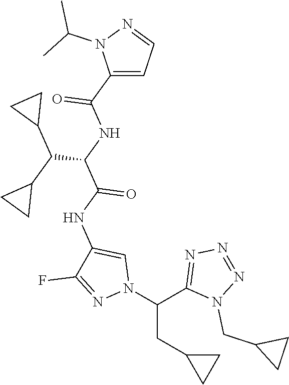 [SMILES: CC(C)n1nccc1C(=O)N[C@H](C(=O)Nc1cn(C(CC2CC2)c2nnnn2CC2CC2)nc1F)C(C1CC1)C1CC1] | Diastereomer 2 of N-[(1S)-2,2- dicyclopropyl-1-[[1-[2- cyclopropyl-1-[1- (cyclopropylmethyl)tetrazol-5- yl]ethyl]-3-fluoro-pyrazol-4- yl]carbamoyl]ethyl]-2- isopropyl-pyrazole-3- carboxamide | 577.5* | 2.14 |

^{#}Diastereomers were separated by chiral SFC
*Method 2 - ACQUITY BEH C18 (50 mm × 2.1 mm) column, 0.05% Formic acid in water, 0.05% Formic acid in MeCN

### Example 77: N-[(1S)-2-[[1-[3,3-difluoro-1-[1-(2,2,2-trifluoroethyl)tetrazol-5-yl]propyl]-3-fluoro-pyrazol-4-yl]amino]-1-(trans-4-methylcyclohexyl)-2-oxo-ethyl]-2-isopropyl-pyrazole-3-carboxamide

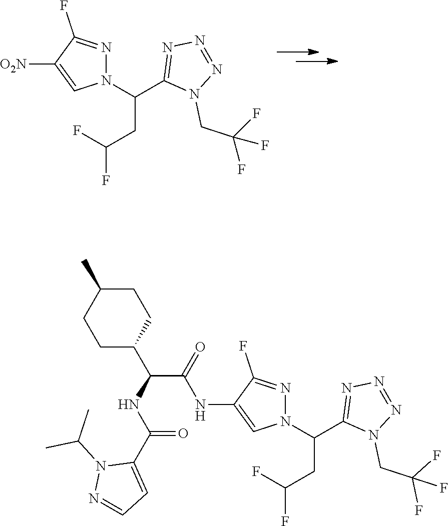 [SMILES: O=[N+]([O-])c1cn(C(CC(F)F)c2nnnn2CC(F)(F)F)nc1F ; CC(C)n1nccc1C(=O)N[C@H](C(=O)Nc1cn(C(CC(F)F)c2nnnn2CC(F)(F)F)nc1F)[C@H]1CC[C@H](C)CC1]

Following the methods of Preparations 11, 12 and 13 and Example 1, Example 77 was prepared from the nitro compound of Preparation 55 and (2S)-2-(tert-butoxycarbonylamino)-2-(trans-4-methylcyclohexyl)acetic acid to give the title compound (9 mg) as a colourless solid. LCMS (ES): m/z 619.270 [M+H]⁺, RT=2.57 min.

Examples 78-80 were synthesised in a similar fashion to Example 77, using the appropriate acid in the final amide coupling step.

| Ex. | | | Mass | LCMS |
|---|---|---|---|---|
| No. | Structure | Name | ion | RT |
| 78 | 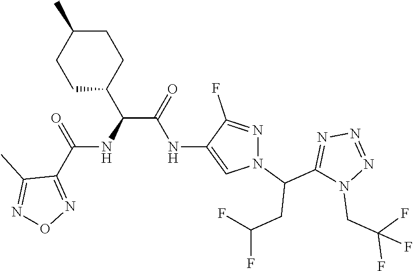 [SMILES: Cc1nonc1C(=O)N[C@H](C(=O)Nc1cn(C(CC(F)F)c2nnnn2CC(F)(F)F)nc1F)[C@H]1CC[C@H](C)CC1] | N-[(1S)-2-[[1-[3,3-difluoro-1-[1- (2,2,2-trifluoroethyl)tetrazol-5- yl]propyl]-3-fluoro-pyrazol-4- yl]amino]-1-(trans-4-methylcyclo- hexyl)-2-oxo-ethyl]-4-methyl-1,2,5- oxadiazole-3-carboxamide | 591.202 [M − H]⁻ | 2.57 |
| 79* | 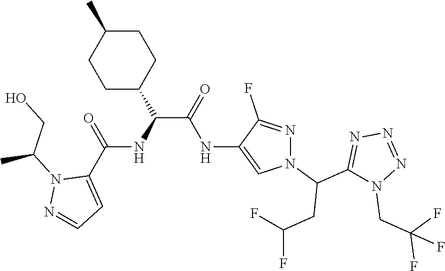 [SMILES: C[C@@H](CO)n1nccc1C(=O)N[C@H](C(=O)Nc1cn(C(CC(F)F)c2nnnn2CC(F)(F)F)nc1F)[C@H]1CC[C@H](C)CC1] | Diastereomer 1 of N-[(1S)-2-[[1- [3,3-difluoro-1-[1-(2,2,2- trifluoroethyl) tetrazol-5-yl]propyl]-3- fluoro-pyrazol-4-yl]amino]-1-(trans- 4-methylcyclohexyl)-2-oxo-ethyl]-2- [(1S)-2-hydroxy-1-methyl- ethyl]pyrazole-3-carboxamide | 635.264 | 2.38 |
| 80* | 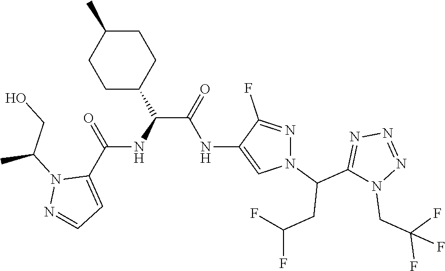 [SMILES: C[C@@H](CO)n1nccc1C(=O)N[C@H](C(=O)Nc1cn(C(CC(F)F)c2nnnn2CC(F)(F)F)nc1F)[C@H]1CC[C@H](C)CC1] | Diastereomer 2 of N-[(1S)-2-[[1- [3,3-difluoro-1-[1-(2,2,2- trifluoroethyl) tetrazol-5-yl]propyl]-3- fluoro-pyrazol-4-yl]amino]-1-(trans- 4-methylcyclohexyl)-2-oxo-ethyl]-2- [(1S)-2-hydroxy-1-methyl- ethyl]pyrazole-3-carboxamide | 635.264 | 2.38 |

*The enantiomers of Preparation 55 were separated by chiral SFC

### Example 81: N-[(15)-1-(tert-butoxymethyl)-2-[[l-[3,3-difluoro-1-[1-(2,2,2-trifluoroethyl)tetrazol-5-yl]propyl]-3-fluoro-pyrazol-4-yl]amino]-2-oxo-ethyl]-2-isopropyl-pyrazole-3-carboxamide

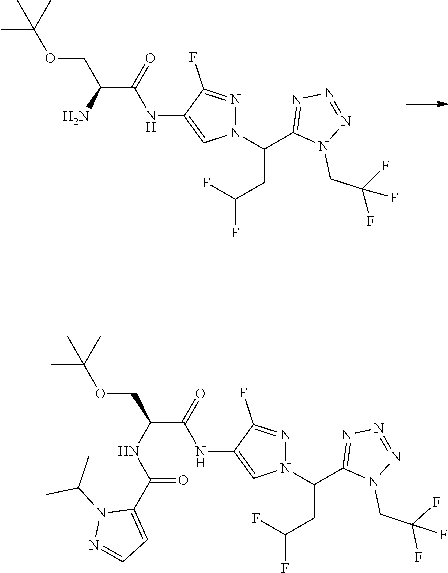 [SMILES: CC(C)(C)OC[C@H](N)C(=O)Nc1cn(C(CC(F)F)c2nnnn2CC(F)(F)F)nc1F ; CC(C)n1nccc1C(=O)N[C@@H](COC(C)(C)C)C(=O)Nc1cn(C(CC(F)F)c2nnnn2CC(F)(F)F)nc1F]

According to the method of Example 1 the compound of Preparation 80 was reacted with 2-isopropylpyrazole-3-carboxylic acid to give the title compound (4.9 mg, 510/Yield). ¹H NMR (400 MHz, DMSO-d6) δ 10.07 (d, J=13.4 Hz, 1H), 8.42 (d, J=8.0 Hz, 1H), 8.38 (dd, J=11.7, 2.0 Hz, 1H), 7.50 (d, J=1.9 Hz, 1H), 6.88 (t, J=1.9 Hz, 1H), 6.36 (t, J=4.6 Hz, 1H), 6.04 (t, J=55.4 Hz, 1H), 5.83-5.55 (m, 2H), 5.40 (m, 1H), 4.68 (q, J=6.8 Hz, 1H), 3.72-3.49 (m, 2H), 3.18-2.78 (m, 2H), 1.39-1.32 (m, 6H), 1.11 (2×s, 9H); LCMS (ES): m/z 609.249 [M+H]⁺, RT=2.46 min.

### Example 82: N-[(15)-1-(tert-butoxymethyl)-2-[[l-[3,3-difluoro-1-[1-(2,2,2-trifluoroethyl)tetrazol-5-yl]propyl]-3-fluoro-pyrazol-4-yl]amino]-2-oxo-ethyl]-4-ethyl-1,2,5-oxadiazole-3-carboxamide

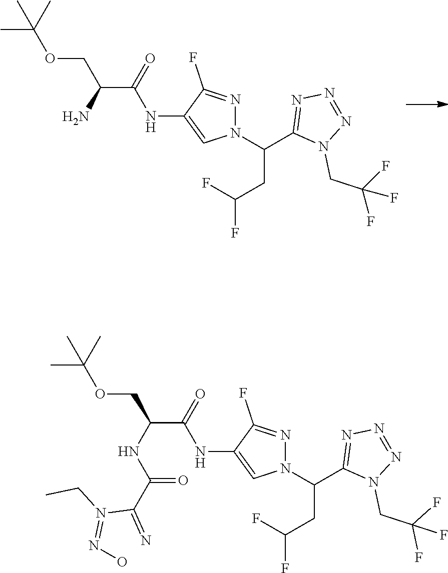

According to the method of Example 1 the compound of Preparation 80 was reacted with 4-ethyl-1,2,5-oxadiazole-3-carboxylic acid to give the title compound (4.8 mg, 510 Yield). H NMR (600 MHz, DMSO-d6) δ 10.13 (s, 1H), 8.99 (s, 1H), 8.39 (dd, J=14.2, 1.8 Hz, 1H), 6.43-6.30 (i, 1H), 6.04 (tq, J=55.4, 3.8 Hz, 1H), 5.83-5.56 (2, 2H), 4.73 (d, J=5.2 Hz, 1H), 3.74-3.55 (m, 2H), 3.18-3.01 (m, 1H), 2.98-2.82 (m, 3H), 1.25 (t, J=7.5 Hz, 3H), 1.11 (2×s, 9H); LCMS (ES): m/z 595.197 [M+H]⁺, RT=2.56 min.

In a similar fashion to the synthesis of Example 10, Examples 83 and 84 were synthesised starting from the enantiomers of the nitropyrazole of Preparation 55 (separated by chiral SFC) and the acid of Preparation 82.

| Ex. | | | Mass | LCMS |
|---|---|---|---|---|
| No. | Structure | Name | ion | RT |
| 83 | 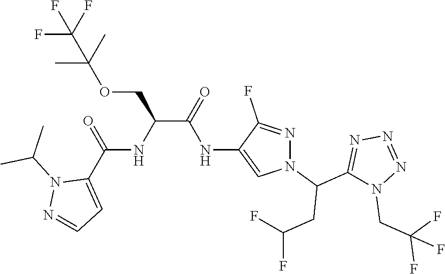 [SMILES: CC(C)n1nccc1C(=O)N[C@@H](COC(C)(C)C(F)(F)F)C(=O)Nc1cn(C(CC(F)F)c2nnnn2CC(F)(F)F)nc1F] | Diastereomer 1 of N-[(1S)-2-[[1- [3,3-difluoro-1-[1-(2,2,2- trifluoroethyl)tetrazol-5-yl]propyl]-3- fluoro-pyrazol-4-yl]amino]-2-oxo-1- [(2,2,2-trifluoro-1,1-dimethyl- ethoxy)methyl]ethyl]-2-isopropyl- pyrazole-3-carboxamide | 663.220 | 2.48 |
| 84 | 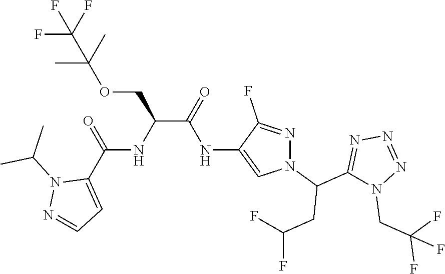 [SMILES: CC(C)n1nccc1C(=O)N[C@@H](COC(C)(C)C(F)(F)F)C(=O)Nc1cn(C(CC(F)F)c2nnnn2CC(F)(F)F)nc1F] | Diastereomer 2 of N-[(1S)-2-[[1- [3,3-difluoro-1-[1-(2,2,2- trifluoroethyl) tetrazol-5-yl]propyl]-3- fluoro-pyrazol-4-yl]amino]-2-oxo-1- [(2,2,2-trifluoro-1,1-dimethyl- ethoxy)methyl]ethyl]-2-isopropyl- pyrazole-3-carboxamide | 663.220 | 2.48 |

### Example 85 N-[(1S)-1-(dicyclopropylmethyl)-2-[[3-[3,3-difluoro-1-[1-(2,2,2-trifluoroethyl)tetrazol-5-yl]propyl]isoxazol-5-yl]amino]-2-oxo-ethyl]-2-isopropyl-pyrazole-3-carboxamide

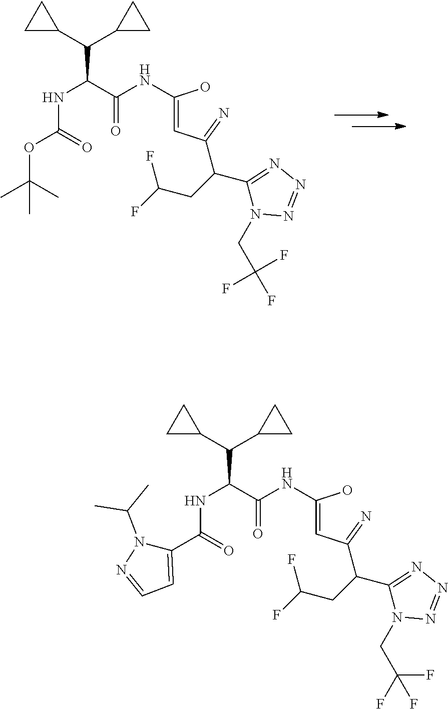 [SMILES: CC(C)(C)OC(=O)N[C@H](C(=O)N/C(O)=C/C(=N)C(CC(F)F)c1nnnn1CC(F)(F)F)C(C1CC1)C1CC1 ; CC(C)n1nccc1C(=O)N[C@H](C(=O)N/C(O)=C/C(=N)C(CC(F)F)c1nnnn1CC(F)(F)F)C(C1CC1)C1CC1]

According to the methods of Preparation 13 and Example 1 the compound of Preparation 91 and 2-isopropylpyrazole-3-carboxylic acid were converted to the title compound (7.4 mg, 51% Yield) as an off-white solid. ¹H NMR (400 MHz, DMSO) δ 11.79 (s, 1H), 8.33 (d, J=8.2 Hz, 1H), 7.31 (d, J=2.0 Hz, 1H), 6.71 (d, J=2.0 Hz, 1H), 6.16 and 6.12 (2×s, 1H), 5.98 and 5.84 (2×t, J=4.3 Hz, 1H), 5.61-5.44 (m, 2H), 5.24-5.09 (m, 1H), 4.93 (t, J=7.2 Hz, 1H), 4.56 (t, J=7.7 Hz, 1H), 2.74-2.56 (m, 2H), 1.20-1.12 (m, 6H), 0.86-0.67 (m, 2H), 0.65-0.48 (m, 2H), 0.34-0.22 (m, 1H), 0.22-0.13 (m, 1H), 0.13-−0.09 (m, 4H), −0.09-−0.24 (m, 1H); LCMS (ES): m/z 600.254 [M+H]⁺, RT=2.49 min.

Examples 86 and 87 were synthesised in a similar fashion to Example 85.

| Ex. | | | Mass | LCMS |
|---|---|---|---|---|
| No. | Structure | Name | ion | RT |
| 86 | 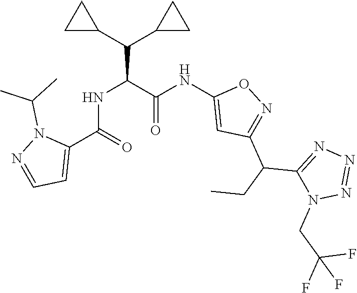 [SMILES: CCC(c1cc(NC(=O)[C@@H](NC(=O)c2ccnn2C(C)C)C(C2CC2)C2CC2)on1)c1nnnn1CC(F)(F)F] | N-[(1S)-1-(dicyclopropylmethyl)-2-oxo-2- [[3-[1-[1-(2,2,2-trifluoroethyl)tetrazol-5- yl]propyl]isoxazol-5-yl]amino]ethyl]-2- isopropyl-pyrazole-3-carboxamide | 564.266 | 2.50 |
| 87 | 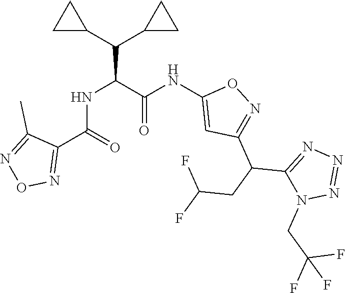 [SMILES: Cc1nonc1C(=O)N[C@H](C(=O)Nc1cc(C(CC(F)F)c2nnnn2CC(F)(F)F)no1)C(C1CC1)C1CC1] | N-[(1S)-1-(dicyclopropylmethyl)-2-[[3- [3,3-difluoro-1-[1-(2,2,2-trifluoro- ethyl)tetrazol-5-yl]propyl]isoxazol-5- yl]amino]-2-oxo-ethyl]-4-methyl-1,2,5- oxadiazole-3-carboxamide | 574.196 | 2.53 |

### Example 88: N-[(1S)-1-(dicyclopropylmethyl)-2-[[1-[(1S)-3,3-difluoro-1-[4-(2,2,2-trifluoroethyl)-1,2,4-triazol-3-yl]propyl]-3-fluoro-pyrazol-4-yl]amino]-2-oxo-ethyl]-2-isopropyl-pyrazole-3-carboxamide

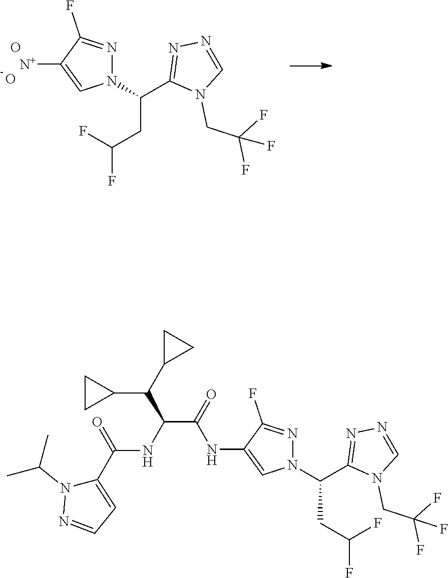

Example 88 was prepared from the compounds of Preparation 5 and 96, according to the methods of Preparations 11, 12 and 24 and Example 1. pH NMR (600 MHz, DMSO) 10.12 (s, 1H), 8.66 (s, 1H), 8.37 (d, J=8.8 Hz, 1H), 8.31 (d, J=1.8 Hz, 1H), 7.49 (d, J=1.9 Hz, 1H), 6.90 (d, J=2.0 Hz, 1H), 6.12-5.89 (m, 2H), 5.39 (hept, J=6.7 Hz, 1H), 5.25 (dq, J=15.7, 9.0 Hz, 1H), 4.96 (dt, J=15.8, 8.9 Hz, 1H), 4.82 (t, J=8.3 Hz, 1H), 3.04-2.84 (m, 2H), 1.37 (d, J=6.6 Hz, 3H), 1.35 (d, J=6.6 Hz, 3H), 0.91-0.83 (m, 1H), 0.79-0.72 (m, 1H), 0.69 (q, J=8.9 Hz, 1H), 0.47-0.41 (m, 1H), 0.38-0.23 (m, 3H), 0.22-0.14 (m, 3H), 0.10-0.04 (in, 1H); LCMS (ES): m/z 616.258 [M+H]⁺, RT=2.34 min.

Examples 89-91 were synthesised in a similar fashion to Example 88 from the compound of Preparation 5 and the indicated nitropyrazole.

| Ex. | Precursor | | | Mass | LCMS |
|---|---|---|---|---|---|
| No. | Prep. number | Structure | Name | ion | RT |
| 89 | 100 | 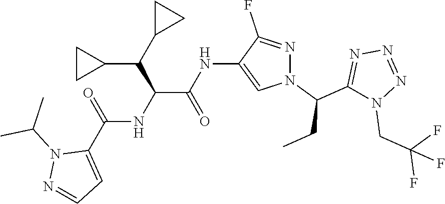 [SMILES: CC[C@H](c1nnnn1CC(F)(F)F)n1cc(NC(=O)[C@@H](NC(=O)c2ccnn2C(C)C)C(C2CC2)C2CC2)c(F)n1] | N-[(1S)-1-(dicyclopropyl- methyl)-2-[[3-fluoro-1-[(1R)- 1-[4-(2,2,2-trifluoroethyl)- 1,2,4-triazol-3-yl]propyl]- pyrazol-4-yl]amino]-2-oxo- ethyl]-2-isopropyl-pyrazole-3- carboxamide | 580.277 | 2.32 |
| 90 | 99 | 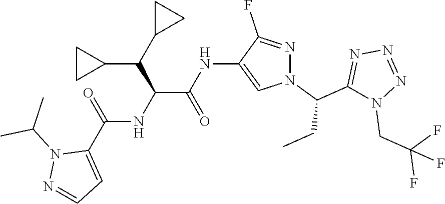 [SMILES: CC[C@@H](c1nnnn1CC(F)(F)F)n1cc(NC(=O)[C@@H](NC(=O)c2ccnn2C(C)C)C(C2CC2)C2CC2)c(F)n1] | N-[(1S)-1-(dicyclopropyl- methyl)-2-[[3-fluoro-1-[(1S)- 1-[4-(2,2,2-trifluoroethyl)- 1,2,4-triazol-3-yl]propyl]- pyrazol-4-yl]amino]-2-oxo- ethyl]-2-isopropyl-pyrazole-3- carboxamide | 580.277 | 2.31 |
| 91 | 96 | 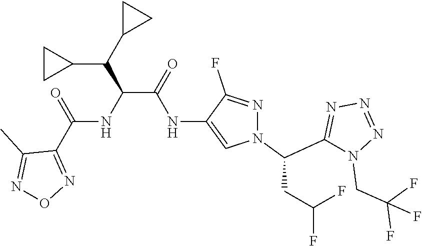 [SMILES: Cc1nonc1C(=O)N[C@H](C(=O)Nc1cn([C@@H](CC(F)F)c2nnnn2CC(F)(F)F)nc1F)C(C1CC1)C1CC1] | N-[(1S)-1-(dicyclopropyl- methyl)-2-[[1-[(1S)-3,3- difluoro-1-[4-(2,2,2- trifluoroethyl)-1,2,4-triazol-3- yl]propyl]-3-fluoro-pyrazol-4- yl]amino]-2-oxo-ethyl]-4- methyl-1,2,5-oxadiazole-3- carboxamide | 590.206 | 2.39 |

Examples 92 and 93 were synthesised in a similar fashion to Example 88 from the compound of Preparation 82 and the indicated nitropyrazole.

| Ex. | Precursor | | | Mass | LCMS |
|---|---|---|---|---|---|
| No. | Prep. number | Structure | Name | ion | RT |
| 92 | 96 | 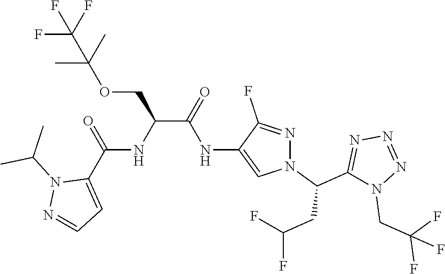 [SMILES: CC(C)n1nccc1C(=O)N[C@@H](COC(C)(C)C(F)(F)F)C(=O)Nc1cn([C@@H](CC(F)F)c2nnnn2CC(F)(F)F)nc1F] | N-[(1S)-2-[1-[(1S)-3,3- difluoro-1-[4-(2,2,2-trifluoro- ethyl)-1,2,4-triazol-3-yl]- propyl]-3-fluoro-pyrazol-4- yl]amino]-2-oxo-1-[(2,2,2- trifluoro-1,1-dimethyl-ethoxy)- methyl]ethyl]-2-isopropyl- pyrazole-3-carboxamide | 662.225 | 2.34 |
| 93 | 101 | 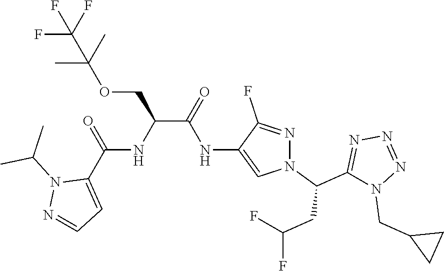 [SMILES: CC(C)n1nccc1C(=O)N[C@@H](COC(C)(C)C(F)(F)F)C(=O)Nc1cn([C@@H](CC(F)F)c2nnnn2CC2CC2)nc1F] | N-[(1S)-2-[[1-[(1S)-1-[4- (cyclopropylmethyl)-1,2,4- triazol-3-yl]-3,3-difluoro- propyl]-3-fluoro-pyrazol-4- yl]amino]-2-oxo-1-[(2,2,2- trifluoro-1,1-dimethyl-ethoxy)- methyl]ethyl]-2-isopropyl- pyrazole-3-carboxamide | 634.269 | 2.32 |

Examples 94 and 95 were prepared according to the methods of Preparations 11, 12 and 13 and Example 1 from the indicated nitropyrazoles.

| Ex. | Precursor | | | Mass | LCMS |
|---|---|---|---|---|---|
| No. | Prep. number | Structure | Name | ion | RT |
| 94 | 98 | 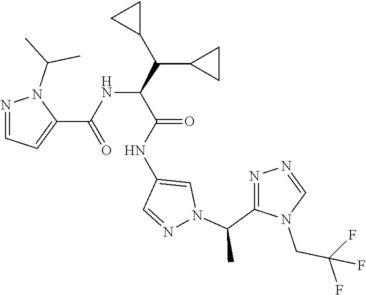 [SMILES: CC(C)n1nccc1C(=O)N[C@H](C(=O)Nc1cnn([C@H](C)c2nncn2CC(F)(F)F)c1)C(C1CC1)C1CC1] | N-[(1S)-1-(dicyclopropyl- methyl)-2-oxo-2-[[1-[(1R)-1- [4-(2,2,2-trifluoroethyl)-1,2,4- triazol-3-yl]ethyl]pyrazol-4- yl]amino]ethyl]-2-isopropyl- pyrazole-3-carboxamide | 548.271 | 2.19 |
| 95 | 97 | 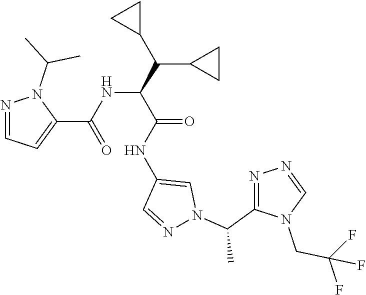 [SMILES: CC(C)n1nccc1C(=O)N[C@H](C(=O)Nc1cnn([C@@H](C)c2nncn2CC(F)(F)F)c1)C(C1CC1)C1CC1] | N-[(1S)-1-(dicyclopropyl- methyl)-2-oxo-2-[[1-[(1S)-1- [4-(2,2,2-trifluoroethyl)-1,2,4- triazol-3-yl]ethyl]pyrazol-4- yl]amino]ethyl]-2-isopropyl- pyrazole-3-carboxamide | 548.271 | 2.19 |

Examples 96-101 were prepared according to the methods of Preparations 11, 12 and 13 and Example 1 from (2S)-2-(tert-butoxycarbonylamino)-2-(trans-4-methylcyclohexyl)acetic acid, the appropriate carboxylic acid and the indicated nitropyrazoles.

| Ex. | Precursor | | | Mass | LCMS |
|---|---|---|---|---|---|
| No. | Prep. number | Structure | Name | ion | RT |
| 96 | 96 | 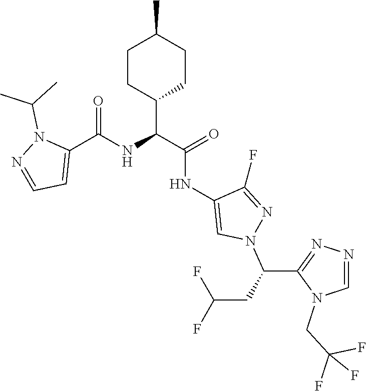 [SMILES: CC(C)n1nccc1C(=O)N[C@H](C(=O)Nc1cn([C@@H](CC(F)F)c2nncn2CC(F)(F)F)nc1F)[C@H]1CC[C@H](C)CC1] | N-[(1S)-2-[[1-[(1S)-3,3- difluoro-1-[4-(2,2,2- trifluoroethyl)-1,2,4-triazol-3- yl]propyl]-3-fluoro-pyrazol-4- yl]amino]-1-(trans-4- methylcyclohexyl)-2-oxo- ethyl]-2-isopropyl-pyrazole-3- carboxamide | 618.274 | 2.43 |
| 97 | 96 | 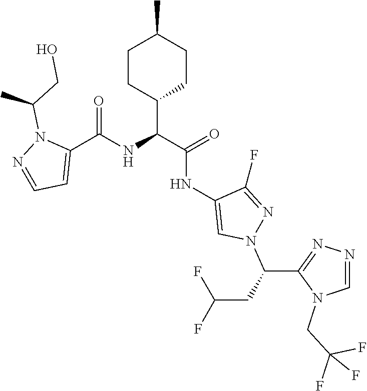 [SMILES: C[C@@H](CO)n1nccc1C(=O)N[C@H](C(=O)Nc1cn([C@@H](CC(F)F)c2nncn2CC(F)(F)F)nc1F)[C@H]1CC[C@H](C)CC1] | N-[(1S)-2-[[1-[(1S)-3,3- difluoro-1-[4-(2,2,2-trifluoro- ethyl)-1,2,4-triazol-3- yl]propyl]-3-fluoro-pyrazol-4- yl]amino]-1-(trans-4- methylcyclohexyl)-2-oxo- ethyl]-2-[(1S)-2-hydroxy-1- methyl-ethyl]-pyrazole-3- carboxamide | 634.269 | 2.22 |
| 98 | 96 | 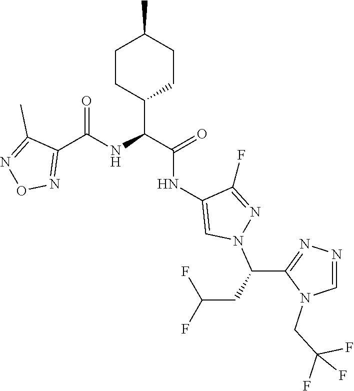 [SMILES: Cc1nonc1C(=O)N[C@H](C(=O)Nc1cn([C@@H](CC(F)F)c2nncn2CC(F)(F)F)nc1F)[C@H]1CC[C@H](C)CC1] | N-[(1S)-2-[1-[(1S)-3,3- difluoro-1-[4-(2,2,2- trifluoroethyl)-1,2,4-triazol-3- yl]propyl]-3-fluoro-pyrazol-4- yl]amino]-1-(trans-4- methylcyclohexyl)-2-oxo- ethyl]-4-methyl-1,2,5- oxadiazole-3-carboxamide | 592.222 | 2.46 |
| 99 | 101 | 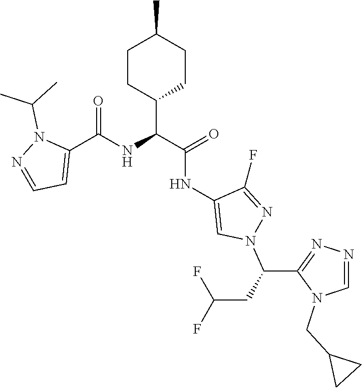 [SMILES: CC(C)n1nccc1C(=O)N[C@H](C(=O)Nc1cn([C@@H](CC(F)F)c2nncn2CC2CC2)nc1F)[C@H]1CC[C@H](C)CC1] | N-[(1S)-2-[[1-[(1S)-1-[4- (cyclopropylmethyl)-1,2,4- triazol-3-yl]-3,3-difluoro- propyl]-3-fluoro-pyrazol-4- yl]amino]-1-(trans-4- methylcyclohexyl)-2-oxo- ethyl]-2-isopropyl-pyrazole-3- carboxamide | 590.318 | 2.41 |
| 100 | 101 | 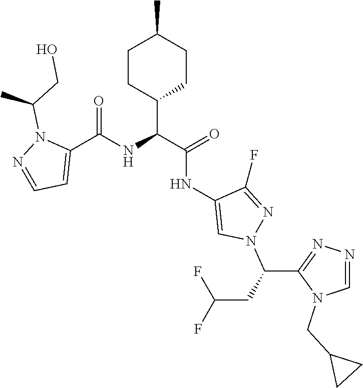 [SMILES: C[C@@H](CO)n1nccc1C(=O)N[C@H](C(=O)Nc1cn([C@@H](CC(F)F)c2nncn2CC2CC2)nc1F)[C@H]1CC[C@H](C)CC1] | N-[(1S)-2-[[1-[(1S)-1-[4- (cyclopropylmethyl)-1,2,4- triazol-3-yl]-3,3-difluoro- propyl]-3-fluoro-pyrazol-4- yl]amino]-1-(trans-4- methylcyclo-hexyl)-2-oxo- ethyl]-2-[(1S)-2-hydroxy-1- methyl-ethyl]pyrazole-3- carboxamide | 606.313 | 2.20 |
| 101 | 101 | 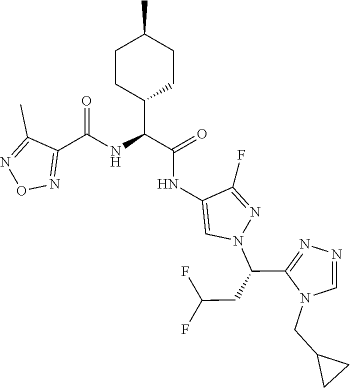 [SMILES: Cc1nonc1C(=O)N[C@H](C(=O)Nc1cn([C@@H](CC(F)F)c2nncn2CC2CC2)nc1F)[C@H]1CC[C@H](C)CC1] | N-[(1S)-2-[1-[(1S)-1-[4- (cyclopropylmethyl)-1,2,4- triazol-3-yl]-3,3-difluoro- propyl]-3-fluoro-pyrazol-4- yl]amino]-1-(trans-4- methylcyclohexyl)-2-oxo- ethyl]-4-methyl-1,2,5- oxadiazole-3-carboxamide | 564.266 | 2.44 |

### Example 102: N-[(1S)-1-(dicyclopropylmethyl)-2-[[3-fluoro-1-[l-[4-(2,2,2-trifluoroethyl)-1,2,4-triazol-3-yl]cyclopropyl]pyrazol-4-yl]amino]-2-oxo-ethyl]-2-isopropyl-pyrazole-3-carboxamide

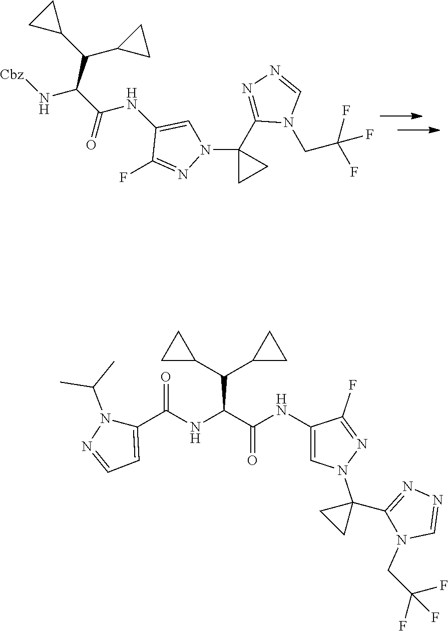 [SMILES: O=C(N[C@H](C(=O)Nc1cn(C2(c3nncn3CC(F)(F)F)CC2)nc1F)C(C1CC1)C1CC1)OCc1ccccc1 ; CC(C)n1nccc1C(=O)N[C@H](C(=O)Nc1cn(C2(c3nncn3CC(F)(F)F)CC2)nc1F)C(C1CC1)C1CC1]

According to the methods of Preparation 24 and Example 1 the compound of Preparation 109 was converted to the title compound (27 mg) as an off-white solid. LCMS (ES): m/z 578.262 [M+H]⁺, RT=2.30 min.

### Example 103: N-[(1S)-1-(dicyclopropylmethyl)-2-oxo-2-[[1-[1-[4-(2,2,2-trifluoroethyl)-1,2,4-triazol-3-yl]cyclopropyl]pyrazol-4-yl]amino]ethyl]-2-isopropyl-pyrazole-3-carboxamide

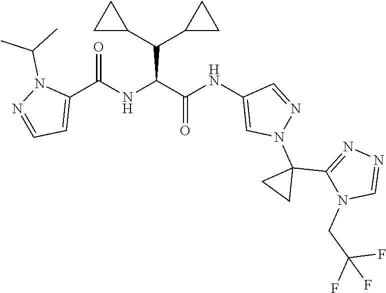 [SMILES: CC(C)n1nccc1C(=O)N[C@H](C(=O)Nc1cnn(C2(c3nncn3CC(F)(F)F)CC2)c1)C(C1CC1)C1CC1]

Example 103 was synthesised from the compound of Preparation 110 according to the methods of Preparations 11, 109 and 24 and Example 1. LCMS (ES): m/z 560.270 [M+H]⁺, RT=2.23 min.

### Example 104: N-[(1S)-1-(4,4-difluorocyclohexyl)-2-oxo-2-[[1-[1-[1-(2,2,2-trifluoroethyl)-tetrazol-5-yl]cyclopropyl]pyrazol-4-yl]amino]ethyl]-2-isopropyl-pyrazole-3-carboxamide

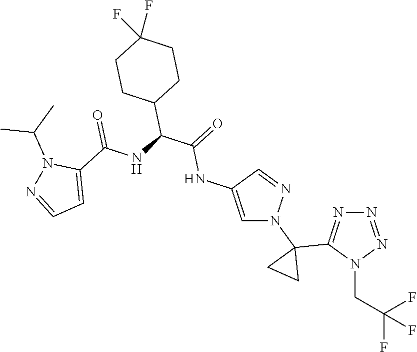 [SMILES: CC(C)n1nccc1C(=O)N[C@H](C(=O)Nc1cnn(C2(c3nnnn3CC(F)(F)F)CC2)c1)C1CCC(F)(F)CC1]

According to the methods of Preparation 11, 109 and 24 and Example 1 the compound of Preparation 111 was converted to the title compound as an off-white solid. LCMS (ES): m/z 585.248 [M+H]⁺, RT=2.36 min.

### Example 105: N-[(1S)-2-[[3-fluoro-1-[1-[5-(2,2,2-trifluoroethyl)-1H-pyrazol-4-yl]propyl]pyrazol-4-yl]amino]-1-(4-methylcyclohexyl)-2-oxo-ethyl]-2-isopropyl-pyrazole-3-carboxamide

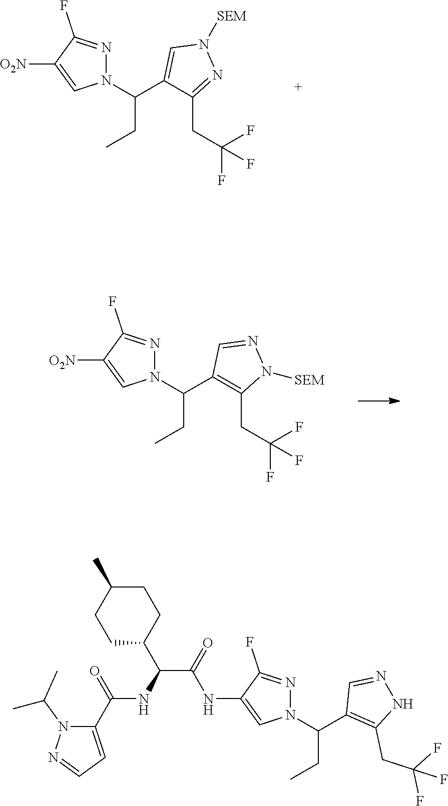 [SMILES: CCC(c1cn(COCC[Si](C)(C)C)nc1CC(F)(F)F)n1cc([N+](=O)[O-])c(F)n1 ; CCC(c1cnn(COCC[Si](C)(C)C)c1CC(F)(F)F)n1cc([N+](=O)[O-])c(F)n1 ; CCC(c1cn[nH]c1CC(F)(F)F)n1cc(NC(=O)[C@@H](NC(=O)c2ccnn2C(C)C)[C@H]2CC[C@H](C)CC2)c(F)n1]

According to the methods of Preparations 11, 12 and 13 and Example 1, Example 105 was prepared from the nitro compounds of Preparation 118 and (2S)-2-(tert-butoxycarbonylamino)-2-(trans-4-methylcyclohexyl)acetic acid to give the title compound (7 mg) as a colourless solid. ¹H NMR (400 MHz, DMSO) δ 12.94 (s, 1H), 9.94 (d, J=5.4 Hz, 1H), 8.39 (d, J=8.2 Hz, 1H), 7.97 (dd, J=6.9, 2.0 Hz, 1H), 7.93-7.82 (m, 1H), 7.48 (d, J=2.0 Hz, 1H), 6.92 (d, J=2.0 Hz, 1H), 5.46-5.31 (m, 1H), 5.13 (t, J=7.7 Hz, 1H), 4.36 (t, J=8.4 Hz, 1H), 3.76-3.50 (m, 2H), 2.22-2.09 (m, 1H), 2.01-1.86 (m, 1H), 1.85-1.62 (m, 4H), 1.59-1.47 (m, 1H), 1.39-1.31 (m, 6H), 1.31-1.21 (m, 1H), 1.21-1.08 (m, 1H), 1.07-0.93 (m, 1H), 0.92-0.79 (m, 5H), 0.79-0.70 (m, 3H); LCMS (ES): m/z 581.298 [M+H]⁺, RT=2.50 min.

### Example 106: N-[(1S)-2-[[3-fluoro-1-[1-[5-(2,2,2-trifluoroethyl)-1H-pyrazol-4-yl]propyl]pyrazol-4-yl]amino]-1-(4-methylcyclohexyl)-2-oxo-ethyl]-4-methyl-1,2,5-oxadiazole-3-carboxamide

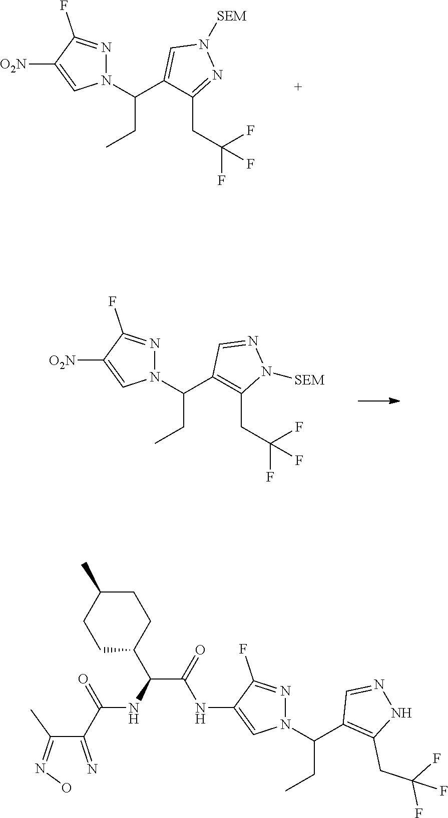 [SMILES: CCC(c1cn(COCC[Si](C)(C)C)nc1CC(F)(F)F)n1cc([N+](=O)[O-])c(F)n1 ; CCC(c1cnn(COCC[Si](C)(C)C)c1CC(F)(F)F)n1cc([N+](=O)[O-])c(F)n1 ; CCC(c1cn[nH]c1CC(F)(F)F)n1cc(NC(=O)[C@@H](NC(=O)C(=N)/C(C)=N\O)[C@H]2CC[C@H](C)CC2)c(F)n1]

According to the methods of Preparations 11, 12 and 13 and Example 1, Example 106 was prepared from the nitro compounds of Preparation 118, (2S)-2-(tert-butoxycarbonylamino)-2-(trans-4-methylcyclohexyl)acetic acid and 4-methyl-1,2,5-oxadiazole-3-carboxylic acid to give the title compound (14 mg) as a colourless solid. LCMS (ES): m/z 553.230 [M+H]⁺, RT=2.54 min.

### Example 107: N-[(1S)-1-(dicyclopropylmethyl)-2-oxo-2-[[1-[[1-(2,2,2-trifluoroethyl)triazol-4-yl]methyl]pyrazol-4-yl]amino]ethyl]-4-methyl-1,2,5-oxadiazole-3-carboxamide

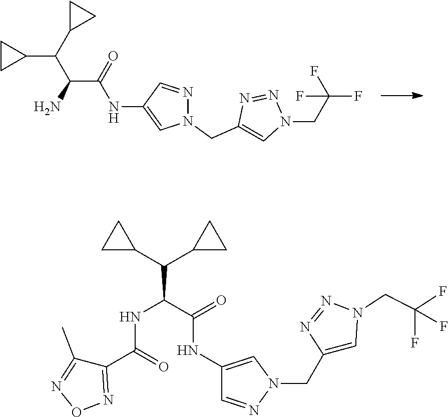 [SMILES: N[C@H](C(=O)Nc1cnn(Cc2cn(CC(F)(F)F)nn2)c1)C(C1CC1)C1CC1 ; Cc1nonc1C(=O)N[C@H](C(=O)Nc1cnn(Cc2cn(CC(F)(F)F)nn2)c1)C(C1CC1)C1CC1]

According to the method of Example 1 the compound of Preparation 13 was reacted with 4-methyl-1,2,5-oxadiazole-3-carboxylic acid to give the title compound. LCMS (ES): m/z 508.203 [M+H]⁺, RT=2.29 min.

### Example 108: (3,3-difluorocyclobutyl)N-[(1S)-1-(dicyclopropylmethyl)-2-oxo-2-[[1-[[1-(2,2,2-trifluoroethyl)triazol-4-yl]methyl]pyrazol-4-yl]amino]ethyl]carbamate

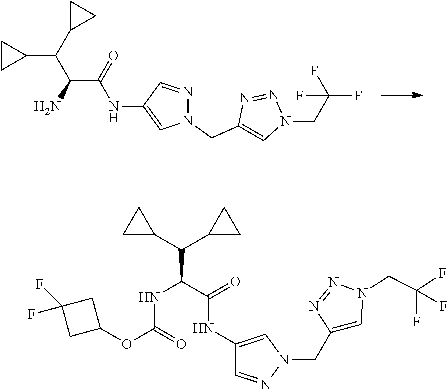 [SMILES: N[C@H](C(=O)Nc1cnn(Cc2cn(CC(F)(F)F)nn2)c1)C(C1CC1)C1CC1 ; O=C(N[C@H](C(=O)Nc1cnn(Cc2cn(CC(F)(F)F)nn2)c1)C(C1CC1)C1CC1)OC1CC(F)(F)C1]

According to the method of Example 4 the compound of Preparation 13 was reacted with the compound of Preparation 19 to give the title compound. LCMS (ES): m/z 532.210 [M+H]⁺, RT=2.27 min.

According to the methods of Preparations 10, 11, 12, 13 and Example 1, Examples 109-112 were synthesised starting from the indicated alcohols.

| Ex. | Precursor | | | Mass | LCMS |
|---|---|---|---|---|---|
| No. | alcohol | Structure | Name | ion | RT |
| 109 | 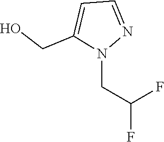 [SMILES: OCc1ccnn1CC(F)F] | 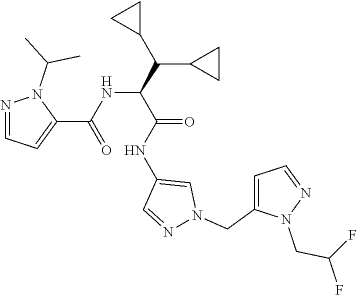 [SMILES: CC(C)n1nccc1C(=O)N[C@H](C(=O)Nc1cnn(Cc2ccnn2CC(F)F)c1)C(C1CC1)C1CC1] | N-[(1S)-1-(dicyclopropyl- methyl)-2-[[1-[[2-(2,2- difluoroethyl)pyrazol-3- yl]methyl]pyrazol-4-yl] amino]-2-oxo-ethyl]- 2-isopropyl- pyrazole-3-carboxamide | 515.269 | 2.25 |
| 110 | 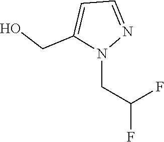 [SMILES: OCc1ccnn1CC(F)F] | 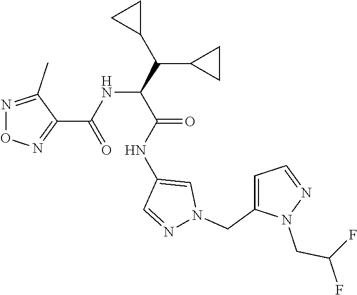 [SMILES: Cc1nonc1C(=O)N[C@H](C(=O)Nc1cnn(Cc2ccnn2CC(F)F)c1)C(C1CC1)C1CC1] | N-[(1S)-1-(dicyclopropyl- methyl)-2-[[1-[[2-(2,2- difluoroethyl)pyrazol-3- yl]methyl]pyrazol-4-yl] amino]-2-oxo-ethyl]- 4-methyl-1,2,5- oxadiazole-3-carboxamide | 489.217 | 2.31 |
| 111 | 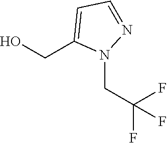 [SMILES: OCc1ccnn1CC(F)(F)F] | 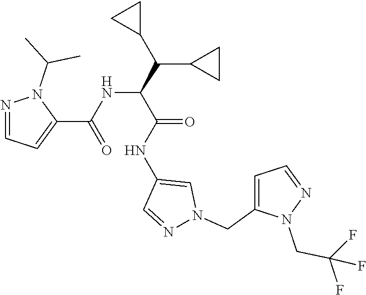 [SMILES: CC(C)n1nccc1C(=O)N[C@H](C(=O)Nc1cnn(Cc2ccnn2CC(F)(F)F)c1)C(C1CC1)C1CC1] | N-[(1S)-1-(dicyclopropyl- methyl)-2-oxo-2-[[1-[[2- (2,2,2-trifluoroethyl) pyrazol-3- yl]methyl]pyrazol-4- yl]amino]ethyl]-2- isopropyl- pyrazole-3-carboxamide | 533.26 | 2.31 |
| 112 | 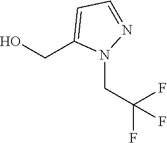 [SMILES: OCc1ccnn1CC(F)(F)F] | 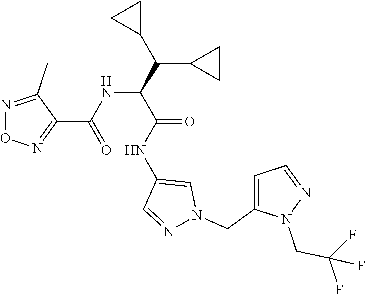 [SMILES: Cc1nonc1C(=O)N[C@H](C(=O)Nc1cnn(Cc2ccnn2CC(F)(F)F)c1)C(C1CC1)C1CC1] | N-[(1S)-1-(dicyclopropyl- methyl)-2-oxo-2-[[1-[[2- (2,2,2-trifluoroethyl) pyrazol-3- yl]methyl]pyrazol-4- yl]amino]ethyl]-4-methyl- 1,2,5-oxadiazole-3- carboxamide | 507.208 | 2.40 |

According to the methods of Preparations 10, 11, 12, 13 and Example 4, Examples 113-114 were synthesised starting from the indicated alcohols and using the compound of Preparation 119 in the final step.

| Ex. | Precursor | | | Mass | LCMS |
|---|---|---|---|---|---|
| No. | alcohol | Structure | Name | ion | RT |
| 113 | 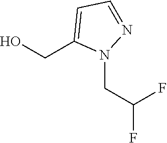 [SMILES: OCc1ccnn1CC(F)F] | 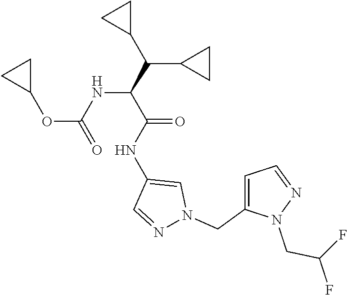 [SMILES: O=C(N[C@H](C(=O)Nc1cnn(Cc2ccnn2CC(F)F)c1)C(C1CC1)C1CC1)OC1CC1] | cyclopropyl N-[(1S)-1-(dicyclo- propylmethyl)-2-[[1-[2-(2,2- difluoroethyl)pyrazol-3- yl]methyl]pyrazol-4-yl]amino]- 2-oxo-ethyl]carbamate | 463.226 | 2.22 |
| 114 | 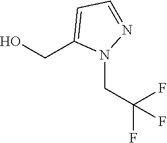 [SMILES: OCc1ccnn1CC(F)(F)F] | 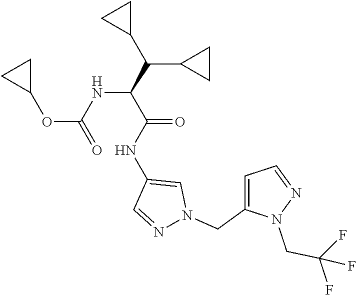 [SMILES: O=C(N[C@H](C(=O)Nc1cnn(Cc2ccnn2CC(F)(F)F)c1)C(C1CC1)C1CC1)OC1CC1] | cyclopropyl N-[(1S)-1- (dicyclopropylmethyl)-2-oxo-2- [[1-[[2-(2,2,2-trifluoroethyl)- pyrazol-3-yl]methyl]pyrazol-4- yl]amino]ethyl]carbamate | 481.217 | 2.25 |

According to the method of Example 5 the compound of Preparation 26 was alkylated with the indicated alkylating agents to give Examples 115-117.

| Ex. | Alkylating | | | Mass | LCMS |
|---|---|---|---|---|---|
| No. | agent | Structure | Name | ion | RT |
| 115 | 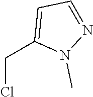 | 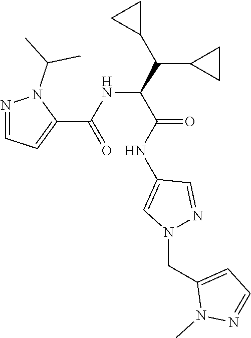 [SMILES: CC(C)n1nccc1C(=O)N[C@H](C(=O)Nc1cnn(Cc2ccnn2C)c1)C(C1CC1)C1CC1] | N-[(1S)-1-(dicyclopropyl- methyl)-2-[[1-[(2- methylpyrazol-3-yl)methyl]- pyrazol-4-yl]amino]-2-oxo- ethyl]-2-isopropyl-pyrazole-3- carboxamide | 465.272 | 2.15 |
| 116 | 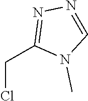 | 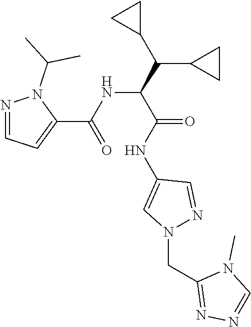 [SMILES: CC(C)n1nccc1C(=O)N[C@H](C(=O)Nc1cnn(Cc2nncn2C)c1)C(C1CC1)C1CC1] | N-[(1S)-1-(dicyclopropyl- methyl)-2-[[1-[(4-methyl- 1,2,4-triazol-3-yl)methyl]- pyrazol-4-yl]amino]-2-oxo- ethyl]-2-isopropyl-pyrazole-3- carboxamide | 466.268 | 2.00 |
| 117 | 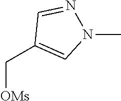 | 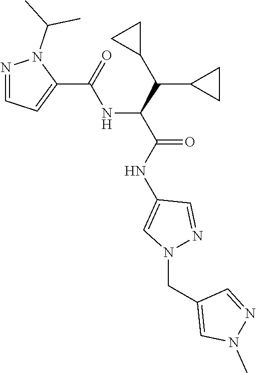 [SMILES: CC(C)n1nccc1C(=O)N[C@H](C(=O)Nc1cnn(Cc2cnn(C)c2)c1)C(C1CC1)C1CC1] | N-[(1S)-1- (dicyclopropylmethyl)-2-[[1- [(1-methylpyrazol-4- yl)methyl]pyrazol-4-yl]amino]- 2-oxo-ethyl]-2-isopropyl- pyrazole-3-carboxamide | 465.272 | 2.21 |

### Example 118: N-[(1S)-1-(dicyclopropylmethyl)-2-[[1-[(3-methyl-1H-pyrazol-5-yl)methyl]pyrazol-4-yl]amino]-2-oxo-ethyl]-2-isopropyl-pyrazole-3-carboxamide

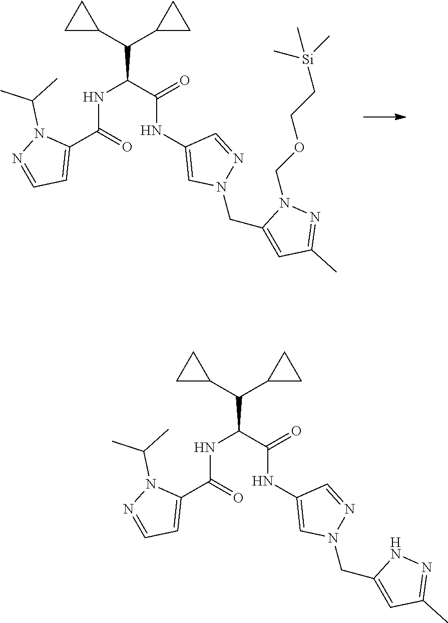

A mixture of the compound of Preparation 123 (80 mg, 0.134 mmol) in TFA:DCM (1 mL:1 mL) was stirred at 50° C. for 1 hour. After cooling to room temperature the solvent was removed in vacuo and the residue was purified by prep. Basic HPLC to give the title compound (28 mg, 450/Yield) as a white solid. LCMS (ES): m/z 465.272 [M+H]⁺, RT=2.13 min.

### Example 119: N-[(1S)-1-(dicyclopropylmethyl)-2-[[1-[(3,5-dimethyl-1H-pyrazol-4-yl)methyl]pyrazol-4-yl]amino]-2-oxo-ethyl]-2-isopropyl-pyrazole-3-carboxamide

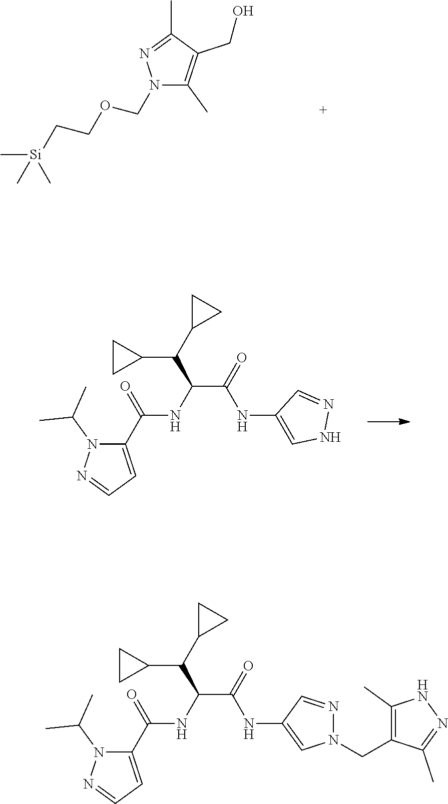 [SMILES: Cc1nn(COCC[Si](C)(C)C)c(C)c1CO ; CC(C)n1nccc1C(=O)N[C@H](C(=O)Nc1cn[nH]c1)C(C1CC1)C1CC1 ; Cc1n[nH]c(C)c1Cn1cc(NC(=O)[C@@H](NC(=O)c2ccnn2C(C)C)C(C2CC2)C2CC2)cn1]

To a solution of the compound of preparation 125 (60 mg, 0.234 mmol) and TEA (0.15 mL, 1.1 mmol) in DCM (3 mL) was added methanesulfonyl chloride (0.05 mL, 0.6 mmol) dropwise at 5° C. The suspension was stirred at this temp for 0.5 hour then the reaction was quenched with 1M HCl (15 mL). The mixture was extracted with DCM (2×10 mL) and the combined organic phases were dried over MgSO₄ and concentrated in vacuo, giving the crude mesylate that was used without further characterisation.

The mesylate from above was dissolved in DMSO (3 mL) and the compound of Preparation 26 (33 mg, 0.089 mmol), and caesium carbonate (100 mg, 0.307 mmol) were added. The reaction mixture was stirred at room temperature for 2 hours to give a complex mixture containing N-[(1S)-1-(dicyclopropylmethyl)-2-[[1-[[3,5-dimethyl-1-(2-trimethylsilylethoxymethyl)pyrazol-4-yl]methyl]pyrazol-4-yl]amino]-2-oxo-ethyl]-2-isopropyl-pyrazole-3-carboxamide, along with several other components including the title compound. Attempted purification by prep. acidic HPLC gave an impure product that was dissolved in TFA (1 mL) and stirred for 1 hour. The solution was concentrated in vacuo and the residue was taken up in sat. aq. NaHCO₃, from which the product precipitated. The aqueous phase was decanted off and the residue was washed with water (2×5 mL) and dried in vacuo. Purification by column chromatography (ethyl acetate/MeOH 25:1) gave the title compound (14 mg, 33% Yield). ¹H NMR (400 MHz, DMSO) δ 10.09 (s, 1H), 8.28 (d, J=8.9 Hz, 1H), 7.76 (s, 1H), 7.49 (d, J=2.0 Hz, 1H), 7.40 (s, 1H), 6.92 (d, J=2.0 Hz, 1H), 5.39 (hept, J=6.6 Hz, 1H), 5.02 (s, 2H), 4.70 (dd, J=8.8, 7.0 Hz, 1H), 2.13 (s, 6H), 1.37 (d, J=6.6 Hz, 3H), 1.34 (d, J=6.6 Hz, 3H), 0.82 (m, 1H), 0.77-0.65 (m, 2H), 0.48-0.12 (m, 7H), 0.10-0.02 (m, 1H); LCMS (ES): m/z 479.288 [M+H]⁺, RT=2.10 min.

Examples 120-122 were prepared according to the methods of Preparations 11, 12 and 13 and Example 1 from the compound of Preparation 130, (2S)-2-(tert-butoxycarbonylamino)-2-(4,4-difluorocyclohexyl)acetic acid and the appropriate carboxylic acid.

| Ex. | | | Mass | LCMS |
|---|---|---|---|---|
| No. | Structure | Name | ion | RT |
| 120 | 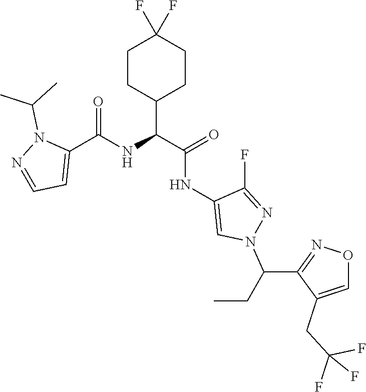 [SMILES: CCC(c1nocc1CC(F)(F)F)n1cc(NC(=O)[C@@H](NC(=O)c2ccnn2C(C)C)C2CCC(F)(F)CC2)c(F)n1] | N-[(1S)-1-(4,4-difluorocyclohexyl)- 2-[[3-fluoro-1-[1-[4-(2,2,2-trifluoro- ethyl)isoxazol-3-yl]propyl]pyrazol-4- yl]amino]-2-oxo-ethyl]-2-isopropyl- pyrazole-3-carboxamide | 604.247 | 2.52 |
| 121 | 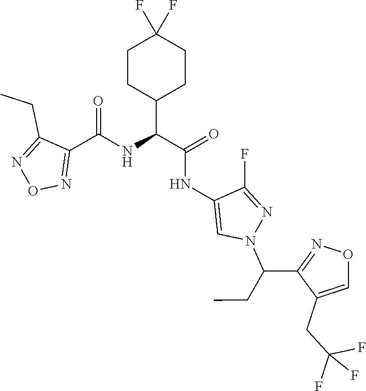 [SMILES: CCc1nonc1C(=O)N[C@H](C(=O)Nc1cn(C(CC)c2nocc2CC(F)(F)F)nc1F)C1CCC(F)(F)CC1] | N-[(1S)-1-(4,4-difluorocyclohexyl)- 2-[[3-fluoro-1-[1-[4-(2,2,2-trifluoro- ethyl)isoxazol-3-yl]propyl]pyrazol-4- yl]amino]-2-oxo-ethyl]-4-ethyl- 1,2,5-oxadiazole-3-carboxamide | 592.212 | 2.60 |
| 122 | 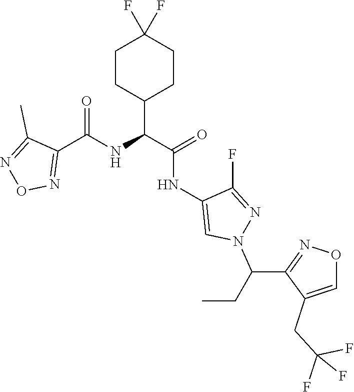 [SMILES: CCC(c1nocc1CC(F)(F)F)n1cc(NC(=O)[C@@H](NC(=O)c2nonc2C)C2CCC(F)(F)CC2)c(F)n1] | N-[(1S)-1-(4,4-difluorocyclohexyl)- 2-[[3-fluoro-1-[1-[4-(2,2,2-trifluoro- ethyl)isoxazol-3-yl]propyl]pyrazol-4- yl]amino]-2-oxo-ethyl]-4-methyl- 1,2,5-oxadiazole-3-carboxamide | 578.196 | 2.55 |

Examples 123-126 were prepared according to the methods of Preparations 11, 12 and 13 and Example 1 from the compounds of Preparation 8, the appropriate carboxylic acid and the indicated nitropyrazoles.

| Ex. | Precursor | | | Mass | LCMS |
|---|---|---|---|---|---|
| No. | Prep. number | Structure | Name | ion | RT |
| 123 | 131 | 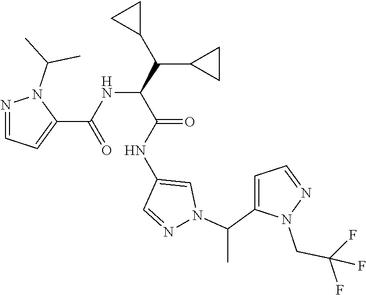 [SMILES: CC(c1ccnn1CC(F)(F)F)n1cc(NC(=O)[C@@H](NC(=O)c2ccnn2C(C)C)C(C2CC2)C2CC2)cn1] | N-[(1S)-1-(dicyclopropyl- methyl)-2-oxo-2-[[1-[1-[2- (2,2,2-trifluoroethyl)pyrazol-3- yl]ethyl]pyrazol-4- yl]amino]ethyl]-2-isopropyl- pyrazole-3-carboxamide | 547.276 | 2.35 |
| 124 | 132 | 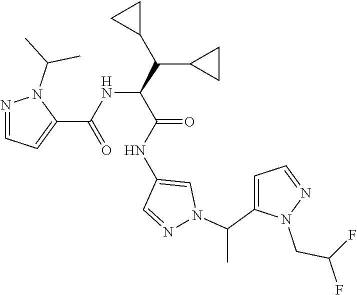 [SMILES: CC(c1ccnn1CC(F)F)n1cc(NC(=O)[C@@H](NC(=O)c2ccnn2C(C)C)C(C2CC2)C2CC2)cn1] | N-[(1S)-1-(dicyclopropyl- methyl)-2-[[1-[1-[2-(2,2- difluoroethyl)pyrazol-3- yl]ethyl]pyrazol-4-yl]amino]-2- oxo-ethyl]-2-isopropyl- pyrazole-3-carboxamide | 529.285 | 2.32 |
| 125 | 133 | 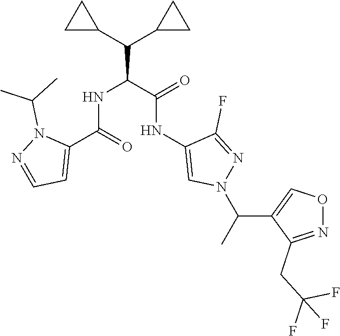 [SMILES: CC(c1conc1CC(F)(F)F)n1cc(NC(=O)[C@@H](NC(=O)c2ccnn2C(C)C)C(C2CC2)C2CC2)c(F)n1] | N-[(1S)-1-(dicyclopropyl- methyl)-2-[[3-fluoro-1-[1-[3- (2,2,2-trifluoroethyl)isoxazol- 4-yl]ethyl]pyrazol-4-yl]amino]- 2-oxo-ethyl]-2-isopropyl- pyrazole-3-carboxamide | 566.250 | 2.51 |
| 126 | 134 | 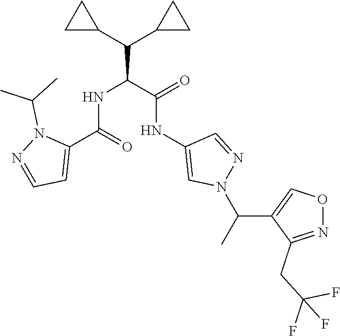 [SMILES: CC(c1conc1CC(F)(F)F)n1cc(NC(=O)[C@@H](NC(=O)c2ccnn2C(C)C)C(C2CC2)C2CC2)cn1] | N-[(1S)-1-(dicyclopropyl- methyl)-2-oxo-2-[[1-[1-[3- (2,2,2-trifluoroethyl)isoxazol- 4-yl]ethyl]pyrazol-4- yl]amino]ethyl]-2-isopropyl- pyrazole-3-carboxamide | 548.260 | 2.43 |

### Example 127: N-[(1N)-1-(dicyclopropylmethyl)-2-[[1-[(3-ethyltriazol-4-yl)methyl]pyrazol-4-yl]amino]-2-oxo-ethyl]-2-isopropyl-pyrazole-3-carboxamide

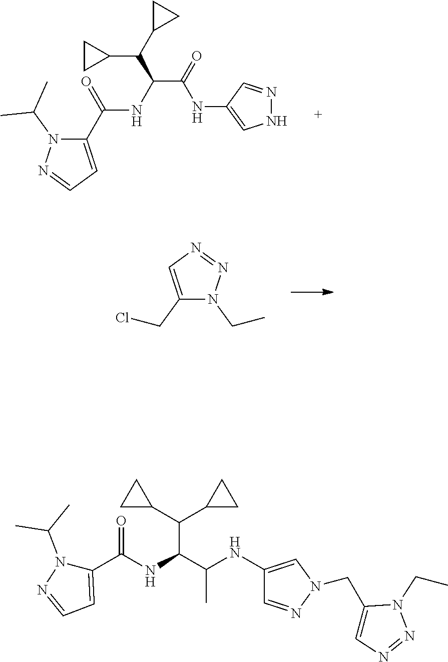

According to the method of Example 5 the compound of Preparation 26 was alkylated with 5-(chloromethyl)-1-ethyl-triazole to give the title compound. LCMS (ES): m/z 480.283 [M+H]⁺, RT=2.16 min.

Examples 128-138 were prepared according to the method of Preparation 31 from the indicated alkynes and the appropriate alkyl azide.

| Ex. | Precursor | | | Mass | LCMS |
|---|---|---|---|---|---|
| No. | Prep. number | Structure | Name | ion | RT |
| 128 | 135 | 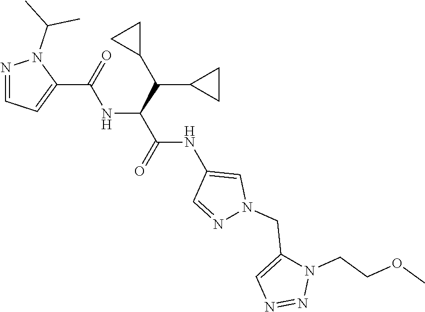 [SMILES: COCCn1nncc1Cn1cc(NC(=O)[C@@H](NC(=O)c2ccnn2C(C)C)C(C2CC2)C2CC2)cn1] | N-[(1S)-1-(dicyclopropyl- methyl)-2-[[1-[[3-(2- methoxyethyl)triazol-4- yl]methyl]pyrazol-4-yl]amino]- 2-oxo-ethyl]-2-isopropyl- pyrazole-3-carboxamide | 510.294 | 2.24 |
| 129 | 135 | 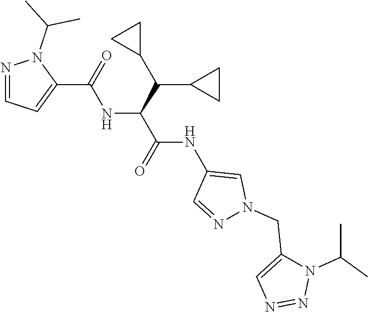 [SMILES: CC(C)n1nncc1Cn1cc(NC(=O)[C@@H](NC(=O)c2ccnn2C(C)C)C(C2CC2)C2CC2)cn1] | N-[(1S)-1-(dicyclopropyl- methyl)-2-[[1-[(3-isopropyl- triazol-4-yl)methyl]pyrazol-4- yl]amino]-2-oxo-ethyl]-2-iso- propyl-pyrazole-3-carboxamide | 494.299 | 2.21 |
| 130 | 135 | 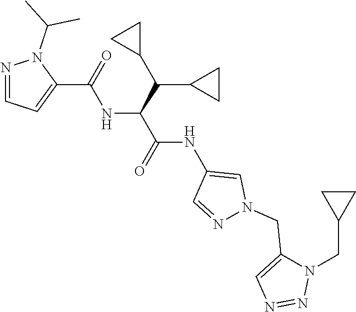 [SMILES: CC(C)n1nccc1C(=O)N[C@H](C(=O)Nc1cnn(Cc2cnnn2CC2CC2)c1)C(C1CC1)C1CC1] | N-[(1S)-2,2-dicyclopropyl-1- [[1-[[3-(cyclopropylmethyl)- triazol-4-yl]methyl]pyrazol-4- yl]carbamoyl]ethyl]-2-iso- propyl-pyrazole-3-carboxamide | 506.299 | 2.24 |
| 131 | 135 | 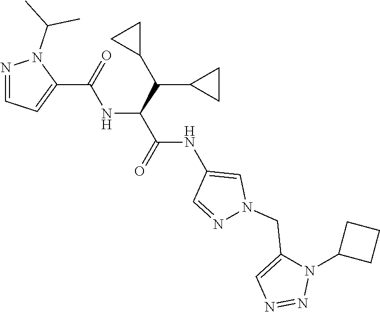 [SMILES: CC(C)n1nccc1C(=O)N[C@H](C(=O)Nc1cnn(Cc2cnnn2C2CCC2)c1)C(C1CC1)C1CC1] | N-[(1S)-1-[[1-[(3-cyclobutyl- triazol-4-yl)methyl]pyrazol-4- yl]carbamoyl]-2,2-dicyclo- propyl-ethyl]-2-isopropyl- pyrazole-3-carboxamide | 506.299 | 2.26 |
| 132 | 136 | 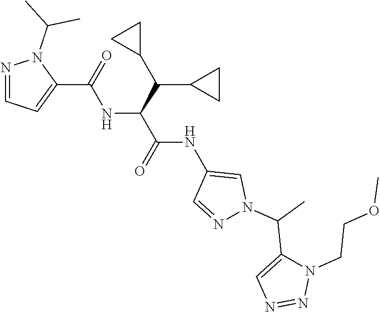 [SMILES: COCCn1nncc1C(C)n1cc(NC(=O)[C@@H](NC(=O)c2ccnn2C(C)C)C(C2CC2)C2CC2)cn1] | N-[(1S)-1-(dicyclopropyl- methyl)-2-[[1-[1-[3-(2- methoxyethyl)triazol-4- yl]ethyl]pyrazol-4-yl]amino]-2- oxo-ethyl]-2-isopropyl- pyrazole-3-carboxamide | 524.309 | 2.20 |
| 133* | 136 | 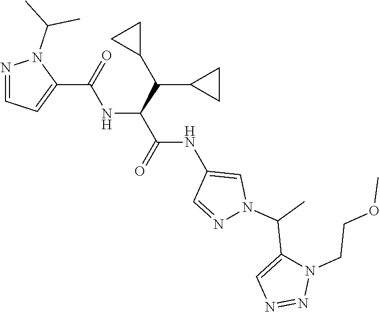 [SMILES: COCCn1nncc1C(C)n1cc(NC(=O)[C@@H](NC(=O)c2ccnn2C(C)C)C(C2CC2)C2CC2)cn1] | Diastereomer 1 of N-[(1S)-1- (dicyclopropyl-methyl)-2-[1- [1-[3-(2-methoxyethyl)triazol- 4-yl]ethyl]pyrazol-4-yl]amino]- 2-oxo-ethyl]-2-isopropyl- pyrazole-3-carboxamide | 524.310 | 2.20 |
| 134* | 136 | 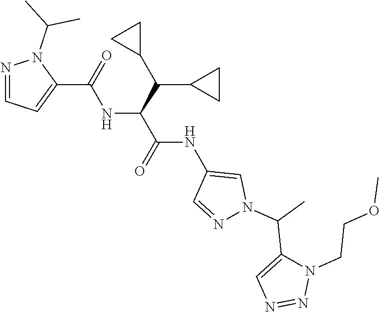 [SMILES: COCCn1nncc1C(C)n1cc(NC(=O)[C@@H](NC(=O)c2ccnn2C(C)C)C(C2CC2)C2CC2)cn1] | Diastereomer 2 of N-[(1S)-1- (dicyclopropyl-methyl)-2-[[1- [1-[3-(2-methoxyethyl)triazol- 4-yl]ethyl]pyrazol-4-yl]amino]- 2-oxo-ethyl]-2-isopropyl- pyrazole-3-carboxamide | 524.310 | 2.20 |
| 135 | 136 | 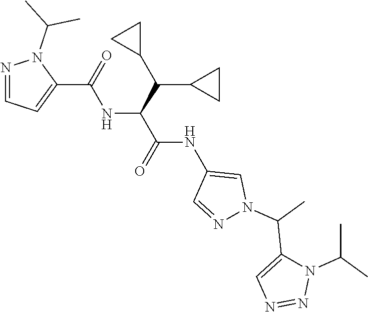 [SMILES: CC(c1cnnn1C(C)C)n1cc(NC(=O)[C@@H](NC(=O)c2ccnn2C(C)C)C(C2CC2)C2CC2)cn1] | N-[(1S)-1-(dicyclopropyl- methyl)-2-[[1-[1-(3-isopropyl- triazol-4-yl)ethyl]pyrazol-4-yl]- amino]-2-oxo-ethyl]-2-iso- propyl-pyrazole-3-carboxamide | 508.315 | 2.25 |
| 136 | 136 | 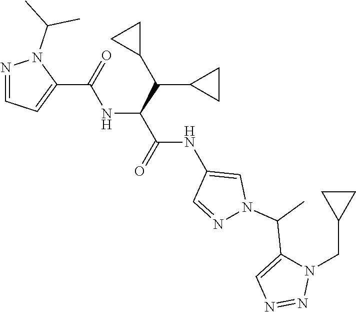 [SMILES: CC(c1cnnn1CC1CC1)n1cc(NC(=O)[C@@H](NC(=O)c2ccnn2C(C)C)C(C2CC2)C2CC2)cn1] | N-[(1S)-2,2-dicyclopropyl-1- [[1-[1-[3-(cyclopropylmethyl)- triazol-4-yl]ethyl]-pyrazol-4- yl]carbamoyl]ethyl]-2-iso- propyl-pyrazole-3-carboxamide | 520.314 | 2.28 |
| 137 | 136 | 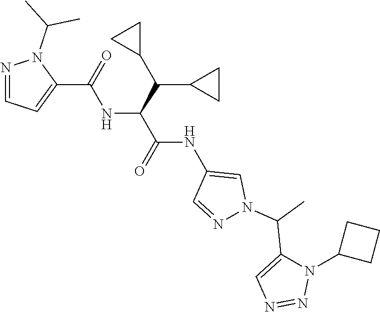 [SMILES: CC(c1cnnn1C1CCC1)n1cc(NC(=O)[C@@H](NC(=O)c2ccnn2C(C)C)C(C2CC2)C2CC2)cn1] | N-[(1S)-1-[[1-[1-(3-cyclo- butyltriazol-4-yl)ethyl]pyrazol- 4-yl]carbamoyl]-2,2-dicyclo- propyl-ethyl]-2-isopropyl- pyrazole-3-carboxamide | 520.314 | 2.29 |
| 138^{#} | 135 | 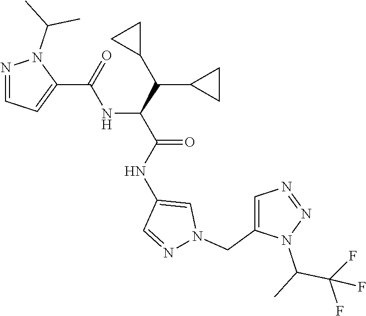 [SMILES: CC(C)n1nccc1C(=O)N[C@H](C(=O)Nc1cnn(Cc2cnnn2C(C)C(F)(F)F)c1)C(C1CC1)C1CC1] | N-[(1S)-1-(dicyclopropyl- methyl)-2-oxo-2-[[1-[[3- (2,2,2-trifluoro-1-methyl- ethyl)triazol-4-yl]methyl]- pyrazol-4-yl]amino]ethyl]-2- isopropyl-pyrazole-3- carboxamide | 548.270 | 2.35 |

*The diastereomers were separated by chiral SFC
^{#}The azide of Preparation 137 was used as a diethyl ether solution

Examples 139-146 were prepared according to the methods of Preparations 11, 12 and 13 and Example 1 from the appropriate Boc protected amino acid, the appropriate carboxylic acid and the indicated nitropyrazoles.

| Ex. | Precursor | | | Mass | LCMS |
|---|---|---|---|---|---|
| No. | Prep. number | Structure | Name | ion | RT |
| 139 | 140 | 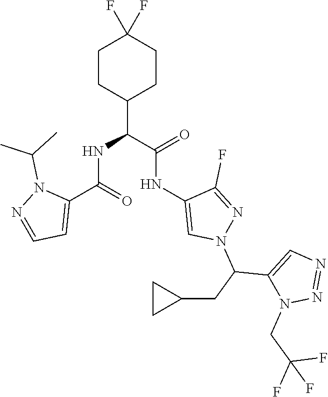 [SMILES: CC(C)n1nccc1C(=O)N[C@H](C(=O)Nc1cn(C(CC2CC2)c2cnnn2CC(F)(F)F)nc1F)C1CCC(F)(F)CC1] | N-[(1S)-2-[[1-[2-cyclopropyl- 1-[3-(2,2,2-trifluoroethyl)- triazol-4-yl]ethyl]-3-fluoro- pyrazol-4-yl]amino]-1-(4,4- difluorocyclohexyl)-2-oxo- ethyl]-2-isopropyl-pyrazole-3- carboxamide | 630.274 | 2.47 |
| 140 | 140 | 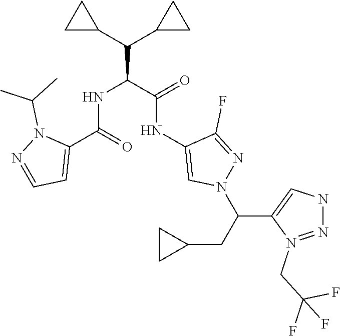 | N-[(1S)-2,2-dicyclopropyl-1- [[1-[2-cyclopropyl-1-[3-(2,2,2- trifluoroethyl)triazol-4- yl]ethyl]-3-fluoro-pyrazol-4- yl]carbamoyl]ethyl]-2-iso- propyl-pyrazole-3-carboxamide | 606.293 | 2.53 |
| 141 | 140 | 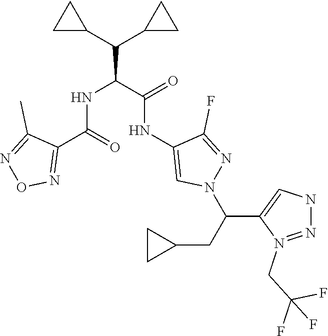 | N-[(1S)-2,2-dicyclopropyl-1- [[1-[2-cyclopropyl-1-[3-(2,2,2- trifluoroethyl)triazol-4-yl]- ethyl]-3-fluoro-pyrazol-4-yl]- carbamoyl]ethyl]-4-methyl- 1,2,5-oxadiazole-3- carboxamide | 580.241 | 2.56 |
| 142 | 140 | 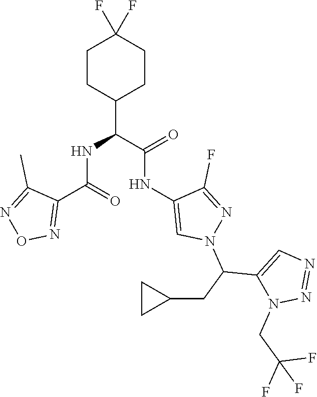 [SMILES: Cc1nonc1C(=O)N[C@H](C(=O)Nc1cn(C(CC2CC2)c2cnnn2CC(F)(F)F)nc1F)C1CCC(F)(F)CC1] | N-[(1S)-2-[[1-[2-cyclopropyl- 1-[3-(2,2,2-trifluoroethyl)- triazol-4-yl]ethyl]-3-fluoro- pyrazol-4-yl]amino]-1-(4,4- difluorocyclohexyl)-2-oxo- ethyl]-4-methyl-1,2,5- oxadiazole-3-carboxamide | 604.222 | 2.50 |
| 143 | 143 | 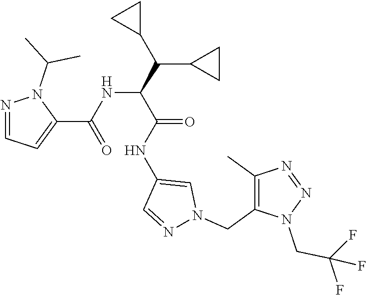 [SMILES: Cc1nnn(CC(F)(F)F)c1Cn1cc(NC(=O)[C@@H](NC(=O)c2ccnn2C(C)C)C(C2CC2)C2CC2)cn1] | N-[(1S)-1-(dicyclopropyl- methyl)-2-[[1-[[5-methyl-3- (2,2,2-trifluoroethyl)triazol-4- yl]methyl]pyrazol-4-yl]amino]- 2-oxo-ethyl]-2-isopropyl- pyrazole-3-carboxamide | 548.271 | 2.38 |
| 144 | 146 | 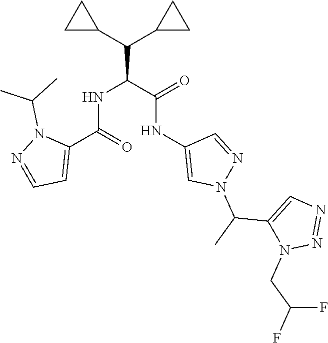 [SMILES: CC(c1cnnn1CC(F)F)n1cc(NC(=O)[C@@H](NC(=O)c2ccnn2C(C)C)C(C2CC2)C2CC2)cn1] | N-[(1S)-1-(dicyclopropyl- methyl)-2-[[1-[1-[3-(2,2- difluoroethyl)triazol-4- yl]ethyl]pyrazol-4-yl]amino]-2- oxo-ethyl]-2-isopropyl- pyrazole-3-carboxamide | 530.281 | 2.33 |
| 145 | 146 | 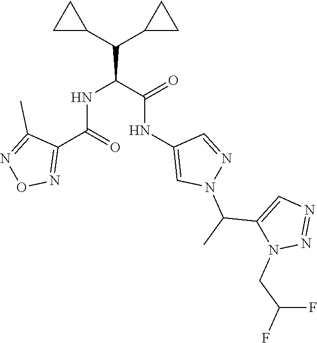 [SMILES: Cc1nonc1C(=O)N[C@H](C(=O)Nc1cnn(C(C)c2cnnn2CC(F)F)c1)C(C1CC1)C1CC1] | N-[(1S)-1-(dicyclopropyl- methyl)-2-[[1-[1-[3-(2,2- difluoroethyl)triazol-4- yl]ethyl]pyrazol-4-yl]amino]-2- oxo-ethyl]-4-methyl-1,2,5- oxadiazole-3-carboxamide | 504.228 | 2.39 |
| 146 | 153 | 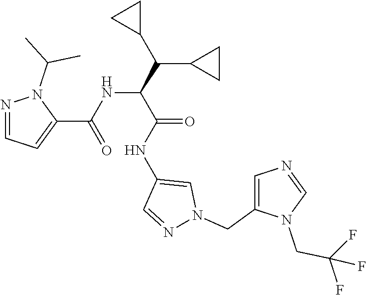 [SMILES: CC(C)n1nccc1C(=O)N[C@H](C(=O)Nc1cnn(Cc2cncn2CC(F)(F)F)c1)C(C1CC1)C1CC1] | N-[(1S)-1-(dicyclopropyl- methyl)-2-oxo-2-[[1-[[3- (2,2,2-trifluoroethyl)imidazol- 4-yl]methyl]pyrazol-4- yl]amino]ethyl]-2-isopropyl- pyrazole-3-carboxamide | 533.260 | 2.20 |

Examples 147-153 were prepared according to the methods of Preparations 11, 33 and 24 and Example 1 from the appropriate CBz protected amino acid, the appropriate carboxylic acid and the indicated nitropyrazoles.

| Ex. | Precursor | | | Mass | LCMS |
|---|---|---|---|---|---|
| No. | Prep. number | Structure | Name | ion | RT |
| 147 | 142 | 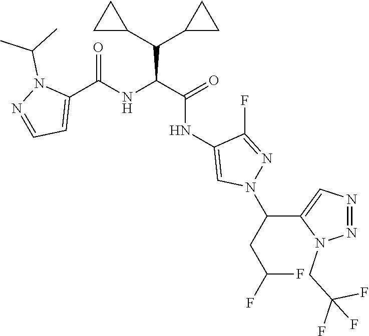 [SMILES: CC(C)n1nccc1C(=O)N[C@H](C(=O)Nc1cn(C(CC(F)F)c2cnnn2CC(F)(F)F)nc1F)C(C1CC1)C1CC1] | N-[(1S)-1-(dicyclopropyl- methyl)-2-[[1-[3,3-difluoro-1- [3-(2,2,2-trifluoroethyl)triazol- 4-yl]propyl]-3-fluoro-pyrazol- 4-yl]amino]-2-oxo-ethyl]-2- isopropyl-pyrazole-3- carboxamide | 616.259 | 2.45 |
| 148 | 142 | 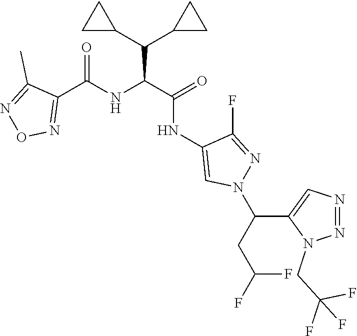 [SMILES: Cc1nonc1C(=O)N[C@H](C(=O)Nc1cn(C(CC(F)F)c2cnnn2CC(F)(F)F)nc1F)C(C1CC1)C1CC1] | N-[(1S)-1-(dicyclopropyl- methyl)-2-[[1-[3,3-difluoro-1- [3-(2,2,2-trifluoroethyl)triazol- 4-yl]propyl]-3-fluoro-pyrazol- 4-yl]amino]-2-oxo-ethyl]-4- methyl-1,2,5-oxadiazole-3- carboxamide | 590.206 | 2.50 |
| 149 | 144 | 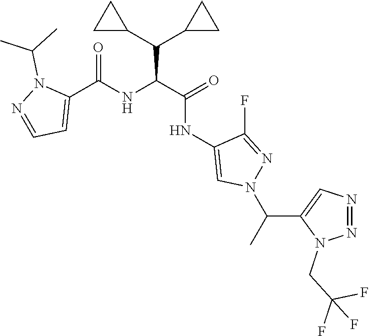 [SMILES: CC(c1cnnn1CC(F)(F)F)n1cc(NC(=O)[C@@H](NC(=O)c2ccnn2C(C)C)C(C2CC2)C2CC2)c(F)n1] | N-[(1S)-1-(dicyclopropyl- methyl)-2-[[3-fluoro-1-[1-[3- (2,2,2-trifluoroethyl)triazol-4- yl]ethyl]pyrazol-4-yl]amino]-2- oxo-ethyl]-2-isopropyl- pyrazole-3-carboxamide | 566.262 | 2.44 |
| 150 | 144 | 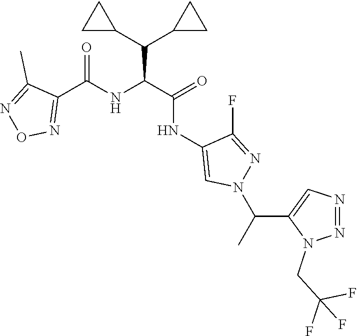 [SMILES: Cc1nonc1C(=O)N[C@H](C(=O)Nc1cn(C(C)c2cnnn2CC(F)(F)F)nc1F)C(C1CC1)C1CC1] | N-[(1S)-1-(dicyclopropyl- methyl)-2-[[3-fluoro-1-[1-[3- (2,2,2-trifluoroethyl)triazol-4- yl]ethyl]pyrazol-4-yl]amino]-2- oxo-ethyl]-4-methyl-1,2,5- oxadiazole-3-carboxamide | 540.209 | 2.51 |
| 151 | 147 | 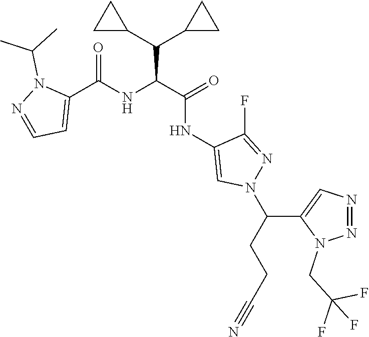 [SMILES: CC(C)n1nccc1C(=O)N[C@H](C(=O)Nc1cn(C(CCC#N)c2cnnn2CC(F)(F)F)nc1F)C(C1CC1)C1CC1] | N-[(1S)-1-[[1-[3-cyano-1-[3- (2,2,2-trifluoroethyl)triazol-4- yl]propyl]-3-fluoro-pyrazol-4- yl]carbamoyl]-2,2-dicyclo- propyl-ethyl]-2-isopropyl- pyrazole-3-carboxamide | 605.273 | 2.37 |
| 152 | 152 | 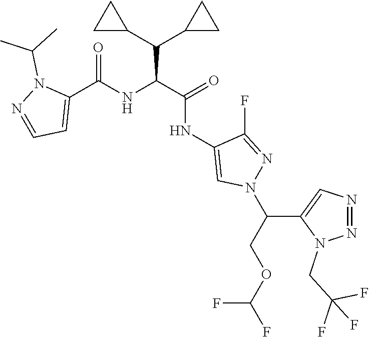 [SMILES: CC(C)n1nccc1C(=O)N[C@H](C(=O)Nc1cn(C(COC(F)F)c2cnnn2CC(F)(F)F)nc1F)C(C1CC1)C1CC1] | N-[(1S)-1-(dicyclopropyl- methyl)-2-[[1-[2-(difluoro- methoxy)-1-[3-(2,2,2- trifluoroethyl)triazol-4-yl]- ethyl]-3-fluoro-pyrazol-4- yl]amino]-2-oxo-ethyl]-2-iso- propyl-pyrazole-3-carboxamide | 632.253 | 2.46 |
| 153 | 152 | 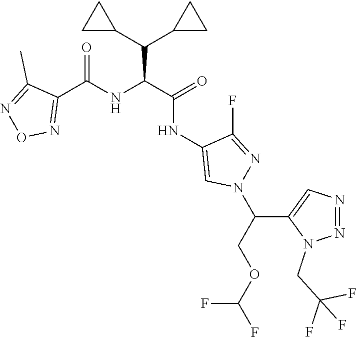 [SMILES: Cc1nonc1C(=O)N[C@H](C(=O)Nc1cn(C(COC(F)F)c2cnnn2CC(F)(F)F)nc1F)C(C1CC1)C1CC1] | N-[(1S)-1-(dicyclopropyl- methyl)-2-[[1-[2-(difluoro- methoxy)-1-[3-(2,2,2-trifluoro- ethyl)triazol-4-yl]ethyl]-3- fluoro-pyrazol-4-yl]amino]-2- oxo-ethyl]-4-methyl-1,2,5- oxadiazole-3-carboxamide | 606.202 | 2.51 |

### Example 154: N-[(15)-1-(dicyclopropylmethyl)-2-[[3-fluoro-1-[2-hydroxy-1-[3-(2,2,2-trifluoroethyl)triazol-4-yl]ethyl]pyrazol-4-yl]amino]-2-oxo-ethyl]-2-isopropyl-pyrazole-3-carboxamide

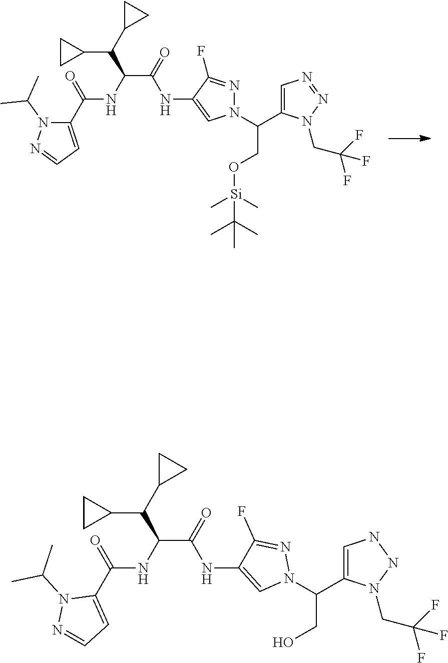 [SMILES: CC(C)n1nccc1C(=O)N[C@H](C(=O)Nc1cn(C(CO[Si](C)(C)C(C)(C)C)c2cnnn2CC(F)(F)F)nc1F)C(C1CC1)C1CC1 ; CC(C)n1nccc1C(=O)N[C@H](C(=O)Nc1cn(C(CO)C2=CNNN2CC(F)(F)F)nc1F)C(C1CC1)C1CC1]

To a solution of the compound of Preparation 150 (6.4 mg, 0.0092 mmol) in 2-methyl-THF (1 mL) was added 3M HCl in 2-Methyl-THF (1 mL, 3 mmol). The solution was stirred at room temperature for 2 hours then concentrated in vacuo. The residue was purified by acidic prep. HPLC to give the title compound (3 mg, 56% Yield) as an oil. LCMS (ES): m/z 582.257 [M+H]⁺, RT=2.34 min.

### Example 155: N-[(1S)-1-[[1-[2-cyano-1-[3-(2,2,2-trifluoroethyl)triazol-4-yl]ethyl]pyrazol-4-yl]carbamoyl]-2,2-dicyclopropyl-ethyl]-2-isopropyl-pyrazole-3-carboxamide

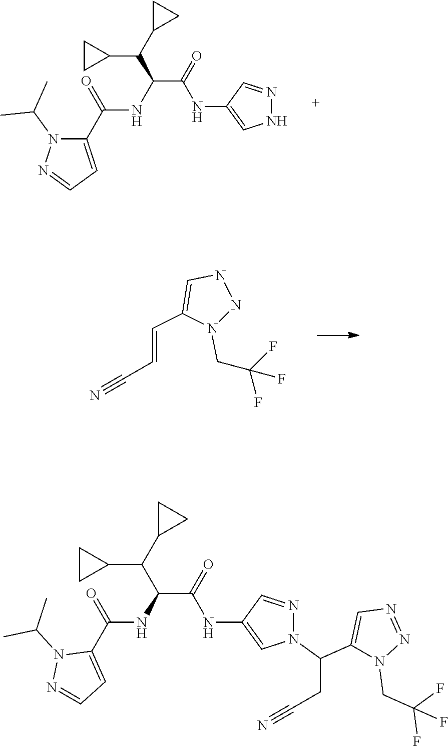 [SMILES: CC(C)n1nccc1C(=O)N[C@H](C(=O)Nc1cn[nH]c1)C(C1CC1)C1CC1 ; N#C/C=C/C1=CNNN1CC(F)(F)F ; CC(C)n1nccc1C(=O)N[C@H](C(=O)Nc1cnn(C(CC#N)c2cnnn2CC(F)(F)F)c1)C(C1CC1)C1CC1]

A mixture of the compound of Preparation 26 (33 mg, 0.089 mmol), (E)-3-[3-(2,2,2-trifluoroethyl)triazol-4-yl]prop-2-enenitrile (12 mg, 0.059 mmol), and DBU (13.5 mg, 0.089 mmol) in MeCN (2 mL) was stirred at room temperature overnight. The reaction mixture was purified directly by prep. acidic HPLC to give the title compound (4 mg, 12% Yield). LCMS (ES): m/z 573.267 [M+H]⁺, RT=2.32 min.

### Example 156: Inhibition of Human IL-17-Induced SEAP Reporter Gene Activity in HEK-Blue™ IL-17 Cells

50 nL test compounds in 100% DMSO were added into each well reserved for test compounds in a 384-well ViewPlates (Perkin Elmer), by the use of acoustic pipetting. The remaining wells received an equal volume of DMSO, as vehicle control, or VETRANAL® (Merck) in DMSO, as a positive control for cytotoxicity. Subsequently, 5 μl of an anti-IL-17A monoclonal antibody (final concentration 150 ng/ml) was added to the positive control wells. All wells containing test compounds and wells prepared to yield maximum stimulation received 5 μL of human TH-17 supernatant corresponding to 2 ng/mL IL-17A final concentration (measured by IL-17A AlphaLisa® SureFire®, Perkin Elmer). Finally, 45 μl HEK-Blue™ IL-17 cells (Invivogen) were added to all the wells resulting in a density of 12500 cells/well and incubated in a humid incubator at 37° C., 5% CO₂, overnight. The HEK-Blue™IL-17 cells, anti-IL-17A antibody and TH-17 supernatant were all diluted in DMEM with high glucose (Sigma) supplemented with 10% FBS, 1% P/S (Life technologies) and HEK-Blue™ selection (Invivogen).

After incubation, 5 μl of the supernatant was transferred from the cell plate to a new Viewplate and 45 μl Quanti-Blue™ solution, a SEAP detection reagent, was added and the Quanti-Blue™/cell supernatant was incubated at 37° C. The plate was inspected for colour development (5 to 60 minutes) and read using Envision, Perkin Elmer, plate reader (absorbance at 620 nm). The SEAP levels were calculated as percent of controls. Reduction of the amount of SEAP indicates decreased IL-17 signalling. Concentration response curves were fitted using a four-parameter logistic equation. Relative IC₅₀ and Emax were reported from curves showing acceptable fit (r2>0.9). Cytotoxicity was measured in the cell-containing Viewplate following addition of 7 μL PrestoBlue (Thermo Fisher) and incubation for 2.5-3 hours at room temperature, by measuring fluorescence at 615 nm (excitation at 535 nm). Fluorescence was directly proportional to the amount of metabolic activity. Reduction of fluorescence signal indicated cytotoxicity.

Compounds of the present invention were tested in the assay of Example 156. The results are summarized in Table 1.

TABLE 1

| | HEK Blue ™ assay |
|---|---|
| Example No. | EC₅₀ (nM) |
| 1 | 7000 |
| 2 | 170 |
| 3 | 2300 |
| 4 | 1100 |
| 5 | 4900 |
| 6 | 65 |
| 7 | 58 |
| 8 | 120 |
| 9 | 35 |
| 10 | 27 |
| 11 | 52 |
| 12 | 51 |
| 13 | 260 |
| 14 | 52 |
| 15 | 330 |
| 16 | 1,000 |
| 17 | 150 |
| 18 | 48 |
| 19 | 210 |
| 20 | 47 |
| 21 | 140 |
| 22 | 18 |
| 23 | 19 |
| 24 | 17 |
| 25 | 5.7 |
| 26 | 35 |
| 27 | 17 |
| 28 | 56 |
| 29 | 11 |
| 30 | 36 |
| 31 | 73 |
| 32 | 16 |
| 33 | 32 |
| 34 | 15 |
| 35 | 21 |
| 36 | 100 |
| 37 | 12 |
| 38 | 20 |
| 39 | 13 |
| 40 | 58 |
| 41 | 19 |
| 42 | 21 |
| 43 | 17 |
| 44 | 29 |
| 45 | 13 |
| 46 | 35 |
| 47 | 230 |
| 48 | Not tested |
| 49 | 26 |
| 50 | 76% at 8.4 μM |
| 51 | 41 |
| 52 | 160 |
| 53 | 130 |
| 54 | 61 |
| 55 | 39 |
| 56 | 64 |
| 57 | 67 |
| 58 | 43 |
| 59 | 44 |
| 60 | 89 |
| 61 | 230 |
| 62 | 110 |
| 63 | 220 |
| 64 | 52 |
| 65 | 86 |
| 66 | 81 |
| 67 | 65 |
| 68 | 37 |
| 69 | 54 |
| 70 | 32 |
| 71 | 35 |
| 72 | 45 |
| 73 | Not tested |
| 74 | Not tested |
| 75 | Not tested |
| 76 | Not tested |
| 77 | 2.6 |
| 78 | 3 |
| 79 | 19 |
| 80 | 18 |
| 81 | 59 |
| 82 | 26 |
| 83 | 40 |
| 84 | 18 |
| 85 | 56 |
| 86 | 95 |
| 87 | 110 |
| 88 | 58 |
| 89 | 37 |
| 90 | 46 |
| 91 | 47 |
| 92 | 55 |
| 93 | 210 |
| 94 | 130 |
| 95 | 160 |
| 96 | 5.2 |
| 97 | 47 |
| 98 | 8.7 |
| 99 | 7.6 |
| 100 | 48 |
| 101 | 25 |
| 102 | 46 |
| 103 | 160 |
| 104 | 400 |
| 105 | 21 |
| 106 | 43 |
| 107 | 7,200 |
| 108 | >10000 |
| 109 | 1,300 |
| 110 | 43% at 10 μM |
| 111 | 1,200 |
| 112 | 2,600 |
| 113 | >10000 |
| 114 | >10000 |
| 115 | 53% at 10 μM |
| 116 | 890 |
| 117 | 2,100 |
| 118 | 1,500 |
| 119 | 930 |
| 120 | 38 |
| 121 | 52 |
| 122 | 90 |
| 123 | 1,000 |
| 124 | 2,500 |
| 125 | 140 |
| 126 | 2,100 |
| 127 | 660 |
| 128 | 3,400 |
| 129 | 1,400 |
| 130 | 490 |
| 131 | 40 |
| 132 | 1,100 |
| 133 | 1,900 |
| 134 | 1,700 |
| 135 | 830 |
| 136 | 150 |
| 137 | 140 |
| 138 | 580 |
| 139 | 27 |
| 140 | 33 |
| 141 | 18 |
| 142 | 16 |
| 143 | 260 |
| 144 | 250 |
| 145 | 690 |
| 146 | 150 |
| 147 | 32 |
| 148 | 36 |
| 149 | 75 |
| 150 | 65 |
| 151 | 59 |
| 152 | Not tested |
| 153 | 18 |
| 154 | 84 |
| 155 | 71 |

### EMBODIMENTS

Embodiment 1. A compound having the formula (I)

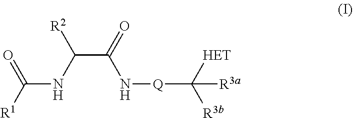

R¹ is selected from the group consisting of (C₁-C₆)alkyl, (C₃-C₇)cycloalkyl, (C₁-C₆)alkoxy, (C₃-C₇)cycloalkoxy, phenyl, phenyl-(C₁-C₄)alkyl, 5- or 6-membered heteroaryl, 9- or 10-membered bicyclic heteroaryl, 4-6-membered heterocycloalkyl and —NR^{c}R^{d}, wherein said (C₁-C₆)alkyl, (C₃-C₇)cycloalkyl, (C₁-C₆)alkoxy, (C₃-C₇)cycloalkoxy, phenyl, phenyl-(C₁-C₄)alkyl, 5- or 6-membered heteroaryl, 9- or 10-membered bicyclic heteroaryl, and 4-6-membered heterocycloalkyl is optionally substituted with one or more substituents independently selected from R^{a};
R^{a} is deuterium, halogen, hydroxy, —NR^{c}R^{d}, (C₁-C₆)alkyl, (C₁-C₆)alkylcarbonyl, (C₃-C₇)cycloalkyl, (C₃-C₇)cycloalkyl-(C₁-C₆)alkyl, phenyl, 5- or 6-membered heteroaryl, 4-6-membered heterocycloalkyl, or 4-6-membered heterocycloalkyl-(C₁-C₆)alkyl, wherein said (C₁-C₆)alkyl, (C₁-C₆)alkylcarbonyl, (C₃-C₇)cycloalkyl, (C₃-C₇)cycloalkyl-(C₁-C₆)alkyl, phenyl, 5- or 6-membered heteroaryl, 4-6-membered heterocycloalkyl or 4-6-membered heterocycloalkyl-(C₁-C₆)alkyl, is optionally substituted with one or more substituents independently selected from deuterium, halogen, hydroxy, cyano, (C₁-C₄)alkyl, (C₃-C₇)cycloalkyl, (C₁-C₄)alkoxy, (C₁-C₄)alkyl-S—, (C₁-C₄)alkyl-SO—, (C₁-C₄)alkyl-SO₂— and —NR^{c}R^{d};
R^{c} and R^{d} each independently are selected from the group consisting of hydrogen and (C₁-C₆)alkyl, or R^{c} and R^{d} together form azetidinyl, pyrrolidinyl or piperidinyl, wherein said (C₁-C₆)alkyl, azetidinyl, pyrrolidinyl or piperidinyl is optionally substituted with one or more substituents independently selected from halogen, cyano and hydroxy;
R² is selected from the group consisting of —CHR⁴R⁵, (C₃-C₁₀)cycloalkyl and G, wherein said (C₃-C₁₀)cycloalkyl and G are optionally substituted with one or more substituents independently selected from deuterium, halogen, cyano, (C₁-C₄)alkyl and halo(C₁-C₄)alkyl;
G is

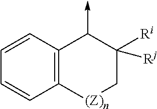

wherein Z is selected from CH, CH₂ and O; Rⁱ and R^{j} are hydrogen or Rⁱ and R^{j} together form a 3- or 4 membered carbocyclic ring; and n is 0 or 1;
R⁴ and R⁵ each independently represent hydrogen, phenyl, (C₁-C₆)alkyl, (C₃-C₇)cycloalkyl, and (C₃-C₇)cycloalkyl(C₁-C₆)alkyl wherein said phenyl, (C₁-C₆)alkyl, (C₃-C₇)cycloalkyl and (C₃-C₇)cycloalkyl(C₁-C₆)alkyl is optionally substituted with one or more substituents independently selected from halogen, cyano, and (C₁-C₄)alkyl; with the proviso that at least one of R⁴ and R⁵ is different from hydrogen; or one of R⁴ and R⁵ is (C₁-C₆)alkoxy, wherein said (C₁-C₆)alkoxy is optionally substituted with one or more fluorines;
R^{3a} is selected from hydrogen, (C₁-C₄)alkyl, (C₃-C₄)cycloalkyl and 4-6-membered heterocycloalkyl, wherein said (C₃-C₄)cycloalkyl may optionally be substituted with one or more substituents independently selected from deuterium, halogen, cyano and hydroxy, and wherein said (C₁-C₄)alkyl may optionally be substituted with one or more substituents independently selected from deuterium, halogen, cyano, hydroxy, (C₃-C₄)cycloalkyl, (C₁-C₃)alkoxy, (C₃-C₄)cycloalkoxy, fluoro(C₃-C₄)cycloalkyl, and fluoro(C₁-C₃)alkoxy and R^{3b} is hydrogen; or R^{3a} and R^{3b} together form a 3 membered carbocyclic ring;
Q is 5-membered heteroaryl, wherein said 5-membered heteroaryl is optionally substituted with one substituent independently selected from R^{e}; R^{e} is deuterium, halogen, (C₁-C₃)alkyl, wherein said (C₁-C₃)alkyl may optionally be substituted with one or more substituents independently selected from deuterium and halogen; and
HET is a 5- or 6-membered heteroaryl, wherein said 5- or 6-membered heteroaryl is substituted with one or more substituents selected from (C₁-C₄)alkyl, (C₁-C₃)alkoxy, (C₃-C₄)cycloalkoxy and (C₃-C₄)cycloalkyl, wherein said (C₁-C₃)alkoxy, (C₃-C₄)cycloalkoxy and (C₃-C₄)cycloalkyl may optionally be substituted with one or more substituents independently selected from deuterium, halogen, cyano and hydroxy, and wherein said (C₁-C₄)alkyl may optionally be substituted with one or more substituents independently selected from deuterium, halogen, cyano, hydroxy, (C₃-C₄)cycloalkyl, (C₁-C₃)alkoxy, (C₃-C₄)cycloalkoxy, fluoro(C₃-C₄)cycloalkyl, and fluoro(C₁-C₃)alkoxy;
or pharmaceutically acceptable salts thereof.

Embodiment 2. The compound according to embodiment 1 having the formula (II)

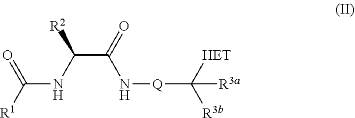

wherein R¹, R², R^{3a}, R^{3a}, Q and HET are as defined in claim 1; or pharmaceutically acceptable salts thereof.

Embodiment 3. A compound according to embodiment 1 having the formula (Ia)

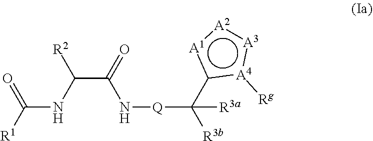

wherein R¹, R², R³, R⁹ and Q are as defined in embodiment 1 A¹, A², and A³ are selected from O, N and CH; A⁴ is selected from C and N provided that at least one of A¹, A², A³, A⁴ is N, and no more than one of A¹, A² and A³ is O;
R⁹ is selected from (C₁-C₄)alkyl, (C₃-C₄)cycloalkyl, wherein said (C₃-C₄)cycloalkyl may optionally be substituted with one or more substituents independently selected from deuterium, halogen, cyano and hydroxy, and wherein said (C₁-C₄)alkyl may optionally be substituted with one or more substituents independently selected from deuterium, halogen, cyano, hydroxy, (C₃-C₄)cycloalkyl, (C₁-C₃)alkoxy, (C₃-C₄)cycloalkoxy, fluoro(C₃-C₄)cycloalkyl, and fluoro(C₁-C₃)alkoxy.
or pharmaceutically acceptable salts thereof.

Embodiment 4. The compound according to embodiment 2 having the formula (IIa)

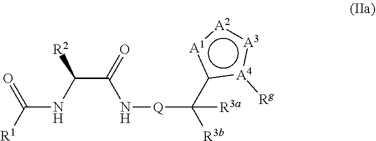

wherein R¹, R², R^{3a}, R^{3b}, R^{g} and Q are as defined in embodiment 1
A¹, A², and A³ are selected from O, N and CH; A⁴ is selected from C and N provided that at least one of A¹, A², A³, A⁴ is N, and no more than one of A¹, A² and A³ is O;
R⁹ is selected from (C₁-C₄)alkyl, (C₃-C₄)cycloalkyl, wherein said (C₃-C₄)cycloalkyl may optionally be substituted with one or more substituents independently selected from deuterium, halogen, cyano and hydroxy, and wherein said (C₁-C₄)alkyl may optionally be substituted with one or more substituents independently selected from deuterium, halogen, cyano, hydroxy, (C₃-C₄)cycloalkyl, (C₁-C₃)alkoxy, (C₃-C₄)cycloalkoxy, fluoro(C₃-C₄)cycloalkyl, and fluoro(C₁-C₃)alkoxy;
or pharmaceutically acceptable salts thereof.

Embodiment 5. The compound according to any one of embodiments 1-4, wherein R² is cyclohexyl, wherein said cyclohexyl is optionally substituted with one or more substituents independently selected from deuterium, halogen, cyano, (C₁-C₄)alkyl and halo(C₁-C₄)alkyl.

Embodiment 6. The compound according to embodiment 1-5, wherein R² is trans 4-methylcyclohexyl.

Embodiment 7. The compound according to embodiment 1-5, wherein R² is 4,4-difluoro-cyclohexyl.

Embodiment 8. The compound according to any one of embodiments 1-4 wherein R² is —CHR⁴R⁵, wherein R⁴ and R⁵ each independently represent hydrogen, phenyl, (C₁-C₆)alkyl, (C₃-C₇)cycloalkyl, and (C₃-C₇)cycloalkyl(C₁-C₆)alkyl wherein said phenyl, (C₁-C₆)alkyl, (C₃-C₇)cycloalkyl and (C₃-C₇)cycloalkyl(C₁-C₆)alkyl is optionally substituted with one or more substituents independently selected from halogen, cyano, and (C₁-C₄)alkyl; with the proviso that at least one of R⁴ and R⁵ is different from hydrogen.

Embodiment 9. The compound according to any one of embodiments 1-4 or 8 wherein R² is —CHR⁴R⁵ and wherein R⁴ and R⁵ are each independently cyclopropyl or cyclobutyl.

Embodiment 10. The compound according to any one of embodiments 1-4 or 9 wherein R² is —CHR⁴R⁵, wherein R⁴ and R⁵ are both cyclopropyl.

Embodiment 11. The compound according to any one of embodiments 1-10 wherein R¹ is selected from pyrazolyl, isoxazolyl and oxadiazolyl, wherein said pyrazolyl, isoxazolyl and oxadiazolyl is optionally substituted with one or more substituents independently selected from deuterium, halogen, (C₁-C₄)alkyl, (C₃-C₄)cycloalkyl, wherein said (C₁-C₃)alkyl or (C₃-C₄)cycloalkyl may optionally be substituted with one or more substituents independently selected from deuterium and halogen.

Embodiment 12. The compound according to embodiment 1-11 wherein R¹ is selected from pyrazol-3-yl and 1,2,5-oxadiazol-4-yl wherein said pyrazol-3-yl and 1,2,5-oxadiazol-4-yl is optionally substituted with one or more substituents independently selected from deuterium, halogen, (C₁-C₃)alkyl, wherein said (C₁-C₃)alkyl may optionally be substituted with one or more substituents independently selected from deuterium and halogen.

Embodiment 13. The compound according to any one of embodiments 1-12, wherein R³ is methyl, methoxymethyl or hydroxymethyl.

Embodiment 14. The compound according to embodiments 1-13, wherein R³ is methoxymethyl.

1. A compound having the formula (I) 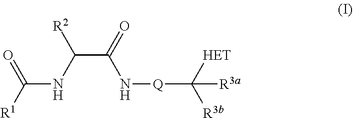
R¹ is selected from the group consisting of (C₁-C₆)alkyl, (C₃-C₇)cycloalkyl, (C₁-C₆)alkoxy, (C₃-C₇)cycloalkoxy, phenyl, phenyl-(C₁-C₄)alkyl, 5- or 6-membered heteroaryl, 9- or 10-membered bicyclic heteroaryl, 4-6-membered heterocycloalkyl and —NR^{c}R^{d}, wherein said (C₁-C₆)alkyl, (C₃-C₇)cycloalkyl, (C₁-C₆)alkoxy, (C₃-C₇)cycloalkoxy, phenyl, phenyl-(C₁-C₄)alkyl, 5- or 6-membered heteroaryl, 9- or 10-membered bicyclic heteroaryl, and 4-6-membered heterocycloalkyl is optionally substituted with one or more substituents independently selected from R^{a};
R^{a} is deuterium, halogen, hydroxy, —NR^{c}R^{d}, (C₁-C₆)alkyl, (C₁-C₆)alkylcarbonyl, (C₃-C₇)cycloalkyl, (C₃-C₇)cycloalkyl-(C₁-C₆)alkyl, phenyl, 5- or 6-membered heteroaryl, 4-6-membered heterocycloalkyl, or 4-6-membered heterocycloalkyl-(C₁-C₆)alkyl, wherein said (C₁-C₆)alkyl, (C₁-C₆)alkylcarbonyl, (C₃-C₇)cycloalkyl, (C₃-C₇)cycloalkyl-(C₁-C₆)alkyl, phenyl, 5- or 6-membered heteroaryl, 4-6-membered heterocycloalkyl or 4-6-membered heterocycloalkyl-(C₁-C₆)alkyl, is optionally substituted with one or more substituents independently selected from deuterium, halogen, hydroxy, cyano, (C₁-C₄)alkyl, (C₃-C₇)cycloalkyl, (C₁-C₄)alkoxy, (C₁-C₄)alkyl-S—, (C₁-C₄)alkyl-SO—, (C₁-C₄)alkyl-SO₂— and —NR^{c}R^{d};
R^{c} and R^{d} each independently are selected from the group consisting of hydrogen and (C₁-C₆)alkyl, or R^{c} and R^{d} together form azetidinyl, pyrrolidinyl or piperidinyl, wherein said (C₁-C₆)alkyl, azetidinyl, pyrrolidinyl or piperidinyl is optionally substituted with one or more substituents independently selected from halogen, cyano and hydroxy;
R² is selected from the group consisting of —CHR⁴R⁵, (C₃-C₁₀)cycloalkyl and G, wherein said (C₃-C₁₀)cycloalkyl and G are optionally substituted with one or more substituents independently selected from deuterium, halogen, cyano, (C₁-C₄)alkyl and halo(C₁-C₄)alkyl;
G is 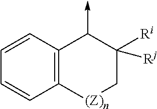
wherein Z is selected from CH, CH₂ and O; Rⁱ and R^{j} are hydrogen or Rⁱ and R^{j} together form a 3- or 4 membered carbocyclic ring; and n is 0 or 1;
R⁴ and R⁵ each independently represent hydrogen, phenyl, (C₁-C₆)alkyl, (C₃-C₇)cycloalkyl, and (C₃-C₇)cycloalkyl(C₁-C₆)alkyl wherein said phenyl, (C₁-C₆)alkyl, (C₃-C₇)cycloalkyl and (C₃-C₇)cycloalkyl(C₁-C₆)alkyl is optionally substituted with one or more substituents independently selected from halogen, cyano, and (C₁-C₄)alkyl; with the proviso that at least one of R⁴ and R⁵ is different from hydrogen; or one of R⁴ and R⁵ is (C₁-C₆)alkoxy, wherein said (C₁-C₆)alkoxy is optionally substituted with one or more fluorines;
R^{3a} is selected from hydrogen, (C₁-C₄)alkyl, (C₃-C₄)cycloalkyl and 4-6-membered heterocycloalkyl, wherein said (C₃-C₄)cycloalkyl may optionally be substituted with one or more substituents independently selected from deuterium, halogen, cyano and hydroxy, and wherein said (C₁-C₄)alkyl may optionally be substituted with one or more substituents independently selected from deuterium, halogen, cyano, hydroxy, (C₃-C₄)cycloalkyl, (C₁-C₃)alkoxy, (C₃-C₄)cycloalkoxy, fluoro(C₃-C₄)cycloalkyl, and fluoro(C₁-C₃)alkoxy and R^{3b} is hydrogen; or R^{3a} and R^{3b} together form a 3 membered carbocyclic ring;
Q is 5-membered heteroaryl, wherein said 5-membered heteroaryl is optionally substituted with one substituent independently selected from R^{e};
R^{e} is deuterium, halogen, (C₁-C₃)alkyl, wherein said (C₁-C₃)alkyl may optionally be substituted with one or more substituents independently selected from deuterium and halogen; and
HET is a 5- or 6-membered heteroaryl, wherein said 5- or 6-membered heteroaryl is substituted with one or more substituents selected from (C₁-C₄)alkyl, (C₁-C₃)alkoxy, (C₃-C₄)cycloalkoxy and (C₃-C₄)cycloalkyl, wherein said (C₁-C₃)alkoxy, (C₃-C₄)cycloalkoxy and (C₃-C₄)cycloalkyl may optionally be substituted with one or more substituents independently selected from deuterium, halogen, cyano and hydroxy, and wherein said (C₁-C₄)alkyl may optionally be substituted with one or more substituents independently selected from deuterium, halogen, cyano, hydroxy, (C₃-C₄)cycloalkyl, (C₁-C₃)alkoxy, (C₃-C₄)cycloalkoxy, fluoro(C₃-C₄)cycloalkyl, and fluoro(C₁-C₃)alkoxy;
or pharmaceutically acceptable salts thereof.

2. The compound according to claim 1 having the formula (II) 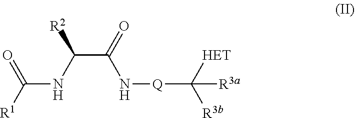
wherein R¹, R², R^{3a}, R^{3a}, Q and HET are as defined in claim 1 or pharmaceutically acceptable salts thereof.

3. The compound according to any one of claims 1-2, wherein HET is selected from triazole, pyrazole, isoxazole, imidazole, oxazole, pyridyl, pyrimidinyl, pyridazinyl and pyrazinyl and wherein said triazole, pyrazole, isoxazole, imidazole, oxazole, pyridyl, pyrimidinyl, pyridazinyl and pyrazinyl is substituted with one or more substituents selected from (C₁-C₄)alkyl, halo(C₁-C₄)alkyl, (C₁-C₃)alkoxy, (C₃-C₄)cycloalkoxy and (C₃-C₄)cycloalkyl, wherein said (C₁-C₃)alkoxy, (C₃-C₄)cycloalkoxy and (C₃-C₄)cycloalkyl may optionally be substituted with one or more substituents independently selected from deuterium, halogen, cyano and hydroxy, and wherein said (C₁-C₄)alkyl may optionally be substituted with one or more substituents independently selected from deuterium, halogen, cyano, hydroxy, (C₃-C₄)cycloalkyl, (C₁-C₃)alkoxy, (C₃-C₄)cycloalkoxy, fluoro(C₃-C₄)cycloalkyl, and fluoro(C₁-C₃)alkoxy.

4. The compound according to any one of claims 1-3, wherein HET is selected from 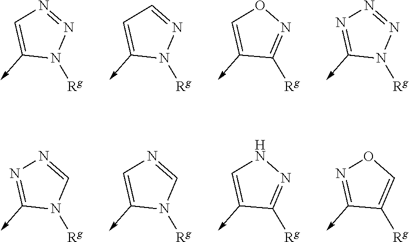
wherein R⁹ is selected from (C₁-C₄)alkyl, (C₃-C₄)cycloalkyl, wherein said (C₃-C₄)cycloalkyl may optionally be substituted with one or more substituents independently selected from deuterium, halogen, cyano and hydroxy, and wherein said (C₁-C₄)alkyl may optionally be substituted with one or more substituents independently selected from deuterium, halogen, cyano, hydroxy, (C₃-C₄)cycloalkyl, (C₁-C₃)alkoxy, (C₃-C₄)cycloalkoxy, fluoro(C₃-C₄)cycloalkyl, and fluoro(C₁-C₃)alkoxy.

5. The compound according to claim 4 wherein R⁹ is 2,2,2-trifluoroethyl.

6. The compound according to any one of claims 1-3, wherein HET is selected from pyridinyl, pyridazinyl and pyrimidyl wherein said pyridinyl, pyridazinyl and pyrimidyl is substituted by a substituent independently selected from hydrogen, (C₁-C₄)alkyl, (C₃-C₄)cycloalkyl, wherein said (C₃-C₄)cycloalkyl may optionally be substituted with one or more substituents independently selected from deuterium, halogen, cyano and hydroxy, and wherein said (C₁-C₄)alkyl may optionally be substituted with one or more substituents independently selected from deuterium, halogen, cyano, hydroxy, (C₃-C₄)cycloalkyl, (C₁-C₃)alkoxy, (C₃-C₄)cycloalkoxy, fluoro(C₃-C₄)cycloalkyl, and fluoro(C₁-C₃)alkoxy.

7. The compound according to any one of claims 1-6, wherein R² is cyclohexyl wherein said cyclohexyl is optionally substituted with one or more substituents independently selected from deuterium, halogen, cyano, (C₁-C₄)alkyl and halo(C₁-C₄)alkyl.

8. The compound according to any one of claims 1-7, wherein R² is trans 4-methylcyclohexyl.

9. The compound according to any one of claims 1-7, wherein R² is 4,4-difluoro-cyclohexyl.

10. The compound according to any one of claims 1-6, wherein R² is —CHR⁴R⁵, wherein R⁴ and R⁵ each independently represent hydrogen, phenyl, (C₁-C₆)alkyl, (C₃-C₇)cycloalkyl, and (C₃-C₇)cycloalkyl(C₁-C₆)alkyl wherein said phenyl, (C₁-C₆)alkyl, (C₃-C₇)cycloalkyl and (C₃-C₇)cycloalkyl(C₁-C₆)alkyl is optionally substituted with one or more substituents independently selected from halogen, cyano, and (C₁-C₄)alkyl; with the proviso that at least one of R⁴ and R⁵ is different from hydrogen.

11. The compound according to any one of claims 1-6 or 10, wherein R² is —CHR⁴R⁵ and wherein R⁴ and R⁵ are each independently cyclopropyl or cyclobutyl.

12. The compound according to any one of claims 1-6 or 10-11, wherein R² is —CHR⁴R⁵, wherein R⁴ and R⁵ are both cyclopropyl.

13. The compound according to any one of claims 1-12, wherein R¹ is selected from pyrazolyl, isoxazolyl and oxadiazolyl, wherein said pyrazolyl, isoxazolyl and oxadiazolyl is optionally substituted with one or more substituents independently selected from deuterium, halogen, (C₁-C₄)alkyl, (C₃-C₄)cycloalkyl, wherein said (C₁-C₄)alkyl or (C₃-C₄)cycloalkyl may optionally be substituted with one or more substituents independently selected from deuterium and halogen.

14. The compound according to any one of claims 1-13, wherein R¹ is selected from pyrazol-3-yl and 1,2,5-oxadiazol-4-yl wherein said pyrazol-3-yl and 1,2,5-oxadiazol-4-yl is optionally substituted with one or more substituents independently selected from deuterium, halogen, (C₁-C₃)alkyl, wherein said (C₁-C₃)alkyl may optionally be substituted with one or more substituents independently selected from deuterium and halogen.

15. The compound according to any one of claims 1-14, wherein R³ is methyl, methoxymethyl or hydroxymethyl.

16. The compound according to claims 1-15, wherein R³ is methoxymethyl.

17. The compound according to claim 1, wherein the compound is selected from:
N-[(1S)-1-(dicyclopropylmethyl)-2-oxo-2-[[1-[[1-(2,2,2-trifluoroethyl)triazol-4-yl]methyl]pyrazol-4-yl]amino]ethyl]-2-isopropyl-pyrazole-3-carboxamide,
N-[(1S)-1-(dicyclopropylmethyl)-2-oxo-2-[[1-[[3-(2,2,2-trifluoroethyl)triazol-4-yl]methyl]pyrazol-4-yl]amino]ethyl]-2-isopropyl-pyrazole-3-carboxamide,
N-[(1S)-1-(dicyclopropylmethyl)-2-oxo-2-[[1-[[3-(2,2,2-trifluoroethyl)triazol-4-yl]methyl]pyrazol-4-yl]amino]ethyl]-4-methyl-1,2,5-oxadiazole-3-carboxamide,
(3,3-difluorocyclobutyl)N-[(1S)-1-(dicyclopropylmethyl)-2-oxo-2-[[1-[[3-(2,2,2-trifluoroethyl)triazol-4-yl]methyl]pyrazol-4-yl]amino]ethyl]carbamate
N-[(1S)-1-(dicyclopropylmethyl)-2-[[1-[[3-(difluoromethyl)triazol-4-yl]methyl]pyrazol-4-yl]amino]-2-oxo-ethyl]-2-isopropyl-pyrazole-3-carboxamide,
N-[(1S)-1-(dicyclopropylmethyl)-2-[[3-fluoro-1-[2-methoxy-1-[3-(2,2,2-trifluoroethyl)triazol-4-yl]ethyl]pyrazol-4-yl]amino]-2-oxo-ethyl]-2-isopropyl-pyrazole-3-carboxamide,
N-[(1S)-1-(dicyclopropylmethyl)-2-[[3-fluoro-1-[2-methoxy-1-[3-(2,2,2-trifluoroethyl)triazol-4-yl]ethyl]pyrazol-4-yl]amino]-2-oxo-ethyl]-4-methyl-1,2,5-oxadiazole-3-carboxamide,
N-[(1S)-1-(4,4-difluorocyclohexyl)-2-[[3-fluoro-1-[2-methoxy-1-[3-(2,2,2-trifluoroethyl)triazol-4-yl]ethyl]pyrazol-4-yl]amino]-2-oxo-ethyl]-2-isopropyl-pyrazole-3-carboxamide,
N-[(1S)-1-(4,4-difluorocyclohexyl)-2-[[3-fluoro-1-[2-methoxy-1-[3-(2,2,2-trifluoroethyl)triazol-4-yl]ethyl]pyrazol-4-yl]amino]-2-oxo-ethyl]-4-methyl-1,2,5-oxadiazole-3-carboxamide,
N-[(1S)-1-(4,4-difluorocyclohexyl)-2-[[3-fluoro-1-[1-[1-(2,2,2-trifluoroethyl)tetrazol-5-yl]propyl]pyrazol-4-yl]amino]-2-oxo-ethyl]-2-isopropyl-pyrazole-3-carboxamide,
N-[(1S)-1-(4,4-difluorocyclo-hexyl)-2-[[3-fluoro-1-[1-[1-(2,2,2-trifluoroethyl)-tetrazol-5-yl]propyl]pyrazol-4-yl]-amino]-2-oxo-ethyl]-4-methyl-1,2,5-oxadiazole-3-carboxamide,
N-[(1S)-2-[[1-[1-(1-cyclo-butyltetrazol-5-yl)-3,3-di-fluoropropyl]pyrazol-4-yl]-amino]-1-(4,4-difluorocyclo-hexyl)-2-oxo-ethyl]-2-iso-propyl-pyrazole-3-carboxamide,
N-[(1S)-2-[[1-[1-(1-cyclo-butyltetrazol-5-yl)-3,3-di-fluoro-propyl]pyrazol-4-yl]-amino]-1-(4,4-difluoro-cyclohexyl)-2-oxo-ethyl]-4-methyl-1,2,5-oxadiazole-3-carboxamide,
N-[(1S)-2-[[1-[1-[1-(cyclopropylmethyl)tetrazol-5-yl]-3,3-difluoro-propyl]pyrazol-4-yl]amino]-1-(4,4-difluorocyclohexyl)-2-oxo-ethyl]-2-isopropyl-pyrazole-3-carboxamide,
N-[(1S)-2-[[1-[1-[1-(cyclopropylmethyl)tetrazol-5-yl]-3,3-difluoro-propyl]pyrazol-4-yl]amino]-1-(4,4-difluorocyclohexyl)-2-oxo-ethyl]-4-methyl-1,2,5-oxadiazole-3-carboxamide,
N-[(1S)-2-[[1-[1-(1-cyclopropyltetrazol-5-yl)-3,3-difluoro-propyl]pyrazol-4-yl]amino]-1-(4,4-difluorocyclohexyl)-2-oxo-ethyl]-4-methyl-1,2,5-oxadiazole-3-carboxamide,
N-[(1S)-2-[[1-[1-(1-cyclo-propyltetrazol-5-yl)-3,3-difluoro-propyl]pyrazol-4-yl]amino]-1-(4,4-difluorocyclo-hexyl)-2-oxo-ethyl]-2-iso-propyl-pyrazole-3-carboxamide,
N-[(1S)-2-[[1-[1-[1-(3,3-difluorocyclobutyl)tetrazol-5-yl]-3,3-difluoro-propyl]pyrazol-4-yl]amino]-1-(4,4-difluorocyclohexyl)-2-oxo-ethyl]-2-isopropyl-pyrazole-3-carboxamide,
N-[(1S)-2-[[1-[1-[1-(3,3-difluorocyclobutyl)tetrazol-5-yl]-3,3-difluoro-propyl]pyrazol-4-yl]amino]-1-(4,4-difluorocyclohexyl)-2-oxo-ethyl]-4-methyl-1,2,5-oxadiazole-3-carboxamide,
N-[(1S)-1-(4,4-difluorocyclo-hexyl)-2-[[1-[1-[1-(2,2-difluoro-ethyl)tetrazol-5-yl]-3,3-difluoro-propyl]pyrazol-4-yl]amino]-2-oxo-ethyl]-2-isopropyl-pyrazole-3-carboxamide,
N-[(1S)-1-(4,4-difluorocyclo-hexyl)-2-[[1-[1-[1-(2,2-difluoro-ethyl)tetrazol-5-yl]-3,3-difluoro-propyl]pyrazol-4-yl]amino]-2-oxo-ethyl]-4-methyl-1,2,5-oxadiazole-3-carboxamide,
N-[(1S)-1-(4,4-difluorocyclohexyl)-2-[[1-[3,3-difluoro-1-[1-(2,2,2-trifluoroethyl)tetrazol-5-yl]propyl]-3-fluoro-pyrazol-4-yl]amino]-2-oxo-ethyl]-2-isopropyl-pyrazole-3-carboxamide,
Diastereomer 1 of N-[(1S)-1-(4,4-difluorocyclohexyl)-2-[[1-[3,3-difluoro-1-[1-(2,2,2-trifluoroethyl)tetrazol-5-yl]propyl]-3-fluoro-pyrazol-4-yl]amino]-2-oxo-ethyl]-2-isopropyl-pyrazole-3-carboxamide,
Diastereomer 2 of N-[(1S)-1-(4,4-difluorocyclohexyl)-2-[[1-[3,3-difluoro-1-[1-(2,2,2-trifluoroethyl)tetrazol-5-yl]propyl]-3-fluoro-pyrazol-4-yl]amino]-2-oxo-ethyl]-2-isopropyl-pyrazole-3-carboxamide,
N-[(1S)-1-(4,4-difluorocyclohexyl)-2-[[1-[3,3-difluoro-1-[1-(2,2,2-trifluoroethyl)tetrazol-5-yl]propyl]-3-fluoro-pyrazol-4-yl]amino]-2-oxo-ethyl]-4-ethyl-1,2,5-oxadiazole-3-carboxamide,
N-[(1S)-1-(4,4-difluorocyclohexyl)-2-[[1-[3,3-difluoro-1-[1-(2,2,2-trifluoroethyl)tetrazol-5-yl]propyl]-3-fluoro-pyrazol-4-yl]amino]-2-oxo-ethyl]-4-methyl-1,2,5-oxadiazole-3-carboxamide,
Diastereomer 1 of N-[(1S)-2-[[1-[cyclopropyl-[1-(2,2,2-trifluoroethyl)tetrazol-5-yl]methyl]pyrazol-4-yl]amino]-1-(4,4-difluorocyclohexyl)-2-oxo-ethyl]-2-isopropyl-pyrazole-3-carboxamide,
Diastereomer 2 of N-[(1S)-2-[[1-[cyclopropyl-[1-(2,2,2-trifluoroethyl)tetrazol-5-yl]methyl]pyrazol-4-yl]amino]-1-(4,4-difluorocyclohexyl)-2-oxo-ethyl]-2-isopropyl-pyrazole-3-carboxamide,
Diastereomer 1 of N-[(1S)-2-[[1-[cyclobutyl-[1-(2,2,2-trifluoroethyl)tetrazol-5-yl]methyl]pyrazol-4-yl]amino]-1-(4,4-difluorocyclohexyl)-2-oxo-ethyl]-2-isopropyl-pyrazole-3-carboxamide,
Diastereomer 2 of N-[(1S)-2-[[1-[cyclobutyl-[1-(2,2,2-trifluoroethyl)tetrazol-5-yl]methyl]pyrazol-4-yl]amino]-1-(4,4-difluorocyclohexyl)-2-oxo-ethyl]-2-isopropyl-pyrazole-3-carboxamide,
Diastereomer 1 of N-[(1S)-2-[[1-[2-cyclopropyl-1-[1-(2,2,2-trifluoroethyl)tetrazol-5-yl]ethyl]pyrazol-4-yl]amino]-1-(4,4-difluorocyclohexyl)-2-oxo-ethyl]-2-isopropyl-pyrazole-3-carboxamide,
Diastereomer 2 of N-[(1S)-2-[[1-[2-cyclopropyl-1-[1-(2,2,2-trifluoroethyl)tetrazol-5-yl]ethyl]pyrazol-4-yl]amino]-1-(4,4-difluorocyclohexyl)-2-oxo-ethyl]-2-isopropyl-pyrazole-3-carboxamide,
Diastereomer 1 of N-[(1S)-1-(4,4-difluorocyclohexyl)-2-oxo-2-[[1-[3,3,3-trifluoro-1-[1-(2,2,2-trifluoroethyl)-tetrazol-5-yl]propyl]pyrazol-4-yl]amino]ethyl]-2-isopropyl-pyrazole-3-carboxamide,
Diastereomer 2 of N-[(1S)-1-(4,4-difluorocyclohexyl)-2-oxo-2-[[1-[3,3,3-trifluoro-1-[1-(2,2,2-trifluoroethyl)-tetrazol-5-yl]propyl]pyrazol-4-yl]amino]ethyl]-2-isopropyl-pyrazole-3-carboxamide,
Diastereomer 1 of N-[(1S)-1-(4,4-difluorocyclohexyl)-2-oxo-2-[[1-[tetrahydropyran-4-yl-[1-(2,2,2-trifluoroethyl)tetrazol-5-yl]methyl]pyrazol-4-yl]amino]ethyl]-2-isopropyl-pyrazole-3-carboxamide,
Diastereomer 2 of N-[(1S)-1-(4,4-difluorocyclohexyl)-2-oxo-2-[[1-[tetrahydropyran-4-yl-[1-(2,2,2-trifluoroethyl)tetrazol-5-yl]methyl]pyrazol-4-yl]amino]ethyl]-2-isopropyl-pyrazole-3-carboxamide,
Diastereomer 1 of N-[(1S)-2-[[1-[cyclopropyl-[1-(2,2,2-trifluoroethyl)tetrazol-5-yl]methyl]-3-fluoro-pyrazol-4-yl]amino]-1-(4,4-difluorocyclohexyl)-2-oxo-ethyl]-2-isopropyl-pyrazole-3-carboxamide,
Diastereomer 2 of N-[(1S)-2-[[1-[cyclopropyl-[1-(2,2,2-trifluoroethyl)tetrazol-5-yl]methyl]-3-fluoro-pyrazol-4-yl]amino]-1-(4,4-difluorocyclohexyl)-2-oxo-ethyl]-2-isopropyl-pyrazole-3-carboxamide,
Diastereomer 1 of N-[(1S)-2-[[1-[cyclopropyl-[1-(cyclopropylmethyl)tetrazol-5-yl]methyl]-3-fluoro-pyrazol-4-yl]amino]-1-(4,4-difluorocyclohexyl)-2-oxo-ethyl]-2-isopropyl-pyrazole-3-carboxamide,
Diastereomer 2 of N-[(1S)-2-[[1-[cyclopropyl-[1-(cyclopropylmethyl)tetrazol-5-yl]methyl]-3-fluoro-pyrazol-4-yl]amino]-1-(4,4-difluorocyclohexyl)-2-oxo-ethyl]-2-isopropyl-pyrazole-3-carboxamide,
Diastereomer 1 of N-[(1S)-2-[[1-[1-[1-(cyclopropyl-methyl)tetrazol-5-yl]-3,3-difluoro-propyl]-3-fluoro-pyrazol-4-yl]amino]-1-(4,4-difluorocyclohexyl)-2-oxo-ethyl]-2-isopropyl-pyrazole-3-carboxamide,
Diastereomer 2 of N-[(1S)-2-[[1-[1-[1-(cyclopropyl-methyl)tetrazol-5-yl]-3,3-difluoro-propyl]-3-fluoro-pyrazol-4-yl]amino]-1-(4,4-difluorocyclohexyl)-2-oxo-ethyl]-2-isopropyl-pyrazole-3-carboxamide,
Diastereomer 1 of N-[(1S)-2-[[1-[cyclopropyl-[1-(3,3-difluorocyclobutyl)tetrazol-5-yl]methyl]-3-fluoro-pyrazol-4-yl]amino]-1-(4,4-difluorocyclohexyl)-2-oxo-ethyl]-2-isopropyl-pyrazole-3-carboxamide,
Diastereomer 2 of N-[(1S)-2-[[1-[cyclopropyl-[1-(3,3-difluorocyclobutyl)tetrazol-5-yl]methyl]-3-fluoro-pyrazol-4-yl]amino]-1-(4,4-difluorocyclo-hexyl)-2-oxo-ethyl]-2-iso-propyl-pyrazole-3-carboxamide,
Diastereomer 1 of N-[(1S)-2-[[1-[cyclopropyl-[1-(2,2-difluoroethyl)tetrazol-5-yl]methyl]-3-fluoro-pyrazol-4-yl]amino]-1-(4,4-difluorocyclohexyl)-2-oxo-ethyl]-2-isopropyl-pyrazole-3-carboxamide,
Diastereomer 2 of N-[(1S)-2-[[1-[cyclopropyl-[1-(2,2-difluoroethyl)tetrazol-5-yl]methyl]-3-fluoro-pyrazol-4-yl]amino]-1-(4,4-difluorocyclohexyl)-2-oxo-ethyl]-2-isopropyl-pyrazole-3-carboxamide,
Diastereomer 1 of N-[(1S)-1-(4,4-difluorocyclohexyl)-2-[[1-[oxetan-3-yl-[1-(2,2,2-trifluoroethyl)tetrazol-5-yl]-methyl]pyrazol-4-yl]amino]-2-oxo-ethyl]-2-isopropyl-pyrazole-3-carboxamide,
Diastereomer 2 of N-[(1S)-1-(4,4-difluorocyclohexyl)-2-[[1-[oxetan-3-yl-[1-(2,2,2-trifluoroethyl)tetrazol-5-yl]-methyl]pyrazol-4-yl]amino]-2-oxo-ethyl]-2-isopropyl-pyrazole-3-carboxamide,
Diastereomer 1 of N-[(1S)-1-(4,4-difluorocyclohexyl)-2-[[1-[1-[1-(2,2-difluoroethyl)tetrazol-5-yl]-3,3-difluoro-propyl]-3-fluoro-pyrazol-4-yl]amino]-2-oxo-ethyl]-2-isopropyl-pyrazole-3-carboxamide,
Diastereomer 2 of N-[(1S)-1-(4,4-difluorocyclohexyl)-2-[[1-[1-[1-(2,2-difluoroethyl)tetrazol-5-yl]-3,3-difluoro-propyl]-3-fluoro-pyrazol-4-yl]amino]-2-oxo-ethyl]-2-isopropyl-pyrazole-3-carboxamide,
N-[(1S)-2,2-dicyclopropyl-1-[[1-[1-[1-(cyclopropylmethyl)tetrazol-5-yl]-3,3-difluoro-propyl]pyrazol-4-yl]carbamoyl]ethyl]-2-isopropyl-pyrazole-3-carboxamide,
N-[(1S)-1-(dicyclopropyl-methyl)-2-oxo-2-[[1-[1-[1-(2,2,2-trifluoroethyl)tetrazol-5-yl]ethyl]pyrazol-4-yl]amino]ethyl]-3-ethyl-isoxazole-4-carboxamide,
N-[(1S)-1-(dicyclopropyl-methyl)-2-oxo-2-[[1-[1-[1-(2,2,2-trifluoroethyl)tetrazol-5-yl]ethyl]pyrazol-4-yl]amino]ethyl]-2-isopropyl-pyrazole-3-carboxamide,
N-[(1S)-1-(dicyclopropyl-methyl)-2-[[1-[3,3-difluoro-1-[1-(2,2,2-trifluoroethyl)-tetrazol-5-yl]propyl]pyrazol-4-yl]amino]-2-oxo-ethyl]-2-iso-propyl-pyrazole-3-carboxamide,
N-[(1S)-1-(dicyclopropyl-methyl)-2-[[1-[3,3-difluoro-1-[1-(2,2,2-trifluoroethyl)-tetrazol-5-yl]propyl]pyrazol-4-yl]amino]-2-oxo-ethyl]-4-methyl-1,2,5-oxadiazole-3-carboxamide,
N-[(1S)-1-(dicyclopropyl-methyl)-2-oxo-2-[[1-[1-[1-(2,2,2-trifluoroethyl)tetrazol-5-yl]propyl]pyrazol-4-yl]amino]ethyl]-2-isopropyl-pyrazole-3-carboxamide,
N-[(1S)-1-(dicyclopropyl-methyl)-2-oxo-2-[[1-[1-[1-(2,2,2-trifluoroethyl)tetrazol-5-yl]propyl]pyrazol-4-yl]amino]-ethyl]-4-methyl-1,2,5-oxadiazole-3-carboxamide,
N-[(1S)-1-(dicyclopropyl-methyl)-2-[[3-fluoro-1-[1-[1-(2,2,2-trifluoroethyl)tetrazol-5-yl]propyl]pyrazol-4-yl]amino]-2-oxo-ethyl]-2-isopropyl-pyrazole-3-carboxamide,
N-[(1S)-1-[[1-[1-(1-cyclobutyltetrazol-5-yl)-3,3-difluoro-propyl]pyrazol-4-yl]carbamoyl]-2,2-dicyclo-propyl-ethyl]-2-isopropyl-pyrazole-3-carboxamide,
N-[(1S)-1-[[1-[1-(1-cyclo-butyltetrazol-5-yl)-3,3-difluoro-propyl]pyrazol-4-yl]carbamoyl]-2,2-dicyclo-propyl-ethyl]-4-methyl-1,2,5-oxadiazole-3-carboxamide,
N-[(1S)-2,2-dicyclopropyl-1-[[1-[1-[1-(cyclopropylmethyl)-tetrazol-5-yl]-3,3-difluoropropyl]pyrazol-4-yl]carbamoyl]ethyl]-4-methyl-1,2,5-oxadiazole-3-carboxamide,
N-[(1S)-2,2-dicyclopropyl-1-[[1-[1-(1-cyclopropyltetrazol-5-yl)-3,3-difluoropropyl]-pyrazol-4-yl]carbamoyl]ethyl]-2-isopropyl-pyrazole-3-carboxamide,
N-[(1S)-2,2-dicyclopropyl-1-[[1-[1-(1-cyclopropyltetrazol-5-yl)-3,3-difluoropropyl]-pyrazol-4-yl]carbamoyl]ethyl]-4-methyl-1,2,5-oxadiazole-3-carboxamide,
N-[(1S)-1-(dicyclopropyl-methyl)-2-[[1-[1-[1-(3,3-difluorocyclobutyl)tetrazol-5-yl]-3,3-difluoro-propyl]pyrazol-4-yl]amino]-2-oxo-ethyl]-2-isopropyl-pyrazole-3-carboxamide,
N-[(1S)-1-(dicyclopropyl-methyl)-2-[[1-[1-[1-(3,3-difluorocyclobutyl)tetrazol-5-yl]-3,3-difluoro-propyl]pyrazol-4-yl]amino]-2-oxo-ethyl]-4-methyl-1,2,5-oxadiazole-3-carboxamide,
N-[(1S)-1-(dicyclopropyl-methyl)-2-[[1-[1-[1-(2,2-difluoroethyl)tetrazol-5-yl]-3,3-difluoro-propyl]pyrazol-4-yl]amino]-2-oxo-ethyl]-2-isopropyl-pyrazole-3-carboxamide,
N-[(1S)-1-(dicyclopropyl-methyl)-2-[[1-[1-[1-(2,2-difluoroethyl)tetrazol-5-yl]-3,3-difluoro-propyl]pyrazol-4-yl]amino]-2-oxo-ethyl]-4-methyl-1,2,5-oxadiazole-3-carboxamide,
Diastereomer 1 of N-[(1S)-2,2-dicyclopropyl-1-[[1-[cyclo-propyl-[1-(2,2,2-trifluoro-ethyl)tetrazol-5-yl]methyl]-3-fluoro-pyrazol-4-yl]-carbamoyl]ethyl]-2-isopropyl-pyrazole-3-carboxamide,
Diastereomer 2 of N-[(1S)-2,2-dicyclopropyl-1-[[1-[cyclo-propyl-[1-(2,2,2-trifluoro-ethyl)tetrazol-5-yl]methyl]-3-fluoro-pyrazol-4-yl]-carbamoyl]ethyl]-2-isopropyl-pyrazole-3-carboxamide,
Diastereomer 1 of N-[(1S)-2,2-dicyclopropyl-1-[[1-[cyclo-propyl-[1-(cyclopropylmethyl)-tetrazol-5-yl]methyl]-3-fluoro-pyrazol-4-yl]carbamoyl]ethyl]-2-isopropyl-pyrazole-3-carboxamide,
Diastereomer 2 of N-[(1S)-2,2-dicyclopropyl-1-[[1-[cyclo-propyl-[1-(cyclopropylmethyl)-tetrazol-5-yl]methyl]-3-fluoro-pyrazol-4-yl]carbamoyl]ethyl]-2-isopropyl-pyrazole-3-carboxamide,
N-[(1S)-2,2-dicyclopropyl-1-[[1-[cyclopropyl-[1-(2,2,2-trifluoroethyl)tetrazol-5-yl]methyl]pyrazol-4-yl]carbamoyl]ethyl]-2-isopropyl-pyrazole-3-carboxamide,
Diastereomer 1 of N-[(1S)-2,2-dicyclopropyl-1-[[1-[2-cyclo-propyl-1-[1-(2,2,2-trifluoroethyl)tetrazol-5-yl]ethyl]-3-fluoro-pyrazol-4-yl]carbamoyl]ethyl]-2-isopropyl-pyrazole-3-carboxamide,
Diastereomer 2 of N-[(1S)-2,2-dicyclopropyl-1-[[1-[2-cyclo-propyl-1-[1-(2,2,2-trifluoroethyl)tetrazol-5-yl]ethyl]-3-fluoro-pyrazol-4-yl]carbamoyl]ethyl]-2-isopropyl-pyrazole-3-carboxamide,
Diastereomer 1 of N-[(1S)-2,2-dicyclopropyl-1-[[1-[2-cyclopropyl-1-[1-(cyclopropylmethyl)tetrazol-5-yl]ethyl]-3-fluoro-pyrazol-4-yl]carbamoyl]ethyl]-2-isopropyl-pyrazole-3-carboxamide,
Diastereomer 2 of N-[(1S)-2,2-dicyclopropyl-1-[[1-[2-cyclopropyl-1-[1-(cyclopropylmethyl)tetrazol-5-yl]ethyl]-3-fluoro-pyrazol-4-yl]carbamoyl]ethyl]-2-isopropyl-pyrazole-3-carboxamide,
N-[(1S)-2-[[1-[3,3-difluoro-1-[1-(2,2,2-trifluoroethyl)tetrazol-5-yl]propyl]-3-fluoro-pyrazol-4-yl]amino]-1-(trans-4-methylcyclohexyl)-2-oxo-ethyl]-2-isopropyl-pyrazole-3-carboxamide,
N-[(1S)-2-[[1-[3,3-difluoro-1-[1-(2,2,2-trifluoroethyl)tetrazol-5-yl]propyl]-3-fluoro-pyrazol-4-yl]amino]-1-(trans-4-methylcyclo-hexyl)-2-oxo-ethyl]-4-methyl-1,2,5-oxadiazole-3-carboxamide,
Diastereomer 1 of N-[(1S)-2-[[1-[3,3-difluoro-1-[1-(2,2,2-trifluoroethyl)tetrazol-5-yl]propyl]-3-fluoro-pyrazol-4-yl]amino]-1-(trans-4-methylcyclohexyl)-2-oxo-ethyl]-2-[(1S)-2-hydroxy-1-methyl-ethyl]pyrazole-3-carboxamide,
Diastereomer 2 of N-[(1S)-2-[[1-[3,3-difluoro-1-[1-(2,2,2-trifluoroethyl)tetrazol-5-yl]propyl]-3-fluoro-pyrazol-4-yl]amino]-1-(trans-4-methylcyclohexyl)-2-oxo-ethyl]-2-[(1S)-2-hydroxy-1-methyl-ethyl]pyrazole-3-carboxamide,
N-[(1S)-1-(tert-butoxymethyl)-2-[[1-[3,3-difluoro-1-[1-(2,2,2-trifluoroethyl)tetrazol-5-yl]propyl]-3-fluoro-pyrazol-4-yl]amino]-2-oxo-ethyl]-2-isopropyl-pyrazole-3-carboxamide
N-[(1S)-1-(tert-butoxymethyl)-2-[[1-[3,3-difluoro-1-[1-(2,2,2-trifluoroethyl)tetrazol-5-yl]propyl]-3-fluoro-pyrazol-4-yl]amino]-2-oxo-ethyl]-4-ethyl-1,2,5-oxadiazole-3-carboxamide,
Diastereomer 1 of N-[(1S)-2-[[1-[3,3-difluoro-1-[1-(2,2,2-trifluoroethyl)tetrazol-5-yl]propyl]-3-fluoro-pyrazol-4-yl]amino]-2-oxo-1-[(2,2,2-trifluoro-1,1-dimethyl-ethoxy)methyl]ethyl]-2-isopropyl-pyrazole-3-carboxamide,
Diastereomer 2 of N-[(1S)-2-[[1-[3,3-difluoro-1-[1-(2,2,2-trifluoroethyl)tetrazol-5-yl]propyl]-3-fluoro-pyrazol-4-yl]amino]-2-oxo-1-[(2,2,2-trifluoro-1,1-dimethyl-ethoxy)methyl]ethyl]-2-isopropyl-pyrazole-3-carboxamide,
N-[(1S)-1-(dicyclopropylmethyl)-2-[[3-[3,3-difluoro-1-[1-(2,2,2-trifluoroethyl)tetrazol-5-yl]propyl]isoxazol-5-yl]amino]-2-oxo-ethyl]-2-isopropyl-pyrazole-3-carboxamide,
N-[(1S)-1-(dicyclopropylmethyl)-2-oxo-2-[[3-[1-[1-(2,2,2-trifluoroethyl)tetrazol-5-yl]propyl]isoxazol-5-yl]amino]ethyl]-2-isopropyl-pyrazole-3-carboxamide,
N-[(1S)-1-(dicyclopropylmethyl)-2-[[3-[3,3-difluoro-1-[1-(2,2,2-trifluoro-ethyl)tetrazol-5-yl]propyl]isoxazol-5-yl]amino]-2-oxo-ethyl]-4-methyl-1,2,5-oxadiazole-3-carboxamide,
N-[(1S)-1-(dicyclopropylmethyl)-2-[[1-[(1S)-3,3-difluoro-1-[4-(2,2,2-trifluoroethyl)-1,2,4-triazol-3-yl]propyl]-3-fluoro-pyrazol-4-yl]amino]-2-oxo-ethyl]-2-isopropyl-pyrazole-3-carboxamide,
N-[(1S)-1-(dicyclopropyl-methyl)-2-[[3-fluoro-1-[(1R)-1-[4-(2,2,2-trifluoroethyl)-1,2,4-triazol-3-yl]propyl]-pyrazol-4-yl]amino]-2-oxo-ethyl]-2-isopropyl-pyrazole-3-carboxamide
N-[(1S)-1-(dicyclopropyl-methyl)-2-[[3-fluoro-1-[(1S)-1-[4-(2,2,2-trifluoroethyl)-1,2,4-triazol-3-yl]propyl]-pyrazol-4-yl]amino]-2-oxo-ethyl]-2-isopropyl-pyrazole-3-carboxamide,
N-[(1S)-1-(dicyclopropyl-methyl)-2-[[1-[(1S)-3,3-difluoro-1-[4-(2,2,2-trifluoroethyl)-1,2,4-triazol-3-yl]propyl]-3-fluoro-pyrazol-4-yl]amino]-2-oxo-ethyl]-4-methyl-1,2,5-oxadiazole-3-carboxamide,
N-[(1S)-2-[[1-[(1S)-3,3-difluoro-1-[4-(2,2,2-trifluoro-ethyl)-1,2,4-triazol-3-yl]-propyl]-3-fluoro-pyrazol-4-yl]amino]-2-oxo-1-[(2,2,2-trifluoro-1,1-dimethyl-ethoxy)-methyl]ethyl]-2-isopropyl-pyrazole-3-carboxamide,
N-[(1S)-2-[[1-[(1S)-1-[4-(cyclopropylmethyl)-1,2,4-triazol-3-yl]-3,3-difluoro-propyl]-3-fluoro-pyrazol-4-yl]amino]-2-oxo-1-[(2,2,2-trifluoro-1,1-dimethyl-ethoxy)-methyl]ethyl]-2-isopropyl-pyrazole-3-carboxamide,
N-[(1S)-1-(dicyclopropyl-methyl)-2-oxo-2-[[1-[(1R)-1-[4-(2,2,2-trifluoroethyl)-1,2,4-triazol-3-yl]ethyl]pyrazol-4-yl]amino]ethyl]-2-isopropyl-pyrazole-3-carboxamide,
N-[(1S)-1-(dicyclopropyl-methyl)-2-oxo-2-[[1-[(1S)-1-[4-(2,2,2-trifluoroethyl)-1,2,4-triazol-3-yl]ethyl]pyrazol-4-yl]amino]ethyl]-2-isopropyl-pyrazole-3-carboxamide,
N-[(1S)-2-[[1-[(1S)-3,3-difluoro-1-[4-(2,2,2-trifluoroethyl)-1,2,4-triazol-3-yl]propyl]-3-fluoro-pyrazol-4-yl]amino]-1-(trans-4-methylcyclohexyl)-2-oxo-ethyl]-2-isopropyl-pyrazole-3-carboxamide,
N-[(1S)-2-[[1-[(1S)-3,3-difluoro-1-[4-(2,2,2-trifluoro-ethyl)-1,2,4-triazol-3-yl]propyl]-3-fluoro-pyrazol-4-yl]amino]-1-(trans-4-methylcyclohexyl)-2-oxo-ethyl]-2-[(1S)-2-hydroxy-1-methyl-ethyl]-pyrazole-3-carboxamide,
N-[(1S)-2-[[1-[(1S)-3,3-difluoro-1-[4-(2,2,2-trifluoroethyl)-1,2,4-triazol-3-yl]propyl]-3-fluoro-pyrazol-4-yl]amino]-1-(trans-4-methylcyclohexyl)-2-oxo-ethyl]-4-methyl-1,2,5-oxadiazole-3-carboxamide,
N-[(1S)-2-[[1-[(1S)-1-[4-(cyclopropylmethyl)-1,2,4-triazol-3-yl]-3,3-difluoro-propyl]-3-fluoro-pyrazol-4-yl]amino]-1-(trans-4-methylcyclohexyl)-2-oxo-ethyl]-2-isopropyl-pyrazole-3-carboxamide,
N-[(1S)-2-[[1-[(1S)-1-[4-(cyclopropylmethyl)-1,2,4-triazol-3-yl]-3,3-difluoro-propyl]-3-fluoro-pyrazol-4-yl]amino]-1-(trans-4-methylcyclo-hexyl)-2-oxo-ethyl]-2-[(1S)-2-hydroxy-1-methyl-ethyl]pyrazole-3-carboxamide,
N-[(1S)-2-[[1-[(1S)-1-[4-(cyclopropylmethyl)-1,2,4-triazol-3-yl]-3,3-difluoro-propyl]-3-fluoro-pyrazol-4-yl]amino]-1-(trans-4-methylcyclohexyl)-2-oxo-ethyl]-4-methyl-1,2,5-oxadiazole-3-carboxamide,
N-[(1S)-1-(dicyclopropylmethyl)-2-[[3-fluoro-1-[1-[4-(2,2,2-trifluoroethyl)-1,2,4-triazol-3-yl]cyclopropyl]pyrazol-4-yl]amino]-2-oxo-ethyl]-2-isopropyl-pyrazole-3-carboxamide,
N-[(1S)-1-(dicyclopropylmethyl)-2-oxo-2-[[1-[1-[4-(2,2,2-trifluoroethyl)-1,2,4-triazol-3-yl]cyclopropyl]pyrazol-4-yl]amino]ethyl]-2-isopropyl-pyrazole-3-carboxamide,
N-[(1S)-1-(4,4-difluorocyclohexyl)-2-oxo-2-[[1-[1-[1-(2,2,2-trifluoroethyl)-tetrazol-5-yl]cyclopropyl]pyrazol-4-yl]amino]ethyl]-2-isopropyl-pyrazole-3-carboxamide,
N-[(1S)-2-[[3-fluoro-1-[1-[5-(2,2,2-trifluoroethyl)-1H-pyrazol-4-yl]propyl]pyrazol-4-yl]amino]-1-(4-methylcyclohexyl)-2-oxo-ethyl]-2-isopropyl-pyrazole-3-carboxamide,
N-[(1S)-2-[[3-fluoro-1-[1-[5-(2,2,2-trifluoroethyl)-1H-pyrazol-4-yl]propyl]pyrazol-4-yl]amino]-1-(4-methylcyclohexyl)-2-oxo-ethyl]-4-methyl-1,2,5-oxadiazole-3-carboxamide,
N-[(1S)-1-(dicyclopropylmethyl)-2-oxo-2-[[1-[[1-(2,2,2-trifluoroethyl)triazol-4-yl]methyl]pyrazol-4-yl]amino]ethyl]-4-methyl-1,2,5-oxadiazole-3-carboxamide,
(3,3-difluorocyclobutyl)N-[(1S)-1-(dicyclopropylmethyl)-2-oxo-2-[[1-[[1-(2,2,2-trifluoroethyl)triazol-4-yl]methyl]pyrazol-4-yl]amino]ethyl]carbamate,
N-[(1S)-1-(dicyclopropyl-methyl)-2-[[1-[[2-(2,2-difluoroethyl)pyrazol-3-yl]methyl]pyrazol-4-yl]amino]-2-oxo-ethyl]-2-isopropyl-pyrazole-3-carboxamide,
N-[(1S)-1-(dicyclopropyl-methyl)-2-[[1-[[2-(2,2-difluoroethyl)pyrazol-3-yl]methyl]pyrazol-4-yl]amino]-2-oxo-ethyl]-4-methyl-1,2,5-oxadiazole-3-carboxamide,
N-[(1S)-1-(dicyclopropyl-methyl)-2-oxo-2-[[1-[[2-(2,2,2-trifluoroethyl)pyrazol-3-yl]methyl]pyrazol-4-yl]amino]ethyl]-2-isopropyl-pyrazole-3-carboxamide,
N-[(1S)-1-(dicyclopropyl-methyl)-2-oxo-2-[[1-[[2-(2,2,2-trifluoroethyl)pyrazol-3-yl]methyl]pyrazol-4-yl]amino]ethyl]-4-methyl-1,2,5-oxadiazole-3-carboxamide,
cyclopropyl N-[(1S)-1-(dicyclo-propylmethyl)-2-[[1-[[2-(2,2-difluoroethyl)pyrazol-3-yl]methyl]pyrazol-4-yl]amino]-2-oxo-ethyl]carbamate,
cyclopropyl N-[(1S)-1-(dicyclopropylmethyl)-2-oxo-2-[[1-[[2-(2,2,2-trifluoroethyl)-pyrazol-3-yl]methyl]pyrazol-4-yl]amino]ethyl]carbamate,
N-[(1S)-1-(dicyclopropyl-methyl)-2-[[1-[(2-methylpyrazol-3-yl)methyl]-pyrazol-4-yl]amino]-2-oxo-ethyl]-2-isopropyl-pyrazole-3-carboxamide,
N-[(1S)-1-(dicyclopropyl-methyl)-2-[[1-[(4-methyl-1,2,4-triazol-3-yl)methyl]-pyrazol-4-yl]amino]-2-oxo-ethyl]-2-isopropyl-pyrazole-3-carboxamide,
N-[(1S)-1-(dicyclopropylmethyl)-2-[[1-[(1-methylpyrazol-4-yl)methyl]pyrazol-4-yl]amino]-2-oxo-ethyl]-2-isopropyl-pyrazole-3-carboxamide,
N-[(1S)-1-(dicyclopropylmethyl)-2-[[1-[(3-methyl-1H-pyrazol-5-yl)methyl]pyrazol-4-yl]amino]-2-oxo-ethyl]-2-isopropyl-pyrazole-3-carboxamide,
N-[(1S)-1-(dicyclopropylmethyl)-2-[[1-[(3,5-dimethyl-1H-pyrazol-4-yl)methyl]pyrazol-4-yl]amino]-2-oxo-ethyl]-2-isopropyl-pyrazole-3-carboxamide,
N-[(1S)-1-(4,4-difluorocyclohexyl)-2-[[3-fluoro-1-[1-[4-(2,2,2-trifluoro-ethyl)isoxazol-3-yl]propyl]pyrazol-4-yl]amino]-2-oxo-ethyl]-2-isopropyl-pyrazole-3-carboxamide,
N-[(1S)-1-(4,4-difluorocyclohexyl)-2-[[3-fluoro-1-[1-[4-(2,2,2-trifluoro-ethyl)isoxazol-3-yl]propyl]pyrazol-4-yl]amino]-2-oxo-ethyl]-4-ethyl-1,2,5-oxadiazole-3-carboxamide,
N-[(1S)-1-(4,4-difluorocyclohexyl)-2-[[3-fluoro-1-[1-[4-(2,2,2-trifluoro-ethyl)isoxazol-3-yl]propyl]pyrazol-4-yl]amino]-2-oxo-ethyl]-4-methyl-1,2,5-oxadiazole-3-carboxamide,
N-[(1S)-1-(dicyclopropyl-methyl)-2-oxo-2-[[1-[1-[2-(2,2,2-trifluoroethyl)pyrazol-3-yl]ethyl]pyrazol-4-yl]amino]ethyl]-2-isopropyl-pyrazole-3-carboxamide,
N-[(1S)-1-(dicyclopropyl-methyl)-2-[[1-[1-[2-(2,2-difluoroethyl)pyrazol-3-yl]ethyl]pyrazol-4-yl]amino]-2-oxo-ethyl]-2-isopropyl-pyrazole-3-carboxamide,
N-[(1S)-1-(dicyclopropyl-methyl)-2-[[3-fluoro-1-[1-[3-(2,2,2-trifluoroethyl)isoxazol-4-yl]ethyl]pyrazol-4-yl]amino]-2-oxo-ethyl]-2-isopropyl-pyrazole-3-carboxamide,
N-[(1S)-1-(dicyclopropyl-methyl)-2-oxo-2-[[1-[1-[3-(2,2,2-trifluoroethyl)isoxazol-4-yl]ethyl]pyrazol-4-yl]amino]ethyl]-2-isopropyl-pyrazole-3-carboxamide,
N-[(1S)-1-(dicyclopropylmethyl)-2-[[1-[(3-ethyltriazol-4-yl)methyl]pyrazol-4-yl]amino]-2-oxo-ethyl]-2-isopropyl-pyrazole-3-carboxamide,
N-[(1S)-1-(dicyclopropyl-methyl)-2-[[1-[[3-(2-methoxyethyl)triazol-4-yl]methyl]pyrazol-4-yl]amino]-2-oxo-ethyl]-2-isopropyl-pyrazole-3-carboxamide,
N-[(1S)-1-(dicyclopropyl-methyl)-2-[[1-[(3-isopropyl-triazol-4-yl)methyl]pyrazol-4-yl]amino]-2-oxo-ethyl]-2-iso-propyl-pyrazole-3-carboxamide,
N-[(1S)-2,2-dicyclopropyl-1-[[1-[[3-(cyclopropylmethyl)-triazol-4-yl]methyl]pyrazol-4-yl]carbamoyl]ethyl]-2-iso-propyl-pyrazole-3-carboxamide,
N-[(1S)-1-[[1-[(3-cyclobutyl-triazol-4-yl)methyl]pyrazol-4-yl]carbamoyl]-2,2-dicyclo-propyl-ethyl]-2-isopropyl-pyrazole-3-carboxamide,
N-[(1S)-1-(dicyclopropyl-methyl)-2-[[1-[1-[3-(2-methoxyethyl)triazol-4-yl]ethyl]pyrazol-4-yl]amino]-2-oxo-ethyl]-2-isopropyl-pyrazole-3-carboxamide,
Diastereomer 1 of N-[(1S)-1-(dicyclopropyl-methyl)-2-[[1-[1-[3-(2-methoxyethyl)triazol-4-yl]ethyl]pyrazol-4-yl]amino]-2-oxo-ethyl]-2-isopropyl-pyrazole-3-carboxamide,
Diastereomer 2 of N-[(1S)-1-(dicyclopropyl-methyl)-2-[[1-[1-[3-(2-methoxyethyl)triazol-4-yl]ethyl]pyrazol-4-yl]amino]-2-oxo-ethyl]-2-isopropyl-pyrazole-3-carboxamide,
N-[(1S)-1-(dicyclopropyl-methyl)-2-[[1-[1-(3-isopropyl-triazol-4-yl)ethyl]pyrazol-4-yl]-amino]-2-oxo-ethyl]-2-iso-propyl-pyrazole-3-carboxamide,
N-[(1S)-2,2-dicyclopropyl-1-[[1-[1-[3-(cyclopropylmethyl)-triazol-4-yl]ethyl]-pyrazol-4-yl]carbamoyl]ethyl]-2-iso-propyl-pyrazole-3-carboxamide,
N-[(1S)-1-[[1-[1-(3-cyclo-butyltriazol-4-yl)ethyl]pyrazol-4-yl]carbamoyl]-2,2-dicyclo-propyl-ethyl]-2-isopropyl-pyrazole-3-carboxamide,
N-[(1S)-1-(dicyclopropyl-methyl)-2-oxo-2-[[1-[[3-(2,2,2-trifluoro-1-methyl-ethyl)triazol-4-yl]methyl]-pyrazol-4-yl]amino]ethyl]-2-isopropyl-pyrazole-3-carboxamide,
N-[(1S)-2-[[1-[2-cyclopropyl-1-[3-(2,2,2-trifluoroethyl)-triazol-4-yl]ethyl]-3-fluoro-pyrazol-4-yl]amino]-1-(4,4-difluorocyclohexyl)-2-oxo-ethyl]-2-isopropyl-pyrazole-3-carboxamide,
N-[(1S)-2,2-dicyclopropyl-1-[[1-[2-cyclopropyl-1-[3-(2,2,2-trifluoroethyl)triazol-4-yl]ethyl]-3-fluoro-pyrazol-4-yl]carbamoyl]ethyl]-2-iso-propyl-pyrazole-3-carboxamide,
N-[(1S)-2,2-dicyclopropyl-1-[[1-[2-cyclopropyl-1-[3-(2,2,2-trifluoroethyl)triazol-4-yl]-ethyl]-3-fluoro-pyrazol-4-yl]-carbamoyl]ethyl]-4-methyl-1,2,5-oxadiazole-3-carboxamide,
N-[(1S)-2-[[1-[2-cyclopropyl-1-[3-(2,2,2-trifluoroethyl)-triazol-4-yl]ethyl]-3-fluoro-pyrazol-4-yl]amino]-1-(4,4-difluorocyclohexyl)-2-oxo-ethyl]-4-methyl-1,2,5-oxadiazole-3-carboxamide,
N-[(1S)-1-(dicyclopropyl-methyl)-2-[[1-[[5-methyl-3-(2,2,2-trifluoroethyl)triazol-4-yl]methyl]pyrazol-4-yl]amino]-2-oxo-ethyl]-2-isopropyl-pyrazole-3-carboxamide,
N-[(1S)-1-(dicyclopropyl-methyl)-2-[[1-[1-[3-(2,2-difluoroethyl)triazol-4-yl]ethyl]pyrazol-4-yl]amino]-2-oxo-ethyl]-2-isopropyl-pyrazole-3-carboxamide,
N-[(1S)-1-(dicyclopropyl-methyl)-2-[[1-[1-[3-(2,2-difluoroethyl)triazol-4-yl]ethyl]pyrazol-4-yl]amino]-2-oxo-ethyl]-4-methyl-1,2,5-oxadiazole-3-carboxamide,
N-[(1S)-1-(dicyclopropyl-methyl)-2-oxo-2-[[1-[[3-(2,2,2-trifluoroethyl)imidazol-4-yl]methyl]pyrazol-4-yl]amino]ethyl]-2-isopropyl-pyrazole-3-carboxamide,
N-[(1S)-1-(dicyclopropyl-methyl)-2-[[1-[3,3-difluoro-1-[3-(2,2,2-trifluoroethyl)triazol-4-yl]propyl]-3-fluoro-pyrazol-4-yl]amino]-2-oxo-ethyl]-2-isopropyl-pyrazole-3-carboxamide,
N-[(1S)-1-(dicyclopropyl-methyl)-2-[[1-[3,3-difluoro-1-[3-(2,2,2-trifluoroethyl)triazol-4-yl]propyl]-3-fluoro-pyrazol-4-yl]amino]-2-oxo-ethyl]-4-methyl-1,2,5-oxadiazole-3-carboxamide,
N-[(1S)-1-(dicyclopropyl-methyl)-2-[[3-fluoro-1-[1-[3-(2,2,2-trifluoroethyl)triazol-4-yl]ethyl]pyrazol-4-yl]amino]-2-oxo-ethyl]-2-isopropyl-pyrazole-3-carboxamide,
N-[(1S)-1-(dicyclopropyl-methyl)-2-[[3-fluoro-1-[1-[3-(2,2,2-trifluoroethyl)triazol-4-yl]ethyl]pyrazol-4-yl]amino]-2-oxo-ethyl]-4-methyl-1,2,5-oxadiazole-3-carboxamide,
N-[(1S)-1-[[1-[3-cyano-1-[3-(2,2,2-trifluoroethyl)triazol-4-yl]propyl]-3-fluoro-pyrazol-4-yl]carbamoyl]-2,2-dicyclo-propyl-ethyl]-2-isopropyl-pyrazole-3-carboxamide,
N-[(1S)-1-(dicyclopropyl-methyl)-2-[[1-[2-(difluoro-methoxy)-1-[3-(2,2,2-trifluoroethyl)triazol-4-yl]-ethyl]-3-fluoro-pyrazol-4-yl]amino]-2-oxo-ethyl]-2-iso-propyl-pyrazole-3-carboxamide,
N-[(1S)-1-(dicyclopropyl-methyl)-2-[[1-[2-(difluoro-methoxy)-1-[3-(2,2,2-trifluoro-ethyl)triazol-4-yl]ethyl]-3-fluoro-pyrazol-4-yl]amino]-2-oxo-ethyl]-4-methyl-1,2,5-oxadiazole-3-carboxamide,
N-[(1S)-1-(dicyclopropylmethyl)-2-[[3-fluoro-1-[2-hydroxy-1-[3-(2,2,2-trifluoroethyl)triazol-4-yl]ethyl]pyrazol-4-yl]amino]-2-oxo-ethyl]-2-isopropyl-pyrazole-3-carboxamide,
N-[(1S)-1-[[1-[2-cyano-1-[3-(2,2,2-trifluoroethyl)triazol-4-yl]ethyl]pyrazol-4-yl]carbamoyl]-2,2-dicyclopropyl-ethyl]-2-isopropyl-pyrazole-3-carboxamide,
or a pharmaceutically acceptable salt thereof.

18. A compound according to any one of claims 1-17 for use in therapy.

19. A compound according to claim 18 for use in the treatment of a disease, disorder or condition, which disease, disorder or condition is responsive of modulation of IL-17.

20. A compound according to claim 18 for use in the treatment of autoimmune diseases.

21. A compound according to claim 18 for use in the treatment of psoriasis, ankylosing spondylitis, spondyloarthritis or psoriatic arthritis.

22. A pharmaceutical composition comprising a compound according to any one of claims 1-17 together with a pharmaceutically acceptable vehicle or excipient or pharmaceutically acceptable carrier(s).

23. The pharmaceutical composition according to claim 22 together with one or more other therapeutically active compound(s).
